SEQUENCE LISTING (226 sequences)

SEQ ID NO: 1 - protein, 142 aa, Homo sapiens
  source 1..142: note = TIR signaling domain of TLR1
LQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAI

SEQ ID NO: 2 - protein, 144 aa, Homo sapiens
  source 1..144: note = TIR signaling domain of TLR2
ICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAI

SEQ ID NO: 3 - protein, 144 aa, Homo sapiens
  source 1..144: note = TIR signaling domain of TLR3
FEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVAL

SEQ ID NO: 4 - protein, 144 aa, Homo sapiens
  source 1..144: note = TIR signaling domain of TLR4
NIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 5 - protein, 146 aa, Homo sapiens
  source 1..146: note = TIR signaling domain of TLR5
YKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDLQDVGWFLHKLSQQI

SEQ ID NO: 6 - protein, 142 aa, Homo sapiens
  source 1..142: note = TIR signaling domain of TLR6
LQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAF

SEQ ID NO: 7 - protein, 145 aa, Homo sapiens
  source 1..145: note = TIR signaling domain of TLR7
CCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNAL

SEQ ID NO: 8 - protein, 145 aa, Homo sapiens
  source 1..145: note = TIR signaling domain of TLR8
TFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVV

SEQ ID NO: 9 - protein, 146 aa, Homo sapiens
  source 1..146: note = TIR signaling domain of TLR9
LPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMAL

SEQ ID NO: 10 - protein, 144 aa, Homo sapiens
  source 1..144: note = TIR signaling domain of TLR10
VRFHAFISYSEHDSLWVKNELIPNLEKEDGSILICLYESYFDPGKSISENIVSFIEKSYKSIFVLSPNFVQNEWCHYEFYFAHHNLFHENSDHIILILLEPIPFYCIPTRYHKLKALLEKKAYLEWPKDRRKCGLFWANLRAAI

SEQ ID NO: 11 - DNA, 432 bp, Homo sapiens
  source 1..432: mol_type = other DNA; note = TLR1 signaling domain
tttgaatatgcagcatatataattcatgcctataaagataaggattgggtctgggaacatttctcttcaatggaaaaggaagaccaatctctcaaattttgtctggaagaaagggactttgaggcgggtgtttttgaactagaagcaattgttaacagcatcaaaagaagcagaaaaattatttttgttataacacaccatctattaaaagacccattatgcaaaagattcaaggtacatcatgcagttcaacaagctattgaacaaaatctggattccattatattggttttccttgaggagattccagattataaactgaaccatgcactctgtttgcgaagaggaatgtttaaatctcactgcatcttgaactggccagttcagaaagaacggataggtgcctttcgtcataaattgcaagtagcactt

SEQ ID NO: 12 - DNA, 435 bp, Homo sapiens
  source 1..435: mol_type = other DNA; note = TLR2 signaling domain
tgttgctatgatgcttttattgtgtatgacactaaagacccagctgtgaccgagtgggttttggctgagctggtggccaaactggaagacccaagagagaaacattttaatttatgtctcgaggaaagggactggttaccagggcagccagttctggaaaacctttcccagagcatacagcttagcaaaaagacagtgtttgtgatgacagacaagtatgcaaagactgaaaattttaagatagcattttacttgtcccatcagaggctcatggatgaaaaagttgatgtgattatcttgatatttcttgagaagccctttcagaagtccaagttcctccagctccggaaaaggctctgtgggagttctgtccttgagtggccaacaaacccgcaagctcacccatacttctggcagtgtctaaagaacgccctg

SEQ ID NO: 13 - DNA, 435 bp, Homo sapiens
  source 1..435: mol_type = other DNA; note = TLR3 signaling domain
actttctatgatgcttacatttcttatgacaccaaagatgcctctgttactgactgggtgataaatgagctgcgctaccaccttgaagagagccgagacaaaaacgttctcctttgtctagaggagagggattgggacccgggattggccatcatcgacaacctcatgcagagcatcaaccaaagcaagaaaacagtatttgttttaaccaaaaaatatgcaaaaagctggaactttaaaacagctttttacttggctttgcagaggctaatggatgagaacatggatgtgattatatttatcctgctggagccagtgttacagcattctcagtatttgaggctacggcagcggatctgtaagagctccatcctccagtggcctgacaacccgaaggcagaaggcttgttttggcaaactctgagaaatgtggtc

SEQ ID NO: 14 - DNA, 438 bp, Homo sapiens
  source 1..438: mol_type = other DNA; note = TLR4 signaling domain
ctgccctacgatgccttcgtggtcttcgacaaaacgcagagcgcagtggcagactgggtgtacaacgagcttcgggggcagctggaggagtgccgtgggcgctgggcactccgcctgtgcctggaggaacgcgactggctgcctggcaaaaccctctttgagaacctgtgggcctcggtctatggcagccgcaagacgctgtttgtgctggcccacacggaccgggtcagtggtctcttgcgcgccagcttcctgctggcccagcagcgcctgctggaggaccgcaaggacgtcgtggtgctggtgatcctgagccctgacggccgccgctcccgctacgtgcggctgcgccagcgcctctgccgccagagtgtcctcctctggccccaccagcccagtggtcagcgcagcttctgggcccagctgggcatggccctg

SEQ ID NO: 15 - DNA, 432 bp, Homo sapiens
  source 1..432: mol_type = other DNA; note = TLR5 signaling domain
atctgctatgatgcatttgtttcttacagtgagcgggatgcctactgggtggagaaccttatggtccaggagctggagaacttcaatccccccttcaagttgtgtcttcataagcgggacttcattcctggcaagtggatcattgacaatatcattgactccattgaaaagagccacaaaactgtctttgtgctttctgaaaactttgtgaagagtgagtggtgcaagtatgaactggacttctcccatttccgtctttttgatgagaacaatgatgctgccattctcattcttctggagcccattgagaaaaaagccattccccagcgcttctgcaagctgcggaagataatgaacaccaagacctacctggagtggcctatggacgaggctcagcgggaaggattttgggtaaatctgagagctgcgata

SEQ ID NO: 16 - DNA, 432 bp, Homo sapiens
  source 1..432: mol_type = other DNA; note = TLR6 signaling domain
aacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcagcaggtggagctgtaccgccttctcagcaggaacacttacctggagtgggaggacagtgtcctggggcggcacatcttctggagacgactcagaaaagccctg

SEQ ID NO: 17 - DNA, 432 bp, Homo sapiens
  source 1..432: mol_type = other DNA; note = TLR7 signaling domain
gtccgattccacgcatttatttcatacagtgaacatgattctctgtgggtgaagaatgaattgatccccaatctagagaaggaagatggttctatcttgatttgcctttatgaaagctactttgaccctggcaaaagcattagtgaaaatattgtaagcttcattgagaaaagctataagtccatctttgttttgtctcccaactttgtccagaatgagtggtgccattatgaattctactttgcccaccacaatctcttccatgaaaattctgatcatataattcttatcttactggaacccattccattctattgcattcccaccaggtatcataaactgaaagctctcctggaaaaaaaagcatacttggaatggcccaaggataggcgtaaatgtgggcttttctgggcaaaccttcgagctgctatt

SEQ ID NO: 18 - DNA, 426 bp, Homo sapiens
  source 1..426: mol_type = other DNA; note = TLR8 signaling domain
ctccagtttcatgcatttatttcatatagtgggcacgattctttctgggtgaagaatgaattattgccaaacctagagaaagaaggtatgcagatttgccttcatgagagaaactttgttcctggcaagagcattgtggaaaatatcatcacctgcattgagaagagttacaagtccatctttgttttgtctcccaactttgtccagagtgaatggtgccattatgaactctactttgcccatcacaatctctttcatgaaggatctaatagcttaatcctgatcttgctggaacccattccgcagtactccattcctagcagttatcacaagctcaaaagtctcatggccaggaggacttatttggaatggcccaaggaaaagagcaaacgtggccttttttgggctaacttaagggcagccatt

SEQ ID NO: 19 - DNA, 426 bp, Homo sapiens
  source 1..426: mol_type = other DNA; note = TLR9 signaling domain
ctccagtttcatgcttttatttcatatagtgaacatgattctgcctgggtgaaaagtgaattggtaccttacctagaaaaagaagatatacagatttgtcttcatgagagaaactttgtccctggcaagagcattgtggaaaatatcatcaactgcattgagaagagttacaagtccatctttgttttgtctcccaactttgtccagagtgagtggtgccattacgaactctattttgcccatcacaatctctttcatgaaggatctaataacttaatcctcatcttactggaacccattccacagaacagcattcccaacaagtaccacaagctgaaggctctcatgacgcagcggacttatttgcagtggcccaaggagaaaagcaaacgtgggctcttttgggctaacattagagccgctttt

SEQ ID NO: 20 - DNA, 438 bp, Homo sapiens
  source 1..438: mol_type = other DNA; note = TLR10 signaling domain
tacaaatatgatgcctatttgtgcttcagcagcaaagacttcacatgggtgcagaatgctttgctcaaacacctggacactcaatacagtgaccaaaacagattcaacctgtgctttgaagaaagagactttgtcccaggagaaaaccgcattgccaatatccaggatgccatctggaacagtagaaagatcgtttgtcttgtgagcagacacttccttagagatggctggtgccttgaagccttcagttatgcccagggcaggtgcttatctgaccttaacagtgctctcatcatggtggtggttgggtccttgtcccagtaccagttgatgaaacatcaatccatcagaggctttgtacagaaacagcagtatttgaggtggcctgaggatctccaggatgttggctggtttcttcataaactctctcaacagata

SEQ ID NO: 21 - protein, 156 aa, Homo sapiens
  source 1..156: note = IL-1R signaling domain
KTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHM

SEQ ID NO: 22 - protein, 148 aa, Homo sapiens
  source 1..148: note = IL-18R signaling domain
KTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLM

SEQ ID NO: 23 - skipped

SEQ ID NO: 24 - DNA, 444 bp, Homo sapiens
  source 1..444: mol_type = other DNA; note = IL-18R signaling domain
aaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatg

SEQ ID NO: 25 - protein, 786 aa, Homo sapiens
  source 1..786: note = human TLR1
MTSIFHFAIIFMLILQIRIQLSEESEFLVDRSKNGLIHVPKDLSQKTTILNISQNYISELWTSDILSLSKLRILIISHNRIQYLDISVFKFNQELEYLDLSHNKLVKISCHPTVNLKHLDLSFNAFDALPICKEFGNMSQLKFLGLSTTHLEKSSVLPIAHLNISKVLLVLGETYGEKEDPEGLQDFNTESLHIVFPTNKEFHFILDVSVKTVANLELSNIKCVLEDNKCSYFLSILAKLQTNPKLSNLTLNNIETTWNSFIRILQLVWHTTVWYFSISNVKLQGQLDFRDFDYSGTSLKALSIHQVVSDVFGFPQSYIYEIFSNMNIKNFTVSGTRMVHMLCPSKISPFLHLDFSNNLLTDTVFENCGHLTELETLILQMNQLKELSKIAEMTTQMKSLQQLDISQNSVSYDEKKGDCSWTKSLLSLNMSSNILTDTIFRCLPPRIKVLDLHSNKIKSIPKQVVKLEALQELNVAFNSLTDLPGCGSFSSLSVLIIDHNSVSHPSADFFQSCQKMRSIKAGDNPFQCTCELGEFVKNIDQVSSEVLEGWPDSYKCDYPESYRGTLLKDFHMSELSCNITLLIVTIVATMLVLAVTVTSLCSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 26 - protein, 784 aa, Homo sapiens
  source 1..784: note = human TLR2
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 27 - protein, 904 aa, Homo sapiens
  source 1..904: note = human TLR3
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 28 - protein, 839 aa, Homo sapiens
  source 1..839: note = human TLR4
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 29 - protein, 858 aa, Homo sapiens
  source 1..858: note = human TLR5
MGDHLDLLLGVVLMAGPVFGIPSCSFDGRIAFYRFCNLTQVPQVLNTTERLLLSFNYIRTVTASSFPFLEQLQLLELGSQYTPLTIDKEAFRNLPNLRILDLGSSKIYFLHPDAFQGLFHLFELRLYFCGLSDAVLKDGYFRNLKALTRLDLSKNQIRSLYLHPSFGKLNSLKSIDFSSNQIFLVCEHELEPLQGKTLSFFSLAANSLYSRVSVDWGKCMNPFRNMVLEILDVSGNGWTVDITGNFSNAISKSQAFSLILAHHIMGAGFGFHNIKDPDQNTFAGLARSSVRHLDLSHGFVFSLNSRVFETLKDLKVLNLAYNKINKIADEAFYGLDNLQVLNLSYNLLGELYSSNFYGLPKVAYIDLQKNHIAIIQDQTFKFLEKLQTLDLRPRTLTTIHFIPSIPDIFLSGNKLVTLPKINLTANLIHLSENRLENLDILYFLLRVPHLQILILNQNRFSSCSGDQTPSENPSLEQLFLGENMLQLAWETELCWDVFEGLSHLQVLYLNHNYLNSLPPGVFSHLTALRGLSLNSNRLTVLSHNDLPANLEILDISRNQLLAPNPDVFVSLSVLDITHNKFICECELSTFINWLNHTNVTIAGPPADIYCVYPDSFSGVSLFSLSTEGCDEEEVLKSLKFSLFIVCTVTLTLFLMTILTVTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDFQDVGWFLHKLSQQILKKEKEKKKDNNIPLQTVATIS

SEQ ID NO: 30 - protein, 796 aa, Homo sapiens
  source 1..796: note = human TLR6
MTKDKEPIVKSFHFVCLMIIIVGTRIQFSDGNEFAVDKSKRGLIHVPKDLPLKTKVLDMSQNYIAELQVSDMSFLSELTVLRLSHNRIQLLDLSVFKFNQDLEYLDLSHNQLQKISCHPIVSFRHLDLSFNDFKALPICKEFGNLSQLNFLGLSAMKLQKLDLLPIAHLHLSYILLDLRNYYIKENETESLQILNAKTLHLVFHPTSLFAIQVNISVNTLGCLQLTNIKLNDDNCQVFIKFLSELTRGSTLLNFTLNHIETTWKCLVRVFQFLWPKPVEYLNIYNLTIIESIREEDFTYSKTTLKALTIEHITNQVFLFSQTALYTVFSEMNIMMLTISDTPFIHMLCPHAPSTFKFLNFTQNVFTDSIFEKCSTLVKLETLILQKNGLKDLFKVGLMTKDMPSLEILDVSWNSLESGRHKENCTWVESIVVLNLSSNMLTDSVFRCLPPRIKVLDLHSNKIKSVPKQVVKLEALQELNVAFNSLTDLPGCGSFSSLSVLIIDHNSVSHPSADFFQSCQKMRSIKAGDNPFQCTCELREFVKNIDQVSSEVLEGWPDSYKCDYPESYRGSPLKDFHMSELSCNITLLIVTIGATMLVLAVTVTSLCIYLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 31 - protein, 1049 aa, Homo sapiens
  source 1..1049: note = human TLR7
MVFPMWTLKRQILILFNIILISKLLGARWFPKTLPCDVTLDVPKNHVIVDCTDKHLTEIPGGIPTNTTNLTLTINHIPDISPASFHRLDHLVEIDFRCNCVPIPLGSKNNMCIKRLQIKPRSFSGLTYLKSLYLDGNQLLEIPQGLPPSLQLLSLEANNIFSIRKENLTELANIEILYLGQNCYYRNPCYVSYSIEKDAFLNLTKLKVLSLKDNNVTAVPTVLPSTLTELYLYNNMIAKIQEDDFNNLNQLQILDLSGNCPRCYNAPFPCAPCKNNSPLQIPVNAFDALTELKVLRLHSNSLQHVPPRWFKNINKLQELDLSQNFLAKEIGDAKFLHFLPSLIQLDLSFNFELQVYRASMNLSQAFSSLKSLKILRIRGYVFKELKSFNLSPLHNLQNLEVLDLGTNFIKIANLSMFKQFKRLKVIDLSVNKISPSGDSSEVGFCSNARTSVESYEPQVLEQLHYFRYDKYARSCRFKNKEASFMSVNESCYKYGQTLDLSKNSIFFVKSSDFQHLSFLKCLNLSGNLISQTLNGSEFQPLAELRYLDFSNNRLDLLHSTAFEELHKLEVLDISSNSHYFQSEGITHMLNFTKNLKVLQKLMMNDNDISSSTSRTMESESLRTLEFRGNHLDVLWREGDNRYLQLFKNLLKLEELDISKNSLSFLPSGVFDGMPPNLKNLSLAKNGLKSFSWKKLQCLKNLETLDLSHNQLTTVPERLSNCSRSLKNLILKNNQIRSLTKYFLQDAFQLRYLDLSSNKIQMIQKTSFPENVLNNLKMLLLHHNRFLCTCDAVWFVWWVNHTEVTIPYLATDVTCVGPGAHKGQSVISLDLYTCELDLTNLILFSLSISVSLFLMVMMTASHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 32 - protein, 1041 aa, Homo sapiens
  source 1..1041: note = human TLR8
MENMFLQSSMLTCIFLLISGSCELCAEENFSRSYPCDEKKQNDSVIAECSNRRLQEVPQTVGKYVTELDLSDNFITHITNESFQGLQNLTKINLNHNPNVQHQNGNPGIQSNGLNITDGAFLNLKNLRELLLEDNQLPQIPSGLPESLTELSLIQNNIYNITKEGISRLINLKNLYLAWNCYFNKVCEKTNIEDGVFETLTNLELLSLSFNSLSHVPPKLPSSLRKLFLSNTQIKYISEEDFKGLINLTLLDLSGNCPRCFNAPFPCVPCDGGASINIDRFAFQNLTQLRYLNLSSTSLRKINAAWFKNMPHLKVLDLEFNYLVGEIASGAFLTMLPRLEILDLSFNYIKGSYPQHINISRNFSKLLSLRALHLRGYVFQELREDDFQPLMQLPNLSTINLGINFIKQIDFKLFQNFSNLEIIYLSENRISPLVKDTRQSYANSSSFQRHIRKRRSTDFEFDPHSNFYHFTRPLIKPQCAAYGKALDLSLNSIFFIGPNQFENLPDIACLNLSANSNAQVLSGTEFSAIPHVKYLDLTNNRLDFPRTSALTELSDLEVLDLSYNSHYFRIAGVTHHLEFIQNFTNLKVLNLSHNNIYTLTDKYNLESKSLVELVFSGNRLDILWNDDDNRYISIFKGLKNLTRLDLSLNRLKHIPNEAFLNLPASLTELHINDNMLKFFNWTLLQQFPRLELLDLRGNKLLFLTDSLSDFTSSLRTLLLSHNRISHLPSGFLSEVSSLKHLDLSSNLLKTINKSALETKTTTKLSMLELHGNPFECTCDIGDFRRWMDEHLNVKIPRLVDVICASPGDQRGKSIVSLELTTCVSDVTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 33 - protein, 1032 aa, Homo sapiens
  source 1..1032: note = human TLR9
MGFCRSALHPLSLLVQAIMLAMTLALGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHAPNPCMECPRHFPQLHPDTFSHLSRLEGLVLKDSSLSWLNASWFRGLGNLRVLDLSENFLYKCITKTKAFQGLTQLRKLNLSFNYQKRVSFAHLSLAPSFGSLVALKELDMHGIFFRSLDETTLRPLARLPMLQTLRLQMNFINQAQLGIFRAFPGLRYVDLSDNRISGASELTATMGEADGGEKVWLQPGDLAPAPVDTPSSEDFRPNCSTLNFTLDLSRNNLVTVQPEMFAQLSHLQCLRLSHNCISQAVNGSQFLPLTGLQVLDLSHNKLDLYHEHSFTELPRLEALDLSYNSQPFGMQGVGHNFSFVAHLRTLRHLSLAHNNIHSQVSQQLCSTSLRALDFSGNALGHMWAEGDLYLHFFQGLSGLIWLDLSQNRLHTLLPQTLRNLPKSLQVLRLRDNYLAFFKWWSLHFLPKLEVLDLAGNQLKALTNGSLPAGTRLRRLDVSCNSISFVAPGFFSKAKELRELNLSANALKTVDHSWFGPLASALQILDVSANPLHCACGAAFMDFLLEVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 34 - protein, 811 aa, Homo sapiens
  source 1..811: note = human TLR10
MRLIRNIYIFCSIVMTAEGDAPELPEERELMTNCSNMSLRKVPADLTPATTTLDLSYNLLFQLQSSDFHSVSKLRVLILCHNRIQQLDLKTFEFNKELRYLDLSNNRLKSVTWYLLAGLRYLDLSFNDFDTMPICEEAGNMSHLEILGLSGAKIQKSDFQKIAHLHLNTVFLGFRTLPHYEEGSLPILNTTKLHIVLPMDTNFWVLLRDGIKTSKILEMTNIDGKSQFVSYEMQRNLSLENAKTSVLLLNKVDLLWDDLFLILQFVWHTSVEHFQIRNVTFGGKAYLDHNSFDYSNTVMRTIKLEHVHFRVFYIQQDKIYLLLTKMDIENLTISNAQMPHMLFPNYPTKFQYLNFANNILTDELFKRTIQLPHLKTLILNGNKLETLSLVSCFANNTPLEHLDLSQNLLQHKNDENCSWPETVVNMNLSYNKLSDSVFRCLPKSIQILDLNNNQIQTVPKETIHLMALRELNIAFNFLTDLPGCSHFSRLSVLNIEMNFILSPSLDFVQSCQEVKTLNAGRNPFRCTCELKNFIQLETYSEVMMVGWSDSYTCEYPLNLRGTRLKDVHLHELSCNTALLIVTIVVIMLVLGLAVAFCCLHFDLPWYLRMLGQCTQTWHRVRKTTQEQLKRNVRFHAFISYSEHDSLWVKNELIPNLEKEDGSILICLYESYFDPGKSISENIVSFIEKSYKSIFVLSPNFVQNEWCHYEFYFAHHNLFHENSDHIILILLEPIPFYCIPTRYHKLKALLEKKAYLEWPKDRRKCGLFWANLRAAINVNVLATREMYELQTFTELNEESRGSTISLMRTDCL

SEQ ID NO: 35 - protein, 569 aa, Homo sapiens
  source 1..569: note = human IL-1R
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 36 - protein, 541 aa, Homo sapiens
  source 1..541: note = human IL-18R
MNCRELPLTLWVLISVSTAESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 37 - protein, 21 aa, Homo sapiens
  source 1..21: note = signal peptide
MALPVTALLLPLALLLHAARP

SEQ ID NO: 38 - protein, 45 aa, Homo sapiens
  source 1..45: note = hinge domain
TTTPAPRPPTPAPTIASQPLSLRPEACRPAAGGAVHTRGLDFACD

SEQ ID NO: 39 - protein, 41 aa, Homo sapiens
  source 1..41: note = hinge domain
CTIEVMYPPPYLDNEKSNGTIIHVKGKHLCPSPLFPGPSKP

SEQ ID NO: 40 - protein, 12 aa, Homo sapiens
  source 1..12: note = hinge domain
ESKYGPPCPPCP

SEQ ID NO: 41 - protein, 29 aa, synthetic construct
  source 1..29: note = Transmembrane domain sequence - aTM
NLFVASWIAVMIIFRIGMAVAIFCCFFFP

SEQ ID NO: 42 - protein, 21 aa, Homo sapiens
  source 1..21: note = Transmembrane domain sequence - DNAM1 TM
GGTVLLLLFVISITTIIVIFL

SEQ ID NO: 43 - protein, 27 aa, Homo sapiens
  source 1..27: note = Transmembrane domain sequence - CD28 TM
FWVLVVVGGVLACYSLLVTVAFIIFWV

SEQ ID NO: 44 - protein, 25 aa, Homo sapiens
  source 1..25: note = Transmembrane domain sequence - IL-2Rbeta
IPWLGHLLVGLSGAFGFIILVYLLI

SEQ ID NO: 45 - protein, 21 aa, Homo sapiens
  source 1..21: note = Transmembrane domain sequence - TLR1 TM
LLIVTIVATMLVLAVTVTSLC

SEQ ID NO: 46 - protein, 21 aa, Homo sapiens
  source 1..21: note = Transmembrane domain sequence - TLR2 TM
ALVSGMCCALFLLILLTGVLC

SEQ ID NO: 47 - protein, 21 aa, Homo sapiens
  source 1..21: note = Transmembrane domain sequence - TLR3 TM
FFMINTSILLIFIFIVLLIHF

SEQ ID NO: 48 - protein, 21 aa, Homo sapiens
  source 1..21: note = Transmembrane domain sequence - TLR4 TM
TIIGVSVLSVLVVSVVAVLVY

SEQ ID NO: 49 - protein, 21 aa, Homo sapiens
  source 1..21: note = Transmembrane domain sequence - TLR5 TM
FSLFIVCTVTLTLFLMTILTV

SEQ ID NO: 50 - protein, 21 aa, Homo sapiens
  source 1..21: note = Transmembrane domain sequence - TLR6 TM
LIVTIGATMLVLAVTVTSLCI

SEQ ID NO: 51 - protein, 21 aa, Homo sapiens
  source 1..21: note = Transmembrane domain sequence - TLR7 TM
LILFSLSISVSLFLMVMMTAS

SEQ ID NO: 52 - protein, 21 aa, Homo sapiens
  source 1..21: note = Transmembrane domain sequence - TLR8 TM
AVILFFFTFFITTMVMLAALA

SEQ ID NO: 53 - protein, 21 aa, Homo sapiens
  source 1..21: note = Transmembrane domain sequence - TLR9 TM
FALSLLAVALGLGVPMLHHLC

SEQ ID NO: 54 - protein, 21 aa, Homo sapiens
  source 1..21: note = Transmembrane domain sequence - TLR10 TM
ALLIVTIVVIMLVLGLAVAFC

SEQ ID NO: 55 - protein, 20 aa, Homo sapiens
  source 1..20: note = Transmembrane domain sequence - IL1R TM
HMIGICVTLTVIIVCSVFIY

SEQ ID NO: 56 - protein, 21 aa, Homo sapiens
  source 1..21: note = Transmembrane domain sequence - IL18R TM
GMIIAVLILVAVVCLVTVCVI

SEQ ID NO: 57 - protein, 204 aa, synthetic construct
  source 1..204: note = Membrane bound interleukin 15 (mbIL-15)
MALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 58 - protein, 162 aa, Homo sapiens
  source 1..162: note = soluble IL15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 59 - protein, 723 aa, synthetic construct
  source 1..723: note = Membrane bound interleukin 18 (mbIL-18)
MWLQSLLLLGTVACSISYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDSGGGSGGGGSGGGGSGGGGSGGGSLQAESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 60 - protein, 193 aa, Homo sapiens
  source 1..193: note = soluble IL18
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 61 - protein, 301 aa, synthetic construct
  source 1..301: note = OX40
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKIDYKDDDDKDYKDDDDKDYKDDDDK

SEQ ID NO: 62 - protein, 423 aa, synthetic construct
  source 1..423: note = Synthetic construct - IL15/IL15R fusion
MLLLVTSLLLCELPHPAFLLIPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLDYKDDDDKDYKDDDDKDYKDDDDK

SEQ ID NO: 63 - protein, 752 aa, synthetic construct
  source 1..752: note = Synthetic construct - IL18/IL18R fusion
MLLLVTSLLLCELPHPAFLLIPYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDSGGGSGGGGSGGGGSGGGGSGGGSLQAESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSESDYKDDDDKDYKDDDDKDYKDDDDK

SEQ ID NO: 64 - protein, 112 aa, Homo sapiens
  source 1..112: note = cytoplasmic domain - CD3z
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 120 aa, Homo sapiens
  source 1..120: note = cytoplasmic domain - 2B4
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 66 - protein, 37 aa, Homo sapiens
  source 1..37: note = cytoplasmic domain - OX40
RRDQRLPPDAHKPPGGGSFRTPIQEEQADAHSTLAKI

SEQ ID NO: 67 - protein, 83 aa, Homo sapiens
  source 1..83: note = cytoplasmic domain - FCER1G
LRKRRDSLSLSTQRTQGPAESARNLEYVSVSPTNNTVYASVTHSNRETEIWTPRENDTITIYSTINHSKESKPTFSRAALDNV

SEQ ID NO: 68 - protein, 41 aa, Homo sapiens
  source 1..41: note = cytoplasmic domain - CD28
RSKRSRLLHSDYMNMTPRRPGPTRKHYQPYAPPRDFAAYRS

SEQ ID NO: 69 - protein, 24 aa, Homo sapiens
  source 1..24: note = cytoplasmic domain - DAP10
LCARPRRSPAQEDGKVYINMPGRG

SEQ ID NO: 70 - protein, 61 aa, Homo sapiens
  source 1..61: note = cytoplasmic domain - DNAM1
NRRRRRERRDLFTESWDTQKAPNNYRSPISTSQPTNQSMDDTREDIYVNYPTFSRRPKTRV

SEQ ID NO: 71 - protein, 111 aa, Homo sapiens
  source 1..111: note = cytoplasmic domain - CD28H
FWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 72 - skipped

SEQ ID NO: 73 - protein, 248 aa, synthetic construct
  source 1..248: note = Synthetic construct - anti-CD19 single chain variable fragment (scFv)
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILT

SEQ ID NO: 74 - protein, 123 aa, synthetic construct
  source 1..123: note = Made in Lab - anti-CD19 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILT

SEQ ID NO: 75 - protein, 110 aa, synthetic construct
  source 1..110: note = Made in Lab - anti-CD19 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGST

SEQ ID NO: 76 - protein, 5 aa, synthetic construct
  source 1..5: note = Made in Labe - CDRH1
DYGVS

SEQ ID NO: 77 - protein, 16 aa, synthetic construct
  source 1..16: note = Made in Lab - CDRH2
VIWGSETTYYNSALKS

SEQ ID NO: 78 - protein, 12 aa, synthetic construct
  source 1..12: note = Made in Lab - CDRH3
HYYYGGSYAMDY

SEQ ID NO: 79 - protein, 11 aa, synthetic construct
  source 1..11: note = Made in Lab - CDRL1
RASQDISKYLN

SEQ ID NO: 80 - protein, 7 aa, synthetic construct
  source 1..7: note = Made in Lab - CDRL2
HTSRLHS

SEQ ID NO: 81 - protein, 9 aa, synthetic construct
  source 1..9: note = Made in Lab - CDRL3
QQGNTLPYT

SEQ ID NO: 82 - protein, 351 aa, synthetic construct
  source 1..351: note = CD8-aTM-TLR2-CD3 amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAITGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 480 aa, synthetic construct
  source 1..480: note = CD8-aTM-2B4-TLR2-CD3 amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAITGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 355 aa, synthetic construct
  source 1..355: note = CD8-aTM-IL18R-CD3 amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 484 aa, synthetic construct
  source 1..484: note = CD8-aTM-2B4-IL18R-CD3 amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 484 aa, synthetic construct
  source 1..484: note = CD8-aTM-2B4-CD3-IL18R amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGSGGSKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLM

SEQ ID NO: 87 - protein, 351 aa, synthetic construct
  source 1..351: note = CD8-aTM-TLR4-CD3 amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 480 aa, synthetic construct
  source 1..480: note = CD8-aTM-2B4-TLR4-CD3 amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 480 aa, synthetic construct
  source 1..480: note = CD8-aTM-2B4-CD3-TLR4 amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGSGGSNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 90 - protein, 351 aa, synthetic construct
  source 1..351: note = CD8-aTM-TLR3-CD3 amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 480 aa, synthetic construct
  source 1..480: note = CD8-aTM-2B4-TLR3-CD3 amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 480 aa, synthetic construct
  source 1..480: note = CD8-aTM-2B4-CD3-TLR3 amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGSGGSFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVAL

SEQ ID NO: 93 - protein, 352 aa, synthetic construct
  source 1..352: note = CD8-aTM-TLR7-CD3 amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 481 aa, synthetic construct
  source 1..481: note = CD8-aTM-2B4-TLR7-CD3 amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 481 aa, synthetic construct
  source 1..481: note = CD8-aTM-2B4-CD3-TLR7 amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGSGGSCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNAL

SEQ ID NO: 96 - protein, 352 aa, synthetic construct
  source 1..352: note = CD8-aTM-TLR8-CD3 amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - protein, 481 aa, synthetic construct
  source 1..481: note = CD8-aTM-2B4-TLR8-CD3 amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 481 aa, synthetic construct
  source 1..481: note = CD8-aTM-2B4-CD3-TLR8 amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGSGGSTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVV

SEQ ID NO: 99 - protein, 484 aa, synthetic construct
  source 1..484: note = CD8-aTM-IL18R-2B4-CD3 amino acid sequence
TTTTPAPRPPTPAPTIASQPLSLRPEACRPAAGGAVHTRGLDFACDASSNLFVASWIAVMIIFRIGMAVAIFCCFFFPSGSGGSGGSKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMSLGGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - protein, 649 aa, synthetic construct
  source 1..649: note = CD8-aTM-TLR2-CD3 amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAITGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 778 aa, synthetic construct
  source 1..778: note = CD8-aTM-2B4-TLR2-CD3 amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAITGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 653 aa, synthetic construct
  source 1..653: note = CD8-aTM-IL18R-CD3 amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - protein, 782 aa, synthetic construct
  source 1..782: note = CD8-aTM-2B4-IL18R-CD3 amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 782 aa, synthetic construct
  source 1..782: note = CD8-aTM-2B4-CD3-IL18R amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGSGGSKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLM

SEQ ID NO: 105 - protein, 649 aa, synthetic construct
  source 1..649: note = CD8-aTM-TLR4-CD3 amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 778 aa, synthetic construct
  source 1..778: note = CD8-aTM-2B4-TLR4-CD3 amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - protein, 778 aa, synthetic construct
  source 1..778: note = CD8-aTM-2B4-CD3-TLR4 amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGSGGSNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 108 - protein, 649 aa, synthetic construct
  source 1..649: note = CD8-aTM-TLR3-CD3 amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - protein, 778 aa, synthetic construct
  source 1..778: note = CD8-aTM-2B4-TLR3-CD3 amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - protein, 778 aa, synthetic construct
  source 1..778: note = CD8-aTM-2B4-CD3-TLR3 amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGSGGSFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVAL

SEQ ID NO: 111 - protein, 650 aa, synthetic construct
  source 1..650: note = CD8-aTM-TLR7-CD3 amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - protein, 779 aa, synthetic construct
  source 1..779: note = CD8-aTM-2B4-TLR7-CD3 amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - protein, 779 aa, synthetic construct
  source 1..779: note = CD8-aTM-2B4-CD3-TLR7 amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGSGGSCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNAL

SEQ ID NO: 114 - protein, 650 aa, synthetic construct
  source 1..650: note = CD8-aTM-TLR8-CD3 amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - protein, 779 aa, synthetic construct
  source 1..779: note = CD8-aTM-2B4-TLR8-CD3 amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - protein, 779 aa, synthetic construct
  source 1..779: note = CD8-aTM-2B4-CD3-TLR8 amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGSGGSTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVV

SEQ ID NO: 117 - protein, 782 aa, synthetic construct
  source 1..782: note = CD8-aTM-IL18R-2B4-CD3 amino acid sequence (with anti-CD19)
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMSLGGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - DNA, 1947 bp, synthetic construct
  source 1..1947: mol_type = other DNA; note = CD8-aTM-TLR2-CD3 polynucleotide sequence
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagcatctgctacgacgcctttgtgagctattccgagcgggatgcctactgggtggagaacctgatggtgcaggagctggagaactttaatccccctttcaagctgtgcctgcacaagagggacttcatccctggcaagtggatcatcgacaacatcatcgattccatcgagaagtctcacaagaccgtgtttgtgctgagcgagaatttcgtgaagtccgagtggtgcaagtatgagctggatttctctcactttcggctgttcgacgagaacaatgatgccgccatcctgatcctgctggagccaatcgagaagaaggccatcccccagcggttttgtaagctgagaaagatcatgaacaccaagacatacctggagtggcctatggacgaggcccagagggagggcttctgggtgaatctgcgcgccgccatcaccggtggcggctctggcggcagcagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcgg

SEQ ID NO: 119 - DNA, 2334 bp, synthetic construct
  source 1..2334: mol_type = other DNA; note = CD8-aTM-2B4-TLR2-CD3 polynucleotide sequence
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagctggcggagaaagcggaaggagaagcagtctgagaccagccccaaggagttcctgacaatctacgaggacgtgaaggatctgaagaccaggcgcaaccacgagcaggagcagacctttcctggcggcggcagcacaatctattccatgatccagtctcagagctccgcccccacctcccaggagcctgcctacacactgtatagcctgatccagccttcccggaagtccggctctcggaagagaaaccacagcccatccttcaattctacaatctacgaagtgatcggcaagagccagccaaaggcacagaaccccgccaggctgagccgcaaggagctggagaattttgacgtgtattcttccctaggaggcggctccggcggctctatctgctacgacgcctttgtgagctattccgagcgggatgcctactgggtggagaacctgatggtgcaggagctggagaactttaatccccctttcaagctgtgcctgcacaagagggacttcatccctggcaagtggatcatcgacaacatcatcgattccatcgagaagtctcacaagaccgtgtttgtgctgagcgagaatttcgtgaagtccgagtggtgcaagtatgagctggatttctctcactttcggctgttcgacgagaacaatgatgccgccatcctgatcctgctggagccaatcgagaagaaggccatcccccagcggttttgtaagctgagaaagatcatgaacaccaagacatacctggagtggcctatggacgaggcccagagggagggcttctgggtgaatctgcgcgccgccatcaccggtggcggctctggcggcagcagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcgg

SEQ ID NO: 120 - DNA, 1959 bp, synthetic construct
  source 1..1959: mol_type = other DNA; note = CD8-aTM-IL18R-CD3 polynucleotide sequence
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagcaagacatacgacgccttcgtgagctatctgaaggagtgccggccagagaacggcgaggagcacacctttgccgtggagatcctgccccgggtgctggagaagcacttcggctacaagctgtgcatctttgagagagacgtggtgcccggaggagcagtggtggatgagatccacagcctgatcgagaagtcccggagactgatcatcgtgctgagcaagtcctacatgtccaatgaggtgaggtacgagctggagtctggactgcacgaggccctggtggagaggaagatcaagatcatcctgatcgagttcacccccgtgacagacttcacctttctgcctcagtctctgaagctgctgaagagccacagggtgctgaagtggaaggccgataagtctctgagctacaactcccgcttttggaagaatctgctgtatctgatgaccggtggcggctccggcggctctagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcgg

SEQ ID NO: 121 - DNA, 2346 bp, synthetic construct
  source 1..2346: mol_type = other DNA; note = CD8-aTM-2B4-IL18R-CD3 polynucleotide sequence
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagctggcggagaaagcggaaggagaagcagtctgagaccagccccaaggagttcctgacaatctacgaggacgtgaaggatctgaagaccaggcgcaaccacgagcaggagcagacctttcctggcggcggcagcacaatctattccatgatccagtctcagagctccgcccccacctcccaggagcctgcctacacactgtatagcctgatccagccttcccggaagtccggctctcggaagagaaaccacagcccatccttcaattctacaatctacgaagtgatcggcaagagccagccaaaggcacagaaccccgccaggctgagccgcaaggagctggagaattttgacgtgtattcttccctaggaggcggctccggcggctctaagacatacgacgccttcgtgagctatctgaaggagtgccggccagagaacggcgaggagcacacctttgccgtggagatcctgccccgggtgctggagaagcacttcggctacaagctgtgcatctttgagagagacgtggtgcccggaggagcagtggtggatgagatccacagcctgatcgagaagtcccggagactgatcatcgtgctgagcaagtcctacatgtccaatgaggtgaggtacgagctggagtctggactgcacgaggccctggtggagaggaagatcaagatcatcctgatcgagttcacccccgtgacagacttcacctttctgcctcagtctctgaagctgctgaagagccacagggtgctgaagtggaaggccgataagtctctgagctacaactcccgcttttggaagaatctgctgtatctgatgaccggtggcggctccggcggctctagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcgg

SEQ ID NO: 122 - DNA, 2346 bp, synthetic construct
  source 1..2346: mol_type = other DNA; note = CD8-aTM-2B4-CD3-IL18R polynucleotide sequence
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagctggcggagaaagcggaaggagaagcagtctgagaccagccccaaggagttcctgacaatctacgaggacgtgaaggatctgaagaccaggcgcaaccacgagcaggagcagacctttcctggcggcggcagcacaatctattccatgatccagtctcagagctccgcccccacctcccaggagcctgcctacacactgtatagcctgatccagccttcccggaagtccggctctcggaagagaaaccacagcccatccttcaattctacaatctacgaagtgatcggcaagagccagccaaaggcacagaaccccgccaggctgagccgcaaggagctggagaattttgacgtgtattcttccctaggaggcggctccggcggctctagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcggaccggtggcggctctggcggcagcaagacatacgacgccttcgtgagctatctgaaggagtgccggccagagaacggcgaggagcacacctttgccgtggagatcctgccccgggtgctggagaagcacttcggctacaagctgtgcatctttgagagagacgtggtgcccggaggagcagtggtggatgagatccacagcctgatcgagaagtcccggagactgatcatcgtgctgagcaagtcctacatgtccaatgaggtgaggtacgagctggagtctggactgcacgaggccctggtggagaggaagatcaagatcatcctgatcgagttcacccccgtgacagacttcacctttctgcctcagtctctgaagctgctgaagagccacagggtgctgaagtggaaggccgataagtctctgagctacaactcccgcttttggaagaatctgctgtatctgatg

SEQ ID NO: 123 - DNA, 1947 bp, synthetic construct
  source 1..1947: mol_type = other DNA; note = CD8-aTM-TLR4-CD3 polynucleotide sequence
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagcaacatctacgacgccttcgtgatctatagctcccaggacgaggattgggtgaggaacgagctggtgaagaatctggaggagggcgtgccccctttccagctgtgcctgcactacagggacttcatcccaggagtggcaatcgccgccaatatcatccacgagggctttcacaagtccaggaaagtgatcgtggtggtgagccagcacttcatccagtcccgctggtgtatctttgagtatgagatcgcccagacctggcagttcctgtctagccgggccggcatcatctttatcgtgctgcagaaggtggagaagaccctgctgagacagcaggtggagctgtacaggctgctgtctcgcaacacatatctggagtgggaggacagcgtgctgggccggcacatcttctggcggagactgagaaaggccctgaccggtggcggctccggcggctctagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcgg

SEQ ID NO: 124 - DNA, 2334 bp, synthetic construct
  source 1..2334: mol_type = other DNA; note = CD8-aTM-2B4-TLR4-CD3 polynucleotide sequence
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagctggcggagaaagcggaaggagaagcagtctgagaccagccccaaggagttcctgacaatctacgaggacgtgaaggatctgaagaccaggcgcaaccacgagcaggagcagacctttcctggcggcggcagcacaatctattccatgatccagtctcagagctccgcccccacctcccaggagcctgcctacacactgtatagcctgatccagccttcccggaagtccggctctcggaagagaaaccacagcccatccttcaattctacaatctacgaagtgatcggcaagagccagccaaaggcacagaaccccgccaggctgagccgcaaggagctggagaattttgacgtgtattcttccctaggaggcggctccggcggctctaacatctacgacgccttcgtgatctatagctcccaggacgaggattgggtgaggaacgagctggtgaagaatctggaggagggcgtgccccctttccagctgtgcctgcactacagggacttcatcccaggagtggcaatcgccgccaatatcatccacgagggctttcacaagtccaggaaagtgatcgtggtggtgagccagcacttcatccagtcccgctggtgtatctttgagtatgagatcgcccagacctggcagttcctgtctagccgggccggcatcatctttatcgtgctgcagaaggtggagaagaccctgctgagacagcaggtggagctgtacaggctgctgtctcgcaacacatatctggagtgggaggacagcgtgctgggccggcacatcttctggcggagactgagaaaggccctgaccggtggcggctccggcggctctagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcgg

SEQ ID NO: 125 - DNA, 2334 bp, synthetic construct
  source 1..2334: mol_type = other DNA; note = CD8-aTM-2B4-CD3-TLR4 polynucleotide sequence
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagctggcggagaaagcggaaggagaagcagtctgagaccagccccaaggagttcctgacaatctacgaggacgtgaaggatctgaagaccaggcgcaaccacgagcaggagcagacctttcctggcggcggcagcacaatctattccatgatccagtctcagagctccgcccccacctcccaggagcctgcctacacactgtatagcctgatccagccttcccggaagtccggctctcggaagagaaaccacagcccatccttcaattctacaatctacgaagtgatcggcaagagccagccaaaggcacagaaccccgccaggctgagccgcaaggagctggagaattttgacgtgtattcttccctaggaggcggctccggcggctctagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcggaccggtggcggctctggcggcagcaacatctacgacgccttcgtgatctatagctcccaggacgaggattgggtgaggaacgagctggtgaagaatctggaggagggcgtgccccctttccagctgtgcctgcactacagggacttcatcccaggagtggcaatcgccgccaatatcatccacgagggctttcacaagtccaggaaagtgatcgtggtggtgagccagcacttcatccagtcccgctggtgtatctttgagtatgagatcgcccagacctggcagttcctgtctagccgggccggcatcatctttatcgtgctgcagaaggtggagaagaccctgctgagacagcaggtggagctgtacaggctgctgtctcgcaacacatatctggagtgggaggacagcgtgctgggccggcacatcttctggcggagactgagaaaggccctg

SEQ ID NO: 126 - DNA, 1947 bp, synthetic construct
  source 1..1947: mol_type = other DNA; note = CD8-aTM-TLR3-CD3 polynucleotide sequence
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagcttcgagtacgccgcctatatcatccacgcctacaaggacaaggattgggtgtgggagcacttcagcagcatggagaaggaggaccagtctctgaagttttgcctggaggagagggatttcgaggccggcgtgtttgagctggaggccatcgtgaactccatcaagaggtctcgcaagatcatcttcgtgatcacccaccacctgctgaaggaccccctgtgcaagcggttcaaggtgcaccacgccgtgcagcaggccatcgagcagaatctggacagcatcatcctggtgtttctggaggagatccccgattataagctgaaccacgccctgtgcctgcggagaggcatgttcaagtcccactgtatcctgaattggcctgtgcagaaggagcggatcggcgcctttagacacaagctgcaggtggccctgaccggtggcggctctggcggcagcagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcgg

SEQ ID NO: 127 - DNA, 2334 bp, synthetic construct
  source 1..2334: mol_type = other DNA; note = CD8-aTM-2B4-TLR3-CD3 polynucleotide sequence
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagctggcggagaaagcggaaggagaagcagtctgagaccagccccaaggagttcctgacaatctacgaggacgtgaaggatctgaagaccaggcgcaaccacgagcaggagcagacctttcctggcggcggcagcacaatctattccatgatccagtctcagagctccgcccccacctcccaggagcctgcctacacactgtatagcctgatccagccttcccggaagtccggctctcggaagagaaaccacagcccatccttcaattctacaatctacgaagtgatcggcaagagccagccaaaggcacagaaccccgccaggctgagccgcaaggagctggagaattttgacgtgtattcttccctaggaggcggctccggcggctctttcgagtacgccgcctatatcatccacgcctacaaggacaaggattgggtgtgggagcacttcagcagcatggagaaggaggaccagtctctgaagttttgcctggaggagagggatttcgaggccggcgtgtttgagctggaggccatcgtgaactccatcaagaggtctcgcaagatcatcttcgtgatcacccaccacctgctgaaggaccccctgtgcaagcggttcaaggtgcaccacgccgtgcagcaggccatcgagcagaatctggacagcatcatcctggtgtttctggaggagatccccgattataagctgaaccacgccctgtgcctgcggagaggcatgttcaagtcccactgtatcctgaattggcctgtgcagaaggagcggatcggcgcctttagacacaagctgcaggtggccctgaccggtggcggctctggcggcagcagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcgg

SEQ ID NO: 128 - DNA, 2334 bp, synthetic construct
  source 1..2334: mol_type = other DNA; note = CD8-aTM-2B4-CD3-TLR3 polynucleotide sequence
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagctggcggagaaagcggaaggagaagcagtctgagaccagccccaaggagttcctgacaatctacgaggacgtgaaggatctgaagaccaggcgcaaccacgagcaggagcagacctttcctggcggcggcagcacaatctattccatgatccagtctcagagctccgcccccacctcccaggagcctgcctacacactgtatagcctgatccagccttcccggaagtccggctctcggaagagaaaccacagcccatccttcaattctacaatctacgaagtgatcggcaagagccagccaaaggcacagaaccccgccaggctgagccgcaaggagctggagaattttgacgtgtattcttccctaggaggcggctccggcggctctagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcggaccggtggcggctctggcggcagcttcgagtacgccgcctatatcatccacgcctacaaggacaaggattgggtgtgggagcacttcagcagcatggagaaggaggaccagtctctgaagttttgcctggaggagagggatttcgaggccggcgtgtttgagctggaggccatcgtgaactccatcaagaggtctcgcaagatcatcttcgtgatcacccaccacctgctgaaggaccccctgtgcaagcggttcaaggtgcaccacgccgtgcagcaggccatcgagcagaatctggacagcatcatcctggtgtttctggaggagatccccgattataagctgaaccacgccctgtgcctgcggagaggcatgttcaagtcccactgtatcctgaattggcctgtgcagaaggagcggatcggcgcctttagacacaagctgcaggtggccctg

SEQ ID NO: 129 - DNA, 1950 bp, synthetic construct
  source 1..1950: mol_type = other DNA; note = CD8-aTM-TLR7-CD3 polynucleotide sequence
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagctgctgttacgacgcctttatcgtgtatgacaccaaggaccccgcagtgacagagtgggtgctggccgagctggtggcaaagctggaggacccaagggagaagcacttcaacctgtgcctcgaggagagggattggctgccaggacagcctgtgctggagaatctgtctcagagcatccagctgagcaagaagaccgtgtttgtgatgaccgataagtacgccaagaccgagaacttcaagatcgccttttatctgtcccaccagcggctgatggacgagaaggtggatgtgatcatcctgatcttcctggagaagccctttcagaagtctaagttcctgcagctgcggaagagactgtgcggcagctccgtgctggagtggccaaccaacccacaggcacacccttacttctggcagtgtctgaagaatgccctgaccggtggcggctccggcggctctagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcgg

SEQ ID NO: 130 - DNA, 2337 bp, synthetic construct
  source 1..2337: mol_type = other DNA; note = CD8-aTM-2B4-TLR7-CD3 polynucleotide sequence
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagctggcggagaaagcggaaggagaagcagtctgagaccagccccaaggagttcctgacaatctacgaggacgtgaaggatctgaagaccaggcgcaaccacgagcaggagcagacctttcctggcggcggcagcacaatctattccatgatccagtctcagagctccgcccccacctcccaggagcctgcctacacactgtatagcctgatccagccttcccggaagtccggctctcggaagagaaaccacagcccatccttcaattctacaatctacgaagtgatcggcaagagccagccaaaggcacagaaccccgccaggctgagccgcaaggagctggagaattttgacgtgtattcttccctaggaggcggctccggcggctcttgctgttacgacgcctttatcgtgtatgacaccaaggaccccgcagtgacagagtgggtgctggccgagctggtggcaaagctggaggacccaagggagaagcacttcaacctgtgcctcgaggagagggattggctgccaggacagcctgtgctggagaatctgtctcagagcatccagctgagcaagaagaccgtgtttgtgatgaccgataagtacgccaagaccgagaacttcaagatcgccttttatctgtcccaccagcggctgatggacgagaaggtggatgtgatcatcctgatcttcctggagaagccctttcagaagtctaagttcctgcagctgcggaagagactgtgcggcagctccgtgctggagtggccaaccaacccacaggcacacccttacttctggcagtgtctgaagaatgccctgaccggtggcggctccggcggctctagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcgg

SEQ ID NO: 131 - DNA, 2337 bp, synthetic construct
  source 1..2337: mol_type = other DNA; note = CD8-aTM-2B4-CD3-TLR7 polynucleotide sequence
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagctggcggagaaagcggaaggagaagcagtctgagaccagccccaaggagttcctgacaatctacgaggacgtgaaggatctgaagaccaggcgcaaccacgagcaggagcagacctttcctggcggcggcagcacaatctattccatgatccagtctcagagctccgcccccacctcccaggagcctgcctacacactgtatagcctgatccagccttcccggaagtccggctctcggaagagaaaccacagcccatccttcaattctacaatctacgaagtgatcggcaagagccagccaaaggcacagaaccccgccaggctgagccgcaaggagctggagaattttgacgtgtattcttccctaggaggcggctccggcggctctagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcggaccggtggcggctctggcggcagctgctgttacgacgcctttatcgtgtatgacaccaaggaccccgcagtgacagagtgggtgctggccgagctggtggcaaagctggaggacccaagggagaagcacttcaacctgtgcctcgaggagagggattggctgccaggacagcctgtgctggagaatctgtctcagagcatccagctgagcaagaagaccgtgtttgtgatgaccgataagtacgccaagaccgagaacttcaagatcgccttttatctgtcccaccagcggctgatggacgagaaggtggatgtgatcatcctgatcttcctggagaagccctttcagaagtctaagttcctgcagctgcggaagagactgtgcggcagctccgtgctggagtggccaaccaacccacaggcacacccttacttctggcagtgtctgaagaatgccctg

SEQ ID NO: 132 - DNA, 1950 bp, synthetic construct
  source 1..1950: mol_type = other DNA; note = CD8-aTM-TLR8-CD3 polynucleotide sequence
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagcaccttctacgacgcctatatcagctacgacaccaaggatgcctccgtgacagattgggtcatcaacgagctgcgctatcacctggaggagtcccgggacaagaatgtgctgctgtgtctagaggagagagactgggaccccggcctggccatcatcgataacctgatgcagtccatcaatcagtctaagaagaccgtgttcgtgctgacaaagaagtatgccaagtcttggaacttcaagacagccttttacctggccctgcagaggctgatggacgagaatatggatgtgatcatctttatcctgctggagcccgtgctgcagcacagccagtacctgcggctgagacagaggatctgtaagagctccatcctgcagtggcccgacaaccctaaggccgagggcctgttttggcagaccctgagaaatgtggtgaccggtggcggctctggcggcagcagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcgg

SEQ ID NO: 133 - DNA, 2337 bp, synthetic construct
  source 1..2337: mol_type = other DNA; note = CD8-aTM-2B4-TLR8-CD3 polynucleotide sequence
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagctggcggagaaagcggaaggagaagcagtctgagaccagccccaaggagttcctgacaatctacgaggacgtgaaggatctgaagaccaggcgcaaccacgagcaggagcagacctttcctggcggcggcagcacaatctattccatgatccagtctcagagctccgcccccacctcccaggagcctgcctacacactgtatagcctgatccagccttcccggaagtccggctctcggaagagaaaccacagcccatccttcaattctacaatctacgaagtgatcggcaagagccagccaaaggcacagaaccccgccaggctgagccgcaaggagctggagaattttgacgtgtattcttccctaggaggcggctccggcggctctaccttctacgacgcctatatcagctacgacaccaaggatgcctccgtgacagattgggtcatcaacgagctgcgctatcacctggaggagtcccgggacaagaatgtgctgctgtgtctagaggagagagactgggaccccggcctggccatcatcgataacctgatgcagtccatcaatcagtctaagaagaccgtgttcgtgctgacaaagaagtatgccaagtcttggaacttcaagacagccttttacctggccctgcagaggctgatggacgagaatatggatgtgatcatctttatcctgctggagcccgtgctgcagcacagccagtacctgcggctgagacagaggatctgtaagagctccatcctgcagtggcccgacaaccctaaggccgagggcctgttttggcagaccctgagaaatgtggtgaccggtggcggctctggcggcagcagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcgg

SEQ ID NO: 134 - DNA, 2337 bp, synthetic construct
  source 1..2337: mol_type = other DNA; note = CD8-aTM-2B4-CD3-TLR8 polynucleotide sequence
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagctggcggagaaagcggaaggagaagcagtctgagaccagccccaaggagttcctgacaatctacgaggacgtgaaggatctgaagaccaggcgcaaccacgagcaggagcagacctttcctggcggcggcagcacaatctattccatgatccagtctcagagctccgcccccacctcccaggagcctgcctacacactgtatagcctgatccagccttcccggaagtccggctctcggaagagaaaccacagcccatccttcaattctacaatctacgaagtgatcggcaagagccagccaaaggcacagaaccccgccaggctgagccgcaaggagctggagaattttgacgtgtattcttccctaggaggcggctccggcggctctagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcggaccggtggcggctctggcggcagcaccttctacgacgcctatatcagctacgacaccaaggatgcctccgtgacagattgggtcatcaacgagctgcgctatcacctggaggagtcccgggacaagaatgtgctgctgtgtctagaggagagagactgggaccccggcctggccatcatcgataacctgatgcagtccatcaatcagtctaagaagaccgtgttcgtgctgacaaagaagtatgccaagtcttggaacttcaagacagccttttacctggccctgcagaggctgatggacgagaatatggatgtgatcatctttatcctgctggagcccgtgctgcagcacagccagtacctgcggctgagacagaggatctgtaagagctccatcctgcagtggcccgacaaccctaaggccgagggcctgttttggcagaccctgagaaatgtggtg

SEQ ID NO: 135 - DNA, 2346 bp, synthetic construct
  source 1..2346: mol_type = other DNA; note = CD8-aTM-IL18R-2B4-CD3 polynucleotide sequence
atggccttaccagtgaccgccttgctcctgccgctggccttgctgctccacgccgccaggccgtacccatacgatgttccagattacgcttatccctacgacgtgcctgattatgcatacccatatgatgtccccgactatgccggctcgagtgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatctggcaagcccggatctggcgagggatctaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctccggaattcttaccaccacaacgcccgctcctcggccaccgacgccagcgccaactattgcgagtcagcctctcagtctgcgacctgaggcttgtcgaccagcagccggaggcgcagtgcacacgagggggctggacttcgcctgtgatgctagctccaacctttttgtggcgtcttggatagcggtgatgataatatttaggattgggatggcggtagcgatattttgctgctttttcttccctagcggatccggaggttctggtggatcaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgtccctaggaggaggttctggtggatcatggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctgggtccaggaagaggaaccacagcccttccttcaatagcactatctatgaagtgattggaaagagtcaacctaaagcccagaaccctgctcgattgagccgcaaagagctggagaactttgatgtttattccaccggtggaggttctggtggatcaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 136 - protein, 7 aa, unidentified
  source 1..7: note = TIR signaling domain motif
FADFISY

SEQ ID NO: 137 - protein, 6 aa, unidentified
  source 1..6: note = TIR signaling domain motif
  VARIANT 3..4: note = X is any amino acid
RDXXPG

SEQ ID NO: 138 - protein, 5 aa, unidentified
  source 1..5: note = TIR signaling domain motif
GYKLC

SEQ ID NO: 139 - protein, 246 aa, synthetic construct
  source 1..246: note = CD20-scFv-4
DIQLTQSPSSLSASVGDRVTITCRASSSVSYIHWFQQKPGKAPKPWIYATSNLASGVPSRFSGSGSGTDYTLTISSLQPEDFATYYCQQWTSNPPTFGGGTKVEIKGGGGSGGGGSGGGGSGGGGSQVQLVQSGAEVKKPGASVKVSCKASGYTFTSYNMHWVRQAPGQGLEWIGAIYPGNGDTSYNQKFKGRATLTADKSTSTAYMELSSLRSEDTAVYYCARSTYYGGDWYFNVWGRGTLVTVS

SEQ ID NO: 140 - protein, 247 aa, synthetic construct
  source 1..247: note = HER2-scFv-2 (VL-VH)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGSGGGSGGGSGGGSGGGSGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 141 - protein, 242 aa, synthetic construct
  source 1..242: note = HER2-scFv-3 (VL-VH)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 142 - protein, 247 aa, synthetic construct
  source 1..247: note = HER2-scFv-5 (VL-VH)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 143 - protein, 161 aa, synthetic construct
  source 1..161: note = IL-1R4 signaling domain
KLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQM

SEQ ID NO: 144 - protein, 156 aa, synthetic construct
  source 1..156: note = IL-1R6 signaling domain
KLYDAYVLYPKPHKESQRHAVDALVLNILPEVLERQCGYKLFIFGRDEFPGQAVANVIDENVKLCRRLIVIVVPESLGFGLLKNLSEEQIAVYSALIQDGMKVILIELEKIEDYTVMPESIQYIKQKHGAIRWHGDFTEQSQCMKTKFWKTVRYHM

SEQ ID NO: 145 - protein, 144 aa, synthetic construct
  source 1..144: note = IL-1R3 signaling domain
KEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAM

SEQ ID NO: 146 - protein, 154 aa, synthetic construct
  source 1..154: note = IL-1R7 signaling domain
KDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHM

SEQ ID NO: 147 - protein, 145 aa, synthetic construct
  source 1..145: note = IL-1R8 signaling domain
KLYDAYVSYSDCPEDRKFVNFILKPQLERRRGYKLFLDDRDLLPRAEPSADLLVNLSRCRRLIVVLSDAFLSRAWCSHSFREGLCRLLELTRRPIFITFEGQRRDPAHPALRLLRQHRHLVTLLLWRPGSVTPSSDFWKEVQLAL

SEQ ID NO: 148 - protein, 157 aa, synthetic construct
  source 1..157: note = IL-1R9 signaling domain
KDYDAYLSYTKVDPDQWNQETGEEERFALEILPDMLEKHYGYKLFIPDRDLIPTGTYIEDVARCVDQSKRLIIVMTPNYVVRRGWSIFELETRLRNMLVTGEIKVILIECSELRGIMNYQEVEALKHTIKLLTVIKWHGPKCNKLNSKFWKRLQYEM

SEQ ID NO: 149 - protein, 157 aa, synthetic construct
  source 1..157: note = IL-1R10 signaling domain
KEYDAYLSYTKVDQDTLDCDNPEEEQFALEVLPDVLEKHYGYKLFIPERDLIPSGTYMEDLTRYVEQSRRLIIVLTPDYILRRGWSIFELESRLHNMLVSGEIKVILIECTELKGKVNCQEVESLKRSIKLLSLIKWKGSKSSKLNSKFWKHLVYEM

SEQ ID NO: 150 - protein, 107 aa, synthetic construct
  source 1..107: note = CD19 scFv_LC
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 151 - protein, 120 aa, synthetic construct
  source 1..120: note = CD19 scFv_HC
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 152 - protein, 242 aa, synthetic construct
  source 1..242: note = CD19 SCFV_Full
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 153 - DNA, 726 bp, synthetic construct
  source 1..726: mol_type = other DNA; note = CD19 SCFV_Full
gacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggaggaggaggcagcggaggaggaggctccggcggcggcggctctgaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtcttcc

SEQ ID NO: 154 - protein, 125 aa, synthetic construct
  source 1..125: note = CD20-scFv_HC-ADI35500
QVQLVQSGAEVKKPGASVKVSCKASGYTFKEYGISWVRQAPGQGLEWMGWISAYSGHTYYAQKLQGRVTMTTDTSTSTAYMELRSLRSDDTAVYYCARGPHYDDWSGFIIWFDPWGQGTLVTVSS

SEQ ID NO: 155 - protein, 107 aa, synthetic construct
  source 1..107: note = CD20-scFv_LC-ADI35500
DIQMTQSPSSLSASVGDRVTITCRASQSISSYLNWYQQKPGKAPKLLIYAASSLQSGVPSRFSGSGSGTDFTLTISSLQPEDFATYYCQQSYRFPPTFGQGTKVEIK

SEQ ID NO: 156 - protein, 250 aa, synthetic construct
  source 1..250: note = CD20_SCFV_Full
QVQLVQSGAEVKKPGASVKVSCKASGYTFKEYGISWVRQAPGQGLEWMGWISAYSGHTYYAQKLQGRVTMTTDTSTSTAYMELRSLRSDDTAVYYCARGPHYDDWSGFIIWFDPWGQGTLVTVSSGSTSGSGKPGSGEGSTKGDIQMTQSPSSLSASVGDRVTITCRASQSISSYLNWYQQKPGKAPKLLIYAASSLQSGVPSRFSGSGSGTDFTLTISSLQPEDFATYYCQQSYRFPPTFGQGTKVEIK

SEQ ID NO: 157 - DNA, 750 bp, synthetic construct
  source 1..750: mol_type = other DNA; note = CD20_SCFV_Full
caggtgcaactggtgcagagcggagccgaagttaagaaaccaggcgcctctgtgaaggtgtcctgcaaagccagcggctacacctttaaagagtacggcatcagctgggtccgacaggctccaggacagggacttgagtggatgggctggatctctgcctatagcggccacacctactacgcccagaaactgcagggcagagtgacaatgaccaccgacacaagcaccagcaccgcctacatggaactgagatccctgagaagcgacgatacagccgtgtactactgtgccagaggaccccactatgacgattggagcggcttcatcatttggttcgacccctggggacagggaaccctggttacagtgtccagcggcagtacaagcggatctggcaaacctggaagcggcgagggaagcacaaagggcgacattcagatgacacagagcccttccagcctgagcgcttctgtgggagacagagtgactatcacatgccgggccagccagagcatctccagctatctcaattggtatcaacagaagcctggcaaggcccctaaactgctcatctatgccgcctctagtctgcagtcaggcgtgccctctagattctctggctctggctccggcacagactttaccctgactatcagctccctgcagcctgaggacttcgccacctactattgtcagcagagctacagattccctccaaccttcggccagggaacaaaggtggaaatcaaa

SEQ ID NO: 158 - protein, 46 aa, synthetic construct
  source 1..46: note = CD8 Hinge
TTTTPAPRPPTPAPTIASQPLSLRPEACRPAAGGAVHTRGLDFACD

SEQ ID NO: 159 - DNA, 138 bp, synthetic construct
  source 1..138: mol_type = other DNA; note = CD8 Hinge
accacaaccacaccagcacctcggccacccaccccagcaccaacaatcgccagccagcctctgtccctgaggccagaggcatgcaggccagcagcaggaggagcagtgcacaccagaggcctggactttgcctgtgat

SEQ ID NO: 160 - DNA, 81 bp, synthetic construct
  source 1..81: mol_type = other DNA; note = CD28 TM
ttctgggttcttgtggtagtaggcggagtgctcgcgtgttactcattgctcgtcacggtcgcgtttatcattttttgggtt

SEQ ID NO: 161 - DNA, 63 bp, synthetic construct
  source 1..63: mol_type = other DNA; note = TLR2TM
gcacttgtgtcaggtatgtgttgtgctttgtttctcctgattttgttgacaggcgtgctttgt

SEQ ID NO: 162 - DNA, 63 bp, synthetic construct
  source 1..63: mol_type = other DNA; note = DNAM1 TM
gggggcaccgtattgttgttgctgttcgtcatatcaattactactattattgttatatttctt

SEQ ID NO: 163 - protein, 31 aa, synthetic construct
  source 1..31: note = aTM
SNLFVASWIAVMIIFRIGMAVAIFCCFFFPS

SEQ ID NO: 164 - DNA, 93 bp, synthetic construct
  source 1..93: mol_type = other DNA; note = aTM
tccaacctgtttgtggcctcttggatcgccgtgatgatcatctttcggatcggcatggccgtggccatcttctgctgtttctttttccccagc

SEQ ID NO: 165 - DNA, 93 bp, synthetic construct
  source 1..93: mol_type = other DNA; note = aTM
tccaatctgttcgtggcttcttggatcgccgtgatgatcattttccggatcggaatggccgtggctatcttctgctgcttcttctttccatcc

SEQ ID NO: 166 - protein, 8 aa, synthetic construct
  source 1..8: note = Linker
GSGGSGGS

SEQ ID NO: 167 - DNA, 24 bp, synthetic construct
  source 1..24: mol_type = other DNA; note = Linker
ggatccggcggctctggcggcagc

SEQ ID NO: 168 - protein, 6 aa, synthetic construct
  source 1..6: note = Linker2
GGSGGS

SEQ ID NO: 169 - DNA, 18 bp, synthetic construct
  source 1..18: mol_type = other DNA; note = Linker2
ggaggttctggtggatca

SEQ ID NO: 170 - protein, 15 aa, synthetic construct
  source 1..15: note = Linker3
GGGGSGGGGSGGGGS

SEQ ID NO: 171 - DNA, 45 bp, synthetic construct
  source 1..45: mol_type = other DNA; note = Linker3
ggaggaggaggcagcggaggaggaggctccggcggcggcggctct

SEQ ID NO: 172 - protein, 18 aa, synthetic construct
  source 1..18: note = Linker4 (Whitlow)
GSTSGSGKPGSGEGSTKG

SEQ ID NO: 173 - DNA, 54 bp, synthetic construct
  source 1..54: mol_type = other DNA; note = Linker4 (Whitlow)
ggctccacctctggatccggcaagcccggatctggcgagggatccaccaagggc

SEQ ID NO: 174 - protein, 26 aa, synthetic construct
  source 1..26: note = Furin-GSG-T2A
RAKRSGSGEGRGSLLTCGDVEENPGP

SEQ ID NO: 175 - DNA, 78 bp, synthetic construct
  source 1..78: mol_type = other DNA; note = Furin-GSG-T2A
agggctaagagatctggctctggcgagggcagaggcagcttgcttacatgtggcgatgtggaggagaaccccgggccc

SEQ ID NO: 176 - protein, 47 aa, synthetic construct
  source 1..47: note = 41BB Costim
RFSVVKRGRKKLLYIFKQPFMRPVQTTQEEDGCSCRFPEEEEGGCEL

SEQ ID NO: 177 - DNA, 141 bp, synthetic construct
  source 1..141: mol_type = other DNA; note = 41BB Costim
agattcagcgttgtgaagagaggccggaagaagctgctgtacatcttcaagcagcccttcatgagacctgtgcagaccacacaggaggaagacggctgcagctgtagattccccgaggaagaggagggcggctgtgagctg

SEQ ID NO: 178 - DNA, 123 bp, synthetic construct
  source 1..123: mol_type = other DNA; note = CD28 Costim
agaagcaagagaagcagactgctccacagcgactacatgaacatgacccccagacggcctggccccaccagaaagcattaccagccctacgctcctcccagagacttcgccgcctacaggagc

SEQ ID NO: 179 - DNA, 360 bp, synthetic construct
  source 1..360: mol_type = other DNA; note = 2B4
tggagaagaaagcgcaaagagaagcagtccgagacaagccccaaagagttcctgaccatctacgaggacgtgaaggacctgaaaacccggcggaatcacgagcaagagcagacatttccaggcggaggctccaccatctacagcatgatccagtctcagagcagcgcccctacaagccaagagcctgcctacacactgtactccctgatccagcctagcagaaagagcggctcccggaagagaaatcactcccctagcttcaacagcacgatctacgaagtgatcggcaagtcccagcctaaggctcagaatcccgccagactgagccggaaagagctggaaaacttcgacgtgtacagc

SEQ ID NO: 180 - DNA, 432 bp, synthetic construct
  source 1..432: mol_type = other DNA; note = TLR2 TIR
atctgctacgacgcctttgtgagctattccgagcgggatgcctactgggtggagaacctgatggtgcaggagctggagaactttaatccccctttcaagctgtgcctgcacaagagggacttcatccctggcaagtggatcatcgacaacatcatcgattccatcgagaagtctcacaagaccgtgtttgtgctgagcgagaatttcgtgaagtccgagtggtgcaagtatgagctggatttctctcactttcggctgttcgacgagaacaatgatgccgccatcctgatcctgctggagccaatcgagaagaaggccatcccccagcggttttgtaagctgagaaagatcatgaacaccaagacatacctggagtggcctatggacgaggcccagagggagggcttctgggtgaatctgcgcgccgccatc

SEQ ID NO: 181 - DNA, 336 bp, synthetic construct
  source 1..336: mol_type = other DNA; note = CD3zeta
agagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcgg

SEQ ID NO: 182 - DNA, 426 bp, synthetic construct; identical to SEQ ID NO: 18
  source 1..426: mol_type = other DNA; note = TLR1

SEQ ID NO: 183 - DNA, 432 bp, synthetic construct; identical to SEQ ID NO: 11
  source 1..432: mol_type = other DNA; note = TLR3

SEQ ID NO: 184 - DNA, 432 bp, synthetic construct; identical to SEQ ID NO: 16
  source 1..432: mol_type = other DNA; note = TLR4

SEQ ID NO: 185 - DNA, 438 bp, synthetic construct; identical to SEQ ID NO: 20
  source 1..438: mol_type = other DNA; note = TLR5

SEQ ID NO: 186 - DNA, 426 bp, synthetic construct; identical to SEQ ID NO: 19
  source 1..426: mol_type = other DNA; note = TLR6

SEQ ID NO: 187 - DNA, 435 bp, synthetic construct; identical to SEQ ID NO: 12
  source 1..435: mol_type = other DNA; note = TLR7

SEQ ID NO: 188 - DNA, 435 bp, synthetic construct; identical to SEQ ID NO: 13
  source 1..435: mol_type = other DNA; note = TLR8

SEQ ID NO: 189 - DNA, 438 bp, synthetic construct; identical to SEQ ID NO: 14
  source 1..438: mol_type = other DNA; note = TLR9

SEQ ID NO: 190 - DNA, 432 bp, synthetic construct; identical to SEQ ID NO: 17
  source 1..432: mol_type = other DNA; note = TLR10

SEQ ID NO: 191 - protein, 161 aa, synthetic construct; identical to SEQ ID NO: 143
  source 1..161: note = IL-1R4

SEQ ID NO: 192 - protein, 156 aa, synthetic construct; identical to SEQ ID NO: 144
  source 1..156: note = IL-1R6

SEQ ID NO: 193 - protein, 144 aa, synthetic construct; identical to SEQ ID NO: 145
  source 1..144: note = IL-1R3

SEQ ID NO: 194 - protein, 154 aa, synthetic construct; identical to SEQ ID NO: 146
  source 1..154: note = IL-1R7

SEQ ID NO: 195 - protein, 145 aa, synthetic construct; identical to SEQ ID NO: 147
  source 1..145: note = IL-1R8

SEQ ID NO: 196 - protein, 157 aa, synthetic construct; identical to SEQ ID NO: 148
  source 1..157: note = IL-1R9

SEQ ID NO: 197 - protein, 157 aa, synthetic construct; identical to SEQ ID NO: 149
  source 1..157: note = IL-1R10

SEQ ID NO: 198 - DNA, 741 bp, synthetic construct
  source 1..741: mol_type = other DNA; note = HER2-2 scFv
gaggtgcagctggtggagtccggaggagggctggtgcagcctggaggctccctgaggctgtcctgcgctgcttccggatttaacatcaaggacacctacattcactgggtgagacaggcccctggaaagggcttggagtgggtggccaggatttaccccacaaatggatacaccagatatgccgatagcgtgaagggcagattcaccatttcagccgacacaagcaagaataccgcctacctgcagatgaacagcttgagagccgaagataccgccgtctactattgcagccggtggggcggcgacggcttctacgccatggactactggggacagggaaccctggtgaccgtgagcagcggaggatcaggaggaggttctggcggagggtctggaggggggagcggaggtgggagcggggacatccagatgacccagagcccctccagcctgtctgcctccgtgggagatcgggtgaccattacatgccgggccagccaggatgtgaacaccgccgtggcctggtaccagcagaagccaggaaaggcccccaagctgctgatttactctgccagtttcctgtactcaggcgtgccttcccgatttagtggcagcaggagcggaactgatttcactctgaccatcagcagcctgcagcccgaggatttcgccacctactactgccagcagcactacaccacccccccaaccttcggccagggcaccaaggtggagattaag

SEQ ID NO: 199 - DNA, 726 bp, synthetic construct
  source 1..726: mol_type = other DNA; note = HER2-3 scFv
gaggtgcagctggtggagtccggaggagggctggtgcagcctggaggctccctgaggctgtcctgcgctgcttccggatttaacatcaaggacacctacattcactgggtgagacaggcccctggaaagggcttggagtgggtggccaggatttaccccacaaatggatacaccagatatgccgatagcgtgaagggcagattcaccatttcagccgacacaagcaagaataccgcctacctgcagatgaacagcttgagagccgaagataccgccgtctactattgcagccggtggggcggcgacggcttctacgccatggactactggggacagggaaccctggtgaccgtgagcagcgggggaggcgggagcggagggggtggtagtgggggaggagggtccgacatccagatgacccagagcccctccagcctgtctgcctccgtgggagatcgggtgaccattacatgccgggccagccaggatgtgaacaccgccgtggcctggtaccagcagaagccaggaaaggcccccaagctgctgatttactctgccagtttcctgtactcaggcgtgccttcccgatttagtggcagcaggagcggaactgatttcactctgaccatcagcagcctgcagcccgaggatttcgccacctactactgccagcagcactacaccacccccccaaccttcggccagggcaccaaggtggagattaag

SEQ ID NO: 200 - DNA, 741 bp, synthetic construct
  source 1..741: mol_type = other DNA; note = HER2-5 scFv
gaggtgcagctggtggagtccggaggagggctggtgcagcctggaggctccctgaggctgtcctgcgctgcttccggatttaacatcaaggacacctacattcactgggtgagacaggcccctggaaagggcttggagtgggtggccaggatttaccccacaaatggatacaccagatatgccgatagcgtgaagggcagattcaccatttcagccgacacaagcaagaataccgcctacctgcagatgaacagcttgagagccgaagataccgccgtctactattgcagccggtggggcggcgacggcttctacgccatggactactggggacagggaaccctggtgaccgtgagcagcgggggaggtggctctggaggcggaggctctgggggtggaggatcagggggtggggggtcagacatccagatgacccagagcccctccagcctgtctgcctccgtgggagatcgggtgaccattacatgccgggccagccaggatgtgaacaccgccgtggcctggtaccagcagaagccaggaaaggcccccaagctgctgatttactctgccagtttcctgtactcaggcgtgccttcccgatttagtggcagcaggagcggaactgatttcactctgaccatcagcagcctgcagcccgaggatttcgccacctactactgccagcagcactacaccacccccccaaccttcggccagggcaccaaggtggagattaag

SEQ ID NO: 201 - protein, 336 aa, synthetic construct
  source 1..336: note = SLNK44
TTTTPAPRPPTPAPTIASQPLSLRPEACRPAAGGAVHTRGLDFACDASSNLFVASWIAVMIIFRIGMAVAIFCCFFFPSGSGGSGGSNRRRRRERRDLFTESWDTQKAPNNYRSPISTSQPTNQSMDDTREDIYVNYPTFSRRPKTRVSLGGGSGGSYKVGFFKRNLKEKMEAGRGVPNGIPAEDSEQLASGQEAGDPGCLKPLHEKDSESGGGKDTGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 202 - protein, 646 aa, synthetic construct
  source 1..646: note = HER2-2 SLNK12
MALPVTALLLPLALLLHAARPGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGSGGGSGGGSGGGSGGGSGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGILTTTTPAPRPPTPAPTIASQPLSLRPEACRPAAGGAVHTRGLDFACDASSNLFVASWIAVMIIFRIGMAVAIFCCFFFPSGSGGSGGSICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAITGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 203 - protein, 809 aa, synthetic construct
  source 1..809: note = HER2-2 SLNK12-sIL15
MALPVTALLLPLALLLHAARPGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGSGGGSGGGSGGGSGGGSGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGILTTTTPAPRPPTPAPTIASQPLSLRPEACRPAAGGAVHTRGLDFACDASSNLFVASWIAVMIIFRIGMAVAIFCCFFFPSGSGGSGGSICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAITGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 204 - protein, 641 aa, synthetic construct
  source 1..641: note = HER2-3 SLNK12
MALPVTALLLPLALLLHAARPGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGILTTTTPAPRPPTPAPTIASQPLSLRPEACRPAAGGAVHTRGLDFACDASSNLFVASWIAVMIIFRIGMAVAIFCCFFFPSGSGGSGGSICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAITGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 205 - protein, 804 aa, synthetic construct
  source 1..804: note = HER2-3 SLNK12-sIL15
MALPVTALLLPLALLLHAARPGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGILTTTTPAPRPPTPAPTIASQPLSLRPEACRPAAGGAVHTRGLDFACDASSNLFVASWIAVMIIFRIGMAVAIFCCFFFPSGSGGSGGSICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAITGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 206 - protein, 646 aa, synthetic construct
  source 1..646: note = HER2-5 SLNK12
MALPVTALLLPLALLLHAARPGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGILTTTTPAPRPPTPAPTIASQPLSLRPEACRPAAGGAVHTRGLDFACDASSNLFVASWIAVMIIFRIGMAVAIFCCFFFPSGSGGSGGSICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAITGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 207 - protein, 809 aa, synthetic construct
  source 1..809: note = HER2-5 SLNK12-sIL15
MALPVTALLLPLALLLHAARPGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGILTTTTPAPRPPTPAPTIASQPLSLRPEACRPAAGGAVHTRGLDFACDASSNLFVASWIAVMIIFRIGMAVAIFCCFFFPSGSGGSGGSICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAITGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 208 - DNA, 486 bp, synthetic construct
  source 1..486: mol_type = other DNA; note = sIL15
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 209 - DNA, 750 bp, synthetic construct
  source 1..750: mol_type = other DNA; note = BCMA/TACI CAR3v1
gacatccagttgacccagtctccatcctccctgtctgcaagcgttggagacagagttactatcacttgccgggcaagtcagagcattagcctatatttaaattggtatcagcagaaaccagggaaagcccctaagttgctgatctatgctgcatctagtttgcaaagtggggtcccatcacgattcagtggcagtggatccgggacagatttcactctcaccatctcgagtctacaacctgaagattttgcaacttactactgtcagcaagtggccgtcgccccttggactttcggcggagggaccaaggttgagatcaaagggagcacaagcggctctgggaaaccgggatctggcgagggatctaccaagggccaggtgcagctggtgcagtctggggctgaggtgaaaaagcctgggtcctcggtgaaggtctcctgcaaggcttctggaggcaccttcgaacactatgctatcagctgggtgcgacaggcccctggacagggacttgagtggatgggggggatcatccccatactaggccgagcaaactacgcacagaagttccagggcagagtcactattaccgcggacgaatcgacgagcacagcctacatggagctgagcagcctgagatctgaggacacggcggtgtattactgcgcccgtgacagaagctgggaaggatctccctatatgtactacggaatggacgtttggggccaagggacaatggttaccgttagcagt

SEQ ID NO: 210 - protein, 250 aa, synthetic construct
  source 1..250: note = BCMA/TACI CAR3v1
DIQLTQSPSSLSASVGDRVTITCRASQSISLYLNWYQQKPGKAPKLLIYAASSLQSGVPSRFSGSGSGTDFTLTISSLQPEDFATYYCQQVAVAPWTFGGGTKVEIKGSTSGSGKPGSGEGSTKGQVQLVQSGAEVKKPGSSVKVSCKASGGTFEHYAISWVRQAPGQGLEWMGGIIPILGRANYAQKFQGRVTITADESTSTAYMELSSLRSEDTAVYYCARDRSWEGSPYMYYGMDVWGQGTMVTVSS

SEQ ID NO: 211 - DNA, 750 bp, synthetic construct
  source 1..750: mol_type = other DNA; note = BCMA/TACI CAR1v2
gacatccagatgacccagtctccatcctccctgtctgcaagcgttggagatagagtcactatcacttgccgggcaagtcagagcattagcagctatttaaattggtatcagcagaaaccagggaaagcccctaagctcctgatctatgctgcatccagtttgcaaagtggggtcccatcaaggttcagtggcagtggatccgggacagatttcactctcaccatcagcagtctgcaacctgaagattttgcaacttactactgtcagcaaagccacatcgccccttggacttttggcggagggaccaaggttgagatcaaagggagcactagcggctctggcaaacctggatctggcgagggatctaccaagggccaggtgcagctggtgcagtctggggctgaggtgaagaagcctgggtcctcggtgaaggtctcctgcaaggcttctggaggcaccttcgcagactatgctatcagctgggtgcgacaggcccctggacaagggcttgagtggatgggagggatcatccctatattgggcagagcaaactacgcacagaagttccagggcagagttacgattaccgcggacgaatccacgagcacagcctacatggagctgagcagcctgagatctgaggacacggcggtgtactactgcgccagagacagagacagcacaagcctgccgtacaaccactactacatggacgtatggggcaagggtacaactgtcactgtctcctct

SEQ ID NO: 212 - protein, 250 aa, synthetic construct
  source 1..250: note = BCMA/TACI CAR1v2
DIQMTQSPSSLSASVGDRVTITCRASQSISSYLNWYQQKPGKAPKLLIYAASSLQSGVPSRFSGSGSGTDFTLTISSLQPEDFATYYCQQSHIAPWTFGGGTKVEIKGSTSGSGKPGSGEGSTKGQVQLVQSGAEVKKPGSSVKVSCKASGGTFADYAISWVRQAPGQGLEWMGGIIPILGRANYAQKFQGRVTITADESTSTAYMELSSLRSEDTAVYYCARDRDSTSLPYNHYYMDVWGKGTTVTVSS

SEQ ID NO: 213 - DNA, 750 bp, synthetic construct
  source 1..750: mol_type = other DNA; note = BCMA/TACI CAR3v2
gacatccagttgacccagtctccatcctccctgtctgcaagcgttggagacagagttactatcacttgccgggcaagtcagagcattagcctatatttaaattggtatcagcagaaaccagggaaagcccctaagctcctgatctatgctgcatccagtttgcaaagtggggtcccatcaaggttcagtggcagtggatccgggacagatttcactctcaccatcagcagtctgcaacctgaagattttgcaacttactactgtcagcaagtggccgtcgccccttggactttcggcggagggaccaaggttgagatcaaagggagcacaagcggctctggcaaacctggatctggcgagggatctaccaagggccaggtgcagctggtgcagtctggggctgaggtgaagaagcctgggtcctcggtgaaggtctcctgcaaggcttctggaggcaccttcgaacactatgctatcagctgggtgcgacaggcccctggacaggggcttgagtggatgggagggatcatccccatattgggccgagcaaactacgcacagaagttccagggcagagtcacgattaccgcggacgaatccacgagcacagcctacatggagctgagcagcctgagatctgaggacacggcggtgtactactgcgccagagacagaagctgggaaggatctccctatatgtactacggaatggacgtttggggccaagggacaatggtcaccgtttcctca

SEQ ID NO: 214 - protein, 250 aa, synthetic construct; identical to SEQ ID NO: 210
  source 1..250: note = BCMA/TACI CAR3v2

SEQ ID NO: 215 - DNA, 750 bp, synthetic construct
  source 1..750: mol_type = other DNA; note = ADI-53419
gacatccagatgacacagagccctagcagcctgtctgcctctgtgggcgatagagtgaccatcacctgtagagccagccagagcatcagcagctacctgaactggtatcagcaaaagcccggcaaggcccctaaactgctgatctatgctgccagctctctgcagtctggcgtgccaagcagattttctggcagcggctctggcaccgacttcaccctgacaatttctagcctgcagcctgaggacttcgccacctactactgccagcagtctcacattgccccttggacctttggcggaggcaccaaggtggaaatcaagggctctacaagcggcagcggaaagcctggatctggcgagggatctacaaaaggccaggtgcagctggtgcagagcggagccgaagtgaagaaacctggcagctctgtgaaggtgtcctgcaaggcctctggcggaacattcgccgattacgccatcagctgggttcgacaggctcctggacaaggcttggagtggatgggcggaatcatccctatcctgggcagagccaactacgcccagaaattccagggccgcgtgacaatcacagccgatgagagcacaagcaccgcctacatggaactgtccagcctgagaagcgaggacaccgccgtgtactactgcgccagagacagagatagcaccagcctgccttacaaccactactacatggacgtgtggggcaagggcaccaccgtgacagttagctct

SEQ ID NO: 216 - protein, 250 aa, synthetic construct; identical to SEQ ID NO: 212
  source 1..250: note = ADI-53419

SEQ ID NO: 217 - protein, 30 aa, synthetic construct
  source 1..30: note = CD28 Stalk (Alternative Coding Sequence)
LDNEKSNGTIIHVKGKHLCPSPLFPGPSKP

SEQ ID NO: 218 - DNA, 81 bp, synthetic construct
  source 1..81: mol_type = other DNA; note = CD28 TM (Alternative Coding Sequence)
ttttgggtactggtggtggtgggaggagtgctggcttgttactctttactggtcaccgtggccttcatcatcttctgggtt

SEQ ID NO: 219 - DNA, 126 bp, synthetic construct
  source 1..126: mol_type = other DNA; note = CD28 Co-Stim (Alternative Coding Sequence)
cgaagcaagaggtctagactgctgcacagcgactacatgaacatgacccccagaagacccggccccaccagaaagcactaccagccttacgcccctccccgcgacttcgccgcctatcgtagcctg

SEQ ID NO: 220 - protein, 42 aa, synthetic construct
  source 1..42: note = CD28 Co-Stim (Alternative Coding Sequence)
RSKRSRLLHSDYMNMTPRRPGPTRKHYQPYAPPRDFAAYRSL

SEQ ID NO: 221 - protein, 480 aa, synthetic construct
  source 1..480: note = CD8-aTM-2B4-CD3-TLR2
TTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGSGGSICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAI

SEQ ID NO: 222 - protein, 778 aa, synthetic construct
  source 1..778: note = CD8-aTM-2B4-CD3-TLR2
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILTTTTPAPRPPTPAPTIASQPLSLRPEACRPAAGGAVHTRGLDFACDASSNLFVASWIAVMIIFRIGMAVAIFCCFFFPSGSGGSGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSLGGGSGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGSGGSICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAI

SEQ ID NO: 223 - DNA, 2334 bp, synthetic construct
  source 1..2334: mol_type = other DNA; note = CD8-aTM-2B4-CD3-TLR2
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccacaaccacaccagcacctcggccacccaccccagcaccaacaatcgccagccagcctctgtccctgaggccagaggcatgcaggccagcagcaggaggagcagtgcacaccagaggcctggactttgcctgtgatgctagctccaacctgtttgtggcctcttggatcgccgtgatgatcatctttcggatcggcatggccgtggccatcttctgctgtttctttttccccagcggatccggcggctctggcggcagctggcggagaaagcggaaggagaagcagtctgagaccagccccaaggagttcctgacaatctacgaggacgtgaaggatctgaagaccaggcgcaaccacgagcaggagcagacctttcctggcggcggcagcacaatctattccatgatccagtctcagagctccgcccccacctcccaggagcctgcctacacactgtatagcctgatccagccttcccggaagtccggctctcggaagagaaaccacagcccatccttcaattctacaatctacgaagtgatcggcaagagccagccaaaggcacagaaccccgccaggctgagccgcaaggagctggagaattttgacgtgtattcttccctaggaggcggctccggcggctctagagtgaagttcagcaggtccgccgacgcacctgcataccagcagggacagaaccagctgtataacgagctgaatctgggccggagagaggagtacgacgtgctggataagaggcggggccgggaccccgagatgggaggcaagccacggagaaagaacccccaggagggcctgtacaatgagctgcagaaggacaagatggccgaggcctattccgagatcggcatgaagggagagaggcgccggggcaagggacacgatggcctgtaccagggcctgtctaccgccacaaaggacacctatgatgccctgcacatgcaggccctgccccctcggaccggtggcggctctggcggcagcatctgctacgacgcctttgtgagctattccgagcgggatgcctactgggtggagaacctgatggtgcaggagctggagaactttaatccccctttcaagctgtgcctgcacaagagggacttcatccctggcaagtggatcatcgacaacatcatcgattccatcgagaagtctcacaagaccgtgtttgtgctgagcgagaatttcgtgaagtccgagtggtgcaagtatgagctggatttctctcactttcggctgttcgacgagaacaatgatgccgccatcctgatcctgctggagccaatcgagaagaaggccatcccccagcggttttgtaagctgagaaagatcatgaacaccaagacatacctggagtggcctatggacgaggcccagagggagggcttctgggtgaatctgcgcgccgccatc

SEQ ID NO: 224 - protein, 336 aa, synthetic construct; identical to SEQ ID NO: 201
  source 1..336: note = CD8-aTM-DNAM1-LFA1-CD3

SEQ ID NO: 225 - protein, 596 aa, synthetic construct
  source 1..596: note = CD8-aTM-DNAM1-LFA1-CD3
MALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGILMALPVTALLLPLALLLHAARPYPYDVPDYAYPYDVPDYAYPYDVPDYAGSSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSGIL

SEQ ID NO: 226 - DNA, 1902 bp, synthetic construct
  source 1..1902: mol_type = other DNA; note = CD8-aTM-DNAM1-LFA1-CD3
atggcactgcctgtgaccgccctgctgctgccactggccctgctgctgcacgccgccaggccttatccatacgacgtgcctgattatgcctacccttatgacgtgccagattacgcctatccctacgacgtgcctgattacgccggctcgagtgacatccagatgacccagaccacatctagcctgtccgcctctctgggcgacagggtgacaatctcctgcagagcctctcaggatatcagcaagtatctgaactggtatcagcagaagcccgatggcaccgtgaagctgctgatctatcacacatccaggctgcactctggcgtgcctagccgcttcagcggctccggctctggcaccgactactccctgacaatctctaacctggagcaggaggatatcgccacctatttctgccagcagggcaataccctgccatacacatttggcggcggcacaaagctggagatcaccggcagcacatccggctctggcaagccaggcagcggagagggctccaccaagggagaggtgaagctgcaggagtccggaccaggcctggtggcacccagccagtccctgtctgtgacctgtacagtgtctggcgtgagcctgcccgactacggcgtgtcttggatcagacagccccctagaaagggcctggagtggctgggcgtgatctggggcagcgagaccacatactataattctgccctgaagagccggctgaccatcatcaaggacaacagcaagtcccaggtgttcctgaagatgaacagcctgcagaccgacgatacagccatctactattgtgccaagcactactattacggcggcagctatgctatggactactggggccagggcacctccgtgacagtgtctggaattcttaccaccacaacgcccgctcctcggccaccgacgccagcgccaactattgcgagtcagcctctcagtctgcgacctgaggcttgtcgaccagcagccggaggcgcagtgcacacgagggggctggacttcgcctgtgatgctagctccaacctttttgtggcgtcttggatagcggtgatgataatatttaggattgggatggcggtagcgatattttgctgctttttcttccctagcggatccggaggttctggtggatcaaacagaaggagaaggagagagagaagagatctatttacagagtcctgggatacacagaaggcacccaataactatagaagtcccatctctaccagtcaacctaccaatcaatccatggatgatacaagagaggatatttatgtcaactatccaaccttctctcgcagaccaaagactagagtttccctaggaggaggttctggtggatcatataaggttgggttctttaagagaaaccttaaagaaaagatggaggcagggcgcggagtaccaaacggaataccagccgaagactctgaacaacttgcctctggccaggaggctggtgatcccggatgcttgaaacccctgcatgaaaaggattccgagtctggcggaggtaaagataccggtggaggttctggtggatcaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

(19) United States
(12) Patent Application Publication
(10) Pub. No.: US 2025/0213615 A1
(43) Pub. Date: Jul. 3, 2025

(54) CHIMERIC ANTIGEN RECEPTORS FOR NATURAL KILLER CELLS AND USES THEREOF IN IMMUNOTHERAPY

(71) Applicant: SHORELINE BIOSCIENCES, INC., San Diego, CA (US)

(72) Inventors: Lee Andrew Boland SWANSON, San Diego, CA (US); David Thomas RODGERS, San Diego, CA (US); Huang ZHU, San Diego, CA (US)

(73) Assignee: SHORELINE BIOSCIENCES, INC., San Diego, CA (US)

(21) Appl. No.: 18/851,949

(22) PCT Filed: Mar. 29, 2023
(86) PCT No.: PCT/US2023/065102

§ 371 Date: Sep. 27, 2024

Related U.S. Application Data
(60) Provisional application No. 63/325,441, filed on Mar. 30, 2022.
(60) Provisional application No. 63/440,169, filed on Jan. 20, 2023.

(51) Int. Cl. A61K 35/17; A61K 40/15; A61K 40/31; A61K 40/42; A61P 35/00; C07K 16/28; C07K 16/32; C12N 5/0783

(52) U.S. Cl. CPC A61K 35/17; A61K 40/15; A61K 40/31; A61K 40/4205; A61K 40/4211; A61K 40/4221; A61P 35/00; C07K 16/2803; C07K 16/2878; C07K 16/2887; C07K 16/32; C12N 5/0646; A61K 2239/13; A61K 2239/17; A61K 2239/21; C07K 2317/565; C07K 2317/622

(57) ABSTRACT

Provided are natural killer cells engineered to express a chimeric antigen receptor (CAR) with Toll/interleukin-1 (IL-1) receptor (TIR) signaling domains, such as a Toll-like receptor (TLR) signaling domain or a IL-1 receptor (IL-1R) subfamily signaling domain. Further provided are compositions and methods for making and using such natural killer cells in immunotherapy for cancer. The CAR-NK cells may be CISH−/−iPSC-NK cells.

### CROSS REFERENCE

This application claims the benefit of U.S. Provisional Application No. 63/325,441, filed Mar. 30, 2022, and U.S. Provisional Application No. 63/440,169, filed Jan. 20, 2023, the entirety of which are hereby incorporated by reference herein in their entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14735-026-228_SEQ LISTING.xml”, was created on Mar. 28, 2023, and is 347,806 bytes in size.

### FIELD OF INVENTION

The invention relates to chimeric antigen receptors (CARs) for natural killer (NK) cells and uses thereof in immunotherapy. In particular, the invention relates to CARs having a cytoplasmic portion that includes a Toll/interleukin-1 (IL-1) receptor (TIR) signaling domain.

### BACKGROUND

NK cells are cytotoxic lymphocytes that constitute a major component of the innate immune system. They were named “natural killers” because of the initial notion that they do not require prior activation in order to kill a target. In humans, a natural killer cell usually expresses the surface markers CD16 (FCyRIII) and CD56. NK cells generally have cytotoxic activity. Endogenous NK cells provide rapid responses to virally infected cells and respond to transformed cells. Small granules in cytoplasm contain perforin, granzymes, and other bioactive molecules. Upon contacting a target cell, the NK cells releases these granules. Perforin forms pores in the cell membrane of the target cell through which the granzymes and associated molecules can enter and act to induce apoptosis of the target cell.

NK cells are considered to be innate lymphoid cells, in contrast to adaptive lymphoid cells (T cells and B cells) and myeloid cells (such as macrophages). Unlike T cells, which specifically detect peptides from pathogens presented by Major Histocompatibility Complex (MHC) molecules on the surface of infected cells, NK cells recognize stressed cells regardless of whether peptides from pathogens are present on MHC molecules. Rather receptors on the cell surface on NK cells that recognize other biomolecules control activation of NK cells.

Artificial NK cells may be made from stem cells, such as induced pluripotent stem cells, by known methods such as those described in Zhu and Kaufman., Methods Mol Biol. 2048:107-119 (2019)). Of particular interest are NK cells having inactivating mutation is the gene encoding cytokine-inducible SH2-containing protein (CISH). These mutations include, but are not limited to substitutions, insertions, and/or deletions. CISH is an important negative regulator of IL-15 signaling. Inactivation of the CISH gene in induced pluripotent stem cells (iPSC) followed by differentiation of iPSC into natural killer (NK) cells results in engineered NK cells having increased persistence and potency relative to NK cells in which CISH has not been inactivated, as described in US 2021/0145883 A1. Inactivation of CISH is also thought to be able to enhance T cell cytotoxicity in cancer therapies.

The innate targeting capacity of NK cells can be redirected to a selected cell population by engineering the NK cells to express a chimeric antigen receptor (CAR). Chimeric antigen receptors have an extracellular portion that includes a ligand-binding domain designed to specifically bind a target antigen; a transmembrane domain; and a cytoplasmic portion that includes one or more signaling domains. Various architectures for the cytoplasmic portion of CARs are known. First generation CARs contained only the signaling domain to the T cell receptor zeta chain, termed a CD3ζ domain. Later generations of CARs incorporated domains from co-stimulatory molecules, notably the signaling domains of Cluster of Differentiation 28 (CD28) and 41BB (also known as CD137).

There remains a need in the art for chimeric antigen receptors for use in engineered natural killer (NK) cells and uses thereof. The present invention addresses that need.

### SUMMARY OF THE INVENTION

Provided are chimeric antigen receptors for use in engineered natural killer (NK) cells and uses thereof. In one aspect, the disclosure provides chimeric antigen receptors (CARs) having a cytoplasmic portion that includes a Toll/interleukin-1 (IL-1) receptor (TIR) signaling domain.

In an aspect, the disclosure provides CARs having a cytoplasmic portion that includes a Toll-like receptor (TLR) signaling domain. As used herein, the term “TLR signaling domain” refers to the TIR domain of a TLR receptor, or a functional variant thereof. The TLR signaling domain may be a TLR1, TLR2, TLR3, TLR5, TLR6, TLR7, TLR8, TLR9, or TLR10 signaling domain—preferably a TLR2 signaling domain.

Accordingly, the cytoplasmic portion of the CAR may comprise a signaling domain having a polypeptide sequence selected from SEQ ID NOs: 1-10, or a functional variant thereof. The signaling domain may have a polypeptide sequence at least 55%, at least 70%, at least 75%, at least 80%, at least 90%, at least 95%, or 100% identical to any one of SEQ ID NOs: 1-10.

In a further aspect, the disclosure provides CARs having a cytoplasmic portion that includes an interleukin-1 receptor (IL-1R) subfamily signaling domain. As used herein, the term “IL-1R subfamily signaling domain” refers to the TIR domain of a member of the IL-1R receptor subfamily, or a functional variant thereof. The IL-1R subfamily signaling domain may an IL-1R signaling domain or an interleukin 18 receptor (IL-18R) signaling domain.

Accordingly, the cytoplasmic portion of the CAR may comprise a signaling domain having a polypeptide sequence selected from SEQ ID NOs: 11 or 12, or a functional variant thereof. The signaling domain may have a polypeptide sequence at least 55%, at least 70%, at least 75%, at least 80%, at least 90%, at least 95%, or 100% identical to any one of SEQ ID NOs: 21-22.

In another aspect, the disclosure provides CARs having a cytoplasmic portion that includes a TIR signaling domain and a CD3ζ domain (also referred to herein as “CD3-zeta”)-preferably a CD3ζ domain at the C-terminus of the CAR. The CARs disclosed herein may further comprise one or more other signaling domains. In variations, the cytoplasmic portion of the CAR may comprise a 2B4 signaling domain or an OX40 signaling domain. In further variations, however, the cytoplasmic portion of CAR lacks a 2B4 signaling domain, lacks an OX40 signaling domain, or lacks both 2B4 and OX40 signaling domains.

The CARs disclosed herein further comprise a transmembrane domain. Various suitable transmembrane domains are known in the art. In variations, the transmembrane domain may be the transmembrane domain of CD8 alpha chain (CD8α), CD28, CD16, NKp44, NKp46, TLR, IL-1R subfamily, or functional variant thereof. The transmembrane domain may be an artificial transmembrane domain (termed “aTM”) having the polypeptide sequence NLFVASWIAVMIIFRIGMAVAIFCCFFFP (SEQ ID NO: 41), or a variant with 1, 2, 3, 4, 5 or more substitutions.

In various embodiments, the disclosure provides natural killer (NK) cells engineered to express these CARs, nucleic acids encoding these CARs, vectors for delivery of such nucleic acids to cells, stem cells comprising such nucleic acids, therapeutic compositions, kits, methods of manufacture, and methods of treatment.

In some embodiments, the disclosure provides a natural killer cell, comprising a chimeric antigen receptor (CAR) comprising an extracellular portion, a transmembrane domain, and a cytoplasmic portion, wherein the cytoplasmic portion comprises a Toll/interleukin-1 (IL-1) receptor (TIR) signaling domain extracellular protein comprises a ligand-binding domain. In certain embodiments, the ligand-binding domain specifically binds a CD19 antigen, a CD20 antigen, a Her2 antigen or a BCMA antigen.

In another aspect, provided herein is a chimeric antigen receptor (CAR) comprising an extracellular portion, a transmembrane domain, and a cytoplasmic portion, wherein the cytoplasmic portion comprises a Toll/interleukin-1 (IL-1) receptor (TIR) signaling domain. In some embodiments, the TIR signaling domain is a Toll-like receptor (TLR) signaling domain. In some embodiments, the TLR signaling domain is a TLR2 signaling domain or a functional variant thereof. In some embodiments, the TLR signaling domain has a polypeptide sequence at least 55%, at least 70%, at least 75%, at least 80%, at least 90%, at least 95%, or 100% identical to SEQ ID NO: 2. In some embodiments, the cytoplasmic portion comprises a CD3ζ signaling domain. In some embodiments, the cytoplasmic portion comprises a 2B4 signaling domain. In some embodiments, the cytoplasmic portion lacks a 2B4 signaling domain. In some embodiments, the cytoplasmic portion consists essentially of, in N- to C-terminal order, a 2B4 signaling domain, an IL-18R signaling domain, and a CD3ζ signaling domain. In some embodiments, the cytoplasmic portion consists essentially of, in N- to C-terminal order, a 2B4 signaling domain, a CD3ζ signaling domain, and a TLR2 signaling domain. In some embodiments, the cytoplasmic portion consists essentially of, in N- to C-terminal order, a 2B4 signaling domain, a TLR2 signaling domain, and a CD3ζ signaling domain.

In some embodiments, the extracellular protein comprises a ligand-binding domain. In some embodiments, the ligand-binding domain comprises an antibody-like domain. In some embodiments, the ligand-binding domain comprises a variable heavy (VH) domain and/or a variable light (VL) domain. In some embodiments, the ligand-binding domain comprises a single-chain variable fragment (scFv). In some embodiments, the ligand-binding domain specifically binds a CD19 antigen, a CD20 antigen, a Her2 antigen, or a BCMA antigen.

In some embodiments, (i) the extracellular domain comprises a polypeptide derived from a CD19 ligand binding domain, a CD20 ligand binding domain, a BCMA binding domain, or a HER2 binding domain; (ii) the transmembrane domain is a transmembrane domain selected from the group consisting of aTM, CD8 alpha chain (CD8α), CD28, CD16, NKp44, NKp46, NKG2, DNAM1, IL-2Rβ, TLR, and IL-1R subfamily; and (iii) the cytoplasmic domain comprises a signaling domain of TLR2, 41BB, or 2B4.

In some embodiments, the cytoplasmic domain further comprises a signaling domain of CD3ζ. In some embodiments, the CAR comprises (i) aTM transmembrane domain, a TLR2 signaling domain, and a CD3zeta signaling domain; or (ii) a CD28 transmembrane domain, a TLR2 signaling domain, and a CD3zeta signaling domain.

In some embodiments, the C-terminus of the extracellular domain is operably linked to the N-terminus or C-terminus of the transmembrane domain without a linker. In some embodiments, the C-terminus of the extracellular domain is operably linked to the N-terminus or C-terminus of the transmembrane domain with a linker or a hinge domain.

In some embodiments, the hinge domain is a CD8a hinge. In some embodiments, the CD8a hinge comprises the amino acid sequence set forth in SEQ ID NO: 158.

In some embodiments, the N-terminus or C-terminus of the transmembrane domain is operably linked to the N-terminus of the cytoplasmic domain, with or without a linker. In some embodiments, the transmembrane domain is operably linked to the cytoplasmic domain with a Gly-Ser linker.

In another aspect, provided herein is a polynucleotide comprising a nucleotide sequence encoding a chimeric antigen receptor of the present disclosure.

In yet another aspect, provided herein is a population of cells comprising an immune cell comprising a chimeric receptor of the present disclosure or a polynucleotide of the present disclosure. In some embodiments, the immune cell is a natural killer (NK) cell. In some embodiments, the NK cell is an induced pluripotent stem cell-derived natural killer (iPSC-NK) cell. In some embodiments, the immune cell comprises homozygous inactivating mutations in a cytokine-inducible SH2-containing protein (CISH) gene. In some embodiments, the immune cell is CISH^{-/-}.

In another aspect, provided herein is a pharmaceutical composition comprising a population of cells of the present disclosure.

In a further aspect, provided herein is a method of killing a target cell comprising contacting a population of target cells with a population of cells of the present disclosure or a pharmaceutical composition of the present disclosure, wherein the ligand-binding domain of the CAR specifically binds on antigen on the target cell, and wherein the cells induce specific killing of the target cells.

In yet another aspect, provided herein is a method of treating cancer in a subject in need thereof, comprising administering a pharmaceutical compositions of the present disclosure to the subject.

Advantages of the invention may include, but are not limited to, increased activity of the disclosed CARs in NK cells or related cell types compared to CARs known in the art.

Other aspects and advantages of the invention, will be apparent from the following description and the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows an illustration of a polynucleotide sequence encoding a chimeric antigen receptor (CAR) with the portions of the CAR labeled. A leader sequence is included to promote membrane integration.

FIG. 1B shows an illustration of a polypeptide sequence of a chimeric antigen receptor (CAR), after cleavage of the N-terminal leader, with the portions of the CAR labeled.

FIG. 2A shows an illustration of an embodiment of a CAR with a generic ligand-binding domain and specified hinge (CD8α) and signaling domains (TLR2 and CD3ζ).

FIG. 2B shows an illustration of an embodiment of a CAR with a generic ligand-binding domain and specified hinge (CD8α) and signaling domains (2B4, TLR2, and CD3ζ).

FIG. 3 shows an illustration of a CAR in the membrane of a cell.

FIG. 4 shows an illustration of an embodiment of CAR in the membrane of a cell with specified hinge (CD8α) and signaling domains (TLR2 and CD3ζ).

FIG. 5A shows nearest neighbor joining phylogenetic tree of the TIR signaling domains generated from an alignment of the polypeptide sequences of the TIR signaling domains

FIG. 5B shows nearest neighbor joining phylogenetic tree of the TLR signaling domains generated from an alignment of the polypeptide sequences of the TLR signaling domains

FIG. 6 shows multiple sequence alignment of the signaling domains of TLR1 (SEQ ID NO: 1), TLR2 (SEQ ID NO: 2), TLR4 (SEQ ID NO: 4), TLR6 (SEQ ID NO: 6), and TLR10 (SEQ ID NO: 10).

FIG. 7 shows a table of pairwise sequence identities of TLR signaling domains.

FIG. 8 shows the sequence of an embodiment of a CAR (SEQ ID NO: 100) divided into the components of the CAR (SEQ ID NOs: 37, 38, 41, 2 and 63).

FIG. 9 shows expression of individual CAR constructs in iPSC-NK cells. Histograms of flow cytometry data showing CAR expression in mature iPSC-derived NK cells. CAR expression was detected using an HA-tag engineered into the extracellular portion of the CAR and an anti-HA antibody (APC).

FIG. 10 shows a bar graph of a CAR expression data. Bar chart showing frequency of CAR expression in iPSC-derived NK cells. Dotted line designates average CAR expression across all constructs.

FIG. 11 shows a long-term killing assay: a line graph showing frequency of NK-specific killing against CD19⁺ Raji target cells using eSight impedance assay. Data is normalized to target cells alone (no NK cells) which is designated by dotted horizontal line. 50% NK cell killing means that there were half as many living cells in the culture at that time point as there were in the untreated (no NK cell) control. An arrow points to the curve for a CAR having an TLR2 signaling domain, and CD3ζ cytoplasmic domain (“TLR2-zeta”).

FIG. 12 shows a 4 hr Caspase 3/7 cytotoxicity assay: a bar graph showing NK-specific killing of CD19⁺ SupB15 target cells after 4 hr co-culture. Dotted line designates specific killing of a reference CAR (“2B4-zeta”). An arrow points to the curve for a CAR having a TLR2 signaling domain, and CD3ζ cytoplasmic domain (“TLR2-zeta”).

FIG. 13A shows illustrations of embodiments of a CAR with a TLR2 signaling domain, optionally a 2B4 signaling domain, and a CD3ζ cytoplasmic domain in three different orders.

FIG. 13B shows a long-term killing assay for the three constructs illustrated in FIG. 13A: a line graph showing frequency of NK-specific killing against CD19⁺ Raji target cells using eSight impedance assay.

FIG. 13C shows a 4 hr Caspase 3/7 cytotoxicity assay for the three constructs illustrated in FIG. 13A.

FIG. 14A shows a bar graph of CAR expression data depicting the frequency of CAR expression following lentivirus transduction of CISH knockout (KO) iPSC-derived NK cells, as detected by recombinant HER2.

FIG. 14B shows a 24 hour killing assay for the CAR constructs referenced in FIG. 14A: a line graph depicts the frequency of NK-specific killing against HER2⁺ target cells using an eSight impedance assay.

FIG. 14C shows a bar graph depicting release of the cytokines IFN-gamma and TNF-alpha after co-culture of HER2⁺ target cells and CISH KO iPSC-derived NK cells expressing the CAR constructs referenced in FIG. 14A.

FIG. 14D shows a bar graph depicting CISH KO iPSC-derived NK killing of a NK cell resistant solid tumor HER2⁺ target cells (2:1 E:T) in a monolayer 24 hours after exposure to CISH KO iPSC-derived NK cells expressing the CAR constructs referenced in FIG. 14A.

FIG. 15A shows a stress test killing assay of CISH KO iPSC-derived NK cells expressing three different CAR constructs each of which comprise the same HER2 binding domain against HER2⁺ target cells using an eSight impedance assay.

FIG. 15B shows a stress test killing assay of CISH KO iPSC-derived NK cells expressing the same CAR construct and three different HER2 binding domains against HER2″ target cells using an eSight impedance assay.

FIG. 16A shows a line graph depicting a caspase 3/7 cytotoxicity assay showing NK killing of HER2⁺ target cells after a 4 hr co-culture with iPSC-derived NK cells expressing the same CAR construct and three different HER2 binding domains.

FIG. 16B shows representative images of HER2+ target cell killing and caspase 3/7 activation after a 30 hr co-culture of NK cells and a NK cell resistant solid tumor target (2:1 E:T).

FIG. 17 shows representative images of HER2+ 3D tumor spheroids after 18 hours and 36 hours of treatment with untransduced iPSC-derived NK cells (no CAR expression) and iPSC-derived NK cells expressing the same CAR construct and three different HER2 binding domains.

FIG. 18 shows a serial killing assay by iPSC-derived NK cells either expressing a CAR having a HER2 binding domain or not expressing a CAR (UT) against HER2⁺ target cells using eSight impedance assay.

FIG. 19 shows representative images of HER2+ 3D tumor spheroids on day 0 and day 3 of treatment with NK cells (“UT”) and NK cells expressing a CAR having a HER2 binding domain.

FIG. 20 shows an illustration of a triple edited (“TE”) iPSC cell according to the present disclosure.

FIG. 21 shows representative histograms of flow cytometry data depicting CAR and NK marker expression in wild type (WT) NK cells, CISH KO iPSC-derived NK cells, and triple edited (“TE”) iPSC-derived NK cells.

FIG. 22A shows a long-term killing assay in HER2+ 3D tumor spheroids depicting the frequency of NK-killing against HER2⁺ target cells by WT NK cells, CISH KO iPSC-derived NK cells, and triple edited (“TE”) iPSC-derived NK cells.

FIG. 22B shows representative images of HER2+ 3D tumor spheroids after day 0 and day 3 of co-culture with either WT NK cells, CISH KO iPSC-derived NK cells, and triple edited (“TE”) iPSC-derived NK cells.

FIG. 23A and FIG. 23B show a synergistic effect of sIL-15 co-expression with a HER2-CAR construct on NK cell killing against HER+ breast cancer line, BT474. Comparison is made to cells expressing an anti-CD20 CAR. CAR expression with and without sIL-15 is shown in FIG. 23A and NK cell killing against BT474 clone 5, with and without sIL-15, is shown in FIG. 23B.

### DETAILED DESCRIPTION OF THE INVENTION

The invention relates to chimeric antigen receptors (CARs) for natural killer (NK) cells and uses thereof in immunotherapy. In some variations, the invention relates to CARs having a cytoplasmic portion that includes a Toll/interleukin-1 (IL-1) receptor (TIR) signaling domain.

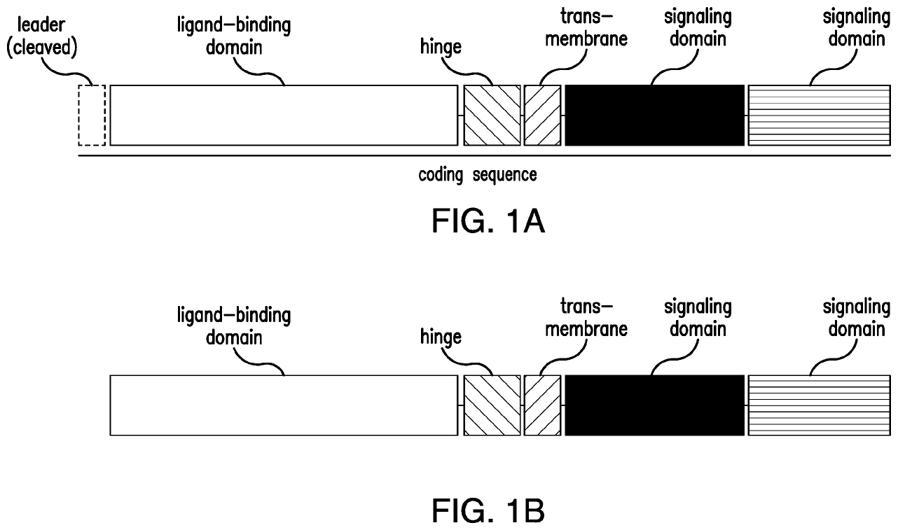

In various embodiments, the disclosure provides natural killer (NK) cells engineered to express the disclosed CARs, nucleic acids encoding these CARs, vectors for delivery of such nucleic acids to cells, stem cells comprising such nucleic acids, therapeutic compositions, kits, methods of manufacture, and methods of treatment. As shown in FIGS. 1A-1B, CARs of the disclosure may be single polypeptides encoding by single polynucleotide sequences. The CAR may have conventional arrangement of components as illustrated in FIG. 1A. After expression of the CAR as a polypeptide, the leader peptide is cleaved from the CAR as shown in FIG. 1B.

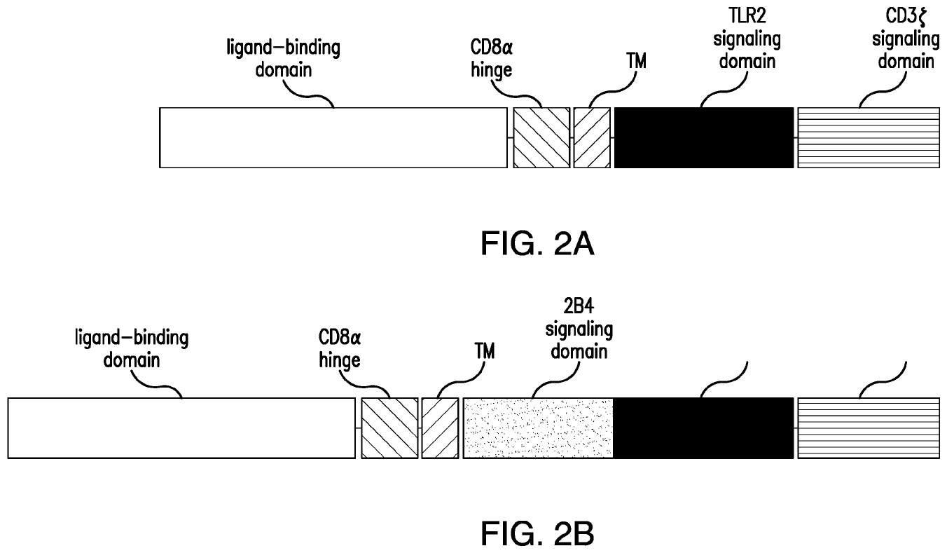

Illustrative embodiments of CARs are shown in FIG. 2A and FIG. 2B. The cytoplasmic portion of the CAR may have a single TIR signaling domain, such as the TLR2 signaling domain shown in FIG. 2A. In this embodiment, the CAR also has a CD3ζ signaling domain. Such a CAR may lack other signaling domains. An embodiment having a 2B4 signaling domain is shown in FIG. 2B.

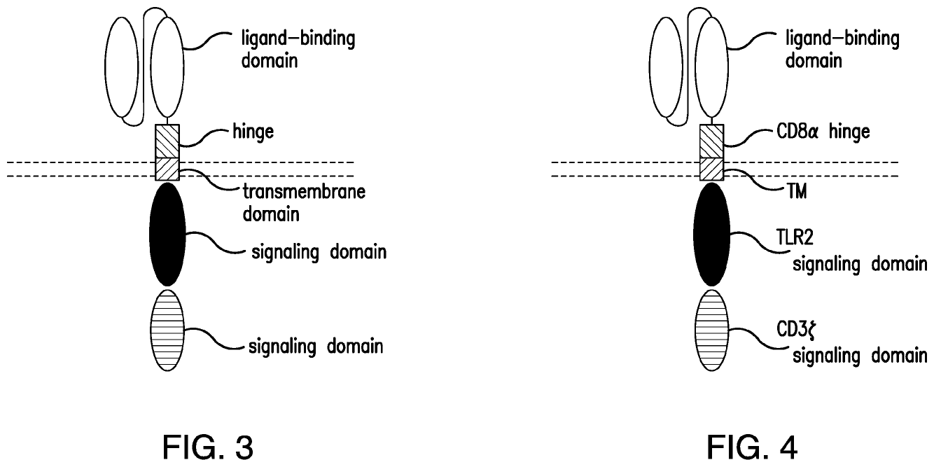

FIG. 3 shows a CAR polypeptide expressing in the membrane of a cell. The ligand-binding domain is depicted as a single-chain variable domain (scFv) though other ligand binding domains may be used. FIG. 4 shows an embodiment of the CAR in which the cytoplasmic portion of the CAR has a single TIR signaling domain, shown as a TLR2 signaling domain, and a CD3ζ signaling domain.

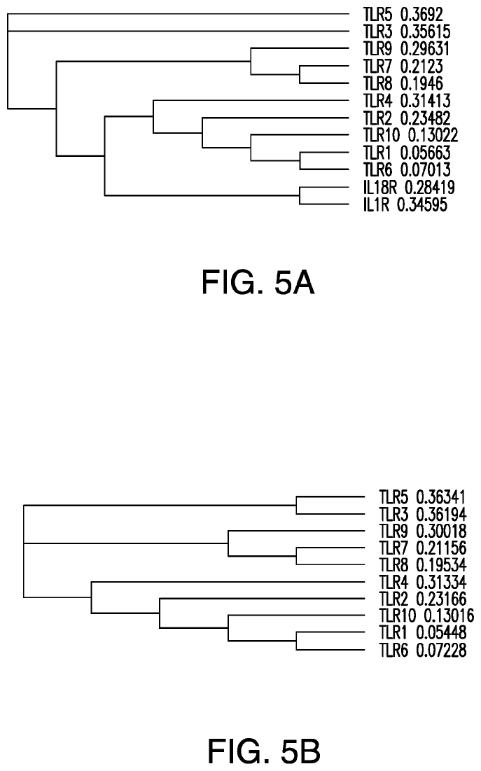

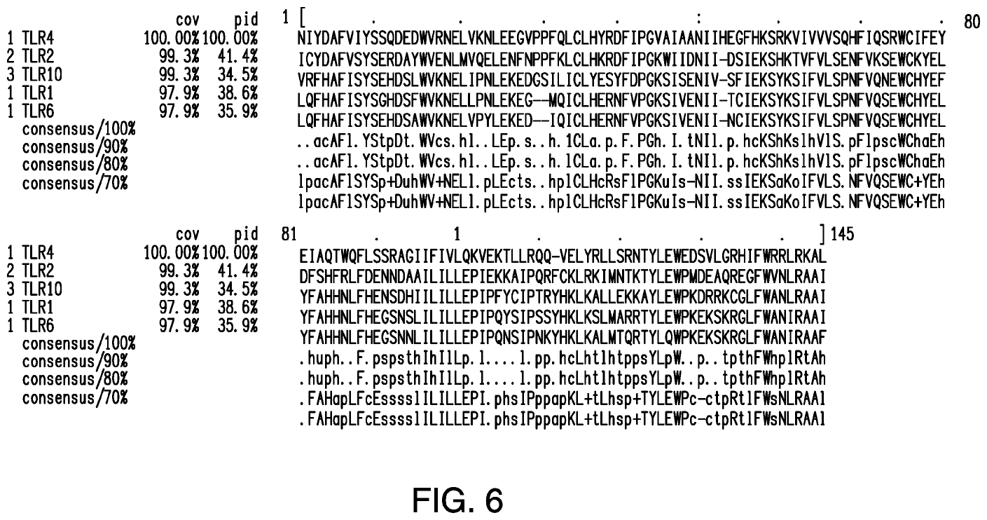

In endogenous receptors, the TIR signaling domains have shared functional attributes. However, they are diverse at the sequence level. FIG. 5A shows a phylogenetic tree to illustrate the sequence divergence between the TIR signaling domains and their clustering into groups. A similar phylogenetic tree for only the TLR signaling domains is shown in FIG. 5B. FIG. 6 shows a sequence alignment of selected TLR signaling domains: TLR1, TLR2, TLR4, TLR6, and TLR10. Functional variants of the TLR signaling domains may be generated using such alignments to select residues that may be varied and conserved residues that should be retained to promote folding and function of the domain.

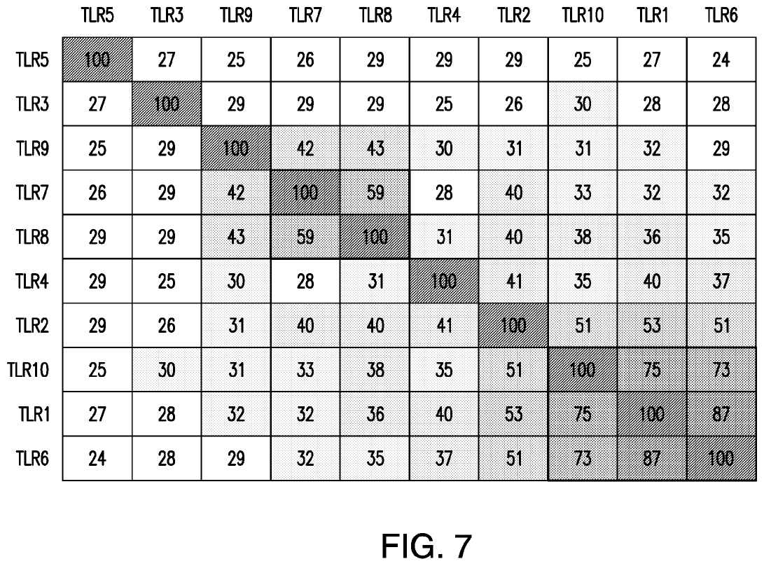

FIG. 7 shows pairwise sequence comparison of TLR signaling domains. Sequence identity between TLR signaling domains is generally less than 55% with few exceptions. For example, sequence identity between TLR2 and TLR4 signaling domains is 41%.

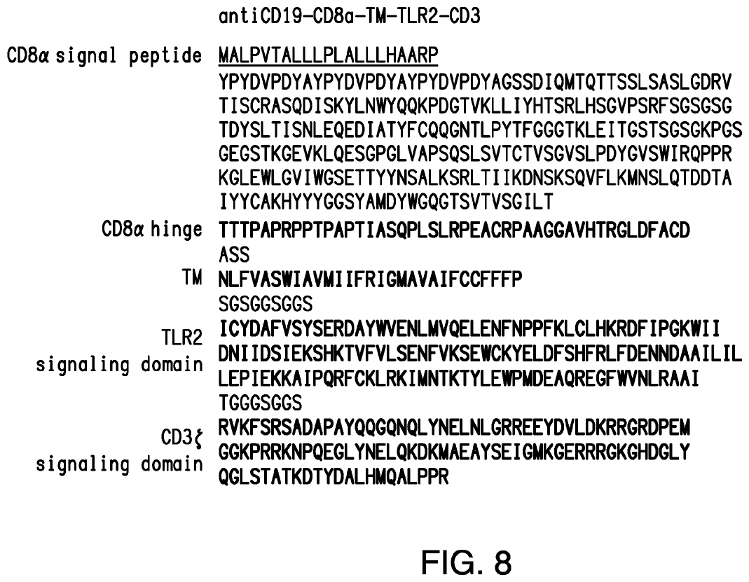

FIG. 8 shows a polypeptide sequence of an embodiment of a CAR. In this embodiment, the CAR comprises a ligand-binding domain specific for CD19. However, as described in detail below, other ligand-binding domains may be used. Furthermore, the disclosure contemplates varying each of the depicted components. Numerous variations upon this embodiment as disclosed.

### Natural Killer Cells

In one aspect, the disclosure provides natural killer (NK) cells engineered to express the disclosed CARs. The NK cells may be primary NK cells. Primary NK cell mays be isolated from umbilical cord blood or peripheral blood using well-known methods. A polynucleotide encoding the CAR may be introduced into the primary NK cell using a viral vector, a non-viral vector, or electroporation. The NK cells may alternatively be induced pluripotent stem cell (iPSC)-derived NK cells or human embryonic stem cell (hESC)-derived NK cells. Methods for making iPSC-NK cells may be obtained by differentiating iPSCs using known methods. In one variation, iPSC-NK cells are made using embryoid bodies. Exemplary methods are provided in US 2013/0287751 A1 and Zhu et al. Methods Mol. Biol. 2048:107-119 (2019). In another variation, hESC-NK cells are made using single cell differentiation, as described, for example, in Woll et al. Blood 113:6094-6101 (2009). In further variations, iPSC-NK cells are made by differentiating single cells. Exemplary methods are provided in US 2021/0024891 A1 and US 2016/0097035 A1. hESC-/iPSC-derived NK cells may be phenotyped by measuring surface marker expression such as CD16, NKG2D, NKp44, NKp46, TRAIL, FasL, or combinations thereof. Killing activity of hESC-/iPSC-derived NK cells may be assessed. Functional assays to assess differentiation into NK cells include direct cytolytic activity tumor cells (such as killing of K562 cells), Caspase-3/7 flow cytometry assay, immunological assays for cytotoxic granule or cytokine release, or antitumor activity in vivo xenograft models. See, e.g., Woll et al. Blood 113:6094-6101 (2009); Hermanson et al. in Hematopoietic differentiation of human pluripotent stem cells. 69-19 (2015).

In some embodiments, the NK cells are CD45+/CD56+ double-positive. In some embodiments, the immune cell is CD45+. In some embodiments, the immune cell is CD56+. In some embodiments the immune cell is CD45+, CD56+, or CD45+/CD56+. Further illustrative methods for making and using engineered cells are provided in U.S. Pat. Appl. Pub. Nos. US 2018/0298101 A1, US 2021/0230548 A1, and US 2021/0145883 A1; and Int'l. Pat. Appl. Pub. Nos. WO 2018/075664 A1 (corresponding to U.S. patent application Ser. No. 17/481,404) and WO 2020/113029 A2 (corresponding to U.S. patent application Ser. No. 17/309,408), the disclosures of which are incorporated by reference herein in their entireties.

The cell population of the disclosure may be a purified cell population. As used herein, a composition containing a “purified cell population” or “purified cell composition” means that at least 30%, 50%, 60%, typically at least 70%, and more preferably 80%, 90%, 95%, 98%, 99%, or more of the cells in the composition are of a similarly identified cell phenotype.

The cell population may be an isolated cell population. As used herein, the term “isolated” means material that is substantially or essentially free from components that normally accompany it in its native state. In particular embodiments, the term “obtained” or “derived” is used synonymously with isolated.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as Molecular Cloning: A Laboratory Manual, second edition (Sambrook el al., 1989) Cold Spring Harbor Press; Oligonucleotide Synthesis (M J. Gait, ed., 1984); Methods in Molecular Biology, Humana Press; Cell Biology: A Laboratory Notebook (J. E. Cellis, ed., 1998) Academic Press; Animal Cell Culture (R. I. Freshney. ed., 1987); Introduction to Cell and Tissue Culture (J. P. Mather and P. E. Roberts, 1998) Plenum Press; Cell and Tissue Culture: Laboratory Procedures (A. Doyle. J. B. Griffiths, and D. G. Newell, eds., 1993-1998) J. Wiley and Sons; Methods in Enzymology (Academic Press, Inc.); Handbook of Experimental Immunology (D. M. Weir and CC. Blackwell, eds.); Gene Transfer Vectors for Mammalian Cells (J. M. Miller and M. P. Calos, eds., 1987); Current Protocols in Molecular Biology (F. M. Ausubel el al., eds., 1987); PCR: The Polymerase Chain Reaction, (Mullis et al., eds., 1994); Current Protocols in Immunology (J. E. Coligan el al., eds., 1991); Short Protocols in Molecular Biology (Wiley and Sons, 1999); Immunobiology (C A. Janeway and P. Travers. 1997); Antibodies (P. Finch. 1997); Antibodies: a practical approach (D. Catty., ed., IRL Press, 1988-1989); Monoclonal antibodies: a practical approach (P. Shepherd and C. Dean, eds., Oxford University Press. 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

Introduction of a polynucleotide encoding a CAR may be achieved by transduction of either the NK cell or an iPSC or hESC precursor with a viral vector, such as a lentiviral vector. Suitable lentiviral vectors are known in the art and are described in, for example, Zufferey et al., (1997); Dull et al., (1998), U.S. Pat. Nos. 6,013,516A, and 5,994,136A. In variations, a non-viral vector, such as a lipid nanoparticle, a transfection reagent, or electroporation may be used to introduce a polynucleotide into a cell.

In an aspect, the disclosure provides cells comprising the a chimeric antigen receptor as described herein and/or a polynucleotide encoding the chimeric antigen receptor. The cell may be a stem cell or a progenitor cell. In some embodiments, the cell is an induced pluripotent stem cell (iPSC). In some embodiments, the cell is an iPSC-derived NK cell.

In some embodiments, the cell comprises homozygous inactivating mutations in the cytokine-inducible SH2-containing protein (CISH) genes of the cell (e.g., CISH^{-/-}). In some embodiments, the cell comprises expression and/or overexpression of a gene encoding a soluble IL-15 or membrane-bound IL-15.

### Chimeric Antigen Receptors

Provided are chimeric antigen receptors for use in engineered natural killer (NK) cells and uses thereof. In one aspect, the disclosure provides chimeric antigen receptors (CARs) having a cytoplasmic portion that includes a Toll-like/interleukin-1 (IL-1) receptor (TIR) signaling domain.

### Toll-Like Receptor Signaling Domains

NK cells are known to express pattern recognitions receptors (PRR), which recognize pathogen-associated molecular patterns (PAMP) on infected cells, including Toll-like Receptors (TLRs). Noh et al. (2020) J. Immunol. Res. 2020:204860.

The Toll/interleukin-1 (IL-1) receptor (TIR) family are defined by have cytoplasmic portions containing structurally homologous signaling domains, termed TIR domains. TIR domains are found in Toll-like Receptors (TLRs) as well as two interleukin receptors, Interleukin-1 Receptor (ILIR) and Interleukin-18 Receptor (IL18), which, though classified as interleukin receptors, participate in signaling pathways similar to those of the TLRs. Accordingly, the TIR family is considered to two subfamilies, defined by the specificity their extracellular domains: (i) the Toll-like receptor (TLR) subfamily and (ii) the IL-1 receptor (IL-1R) subfamily.

In response to extracellular signals, both TLRs can form dimeric receptor complexes, which recruit accessory proteins. Martin et al. (2002) Biochimica et Biophysica Acta 1592:265-280. Humans have ten TLRs, TLR1-TLR10. Signaling by the TLRs depends variously on heterodimerization or homodimerization. TLR4 is known to signal through homodimerization. TLR2 signaling is triggered by heterodimerization with TLR1 or TLR6. It is known that, unlike TLR4, TLR2 does not signal as a homodimer. Zhang, et al. (2002) FEBS Lett. 532:171.

In an aspect, the disclosure provides CARs having a cytoplasmic portion that includes a Toll-like receptor (TLR) signaling domain. As used herein, the term “TLR signaling domain” refers to the TIR domain of a TLR receptor, or a functional variant thereof. The TLR signaling domain may be a TLR1, TLR2, TLR3, TLR5, TLR6, TLR7, TLR8, TLR9, or TLR10 signaling domain-preferably a TLR2 signaling domain.

Accordingly, the cytoplasmic portion of the CAR may comprise a signaling domain having a polypeptide sequence selected from SEQ ID NOs: 1-10, or a functional variant thereof. The signaling domain may have a polypeptide sequence at least 55%, at least 70%, at least 75%, at least 80%, at least 90%, at least 95%, or 100% identical to any one of SEQ ID NOs: 1-10.

When receptors containing Toll/interleukin-1 (IL-1) Receptor (TIR) domains are activated, the TIR domain(s) trigger conserved cellular signal transduction pathways control cell function, through recruitment of cytosolic signaling molecules include TIR domain-containing cytosolic adaptor proteins. TIR signaling domains have been characterized, generally, as comprising three conserved regions (e.g., Box 1 motif: (F/Y) DA or FADFISY (SEQ ID NO: 136), Box 2 motif: RDXXPG (SEQ ID NO: 137) or GYKLC-RD-PG (SEQ ID NO: 138), and Box 3 motif: FW or a conserved W neighbored by basic residues) responsible for mediating protein-protein interactions between toll-like receptors (TLRs) and signal-transduction components (Slack, et al., 2000). Each TIR signaling domain comprises five-stranded parallel beta-sheets (A-through-E) and six surrounding alpha-helices (A-through-E) with respective connecting loop structures (Bovijen, et al., 2013). Conserved residues are located in the hydrophobic core and large insertions and deletions may be present in various loop regions of different TIR signaling domains. For example, differences in the Box 2 motif, comprising the “BB loop” region of the signaling domain, are believed to play a role in specificity of TIR-TIR interactions (Xu, et al., 2000; Poltorak, et al., 1998). About twenty receptors that have TIR domains have been identified in humans, including the Toll-Like Receptors (TLRs) and Interleukin-1 Receptors (IL-1Rs). The general structure of the TIR signaling domain comprises conserved residues within a core sequence that ranges from about 125 to about 200 amino acid residues, however, sequence conservation between these domains can be as low as only 20-30%. The exterior residues between TIR domains vary greatly, resulting in differential distribution of electrostatic potential and resulting signaling functionality (Dunne, et al. 2003). Sequence variation between TIR signaling domains may comprise insertions, deletions, and/or mutations in one or more of the alpha-helices, beta-sheets, loops, or box motifs.

In some embodiments, the TIR signaling domain is a Toll-like receptor (TLR) signaling domain. The human Toll-Like Receptors (TLRs 1-10) are single-pass transmembrane proteins that each recognizing unique ligands, which activates intracellular signaling (Armant, M and Fenton, M, 2002) through TIR signaling domains (Xu et al., 2000; Horng et al., 2002; Brown et al., 2006). TLRs 1, 2, 4, 5, 6, and 10 reside in the plasma membrane and recognize extracellular Pathogen Associated Molecular Patterns (PAMPs). TLRs 3, 7, 8, and 9 reside in intracellular endosomes recognize internalized PAMPs (Akira, et al., 2006; Kawai, T. and Akira, S., 2007). TLRs undergo homo- or hetero-dimerization, which initiates their activation (Gay, N. and Gangloff, M., 2007). The signal cascade is followed by TLR signaling via conserved cytosolic TIR domain present in both the cytoplasmic signaling domain and in the TLR adaptor proteins, such as myeloid differentiation factor 88 (MyD88), MyD88-adaptor-like (MAL/TIRAP), the TRIF-related adaptor molecule (TRAM/TICAM2), TIR domain-containing adaptor protein inducing interferon-β (TRIF/TICAM1), and the sterile α- and armadillo-motif containing protein (SARM) (O'Neill, L. and Bowie, A. 2007). The MyD88-dependent pathway signals MyD88 to the phosphorylation of interleukin-1 receptor-associated kinase (IRAK) 4, then to IRAK1 and IRAK2 (Lin et al., 2010). MyD88 and TRIF interact with kinases TRAM and MAL (Yamamoto, et al., 2003; Yamamoto, et al., 2002). The TRAM and TIRAP pathways recruit MyD88 and TRIF to their respective TLRs. All TLRs, except for TLR3, activate the MyD88-dependent pathway (Horng et al., 2002; Kawai and Akira, 2010). The TRIF-dependent pathway signals through downstream kinases, TANK binding kinase 1 (TBK1) and IKKε, to activate IRF3 and subsequently produce type 1 interferons (Yamamoto et al., 2002b; Oshiumi et al., 2003). The SARM pathway functions as a negative regulator of TRIF-dependent Toll-like receptor signaling (Carty, et al. 2006).

The TLR signaling domain may be a TLR2 signaling domain or a functional variant thereof. TLR2 signaling domains are derived from Toll-Like Receptor 2 (TLR2), a type of TLR that interacts with TLR1, TLR6, or TLR10 prior to stimulation with a ligand (Jin, et al, 2007; Toshchakov, V. and Neuwald, A., 2020). Heterodimer formation facilitates interaction with intracellular TIR domains to promote signal initiation. TLR2 acts via MYD88 and TRAF6, leading to activation of transcription factor NF-kappa-B, cytokine secretion and inflammatory response; and may also promote apoptosis in response to lipid moieties of lipoproteins (Brightbill, et al., 1999; Aliprantis, et al., 1999). The TLR2 signaling domain may comprise residues 639-782 (144 aa) of human TLR2 (SEQ ID NO: 26) or a functional variant thereof (ICYDAFVSYSERDAYWVENLMVQELENFNPPFKLCLHKRDFIPGKWIIDNIIDSIEKSHK TVFVLSENFVKSEWCKYELDFSHFRLFDENNDAAILILLEPIEKKAIPQRFCKLRKIMNTK TYLEWPMDEAQREGFWVNLRAAI (SEQ ID NO: 2)). The TIR signaling domain of TLR2 may comprise the polypeptide sequence: ICYDAFVSYSERDAYWVENLMVQELENFNPPFKLCLHKRDFIPGKWIIDNIIDSIEKSHKT VFVLSENFVKSEWCKYELDFSHFRLFDENNDAAILILLEPIEKKAIPQRFCKLRKIMNTKT YLEWPMDEAQREGFWVNLRAAI (SEQ ID NO: 2) or a functional variant thereof. In some embodiments, the TIR signaling domain of TLR2 is encoded by the nucleotide sequence set forth in SEQ ID NO: 180. In some embodiments, the TIR signaling domain of TLR2 is encoded by a nucleotide sequence having at least 80%, 85%, 90%, 91%, 92%, 93%, 94%, 95%, 96%, 97%, 98%, or 99% identity to SEQ ID NO: 180.

The TLR signaling domain may have a polypeptide sequence at least 55%, at least 70%, at least 75%, at least 80%, at least 90%, at least 95%, or 100% identical to SEQ ID NO: 2. Functional variants and/or some or all of the conserved residues of the TLR2 signaling domain may be retained. Functional variants may include N to C terminal truncations, insertions, and/or mutations. Functional variants may comprise a polypeptide sequence sufficient to promote signal propagation using the TLR2 signaling pathway. Conserved residues of the TLR2 TIR signaling domain may comprise residues 698-707 (Toshchakov, V. and Neuwald, A., 2020). Further conserved residues of the TLR2 TIR may comprise P681, K709, K714, KK742-743, K751, K754, S636, R677, Y715, and/or R753 (Xu, et al., 2000; Mckelvey, et al., 2016; Ben-Ali, et al., 2011; Kang, et al., 2001; Bochud, et al., 2003; Georgel, et al. 2009).

The TLR signaling domain may be a TLR8 signaling domain or a functional variant thereof. TLR8 signaling domains are derived from Toll-Like Receptor 8 (TLR8), a type of TLR that functions as an endosomal, nucleic acid-sensing, TLR capable of undergoing dimerization in the recruitment of TIR-containing downstream adapter MYD88 via homotypic interaction (Tanji, et al., 2013; Tanji, et al., 2015). Resulting signal propagation through the Myddosome is generated by IRAK4, IRAK1, TRAF6, TRAF3 activating NF-kappa-B and IRF7 inducing proinflammatory cytokines and interferons (Qin, et al. 2006; Doyle, et al. 2007; Zhang, et al., 2018). The TLR8 signaling domain may comprise residues 878-1022 of human TLR8 (SEQ ID NO: 32). The TIR signaling domain of TLR8 may comprise the polypeptide sequence: TFYDAYISYDTKDASVTDWVINELRYHLEESRDKNVLLCLEERDWDPGLAIIDNLMQSI NQSKKTVFVLTKKYAKSWNFKTAFYLALQRLMDENMDVIIFILLEPVLQHSQYLRLRQR ICKSSILQWPDNPKAEGLFWQTLRNVV (SEQ ID NO: 8) or a functional variant thereof. In some embodiments, the TIR signaling domain of TLR8 is encoded by the nucleotide sequence set forth in SEQ ID NO: 188. In some embodiments, the TIR signaling domain of TLR8 is encoded by a nucleotide sequence having at least 80%, 85%, 90%, 91%, 92%, 93%, 94%, 95%, 96%, 97%, 98%, or 99% identity to SEQ ID NO: 188.

The TLR signaling domain may have a polypeptide sequence at least 55%, at least 70%, at least 75%, at least 80%, at least 90%, at least 95%, or 100% identical to SEQ ID NO: 8. Functional variants and/or some or all of the conserved residues of the TLR8 signaling domain may be retained. Functional variants may include N to C terminal truncations, insertions, and/or mutations. Functional variants may comprise a polypeptide sequence sufficient to promote signal propagation using the TLR8 signaling pathway. Conserved residues of the TLR8 TIR signaling domain may comprise conserved pattern in the N-terminal half of the BB loop, -E₉E₉R₉D₉W_{9X}P₉G₉-, wherein the glutamate and tryptophan residues remain present; D9 at the C-terminal end of the Beta-sheet A; and/or conserved residues within alpha-helix C and alpha-helix D (Toshchakov, V. and Neuwald, A., 2020)

In some embodiments, the TLR signaling domain is a TLR3 signaling domain or a functional variant thereof. TLR3 signaling domains are derived from Toll-Like Receptor 3 (TLR3), an endosomal, nucleic acid-sensing, TLR that undergoes dimerization, wherein ligand binding results in interaction with TRIF/TICAM1 adapter, resulting in activation of NF-kappa-B, IRF3 nuclear translocation, cytokine secretion and inflammatory responses (Oshiumi, et al. 2003). The TLR3 signaling domain may comprise residues 754-897 of human TLR3 (SEQ ID NO: 27) or a functional variant thereof. The TIR signaling domain of TLR3 may comprise the polypeptide sequence: FEYAAYIIHAYKDKDWVWEHFSSMEKEDQSLKFCLEERDFEAGVFELEAIVNS IKRSRKIIFVITHHLLKDPLCKRFKVHHAVQQAIEQNLDSIILVFLEEIPDYKLNHALCLRR GMFKSHCILNWPVQKERIGAFRHKLQVAL (SEQ ID NO: 3) or a functional variant thereof.

In some embodiments, the TIR signaling domain of TLR3 is encoded by the nucleotide sequence set forth in SEQ ID NO: 183. In some embodiments, the TIR signaling domain of TLR3 is encoded by a nucleotide sequence having at least 80%, 85%, 90%, 91%, 92%, 93%, 94%, 95%, 96%, 97%, 98%, or 99% identity to SEQ ID NO: 183.

The TLR signaling domain may have a polypeptide sequence at least 55%, at least 70%, at least 75%, at least 80%, at least 90%, at least 95%, or 100% identical to SEQ ID NO: 3. Functional variants and/or some or all of the conserved residues of the TLR3 signaling domain may be retained. Functional variants may include N to C terminal truncations, insertions, and/or mutations. Functional variants may comprise a polypeptide sequence sufficient to promote signal propagation using the TLR3 signaling pathway. Conserved residues of the TLR3 TIR signaling domain may comprise N759, Y858, R867, and/or M870 (Sakar, et al., 2007; Hu, et al., 2015; Zhang, et al., 2020).

In some embodiments, the TLR signaling domain is a TLR7 signaling domain or functional variant thereof. TLR7 signaling domains are derived from Toll-Like Receptor 7 (TLR7), an endosomal receptor capable of responding to uridine-containing single strand RNAs or guanosine analogs (Davenne, et al., 2020; Lee, et al., 2003). Association with an agonist results in dimerization allowing for recruitment of TIR-containing adapter MYD88 via homotypic interaction (Zhang, et al. 2016). Myddosome signaling complex interacts with IRAK4, IRAK1, TRAF6, and TRAF3 generating activation of NF-kappa-B and IRF7 to induce cytokines and interferons (Zhang, et al. 2016). The TLR7 signaling domain may comprise residues 889-1033 (145 aa) of human TLR7 (SEQ ID NO: 31). The TIR signaling domain of TLR 7 may comprise the polypeptide sequence: CCYDAFIVYDTKDPAVTEWVLAELVAKLEDPREKHENLCLEE RDWLPGQPVLENLSQSIQLSKKTVFVMTDKYAKTENFKIAFYLSHQRLMDEKVDVIILIF LEKPFQKSKFLQLRKRLCGSSVLEWPTNPQAHPYFWQCLKNAL (SEQ ID NO: 7) or a functional variant thereof. In some embodiments, the TIR signaling domain of TLR7 is encoded by the nucleotide sequence set forth in SEQ ID NO: 187. In some embodiments, the TIR signaling domain of TLR7 is encoded by a nucleotide sequence having at least 80%, 85%, 90%, 91%, 92%, 93%, 94%, 95%, 96%, 97%, 98%, or 99% identity to SEQ ID NO: 187.

The TLR signaling domain may have a polypeptide sequence at least 55%, at least 70%, at least 75%, at least 80%, at least 90%, at least 95%, or 100% identical to SEQ ID NO: 7. Functional variants and/or some or all of the conserved residues of the TLR7 signaling domain may be retained. Functional variants may include N to C terminal truncations, insertions, and/or mutations. Functional variants may comprise a polypeptide sequence sufficient to promote signal propagation using the TLR7 signaling pathway. Conserved residues of the TLR7 TIR signaling domain may comprise >90% conserved pattern in the N-terminal half of the BB loop; -E₉E₉R₉D₉W_{9X}P₉G₉-, wherein the glutamate and tryptophan residues remain present D9 at the C-terminal end of the Beta-sheet A; and/or conserved residues within alpha-helix C and alph-helix D (Toshchakov, V. and Neuwald, A., 2020)

The TLR signaling domain may be a TLR4 signaling domain or functional variant thereof. TLR4 signaling domains are derived from Toll-Like Receptor 4 (TLR4), a type of TLR that functions via interacting with K96 and CD14 via homodimerization to initiate response to lipopolysaccharides (LPS) (Tatematsu, et al., 2016). TLR4 interacts with MYD88, TIRAP, and TRAF6, resulting in activation of NF-kappa-B, cytokine secretion, and inflammatory response (Medzhitov, et al, 1997; Arbour, et al, 2000; Tatematsu, et al., 2016). TLR4 may also function via LPS-independent response, wherein activation is triggered by free fatty acids and Ni (2⁺), which are non-conserved and species-specific (Marc, et al. 2010). The TLR4 signaling domain may comprise residues 672-815 (144 aa) of human TLR4 (SEQ ID NO: 28) or a functional variant thereof. The TIR signaling domain of TLR4 may comprise the polypeptide sequence: NIYDAFVIYSSQDEDWVRNELVKNLEEGVPPFQLCLHYRDFIPGVAIAANIIHEGFHKSR KVIVVVSQHFIQSRWCIFEYEIAQTWQFLSSRAGIIFIVLQKVEKTLLRQQVELYRLLSRN TYLEWEDSVLGRHIFWRRLRKAL (SEQ ID NO: 4) or a functional variant thereof. In some embodiments, the TIR signaling domain of TLR4 is encoded by the nucleotide sequence set forth in SEQ ID NO: 184. In some embodiments, the TIR signaling domain of TLR4 is encoded by a nucleotide sequence having at least 80%, 85%, 90%, 91%, 92%, 93%, 94%, 95%, 96%, 97%, 98%, or 99% identity to SEQ ID NO: 184.

The TLR signaling domain may have a polypeptide sequence at least 55%, at least 70%, at least 75%, at least 80%, at least 90%, at least 95%, or 100% identical to SEQ ID NO: 4. Functional variants and/or some or all of the conserved residues of the TLR4 signaling domain may be retained. Functional variants may include N to C terminal truncations, insertions, and/or mutations. Functional variants may comprise a polypeptide sequence sufficient to promote signal propagation using the TLR4 signaling pathway. Variations of the TLR4 TIR signaling domain may comprise R763H and/or Q834H (Smirnova, et al., 2001). Conserved residues of the TLR4 TIR signaling domain may comprise C747 on alpha-helix C; the presence of a WXC₇₄₇XXE motif; and/or P712 of the BB loop (Shirey, et al. 2020; Poltorak, et al., 1998). Further conserved residues of the TLR4 TIR may comprise residues between the BB loop and alpha-helix E (Toshchakov, et al., 2011).

The TLR signaling domain may be a TLR1 signaling domain or functional variant thereof. TLR1 signaling domains are derived from Toll-Like Receptor 1 (TLR1), a type of TLR that interacts with TLR2 to regulate immune response stimulated by the presence of particular lipopeptides (Lancioni, et al., 2011). The TLR1 signaling domain may comprise residues 635-776 (142 aa) of human TLR1 (SEQ ID NO: 25) or a functional variant thereof (LQFHAFISYSGHDSFWVKNELLPNLEKEGMQICLHERNFVPGKSIVENIITCIEKSYKSIF VLSPNFVQSEWCHYELYFAHHNLFHEGSNSLILILLEPIPQYSIPSSYHKLKSLMARRTYL EWPKEKSKRGLFWANLRAAI (SEQ ID NO: 1)). The TIR signaling domain of TLR1 may comprise the polypeptide sequence: LQFHAFISYSGHDSFWVKNELLPNLEKEGMQICLHERNFVPGKSIVENIITCIEKSYKSIFV LSPNFVQSEWCHYELYFAHHNLFHEGSNSLILILLEPIPQYSIPSSYHKLKSLMARRTYLE WPKEKSKRGLFWANLRAAI (SEQ ID NO: 1) or a functional variant thereof. In some embodiments, the TIR signaling domain of TLR1 is encoded by the nucleotide sequence set forth in SEQ ID NO: 182. In some embodiments, the TIR signaling domain of TLR1 is encoded by a nucleotide sequence having at least 80%, 85%, 90%, 91%, 92%, 93%, 94%, 95%, 96%, 97%, 98%, or 99% identity to SEQ ID NO: 182.

The TLR signaling domain may have a polypeptide sequence at least 55%, at least 70%, at least 75%, at least 80%, at least 90%, at least 95%, or 100% identical to SEQ ID NO: 1. Functional variants and/or some or all of the conserved residues of the TLR1 signaling domain may be retained. Functional variants may include N to C terminal truncations, insertions, and/or mutations. Functional variants may comprise a polypeptide sequence sufficient to promote signal propagation using the TLR1 signaling pathway.

The TLR signaling domain may be a TLR5 signaling domain or functional variant thereof. TLR5 signaling domains are derived from Toll-Like Receptor 5 (TLR5), a type of TLR that has been shown to play a role in immune response via recognition of unique molecular motifs (Bielaszewska, et al., 2018). The TLR5 signaling domain may comprise residues 691-836 (146 aa) of human TLR5 (SEQ ID NO: 29) or a functional variant thereof. The TIR signaling domain of TLR5 may comprise the polypeptide sequence: YKYDAYLCFSSKDFTWVQNALLKHLDTQYSDQNRFNLCFEERDFVPGENRIANIQDAI WNSRKIVCLVSRHFLRDGWCLEAFSYAQGRCLSDLNSALIMVVVGSLSQYQLMKHQSI RGFVQKQQYLRWPEDLQDVGWFLHKLSQQI (SEQ ID NO: 5) or a functional variant thereof. In some embodiments, the TIR signaling domain of TLR5 is encoded by the nucleotide sequence set forth in SEQ ID NO: 185. In some embodiments, the TIR signaling domain of TLR5 is encoded by a nucleotide sequence having at least 80%, 85%, 90%, 91%, 92%, 93%, 94%, 95%, 96%, 97%, 98%, or 99% identity to SEQ ID NO: 185.

The TLR signaling domain may have a polypeptide sequence at least 55%, at least 70%, at least 75%, at least 80%, at least 90%, at least 95%, or 100% identical to SEQ ID NO: 5. Functional variants and/or some or all of the conserved residues of the TLR5 signaling domain may be retained. Functional variants may include N to C terminal truncations, insertions, and/or mutations. Functional variants may comprise a polypeptide sequence sufficient to promote signal propagation using the TLR5 signaling pathway.

The TLR signaling domain may be a TLR6 signaling domain or functional variant thereof. TLR6 signaling domains are derived from Toll-Like Receptor 6 (TLR6), a type of TLR that has been shown to play a role in recognition of diacylated and triacylated lipopeptides via interaction with TLR2 (Stewart, et al., 2010; Triantafilou, et al., 2006). The TLR6 signaling domain may comprise residues 640-781 (142 aa) of human TLR6 (SEQ ID NO: 30) or a functional variant thereof. The TIR signaling domain of TLR6 may comprise the polypeptide sequence: LQFHAFISYSEHDSAWVKSELVPYLEKEDIQICLHERNFVPGKSIVENIINCIEKSYKSIFVL SPNFVQSEWCHYELYFAHHNLFHEGSNNLILILLEPIPQNSIPNKYHKLKALMTQRTYLQ WPKEKSKRGLFWANIRAAF (SEQ ID NO: 6) or a functional variant thereof. In some embodiments, the TIR signaling domain of TLR6 is encoded by the nucleotide sequence set forth in SEQ ID NO: 186. In some embodiments, the TIR signaling domain of TLR6 is encoded by a nucleotide sequence having at least 80%, 85%, 90%, 91%, 92%, 93%, 94%, 95%, 96%, 97%, 98%, or 99% identity to SEQ ID NO: 186.

The TLR signaling domain may have a polypeptide sequence at least 55%, at least 70%, at least 75%, at least 80%, at least 90%, at least 95%, or 100% identical to SEQ ID NO: 6. Functional variants and/or some or all of the conserved residues of the TLR6 signaling domain may be retained. Functional variants may include N to C terminal truncations, insertions, and/or mutations. Functional variants may comprise a polypeptide sequence sufficient to promote signal propagation using the TLR6 signaling pathway.

The TLR signaling domain may be a TLR9 signaling domain or functional variant thereof. TLR9 signaling domains are derived from Toll-Like Receptor 9 (TLR9), a type of TLR that has been shown to play a role in nucleotide-sensing, stimulated by the presence of unmethylated cytidine-phosphate-guanosine (CpG) dinucleotides (Takeshita, et al., 2001; Doyle, et al., 2007). The TLR9 signaling domain may comprise residues 868-1013 (146 aa) of human TLR9 (SEQ ID NO: 33) or a functional variant thereof. The TIR signaling domain of TLR9 may comprise the polypeptide sequence: LPYDAFVVFDKTQSAVADWVYNELRGQLEECRGRWALRLCLEERDWLPGKTLFENLW ASVYGSRKTLFVLAHTDRVSGLLRASFLLAQQRLLEDRKDVVVLVILSPDGRRSRYVRL RQRLCRQSVLLWPHQPSGQRSFWAQLGMAL (SEQ ID NO: 9) or a functional variant thereof. In some embodiments, the TIR signaling domain of TLR9 is encoded by the nucleotide sequence set forth in SEQ ID NO: 189. In some embodiments, the TIR signaling domain of TLR9 is encoded by a nucleotide sequence having at least 80%, 85%, 90%, 91%, 92%, 93%, 94%, 95%, 96%, 97%, 98%, or 99% identity to SEQ ID NO: 189.

The TLR signaling domain may have a polypeptide sequence at least 55%, at least 70%, at least 75%, at least 80%, at least 90%, at least 95%, or 100% identical to SEQ ID NO: 9. Functional variants and/or some or all of the conserved residues of the TLR9 signaling domain may be retained. Functional variants may include N to C terminal truncations, insertions, and/or mutations. Functional variants may comprise a polypeptide sequence sufficient to promote signal propagation using the TLR9 signaling pathway.

The TLR signaling domain may be a TLR10 signaling domain or functional variant thereof. TLR10 signaling domains are derived from Toll-Like Receptor 10 (TLR10), a member of the TLR receptor family involved in detection of unique molecules and regulating immune response. The TLR10 signaling domain may comprise residues 632-775 (144 aa) of human TLR10 (SEQ ID NO: 34) or a functional variant thereof. The TIR signaling domain of TLR 10 may comprise the polypeptide sequence: VRFHAFISYSEHDSLWVKNELIPNLEKEDGSILICLYESYFDPGKSISENIVSFIEKSYKSIF VLSPNFVQNEWCHYEFYFAHHNLFHENSDHIILILLEPIPFYCIPTRYHKLKALLEKKAYL EWPKDRRKCGLFWANLRAAI (SEQ ID NO: 10) or a functional variant thereof. In some embodiments, the TIR signaling domain of TLR10 is encoded by the nucleotide sequence set forth in SEQ ID NO: 190. In some embodiments, the TIR signaling domain of TLR10 is encoded by a nucleotide sequence having at least 80%, 85%, 90%, 91%, 92%, 93%, 94%, 95%, 96%, 97%, 98%, or 99% identity to SEQ ID NO: 190.

The TLR signaling domain may have a polypeptide sequence at least 55%, at least 70%, at least 75%, at least 80%, at least 90%, at least 95%, or 100% identical to SEQ ID NO: 10. Functional variants and/or some or all of the conserved residues of the TLR10 signaling domain may be retained. Functional variants may include N to C terminal truncations, insertions, and/or mutations. Functional variants may comprise a polypeptide sequence sufficient to promote signal propagation using the TLR10 signaling pathway.

### Interleukin-1 Receptor (IL-1R) Subfamily Signaling Domains

In an aspect, the disclosure provides CARs having a cytoplasmic portion that includes an interleukin-1 receptor (IL-1R) subfamily signaling domain. As used herein, the term “IL-1R subfamily signaling domain” refers to the TIR domain of a member of the IL-1R receptor subfamily, or a functional variant thereof. Currently, in humans, ten IL-1R subfamily receptors have been identified (Fields, et al., 2019), nine of which possess a TIR domain. The IL-1R subfamily signaling domain may be an IL-1R signaling domain or an interleukin 18 receptor (IL-18R) signaling domain. In some embodiments, the IL-1R subfamily signaling domain may be an IL-1R4 (IL-33R, ST2) signaling domain, IL-1R6 (IL-36R, IL1-Rrp2) signaling domain, IL-1R3 (IL-1RAcp) signaling domain, IL-1R7 (IL-18Rbeta, AcPl) signaling domain, IL-1R8 (SIGGIR, TIR8) signaling domain, IL-1R9 (IL-1RAPL1, TIGIRR-2) signaling domain, or IL-1R10 (IL-1RAPL2) signaling domain.

Accordingly, the cytoplasmic portion of the CAR may comprise a signaling domain having a polypeptide sequence selected from SEQ ID NOs: 21 or 22, or a functional variant thereof. The signaling domain may have a polypeptide sequence at least 55%, at least 70%, at least 75%, at least 80%, at least 90%, at least 95%, or 100% identical to any one of SEQ ID NOs: 21-22.

The cytoplasmic portion of the CAR may comprise a signaling domain having a polypeptide sequence selected from SEQ ID NOs: 143, 144, 145, 146, 147, 148, or 149, or a functional variant thereof. The signaling domain may have a polypeptide sequence at least 55%, at least 70%, at least 75%, at least 80%, at least 90%, at least 95%, or 100% identical to any one of SEQ ID NOs: 143, 144, 145, 146, 147, 148, or 149.

The IL-1R subfamily signaling domains derive from IL-1R, a superfamily characterized by extracellular immunoglobulin-like (Ig-like) domains and an intracellular Toll/Interleukin 1R (TIR) domain. TIR domains of the IL-1R subfamily share a degree of homology with TIR domains of TLRs and function similarly with regard to signal transduction. IL-1R family members function as heterodimers that recognize cognate cytokines and activate downstream signaling molecules (Boraschi, D. and Tagliabue, A., 2013). Ligand binding results in oligomerization of TIR domains on the receptor, followed by interaction of accessory proteins and adaptor molecules (e.g., MYD88). Heterodimerization with TIR domains results in transduction of downstream cytoplasmic kinases (e.g., IRAKs), and NF-kB activation, which produces an immune response (e.g., cytokine secretion).

The IL-1R subfamily signaling domain may be an IL-1R signaling domain or functional variant thereof. IL-1R signaling domains are derived from Interleukin-1 Receptor (IL-1R), a cytokine receptor that functions by binding interleukin-1 and associates with accessory protein (IL-1RAcP) to form a complex which mediates interaction with TOLLIP, MYD88, and IRAK resulting in MAPK signal transduction and activation of NF-kappa-B (Greenfeder, et al., 1995; Slack, et al., 2000). The IL-1R signaling domain may comprise residues 357-569 (213 aa) of human IL-1R (SEQ ID NO: 35) or functional variant thereof. The IL-1R signaling domain of IL-1R may comprise the polypeptide sequence: KTYDAYILYPKTVGEGSTSDCDIFVFKVLPEVLEKQCGYKLFIYGRDDYVGEDIVEVINE NVKKSRRLIIILVRETSGFSWLGGSSEEQIAMYNALVQDGIKVVLLELEKIQDYEKMPESI KFIKQKHGAIRWSGDFTQGPQSAKTRFWKNVRYHM (SEQ ID NO: 21).

The IL-1R signaling domain may have a polypeptide sequence at least 55%, at least 70%, at least 75%, at least 80%, at least 90%, at least 95%, or 100% identical to SEQ ID NO: 21. Functional variants and/or some or all of the conserved residues of the IL-1R signaling domain may be retained. Functional variants may include N to C terminal truncations, insertions, and/or mutations. Functional variants may comprise a polypeptide sequence sufficient to promote signal propagation using the IL-1R signaling pathway. Conserved residues of the IL-1R signaling domain may comprise D369A and/or R428A (Slack, et al. 2000).

The IL-1R subfamily signaling domain may be an Interleukin-18 Receptor (IL-18R) signaling domain. IL-18R signaling domains are derived from Interleukin-18 Receptor (IL-18R), a cytokine receptor that functions by binding a cognate cytokine, associating as a complex with IL-18RAcP accessory protein, and engaging with one or more adaptor proteins, resulting in the signal transduction (Loiarro, et al., 2010). The IL-18R signaling domain may comprise residues 373-520 (148 aa) of human IL-18R (SEQ ID NO: 36) or a functional variant thereof. The IL-18R signaling domain may comprise the polypeptide sequence: KTYDAFVSYLKECRPENGEEHTFAVEILPRVLEKHFGYKLCIFERDVVPGGAVVDEIHSL IEKSRRLIIVLSKSYMSNEVRYELESGLHEALVERKIKIILIEFTPVTDFTFLPQSLKLLKSH RVLKWKADKSLSYNSRFWKNLLYLM (SEQ ID NO: 22) or a functional variant thereof.

The IL-18R signaling domain may have a polypeptide sequence at least 55%, at least 70%, at least 75%, at least 80%, at least 90%, at least 95%, or 100% identical to SEQ ID NO: 22. Functional variants and/or some or all of the conserved residues of the IL-18R signaling domain may be retained. Functional variants may include N to C terminal truncations, insertions, and/or mutations. Functional variants may comprise a polypeptide sequence sufficient to promote signal propagation using the IL-18R signaling pathway. Conserved residues of the IL-18R signaling domain may comprise conserved sequences within the BB loop (Ota, et al., 2012).

### Other Components of CAR Cytoplasmic Portion

In another aspect, the disclosure provides CARs having a cytoplasmic portion that includes a TIR signaling domain and a CD3ζ domain—preferably a CD3ζ domain at the C-terminus of the CAR. The CARs disclosed herein may further comprise one or more other signaling domains. In variations, the cytoplasmic portion of the CAR may comprise a 2B4 signaling domain or an OX40 signaling domain. In further variations, however, the cytoplasmic portion of CAR lacks a 2B4 signaling domain, lacks an OX40 signaling domain, or lacks both 2B4 and OX40 signaling domains.

The cytoplasmic portion may comprise the polypeptide sequence: RVKFSRSADAPAYQQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR (SEQ ID NO: 64) or a functional variant thereof. The cytoplasmic portion may have a polypeptide sequence at least 55%, at least 70%, at least 75%, at least 80%, at least 90%, at least 95%, or 100% identical to SEQ ID NO: 64.

The cytoplasmic portion may comprise the polypeptide sequence: WRRKRKEKQSETSPKEFLTIYEDVKDLKTRRNHEQEQTFPGGGSTIYSMIQSQSSAPTSQ EPAYTLYSLIQPSRKSGSRKRNHSPSFNSTIYEVIGKSQPKAQNPARLSRKELENFDVYS (SEQ ID NO: 65) or a functional variant thereof. The cytoplasmic portion may have a polypeptide sequence at least 55%, at least 70%, at least 75%, at least 80%, at least 90%, at least 95%, or 100% identical to SEQ ID NO: 65.

The cytoplasmic portion may comprise the polypeptide sequence: RRDQRLPPDAHKPPGGGSFRTPIQEEQADAHSTLAKI (SEQ ID NO: 66) or a functional variant thereof. The cytoplasmic portion may have a polypeptide sequence at least 55%, at least 70%, at least 75%, at least 80%, at least 90%, at least 95%, or 100% identical to SEQ ID NO: 66.

Further cytoplasmic domains that may be used in CARs are provided in U.S. Pat. Pub. US 2017/0233454 and U.S. Pat. No. 10,415,017.

### Ligand-Binding Domains

In an aspect, the disclosure provides CARs having an extracellular portion comprising a ligand-binding domain. The ligand-binding domain of the instant disclosure may comprise an antibody-based binding agent that specifically binds an antigen of interest via an antigen or antigenic fragment thereof. As used herein, the term “antigen” refers to a peptide, polypeptide, glycoprotein, glycan, lipid, glycolipid, or other biomolecule that identifies target cells for treatment by immunotherapy with NK cells.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response. The term “antigen-binding fragment” refers to a portion of an antibody that specifically bind to an antigen. Specific binding is considered to mean any level of specificity that has clinical utility. Specific binding may mean a binding constant at least 10³ M⁻¹ greater, at least 10⁴ M⁻¹ greater, or at least 10⁵ M⁻¹ greater than a binding constant for other molecules in a tissue. The antigen-binding fragment may be an artificial fragment such as humanized or chimeric antigen-binding fragment.

The term “antibody-like domain” refers to recombinant antibody fragments (such as sFv fragments, dsFv fragments, bispecific sFv fragments, bispecific dsFv fragments, F (ab)′2 fragments, single chain Fv proteins (“scFv”), disulfide stabilized Fv proteins (“dsFv”), diabodies, and triabodies (as are known in the art), and camelid antibodies, that retain antigen-binding properties (see, for example, U.S. Pat. Nos. 6,015,695; 6,005,079; 5,874,541; 5,840,526; 5,800,988; and 5,759,808). The antibody-like domain may be expressed as a fusion protein to form the singe-chain CAR, or it may be associated with the other polypeptide(s) through other known means, forming a multipartite CAR. An illustrative example of a multipartite CAR as described, e.g., in Int'l Pat. Pub. No. PCT/US2021/05676.

The antibody-like domain may be a single-chain variable fragment (scFv). An scFv protein is a fusion protein in which a light chain variable region (VL) of an antibody and a heavy chain variable region (VH) of an antibody expressed as fusion polypeptide, optionally with an intervening link, such as a Gly-Ser linker, in either VH-VL or VL-VH orientation. The scFv may be an dsFvs, the chains have been mutated to introduce a disulfide bond to stabilize the association of the chains.

When the ligand-binding domain is an antigen-like domain, it may comprise the complementarity determine regions CDR-H1, CDR-H2, CDR-H3, CDR-L1, CDR-L2, and CDR-L3. In some embodiments, the binding domain comprises VH and/or VL chains. In some embodiments, the ligand-binding domain comprises a single polypeptide chain. The ligand binding domain may be an scFv.

Ligand Binding Domain that Specifically Binds CD19

In an aspect, the disclosure provides chimeric antigen receptors that specifically bind CD19. The ligand binding domain may be an anti-CD19 scFv comprising the polypeptide sequence set forth in SEQ ID NO: 73 or a functional variant thereof having a polypeptide sequence at least 55%, at least 70%, at least 75%, at least 80%, at least 90%, at least 95%, or 100% identical to SEQ ID NO: 73, or other scFv's or VH/VL pairs generated from SEQ ID NO: 73. The ligand binding domain may comprise a VH sequence according to SEQ ID NO: 74 and/or a VL sequence according to SEQ ID NO: 75, or a functional variants thereof having a polypeptide sequence at least 55%, at least 70%, at least 75%, at least 80%, at least 90%, at least 95%, or 100% identical to SEQ ID NO: 74 and SEQ ID NO: 75, respectively.

The ligand binding domain (e.g., an scFv) may comprise the CDR sequences in Table 1, or functional variants with 1, 2, 3 or more substitutions.

TABLE 1
Anti-CD19 Ligand Binding Domain Sequences

| | | SEQ ID |
|---|---|---|
| | Polypeptide Sequence | NO: |
| scFv | DIQMTQTTSSLSASLGDRVTISCRASQDISKYLNWYQQKPDGT | 73 |
| (VL-VH) | VKLLIYHTSRLHSGVPSRFSGSGSGTDYSLTISNLEQEDIATY | |
| | FCQQGNTLPYTFGGGTKLEITGSTSGSGKPGSGEGSTKGEVKL | |
| | QESGPGLVAPSQSLSVTCTVSGVSLPDYGVSWIRQPPRKGLEW | |
| | LGVIWGSETTYYNSALKSRLTIIKDNSKSQVFLKMNSLQTDDT | |
| | AIYYCAKHYYYGGSYAMDYWGQGTSVTVSGILT | |
| VH | EVKLQESGPGLVAPSQSLSVTCTVSGVSLPDYGVSWIRQPPRK | 74 |
| | GLEWLGVIWGSETTYYNSALKSRLTIIKDNSKSQVFLKMNSL | |
| | QTDDTAIYYCAKHYYYGGSYAMDYWGQGTSVTVSGILT | |
| VH | EVKLQESGPGLVAPSQSLSVTCTVSGVSLPDYGVSWIRQPPRK | 151 |
| | GLEWLGVIWGSETTYYNSALKSRLTIIKDNSKSQVFLKMNSL | |
| | QTDDTAIYYCAKHYYYGGSYAMDYWGQGTSVTVSS | |
| VL | DIQMTQTTSSLSASLGDRVTISCRASQDISKYLNWYQQKPDGT | 75 |
| | VKLLIYHTSRLHSGVPSRFSGSGSGTDYSLTISNLEQEDIATY | |
| | FCQQGNTLPYTFGGGTKLEITGST | |
| VL | DIQMTQTTSSLSASLGDRVTISCRASQDISKYLNWYQQKPDGT | 150 |
| | VKLLIYHTSRLHSGVPSRFSGSGSGTDYSLTISNLEQEDIATY | |
| | FCQQGNTLPYTFGGGTKLEIT | |
| CDRH1 | DYGVS | 76 |
| CDRH2 | VIWGSETTYYNSALKS | 77 |
| CDRH3 | HYYYGGSYAMDY | 78 |
| CDRL1 | RASQDISKYLN | 79 |
| CDRL2 | HTSRLHS | 80 |
| CDRL2 | QQGNTLPYT | 81 |

In variations, the ligand binding domain may comprise CDRs extracted from SEQ ID NO: 109 by known methods (e.g., Chothia, Martin, Kabat, AHo and IMGT numbering available at, for example abysis.org; see Swindells et al. J. Mol. Bio. 429 (3): 356-365 (2017)).

In some embodiments, the extracellular domain of a chimeric receptor comprises an amino acid sequence set forth in SEQ ID NO: 152. In some embodiments, the extracellular domain of a chimeric receptor consists of an amino acid sequence set forth in SEQ ID NO: 152. In some embodiments, the extracellular domain of a chimeric receptor comprises an amino acid sequence having at least 80%, 85%, 90%, 91%, 92%, 93%, 94%, 95%, 96%, 97%, 98% or 99% identity to SEQ ID NO: 152.

In some embodiments, the extracellular domain of a chimeric receptor is encoded by the nucleotide sequence set forth in SEQ ID NO: 153. In some embodiments, the extracellular domain of a chimeric receptor is encoded by a nucleotide sequence having at least 80%, 85%, 90%, 91%, 92%, 93%, 94%, 95%, 96%, 97%, 98% or 99% identity to SEQ ID NO: 153.

Ligand Binding Domain that Specifically Binds CD20

CD20 is a B-cell lineage-specific antigens expressed on the cell surface of most B-cell lymphomas, particularly in late stages of B-cell lymphogenesis. In an aspect, the disclosure provides chimeric antigen receptors that specifically bind CD20. The ligand binding domain may be an anti-CD20 scFv comprising the polypeptide sequence set forth in SEQ ID NO: 139 or a functional variant thereof having a polypeptide sequence at least 55%, at least 70%, at least 75%, at least 80%, at least 90%, at least 95%, or 100% identical to SEQ ID NO: 139, or other scFv's or VH/VL pairs generated from SEQ ID NO: 139.

CD20-scFv-4 (SEQ ID NO: 139):
DIQLTQSPSSLSASVGDRVTITCRASS
SVSYIHWFQQKPGKAPKPWIYATSNLA
SGVPSRFSGSGSGTDYTLTISSLQPED
FATYYCQQWTSNPPTFGGGTKVEIKGG
GGSGGGGSGGGGSGGGGSQVQLVQSGA
EVKKPGASVKVSCKASGYTFTSYNMHW
VRQAPGQGLEWIGAIYPGNGDTSYNQK
FKGRATLTADKSTSTAYMELSSLRSED
TAVYYCARSTYYGGDWYFNVWGRGTLV
TVS

In variations, the ligand binding domain may comprise CDRs extracted from SEQ ID NO: 139 by known methods (e.g., Chothia, Martin, Kabat, AHo and IMGT numbering available at, for example abysis.org; see Swindells et al. J. Mol. Bio. 429 (3): 356-365 (2017)). In some embodiments, the ligand binding domain may be an anti-CD20 scFv comprising SEQ ID NO: 139.

In some embodiments, the extracellular domain of a chimeric receptor comprises an amino acid sequence set forth in SEQ ID NO: 156. In some embodiments, the extracellular domain of a chimeric receptor consists of an amino acid sequence set forth in SEQ ID NO: 156. In some embodiments, the extracellular domain of a chimeric receptor comprises an amino acid sequence having at least 80%, 85%, 90%, 91%, 92%, 93%, 94%, 95%, 96%, 97%, 98% or 99% identity to SEQ ID NO: 156. The ligand binding domain may comprise a VH sequence according to SEQ ID NO: 154 and/or a VL sequence according to SEQ ID NO: 155, or a functional variants thereof having a polypeptide sequence at least 55%, at least 70%, at least 75%, at least 80%, at least 90%, at least 95%, or 100% identical to SEQ ID NO: 154 and SEQ ID NO: 155, respectively.

In some embodiments, the extracellular domain of a chimeric receptor is encoded by the nucleotide sequence set forth in SEQ ID NO: 157. In some embodiments, the extracellular domain of a chimeric receptor is encoded by a nucleotide sequence having at least 80%, 85%, 90%, 91%, 92%, 93%, 94%, 95%, 96%, 97%, 98% or 99% identity to SEQ ID NO: 157.

Ligand Binding Domain that Specifically Binds HER2

In certain embodiments, the ligand-binding domain may bind a cancer antigen. In specific embodiments, the ligand-binding domain may bind an antigen associated with a solid cancer, e.g., an antigen present on a tumor cell. For example, functional activation of human epidermal growth factor receptor 2 (HER2) has been shown to strongly promote carcinogenesis. In an aspect, the disclosure provides chimeric antigen receptors that specifically bind HER2. The ligand binding domain may be an anti-HER2 scFv comprising the polypeptide sequence set forth in SEQ ID NOs: 140-142 or a functional variant thereof having a polypeptide sequence at least 55%, at least 70%, at least 75%, at least 80%, at least 90%, at least 95%, or 100% identical to SEQ ID NOs: 140-142, or other scFv's or VH/VL pairs generated from SEQ ID NOs: 140-142. The ligand binding domain (e.g., an scFv) may comprise CDR sequences in or functional variants with 1, 2, 3 or more substitutions.

In some embodiments, the extracellular domain of a chimeric receptor comprises an amino acid sequence set forth in SEQ ID NOs: 140, 141, or 142. In some embodiments, the extracellular domain of a chimeric receptor consists of an amino acid sequence set forth in SEQ ID NOs: 140, 141, or 142. In some embodiments, the extracellular domain of a chimeric receptor comprises an amino acid sequence having at least 80%, 85%, 90%, 91%, 92%, 93%, 94%, 95%, 96%, 97%, 98% or 99% identity to SEQ ID NOs: 140, 141, or 142.

In some embodiments, the extracellular domain of a chimeric receptor is encoded by the nucleotide sequence set forth in SEQ ID NOs: 198,199, or 200. In some embodiments, the extracellular domain of a chimeric receptor is encoded by a nucleotide sequence having at least 80%, 85%, 90%, 91%, 92%, 93%, 94%, 95%, 96%, 97%, 98% or 99% identity to SEQ ID NO:: 198, 199, or 200.

TABLE 2
Anti-HER2 Ligand Binding Domain Sequences

| | | SEQ ID |
|---|---|---|
| | Polypeptide Sequence | NO: |
| HER2- | EVQLVESGGGLVQPGGSLRLSCAASGFNIKDTYIHWVRQAPG | 140 |
| scFv-2 | KGLEWVARIYPTNGYTRYADSVKGRFTISADTSKNTAYLQM | |
| (VL-VH) | NSLRAEDTAVYYCSRWGGDGFYAMDYWGQGTLVTVSSGGSG | |
| | GGSGGGSGGGSGGGSGDIQMTQSPSSLSASVGDRVTITCRA | |
| | SQDVNTAVAWYQQKPGKAPKLLIYSASFLYSGVPSRFSGSR | |
| | SGTDFTLTISSLQPEDFATYYCQQHYTTPPTFGQGTKVEIK | |
| HER2- | EVQLVESGGGLVQPGGSLRLSCAASGFNIKDTYIHWVRQAP | 141 |
| scFv-3 | GKGLEWVARIYPTNGYTRYADSVKGRFTISADTSKNTAYLQ | |
| (VL-VH) | MNSLRAEDTAVYYCSRWGGDGFYAMDYWGQGTLVTVSSGGG | |
| | GSGGGGSGGGGSDIQMTQSPSSLSASVGDRVTITCRASQDV | |
| | NTAVAWYQQKPGKAPKLLIYSASFLYSGVPSRFSGSRSGTD | |
| | FTLTISSLQPEDFATYYCQQHYTTPPTFGQGTKVEIK | |
| HER2- | EVQLVESGGGLVQPGGSLRLSCAASGFNIKDTYIHWVRQAPG | 142 |
| scFv-5 | KGLEWVARIYPTNGYTRYADSVKGRFTISADTSKNTAYLQMN | |
| (VL-VH) | SLRAEDTAVYYCSRWGGDGFYAMDYWGQGTLVTVSSGGGGSG | |
| | GGGSGGGGSGGGGSDIQMTQSPSSLSASVGDRVTITCRASQD | |
| | VNTAVAWYQQKPGKAPKLLIYSASFLYSGVPSRFSGSRSGTD | |
| | FTLTISSLQPEDFATYYCQQHYTTPPTFGQGTKVEIK | |

In variations, the ligand binding domain may comprise CDRs extracted from SEQ ID NOs: 140-142 by known methods (e.g., Chothia, Martin, Kabat, AHo and IMGT numbering available at, for example abysis.org; see Swindells et al. J. Mol. Bio. 429 (3): 356-365 (2017)). In some embodiments, the ligand binding domain may be an anti-HER2 scFv comprising SEQ ID NO: 140. In some other embodiments, the ligand binding domain may be an anti-HER2 scFv comprising SEQ ID NO: 141. In some embodiments, the ligand binding domain may be an anti-HER2 scFv comprising SEQ ID NO: 142.

In some embodiments, the CAR comprises an amino acid sequence having at least 80%, 85%, 90%, 91%, 92%, 93%, 94%, 95%, 96%, 97%, 98% or 99% identity to SEQ ID NOs: 202-207. In some embodiments, the CAR comprises an amino acid sequence having at least 80%, 85%, 90%, 91%, 92%, 93%, 94%, 95%, 96%, 97%, 98% or 99% identity to SEQ ID NO: 202 (HER2-2-CD8-aTM-TLR2-CD3ζ without sIL15). In some embodiments, the CAR comprises an amino acid sequence having at least 80%, 85%, 90%, 91%, 92%, 93%, 94%, 95%, 96%, 97%, 98% or 99% identity to SEQ ID NO: 203 (HER2-2-CD8-aTM-TLR2-CD3ζ-sIL15). In some embodiments, the CAR comprises an amino acid sequence having at least 80%, 85%, 90%, 91%, 92%, 93%, 94%, 95%, 96%, 97%, 98% or 99% identity to SEQ ID NO: 204 (HER2-3-CD8-aTM-TLR2-CD3ζ without sIL15). In some embodiments, the CAR comprises an amino acid sequence having at least 80%, 85%, 90%, 91%, 92%, 93%, 94%, 95%, 96%, 97%, 98% or 99% identity to SEQ ID NO: 205 (HER2-3-CD8-aTM-TLR2-CD3ζ-sIL15). In some embodiments, the CAR comprises an amino acid sequence having at least 80%, 85%, 90%, 91%, 92%, 93%, 94%, 95%, 96%, 97%, 98% or 99% identity to SEQ ID NO: 206 (HER2-5-CD8-aTM-TLR2-CD3ζ without sIL 15). In some embodiments, the CAR comprises an amino acid sequence having at least 80%, 85%, 90%, 91%, 92%, 93%, 94%, 95%, 96%, 97%, 98% or 99% identity to SEQ ID NO: 207 (HER2-5-CD8-aTM-TLR2-CD3ζ-sIL15).

Ligand Binding Domain that Specifically Binds B-Cell Maturation Antigen (BCMA)

In some embodiments, a chimeric receptor described herein comprises an extracellular domain comprising an ligand binding domain or binding fragment thereof of BCMA. BCMA is preferentially expressed in mature B lymphocytes and is important for B cell development.

In some embodiments, a chimeric receptor as described herein comprises an extracellular domain comprising a ligand binding domain or binding fragment thereof of BCMA.

In some embodiments, the extracellular domain of a chimeric receptor comprises an amino acid sequence set forth in SEQ ID NO: 210,212, 214, or 216. In some embodiments, the extracellular domain of a chimeric receptor consists of an amino acid sequence set forth in SEQ ID NOs: 210, 212, 214, or 216. In some embodiments, the extracellular domain of a chimeric receptor comprises an amino acid sequence having at least 80%, 85%, 90%, 91%, 92%, 93%, 94%, 95%, 96%, 97%, 98% or 99% identity to SEQ ID NOs: 210, 212,214, or 216.

In some embodiments, the extracellular domain of a chimeric receptor is encoded by the nucleotide sequence set forth in SEQ ID NOs: 209, 211, 213, or 215. In some embodiments, the extracellular domain of a chimeric receptor is encoded by a nucleotide sequence having at least 80%, 85%, 90%, 91%, 92%, 93%, 94%, 95%, 96%, 97%, 98% or 99% identity to SEQ ID NOs: 209, 211, 213, or 215.

### Signal Peptides

The chimeric antigen receptor may be expressed from a polynucleotide sequence that encodes an N-terminal signal peptide. Signal peptides may direct their fusion partners to be integrated into the plasma membrane of the cell. For example, the signal peptide comprises the sequence MALPVTALLLPLALLLHAARP (SEQ ID NO: 37), or a functional variant with 1, 2, 3 or more amino acid substitutions.

### Transmembrane Domains

In an aspect, the disclosure provides CARs having a transmembrane portion that includes a transmembrane domain. The CAR can be designed to comprise a transmembrane domain that is fused to the antigen-binding domain of the CAR. In some embodiments, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex. In some embodiments, the transmembrane domain is operably linked to the extracellular domain with or without a linker. In some embodiments, the transmembrane domain is operably linked to the extracellular domain via a hinge domain as described herein. In some embodiments, the transmembrane domain is a naturally occurring transmembrane domain derived from a protein. In some embodiments, the transmembrane domain is a synthetic transmembrane domain. In some embodiments, the transmembrane domain comprises a full transmembrane domain of a polypeptide. In some embodiments, the transmembrane domain comprises a fragment of a transmembrane domain of a polypeptide.

The transmembrane domain may be derived either from a natural or from a synthetic source. Transmembrane regions that may be used include the transmembrane domains of CD28, CD3 epsilon, CD4, CD5, CD8, CD9, CD16, CD22, CD33, CD37, CD45, CD64, CD80, CD86, CD134, CD137, CD154, TCR alpha, TCR beta, or CD3ζ, or a functional variant thereof. In variations, the transmembrane domain may be the transmembrane domain of CD8 alpha chain (CD8α), CD28, CD16, NKp44, NKp46, NKG2, DNAM1, IL-2Rβ, TLR, IL-1R subfamily, or functional variant thereof.

Illustrative transmembrane domains are provided in Table 3.

TABLE 3
Transmembrane (TM) domains

| | | SEQ |
|---|---|---|
| | | ID |
| Abbreviation | Polypeptide Sequence | NO: |
| aTM | NLFVASWIAVMIIFRIGMAVAI | 41 |
| | FCCFFFP | |
| DNAM1 TM | GGTVLLLLFVISITTIIVIFL | 42 |
| CD28 TM | FWVLVVVGGVLACYSLLVTVA | 43 |
| | FIIFWV | |
| IL-2Rbeta | IPWLGHLLVGLSGAFGFIILV | 44 |
| | YLLI | |
| TLR1 TM | LLIVTIVATMLVLAVTVTSLC | 45 |
| TLR2 TM | ALVSGMCCALFLLILLTGVLC | 46 |
| TLR3 TM | FFMINTSILLIFIFIVLLIHF | 47 |
| TLR4 TM | TIIGVSVLSVLVVSVVAVLVY | 48 |
| TLR5 TM | FSLFIVCTVTLTLFLMTILTV | 49 |
| TLR6 TM | LIVTIGATMLVLAVTVTSLCI | 50 |
| TLR7 TM | LILFSLSISVSLFLMVMMTAS | 51 |
| TLR8 TM | AVILFFFTFFITTMVMLAALA | 52 |
| TLR9 TM | FALSLLAVALGLGVPMLHHLC | 53 |
| TLR10 TM | ALLIVTIVVIMLVLGLAVAFC | 54 |
| IL1R TM | HMIGICVTLTVIIVCSVFIY | 55 |
| IL18R TM | GMIIAVLILVAVVCLVTVCVI | 56 |

In some embodiments, the transmembrane domain is a transmembrane domain of CD28. In some embodiments, the transmembrane domain is a fragment of a CD28 transmembrane domain. In some embodiments, a CD28 transmembrane domain comprises the amino acid sequence set forth in SEQ ID NO: 43. In some embodiments, a CD28 transmembrane domain consists of the amino acid sequence set forth in SEQ ID NO: 43. In some embodiments, a CD28 transmembrane domain comprises an amino acid sequence having at least 80%, 85%, 90%, 91%, 92%, 93%, 94%, 95%, 96%, 97%, 98% or 99% identity to SEQ ID NO: 43.

In some embodiments, a CD28 transmembrane domain is encoded by the nucleotide sequence set forth in SEQ ID NO: 160. In some embodiments, a CD28 transmembrane domain is encoded by a nucleotide sequence having at least 80%, 85%, 90%, 91%, 92%, 93%, 94%, 95%, 96%, 97%, 98%, or 99% identity to SEQ ID NO: 160. In some embodiments, a CD28 transmembrane domain is encoded by the nucleotide sequence set forth in SEQ ID NO: 218. In some embodiments, a CD28 transmembrane domain is encoded by a nucleotide sequence having at least 80%, 85%, 90%, 91%, 92%, 93%, 94%, 95%, 96%, 97%, 98%, or 99% identity to SEQ ID NO: 218.

In some embodiments, the transmembrane domain is a transmembrane domain of TLR2. In some embodiments, the transmembrane domain is a fragment of a TLR2 transmembrane domain. In some embodiments, a TLR2 transmembrane domain comprises the amino acid sequence set forth in SEQ ID NO: 46. In some embodiments, a TLR2 transmembrane domain consists of the amino acid sequence set forth in SEQ ID NO: 46. In some embodiments, a TLR2 transmembrane domain comprises an amino acid sequence having at least 80%, 85%, 90%, 91%, 92%, 93%, 94%, 95%, 96%, 97%, 98% or 99% identity to SEQ ID NO: 46.

In some embodiments, a TLR2 transmembrane domain is encoded by the nucleotide sequence set forth in SEQ ID NO: 161. In some embodiments, a TLR2 transmembrane domain is encoded by a nucleotide sequence having at least 80%, 85%, 90%, 91%, 92%, 93%, 94%, 95%, 96%, 97%, 98%, or 99% identity to SEQ ID NO: 161.

In some embodiments, the transmembrane domain is an aTM domain. In some embodiments, the transmembrane domain is a fragment of an aTM domain. In some embodiments, an aTM domain comprises the amino acid sequence set forth in SEQ ID NO: 41 or SEQ ID NO: 163. In some embodiments, an aTM domain consists of the amino acid sequence set forth in SEQ ID NO: 41. In some embodiments, an aTM domain consists of the amino acid sequence set forth in SEQ ID NO: 163. In some embodiments, an aTM domain comprises an amino acid sequence having at least 80%, 85%, 90%, 91%, 92%, 93%, 94%, 95%, 96%, 97%, 98% or 99% identity to SEQ ID NO: 41. In some embodiments, an aTM domain comprises an amino acid sequence having at least 80%, 85%, 90%, 91%, 92%, 93%, 94%, 95%, 96%, 97%, 98% or 99% identity to SEQ ID NO: 163.

In some embodiments, an aTM domain is encoded by the nucleotide sequence set forth in SEQ ID NO: 164 or 165. In some embodiments, an aTM domain is encoded by a nucleotide sequence having at least 80%, 85%, 90%, 91%, 92%, 93%, 94%, 95%, 96%, 97%, 98%, or 99% identity to SEQ ID NO: 164 or 165.

In some embodiments, the transmembrane domain is a transmembrane domain of DNAM1. In some embodiments, the transmembrane domain is a fragment of a DNAM1 transmembrane domain. In some embodiments, a DNAM1 transmembrane domain comprises the amino acid sequence set forth in SEQ ID NO: 42. In some embodiments, a DNAM1 transmembrane domain consists of the amino acid sequence set forth in SEQ ID NO: 42. In some embodiments, a DNAM1 transmembrane domain comprises an amino acid sequence having at least 80%, 85%, 90%, 91%, 92%, 93%, 94%, 95%, 96%, 97%, 98% or 99% identity to SEQ ID NO: 42.

In some embodiments, a DNAM1 transmembrane domain is encoded by the nucleotide sequence set forth in SEQ ID NO: 162. In some embodiments, a DNAM1 transmembrane domain is encoded by a nucleotide sequence having at least 80%, 85%, 90%, 91%, 92%, 93%, 94%, 95%, 96%, 97%, 98%, or 99% identity to SEQ ID NO: 162.

### Hinge Domains

In an aspect, the disclosure provides CARs having a portion that includes a hinge domain. The hinge domain of the CAR of the disclosure may comprise a linker or spacer sequence between the extracellular antigen binding domain and the transmembrane domain. One of ordinary skill in the art will appreciate that a hinge domain (e.g., sequence) is a polypeptide segment that, in at least some instances, facilitates flexibility (see, e.g., Woof et al., Nat. Rev. Immunol., 4 (2): 89-99 (2004)). The hinge sequence can be any suitable sequence derived or obtained from any suitable molecule.

The hinge may be derived from or include at least a portion of an immunoglobulin Fc region, for example, an IgG1 Fc region, an IgG2 Fc region, an IgG3 Fc region, an IgG4 Fc region, an IgE Fc region, an IgM Fc region, or an IgA Fc region. In certain embodiments, the spacer domain includes at least a portion of an IgG1, an IgG2, an IgG3, an IgG4, an IgE, an IgM, or an IgA immunoglobulin Fc region that falls within its CH2 and CH3 domains. In some embodiments, the spacer domain may also include at least a portion of a corresponding immunoglobulin hinge region.

In some embodiments, a portion of the immunoglobulin constant region serves as a hinge region between the antigen binding domain, e.g., scFv, and transmembrane domain. The spacer can be of a length that provides for increased responsiveness of the cell following antigen binding, as compared to in the absence of the spacer. Exemplary hinges include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a hinge has about 12 amino acids or less, about 119 amino acids or less, or about 229 amino acids or less. In some embodiments, the hinge is at or about 12 amino acids in length. Exemplary spacers include, but are not limited to, those described in Hudecek et al. (2013) Clin. Cancer Res., 19:3153, international patent application publication number WO2014031687, U.S. Pat. Pub. No. US 2014/0271635.

The CARs of the disclosure may comprise a hinge domain isolated or derived from CD4, CD8α, CD28, IgG1, IgG2, IgG4, or IgD.

In some embodiments, the hinge domain is isolated or derived from the human CD8a molecule. The hinge domain may comprise the polypeptide sequence: TTTPAPRPPTPAPTIASQPLSLRPEACRPAAGGAVHTRGLDFACD (SEQ ID NO: 38) or a variant with 1, 2, 3, 4, 5 or more substitutions.

In some embodiments, the hinge domain is isolated or derived from the human CD28 molecule. The hinge domain may comprise the polypeptide sequence: CTIEVMYPPPYLDNEKSNGTIIHVKGKHLCPSPLFPGPSKP (SEQ ID NO: 39) or a variant with 1, 2, 3, 4, 5 or more substitutions. The hinge domain may comprise the polypeptide sequence: LDNEKSNGTIIHVKGKHLCPSPLFPGPSKP (SEQ ID NO: 217) or a variant with 1, 2, 3, 4, 5 or more substitutions.

In some embodiments, the hinge domain is isolated or derived from the human IgG4 molecule. The hinge domain may comprise the polypeptide sequence: ESKYGPPCPPCP (SEQ ID NO: 40) or a variant with 1, 2, 3, 4, 5 or more substitutions.

In some embodiments, the hinge domain is isolated or derived from the human CD8 molecule. The hinge domain may comprise the polypeptide sequence: TTTTPAPRPPTPAPTIASQPLSLRPEACRPAAGGAVHTRGLDFACD (SEQ ID NO: 158) or a variant with 1, 2, 3, 4, 5 or more substitutions.

### Linkers

In some embodiments, a chimeric receptor described herein comprises one or more linkers between the components of the receptors. In some embodiments, a chimeric receptor described herein comprises a linker between the extracellular domain and the transmembrane domain. In some embodiments, a chimeric receptor described herein comprises a linker between the transmembrane domain and the cytoplasmic domain. In some embodiments, a chimeric receptor described herein comprises a linker between two or more intracellular signaling domains of the cytoplasmic domain.

### Gly-Ser Linkers

Linkers suitable for joining polypeptide sequences are known to those of skill in the art. Exemplary linkers include gly-ser polypeptide linkers, glycine-proline polypeptide linkers, and proline-alanine polypeptide linkers. In certain embodiments, the linker is a gly-ser polypeptide linker, i.e., a peptide that consists of glycine and serine residues.

As used herein, the term “gly-ser linker” refers to a peptide that consists of glycine and serine residues. An exemplary gly-ser polypeptide linker comprises the amino acid sequence Ser (Gly₄Ser)n. In some embodiments, n=1. In certain embodiments, n=2. In certain embodiments, n=3, i.e., Ser (Gly₄Ser) 3. In some embodiments, n=4, i.e., Ser (Gly₄Ser) 4. In certain embodiments, n=5. In certain embodiments, n=6. In some embodiments, n=7. In some embodiments, n=8. In some embodiments, n=9. In certain embodiments, n=10. Another exemplary gly-ser polypeptide linker comprises the amino acid sequence (Gly₄Ser)n. In some embodiments, n=1. In certain embodiments, n=2. In certain embodiments, n=3. In some embodiments, n=4. In certain embodiments, n=5. In certain embodiments, n=6. Another exemplary gly-ser polypeptide linker comprises the amino acid sequence (Gly₃Ser)n. In some embodiments, n=1. In some embodiments, n=2. In some embodiments, n=3. In some embodiments, n=4. In some embodiments, n=5. In some embodiments, n=6. Exemplary gly-ser polypeptide linkers comprise the amino acid sequence Ser (Gly₄Ser)n. In some embodiments, n=1. In some embodiments, n=2. In some embodiments, n=3, i.e., Ser (Gly₄Ser) 3. In some embodiments, n=4, i.e., Ser (Gly₄Ser) 4. In some embodiments, n=5. In certain embodiments, n=6. In some embodiments, n=7. In some embodiments, n=8. In certain embodiments, n=9. In some embodiments, n=10. Another exemplary gly-ser polypeptide linker comprises the amino acid sequence Ser (Gly₄Ser)n. In certain embodiments, n=1. In some embodiments, n=2. In some embodiments, n=3. In certain embodiments, n=4. In some embodiments, n=5. In certain embodiments, n=6. Another exemplary gly-ser polypeptide linker comprises (Gly₄Ser)n. In some embodiments, n=1. In some embodiments, n=2. In certain embodiments, n=3. In some embodiments, n=4. In some embodiments, n=5. In certain embodiments, n=6. Another exemplary gly-ser polypeptide linker comprises (Gly₃Ser)n. In some embodiments, n=1. In some embodiments, n=2. In certain embodiments, n=3. In certain embodiments, n=4. In some embodiments, n=5. In some embodiments n=6.

In some embodiments, a chimeric receptor described herein comprises a gly-ser linker as set forth in SEQ ID NO: 166. In some embodiments, the gly-ser linker is encoded by the nucleotide sequence of SEQ ID NO: 167. In some embodiments, a chimeric receptor described herein comprises a gly-ser linker as set forth in SEQ ID NO: 168. In some embodiments, the gly-ser linker is encoded by the nucleotide sequence of SEQ ID NO: 169. In some embodiments, a chimeric receptor described herein comprises a gly-ser linker as set forth in SEQ ID NO: 170. In some embodiments, the gly-ser linker is encoded by the nucleotide sequence of SEQ ID NO: 171. In some embodiments, a chimeric receptor described herein comprises a gly-ser linker as set forth in SEQ ID NO: 172. In some embodiments, the gly-ser linker is encoded by the nucleotide sequence of SEQ ID NO: 173.

### Additional Linkers

Additional inkers suitable for joining polypeptide sequences to allow bi-cistronic expression are known to those of skill in the art. Exemplary linkers include Furin GSG-T2A.

In some embodiments, a chimeric receptor described herein comprises a gly-ser linker as set forth in SEQ ID NO: 174. In some embodiments, the gly-ser linker is encoded by the nucleotide sequence of SEQ ID NO: 175.

### CAR Polypeptide and Polynucleotide Sequences

While the disclosure contemplates the combination of the signaling domains disclosure in any order and combined with any transmembrane and extracellular domains, the disclosure provides, at least, the constructs listed in Table 4.

TABLE 4
TLR domain constructs

| | | Amino acid sequence | |
|---|---|---|---|
| Construct | Amino acid sequence | (with anti-CD19) | Polynucleotide sequence |
| CD8-aTM- | TTTTPAPRPPTPAPTIASQPLSL | MALPVTALLLPLALLLHAARP | atggcactgcctgtgaccgccctgctgctgcca |
| TLR2-CD3 | RPEACRPAAGGAVHTRGLDF | YPYDVPDYAYPYDVPDYAYP | ctggccctgctgctgcacgccgccaggccttat |
| | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | ICYDAFVSYSERDAYWVENL | NWYQQKPDGTVKLLIYHTSRL | ctgattacgccggctcgagtgacatccagatga |
| | MVQELENFNPPFKLCLHKRDF | HSGVPSRFSGSGSGTDYSLTIS | cccagaccacatctagcctgtccgcctctctgg |
| | IPGKWIIDNIIDSIEKSHKTVFV | NLEQEDIATYFCQQGNTLPYT | gcgacagggtgacaatctcctgcagagcctct |
| | LSENFVKSEWCKYELDFSHFR | FGGGTKLEITGSTSGSGKPGSG | caggatatcagcaagtatctgaactggtatcag |
| | LFDENNDAAILILLEPIEKKAIP | EGSTKGEVKLQESGPGLVAPS | cagaagcccgatggcaccgtgaagctgctgat |
| | QRFCKLRKIMNTKTYLEWPM | QSLSVTCTVSGVSLPDYGVSW | ctatcacacatccaggctgcactctggcgtgcc |
| | DEAQREGFWVNLRAAITGGG | IRQPPRKGLEWLGVIWGSETT | tagccgcttcagcggctccggctctggcaccg |
| | SGGSRVKFSRSADAPAYQQGQ | YYNSALKSRLTIIKDNSKSQVF | actactccctgacaatctctaacctggagcagg |
| | NQLYNELNLGRREEYDVLDK | LKMNSLQTDDTAIYYCAKHY | aggatatcgccacctatttctgccagcagggca |
| | RRGRDPEMGGKPRRKNPQEG | YYGGSYAMDYWGQGTSVTV | ataccctgccatacacatttggggcggcacaa |
| | LYNELQKDKMAEAYSEIGMK | SGILTTTTPAPRPPTPAPTIASQ | agctggagatcaccggcagcacatccggctct |
| | GERRRGKGHDGLYQGLSTAT | PLSLRPEACRPAAGGAVHTRG | ggcaagccaggcagcggagagggctccacc |
| | KDTYDALHMQALPPR (SEQ ID | LDFACDASSNLFVASWIAVMII | aagggagaggtgaagctgcaggagtccggac |
| | NO: 82) | FRIGMAVAIFCCFFFPSGSGGS | caggcctggtggcacccagccagtccctgtct |
| | | GGSICYDAFVSYSERDAYWVE | gtgacctgtacagtgtctggcgtgagcctgccc |
| | | NLMVQELENFNPPFKLCLHKR | gactacggcgtgtcttggatcagacagccccct |
| | | DFIPGKWIIDNIIDSIEKSHKTV | agaaagggcctggagtggctgggcgtgatctg |
| | | FVLSENFVKSEWCKYELDFSH | gggcagcgagaccacatactataattctgccct |
| | | FRLFDENNDAAILILLEPIEKK | gaagagccggctgaccatcatcaaggacaac |
| | | AIPQRFCKLRKIMNTKTYLEW | agcaagtcccaggtgttcctgaagatgaacag |
| | | PMDEAQREGFWVNLRAAITG | cctgcagaccgacgatacagccatctactattg |
| | | GGSGGSRVKFSRSADAPAYQQ | tgccaagcactactattacggcggcagctatgc |
| | | GQNQLYNELNLGRREEYDVL | tatggactactggggccagggcacctccgtga |
| | | DKRRGRDPEMGGKPRRKNPQ | cagtgtctggaattcttaccacaaccacaccag |
| | | EGLYNELQKDKMAEAYSEIG | cacctcggccacccaccccagcaccaacaatc |
| | | MKGERRRGKGHDGLYQGLST | gccagccagcctctgtccctgaggccagagg |
| | | ATKDTYDALHMQALPPR | catgcaggccagcagcaggaggagcagtgc |
| | | (SEQ ID NO: 100) | acaccagaggcctggactttgcctgtgatgcta |
| | | | gctccaacctgtttgtggcctcttggatcgccgt |
| | | | gatgatcatctttcggatcggcatggccgtggc |
| | | | catcttctgctgtttctttttccccagcggatccg |
| | | | gcggctctggcggcagcatctgctacgacgcctt |
| | | | tgtgagctattccgagcgggatgcctactgggt |
| | | | ggagaacctgatggtgcaggagctggagaac |
| | | | tttaatccccctttcaagctgtgcctgcacaaga |
| | | | gggacttcatccctggcaagtggatcatcgaca |
| | | | acatcatcgattccatcgagaagtctcacaaga |
| | | | ccgtgtttgtgctgagcgagaatttcgtgaagtc |
| | | | cgagtggtgcaagtatgagctggatttctctcac |
| | | | tttcggctgttcgacgagaacaatgatgccgcc |
| | | | atcctgatcctgctggagccaatcgagaagaa |
| | | | ggccatcccccagcggttttgtaagctgagaaa |
| | | | gatcatgaacaccaagacatacctggagtggc |
| | | | ctatggacgaggcccagagggagggcttctg |
| | | | ggtgaatctgcgcgccgccatcaccggtggcg |
| | | | gctctggcggcagcagagtgaagttcagcag |
| | | | gtccgccgacgcacctgcataccagcaggga |
| | | | cagaaccagctgtataacgagctgaatctggg |
| | | | ccggagagaggagtacgacgtgctggataag |
| | | | aggcggggccgggaccccgagatgggaggc |
| | | | aagccacggagaaagaacccccaggagggc |
| | | | ctgtacaatgagctgcagaaggacaagatggc |
| | | | cgaggcctattccgagatcggcatgaagggag |
| | | | agaggcgccggggcaagggacacgatggcc |
| | | | tgtaccagggcctgtctaccgccacaaaggac |
| | | | acctatgatgccctgcacatgcaggccctgccc |
| | | | cctcgg (SEQ ID NO: 118) |
| CD8-aTM- | TTTTPAPRPPTPAPTIASQPLSL | MALPVTALLLPLALLLHAARP | atggcactgcctgtgaccgccctgctgctgcca |
| 2B4-TLR2- | RPEACRPAAGGAVHTRGLDF | YPYDVPDYAYPYDVPDYAYP | ctggccctgctgctgcacgccgccaggccttat |
| CD3 | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | WRRKRKEKQSETSPKEFLTIY | NWYQQKPDGTVKLLIYHTSRL | ctgattacgccggctcgagtgacatccagatga |
| | EDVKDLKTRRNHEQEQTFPGG | HSGVPSRFSGSGSGTDYSLTIS | cccagaccacatctagcctgtccgcctctctgg |
| | GSTIYSMIQSQSSAPTSQEPAY | NLEQEDIATYFCQQGNTLPYT | gcgacagggtgacaatctcctgcagagcctct |
| | TLYSLIQPSRKSGSRKRNHSPS | FGGGTKLEITGSTSGSGKPGSG | caggatatcagcaagtatctgaactggtatcag |
| | FNSTIYEVIGKSQPKAQNPARL | EGSTKGEVKLQESGPGLVAPS | cagaagcccgatggcaccgtgaagctgctgat |
| | SRKELENFDVYSSLGGGSGGSI | QSLSVTCTVSGVSLPDYGVSW | ctatcacacatccaggctgcactctggcgtgcc |
| | CYDAFVSYSERDAYWVENLM | IRQPPRKGLEWLGVIWGSETT | tagccgcttcagcggctccggctctggcaccg |
| | VQELENFNPPFKLCLHKRDFIP | YYNSALKSRLTIIKDNSKSQVF | actactccctgacaatctctaacctggagcagg |
| | GKWIIDNIIDSIEKSHKTVFVLS | LKMNSLQTDDTAIYYCAKHY | aggatatcgccacctatttctgccagcagggca |
| | ENFVKSEWCKYELDFSHFRLF | YYGGSYAMDYWGQGTSVTV | ataccctgccatacacatttggcggcggcacaa |
| | DENNDAAILILLEPIEKKAIPQR | SGILTTTTPAPRPPTPAPTIASQ | agctggagatcaccggcagcacatccggctct |
| | FCKLRKIMNTKTYLEWPMDE | PLSLRPEACRPAAGGAVHTRG | ggcaagccaggcagcggagagggctccacc |
| | AQREGFWVNLRAAITGGGSG | LDFACDASSNLFVASWIAVMII | aagggagaggtgaagctgcaggagtccggac |
| | GSRVKFSRSADAPAYQQGQN | FRIGMAVAIFCCFFFPSGSGGS | caggcctggtggcacccagccagtccctgtct |
| | QLYNELNLGRREEYDVLDKR | GGSWRRKRKEKQSETSPKEFL | gtgacctgtacagtgtctggcgtgagcctgccc |
| | RGRDPEMGGKPRRKNPQEGL | TIYEDVKDLKTRRNHEQEQTF | gactacggcgtgtcttggatcagacagccccct |
| | YNELQKDKMAEAYSEIGMKG | PGGGSTIYSMIQSQSSAPTSQE | agaaagggcctggagtggctgggcgtgatctg |
| | ERRRGKGHDGLYQGLSTATK | PAYTLYSLIQPSRKSGSRKRNH | gggcagcgagaccacatactataattctgccct |
| | DTYDALHMQALPPR (SEQ ID | SPSFNSTIYEVIGKSQPKAQNP | gaagagccggctgaccatcatcaaggacaac |
| | NO: 83) | ARLSRKELENFDVYSSLGGGS | agcaagtcccaggtgttcctgaagatgaacag |
| | | GGSICYDAFVSYSERDAYWVE | cctgcagaccgacgatacagccatctactattg |
| | | NLMVQELENFNPPFKLCLHKR | tgccaagcactactattacggcggcagctatgc |
| | | DFIPGKWIIDNIIDSIEKSHKTV | tatggactactggggccagggcacctccgtga |
| | | FVLSENFVKSEWCKYELDFSH | cagtgtctggaattcttaccacaaccacaccag |
| | | FRLFDENNDAAILILLEPIEKK | cacctcggccacccaccccagcaccaacaatc |
| | | AIPQRFCKLRKIMNTKTYLEW | gccagccagcctctgtccctgaggccagagg |
| | | PMDEAQREGFWVNLRAAITG | catgcaggccagcagcaggaggagcagtgc |
| | | GGSGGSRVKFSRSADAPAYQQ | acaccagaggcctggactttgcctgtgatgcta |
| | | GQNQLYNELNLGRREEYDVL | gctccaacctgtttgtggcctcttggatcgccgt |
| | | DKRRGRDPEMGGKPRRKNPQ | gatgatcatctttcggatcggcatggccgtggc |
| | | EGLYNELQKDKMAEAYSEIG | catcttctgctgtttctttttccccagcggatccg |
| | | MKGERRRGKGHDGLYQGLST | gcggctctggcggcagctggcggagaaagcg |
| | | ATKDTYDALHMQALPPR | gaaggagaagcagtctgagaccagccccaag |
| | | (SEQ ID NO: 101) | gagttcctgacaatctacgaggacgtgaaggat |
| | | | ctgaagaccaggcgcaaccacgagcaggag |
| | | | cagacctttcctggcggcggcagcacaatctat |
| | | | tccatgatccagtctcagagctccgcccccacc |
| | | | tcccaggagcctgcctacacactgtatagcctg |
| | | | atccagccttcccggaagtccggctctcggaa |
| | | | gagaaaccacagcccatccttcaattctacaat |
| | | | ctacgaagtgatcggcaagagccagccaaag |
| | | | gcacagaaccccgccaggctgagccgcaag |
| | | | gagctggagaattttgacgtgtattcttccctag |
| | | | gaggcggctccggcggctctatctgctacgac |
| | | | gcctttgtgagctattccgagcgggatgcctact |
| | | | gggtggagaacctgatggtgcaggagctgga |
| | | | gaactttaatccccctttcaagctgtgcctgcac |
| | | | aagagggacttcatccctggcaagtggatcatc |
| | | | gacaacatcatcgattccatcgagaagtctcac |
| | | | aagaccgtgtttgtgctgagcgagaatttcgtga |
| | | | agtccgagtggtgcaagtatgagctggatttctc |
| | | | tcactttcggctgttcgacgagaacaatgatgcc |
| | | | gccatcctgatcctgctggagccaatcgagaa |
| | | | gaaggccatcccccagcggttttgtaagctgag |
| | | | aaagatcatgaacaccaagacatacctggagt |
| | | | ggcctatggacgaggcccagagggagggctt |
| | | | ctgggtgaatctgcgcgccgccatcaccggtg |
| | | | gcggctctggcggcagcagagtgaagttcag |
| | | | caggtccgccgacgcacctgcataccagcag |
| | | | ggacagaaccagctgtataacgagctgaatct |
| | | | gggccggagagaggagtacgacgtgctggat |
| | | | aagaggcggggccgggaccccgagatggga |
| | | | ggcaagccacggagaaagaacccccaggag |
| | | | ggcctgtacaatgagctgcagaaggacaagat |
| | | | ggccgaggcctattccgagatcggcatgaagg |
| | | | gagagaggcgccggggcaagggacacgatg |
| | | | gcctgtaccagggcctgtctaccgccacaaag |
| | | | gacacctatgatgccctgcacatgcaggccct |
| | | | gccccctcgg (SEQ ID NO: 119) |
| CD8-aTM- | TTTTPAPRPPTPAPTIASQPLSL | MALPVTALLLPLALLLHAARP | atggcactgcctgtgaccgccctgctgctgcca |
| IL18R-CD3 | RPEACRPAAGGAVHTRGLDF | YPYDVPDYAYPYDVPDYAYP | ctggccctgctgctgcacgccgccaggccttat |
| | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | KTYDAFVSYLKECRPENGEEH | NWYQQKPDGTVKLLIYHTSRL | ctgattacgccggctcgagtgacatccagatga |
| | TFAVEILPRVLEKHFGYKLCIF | HSGVPSRFSGSGSGTDYSLTIS | cccagaccacatctagcctgtccgcctctctgg |
| | ERDVVPGGAVVDEIHSLIEKSR | NLEQEDIATYFCQQGNTLPYT | gcgacagggtgacaatctcctgcagagcctct |
| | RLIIVLSKSYMSNEVRYELESG | FGGGTKLEITGSTSGSGKPGSG | caggatatcagcaagtatctgaactggtatcag |
| | LHEALVERKIKIILIEFTPVTDF | EGSTKGEVKLQESGPGLVAPS | cagaagcccgatggcaccgtgaagctgctgat |
| | TFLPQSLKLLKSHRVLKWKAD | QSLSVTCTVSGVSLPDYGVSW | ctatcacacatccaggctgcactctggcgtgcc |
| | KSLSYNSRFWKNLLYLMTGG | IRQPPRKGLEWLGVIWGSETT | tagccgcttcagcggctccggctctggcaccg |
| | GSGGSRVKFSRSADAPAYQQG | YYNSALKSRLTIIKDNSKSQVF | actactccctgacaatctctaacctggagcagg |
| | QNQLYNELNLGRREEYDVLD | LKMNSLQTDDTAIYYCAKHY | aggatatcgccacctatttctgccagcagggca |
| | KRRGRDPEMGGKPRRKNPQE | YYGGSYAMDYWGQGTSVTV | ataccctgccatacacatttggcggcggcacaa |
| | GLYNELQKDKMAEAYSEIGM | SGILTTTTPAPRPPTPAPTIASQ | agctggagatcaccggcagcacatccggctct |
| | KGERRRGKGHDGLYQGLSTA | PLSLRPEACRPAAGGAVHTRG | ggcaagccaggcagcggagagggctccacc |
| | TKDTYDALHMQALPPR (SEQ | LDFACDASSNLFVASWIAVMII | aagggagaggtgaagctgcaggagtccggac |
| | ID NO: 84) | FRIGMAVAIFCCFFFPSGSGGS | caggcctggtggcacccagccagtccctgtct |
| | | GGSKTYDAFVSYLKECRPENG | gtgacctgtacagtgtctggcgtgagcctgccc |
| | | EEHTFAVEILPRVLEKHFGYKL | gactacggcgtgtcttggatcagacagccccct |
| | | CIFERDVVPGGAVVDEIHSLIE | agaaagggcctggagtggctgggcgtgatctg |
| | | KSRRLIIVLSKSYMSNEVRYEL | gggcagcgagaccacatactataattctgccct |
| | | ESGLHEALVERKIKIILIEFTPV | gaagagccggctgaccatcatcaaggacaac |
| | | TDFTFLPQSLKLLKSHRVLKW | agcaagtcccaggtgttcctgaagatgaacag |
| | | KADKSLSYNSRFWKNLLYLM | cctgcagaccgacgatacagccatctactattg |
| | | TGGGSGGSRVKFSRSADAPAY | tgccaagcactactattacggcggcagctatgc |
| | | QQGQNQLYNELNLGRREEYD | tatggactactggggccagggcacctccgtga |
| | | VLDKRRGRDPEMGGKPRRKN | cagtgtctggaattcttaccacaaccacaccag |
| | | PQEGLYNELQKDKMAEAYSEI | cacctcggccacccaccccagcaccaacaatc |
| | | GMKGERRRGKGHDGLYQGLS | gccagccagcctctgtccctgaggccagagg |
| | | TATKDTYDALHMQALPPR | catgcaggccagcagcaggaggagcagtgc |
| | | (SEQ ID NO: 102) | acaccagaggcctggactttgcctgtgatgcta |
| | | | gctccaacctgtttgtggcctcttggatcgccgt |
| | | | gatgatcatctttcggatcggcatggccgtggc |
| | | | catcttctgctgttttttttccccagcggatccgg |
| | | | cggctctggcggcagcaagacatacgacgcc |
| | | | ttcgtgagctatctgaaggagtgccggccaga |
| | | | gaacggcgaggagcacacctttgccgtggag |
| | | | atcctgccccgggtgctggagaagcacttcgg |
| | | | ctacaagctgtgcatctttgagagagacgtggt |
| | | | gcccggaggagcagtggtggatgagatccac |
| | | | agcctgatcgagaagtcccggagactgatcat |
| | | | cgtgctgagcaagtcctacatgtccaatgaggt |
| | | | gaggtacgagctggagtctggactgcacgag |
| | | | gccctggtggagaggaagatcaagatcatcct |
| | | | gatcgagttcacccccgtgacagacttcaccttt |
| | | | ctgcctcagtctctgaagctgctgaagagccac |
| | | | agggtgctgaagtggaaggccgataagtctct |
| | | | gagctacaactcccgcttttggaagaatctgctg |
| | | | tatctgatgaccggtggcggctccggcggctct |
| | | | agagtgaagttcagcaggtccgccgacgcac |
| | | | ctgcataccagcagggacagaaccagctgtat |
| | | | aacgagctgaatctgggccggagagaggagt |
| | | | acgacgtgctggataagaggggggccggg |
| | | | accccgagatgggaggcaagccacggagaa |
| | | | agaacccccaggagggcctgtacaatgagct |
| | | | gcagaaggacaagatggccgaggcctattcc |
| | | | gagatcggcatgaagggagagaggcgccgg |
| | | | ggcaagggacacgatggcctgtaccagggcc |
| | | | tgtctaccgccacaaaggacacctatgatgccc |
| | | | tgcacatgcaggccctgccccctcgg (SEQ |
| | | | ID NO: 120) |
| CD8-aTM- | TTTTPAPRPPTPAPTIASQPLSL | MALPVTALLLPLALLLHAARP | atggcactgcctgtgaccgccctgctgctgcca |
| 2B4-IL18R- | RPEACRPAAGGAVHTRGLDF | YPYDVPDYAYPYDVPDYAYP | ctggccctgctgctgcacgccgccaggccttat |
| CD3 | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | WRRKRKEKQSETSPKEFLTIY | NWYQQKPDGTVKLLIYHTSRL | ctgattacgccggctcgagtgacatccagatga |
| | EDVKDLKTRRNHEQEQTFPGG | HSGVPSRFSGSGSGTDYSLTIS | cccagaccacatctagcctgtccgcctctctgg |
| | GSTIYSMIQSQSSAPTSQEPAY | NLEQEDIATYFCQQGNTLPYT | gcgacagggtgacaatctcctgcagagcctct |
| | TLYSLIQPSRKSGSRKRNHSPS | FGGGTKLEITGSTSGSGKPGSG | caggatatcagcaagtatctgaactggtatcag |
| | FNSTIYEVIGKSQPKAQNPARL | EGSTKGEVKLQESGPGLVAPS | cagaagcccgatggcaccgtgaagctgctgat |
| | SRKELENFDVYSSLGGGSGGS | QSLSVTCTVSGVSLPDYGVSW | ctatcacacatccaggctgcactctggcgtgcc |
| | KTYDAFVSYLKECRPENGEEH | IRQPPRKGLEWLGVIWGSETT | tagccgcttcagcggctccggctctggcaccg |
| | TFAVEILPRVLEKHFGYKLCIF | YYNSALKSRLTIIKDNSKSQVF | actactccctgacaatctctaacctggagcagg |
| | ERDVVPGGAVVDEIHSLIEKSR | LKMNSLQTDDTAIYYCAKHY | aggatatcgccacctatttctgccagcagggca |
| | RLIIVLSKSYMSNEVRYELESG | YYGGSYAMDYWGQGTSVTV | ataccctgccatacacatttggcggcggcacaa |
| | LHEALVERKIKIILIEFTPVTDF | SGILTTTTPAPRPPTPAPTIASQ | agctggagatcaccggcagcacatccggctct |
| | TFLPQSLKLLKSHRVLKWKAD | PLSLRPEACRPAAGGAVHTRG | ggcaagccaggcagcggagagggctccacc |
| | KSLSYNSRFWKNLLYLMTGG | LDFACDASSNLFVASWIAVMII | aagggagaggtgaagctgcaggagtccggac |
| | GSGGSRVKFSRSADAPAYQQG | FRIGMAVAIFCCFFFPSGSGGS | caggcctggtggcacccagccagtccctgtct |
| | QNQLYNELNLGRREEYDVLD | GGSWRRKRKEKQSETSPKEFL | gtgacctgtacagtgtctggcgtgagcctgccc |
| | KRRGRDPEMGGKPRRKNPQE | TIYEDVKDLKTRRNHEQEQTF | gactacggcgtgtcttggatcagacagccccct |
| | GLYNELQKDKMAEAYSEIGM | PGGGSTIYSMIQSQSSAPTSQE | agaaagggcctggagtggctgggcgtgatctg |
| | KGERRRGKGHDGLYQGLSTA | PAYTLYSLIQPSRKSGSRKRNH | gggcagcgagaccacatactataattctgccct |
| | TKDTYDALHMQALPPR (SEQ | SPSFNSTIYEVIGKSQPKAQNP | gaagagccggctgaccatcatcaaggacaac |
| | ID NO: 85) | ARLSRKELENFDVYSSLGGGS | agcaagtcccaggtgttcctgaagatgaacag |
| | | GGSKTYDAFVSYLKECRPENG | cctgcagaccgacgatacagccatctactattg |
| | | EEHTFAVEILPRVLEKHFGYKL | tgccaagcactactattacggcggcagctatgc |
| | | CIFERDVVPGGAVVDEIHSLIE | tatggactactggggccagggcacctccgtga |
| | | KSRRLIIVLSKSYMSNEVRYEL | cagtgtctggaattcttaccacaaccacaccag |
| | | ESGLHEALVERKIKIILIEFTPV | cacctggccacccaccccagcaccaacaatc |
| | | TDFTFLPQSLKLLKSHRVLKW | gccagccagcctctgtccctgaggccagagg |
| | | KADKSLSYNSRFWKNLLYLM | catgcaggccagcagcaggaggagcagtgc |
| | | TGGGSGGSRVKFSRSADAPAY | acaccagaggcctggactttgcctgtgatgcta |
| | | QQGQNQLYNELNLGRREEYD | gctccaacctgtttgtggcctcttggatcgccgt |
| | | VLDKRRGRDPEMGGKPRRKN | gatgatcatctttcggatcggcatggccgtggc |
| | | PQEGLYNELQKDKMAEAYSEI | catcttctgctgtttctttttccccagcggatccg |
| | | GMKGERRRGKGHDGLYQGLS | gcggctctggcggcagctggcggagaaagcg |
| | | TATKDTYDALHMQALPPR | gaaggagaagcagtctgagaccagccccaag |
| | | (SEQ ID NO: 103) | gagttcctgacaatctacgaggacgtgaaggat |
| | | | ctgaagaccaggcgcaaccacgagcaggag |
| | | | cagacctttcctggcggcggcagcacaatctat |
| | | | tccatgatccagtctcagagctccgcccccacc |
| | | | tcccaggagcctgcctacacactgtatagcctg |
| | | | atccagccttcccggaagtccggctctcggaa |
| | | | gagaaaccacagcccatccttcaattctacaat |
| | | | ctacgaagtgatcggcaagagccagccaaag |
| | | | gcacagaaccccgccaggctgagccgcaag |
| | | | gagctggagaattttgacgtgtattcttccctag |
| | | | gaggcggctccggcggctctaagacatacga |
| | | | cgccttcgtgagctatctgaaggagtgccggc |
| | | | cagagaacggcgaggagcacacctttgccgt |
| | | | ggagatcctgccccgggtgctggagaagcact |
| | | | tcggctacaagctgtgcatctttgagagagacg |
| | | | tggtgcccggaggagcagtggtggatgagatc |
| | | | cacagcctgatcgagaagtcccggagactgat |
| | | | catcgtgctgagcaagtcctacatgtccaatga |
| | | | ggtgaggtacgagctggagtctggactgcacg |
| | | | aggccctggtggagaggaagatcaagatcatc |
| | | | ctgatcgagttcacccccgtgacagacttcacc |
| | | | tttctgcctcagtctctgaagctgctgaagagcc |
| | | | acagggtgctgaagtggaaggccgataagtct |
| | | | ctgagctacaactcccgcttttggaagaatctgc |
| | | | tgtatctgatgaccggtggcggctccggcggct |
| | | | ctagagtgaagttcagcaggtccgccgacgca |
| | | | cctgcataccagcagggacagaaccagctgta |
| | | | taacgagctgaatctgggccggagagaggag |
| | | | tacgacgtgctggataagaggcggggccggg |
| | | | accccgagatgggaggcaagccacggagaa |
| | | | agaacccccaggagggcctgtacaatgagct |
| | | | gcagaaggacaagatggccgaggcctattcc |
| | | | gagatcggcatgaagggagagaggcgccgg |
| | | | ggcaagggacacgatggcctgtaccagggcc |
| | | | tgtctaccgccacaaaggacacctatgatgccc |
| | | | tgcacatgcaggccctgccccctcgg (SEQ |
| | | | ID NO: 121) |
| CD8-aTM- | TTTTPAPRPPTPAPTIASQPLSL | MALPVTALLLPLALLLHAARP | atggcactgcctgtgaccgccctgctgctgcca |
| 2B4-CD3- | RPEACRPAAGGAVHTRGLDF | YPYDVPDYAYPYDVPDYAYP | ctggccctgctgctgcacgccgccaggccttat |
| IL18R | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | WRRKRKEKQSETSPKEFLTIY | NWYQQKPDGTVKLLIYHTSRL | ctgattacgccggctcgagtgacatccagatga |
| | EDVKDLKTRRNHEQEQTFPGG | HSGVPSRFSGSGSGTDYSLTIS | cccagaccacatctagcctgtccgcctctctgg |
| | GSTIYSMIQSQSSAPTSQEPAY | NLEQEDIATYFCQQGNTLPYT | gcgacagggtgacaatctcctgcagagcctct |
| | TLYSLIQPSRKSGSRKRNHSPS | FGGGTKLEITGSTSGSGKPGSG | caggatatcagcaagtatctgaactggtatcag |
| | FNSTIYEVIGKSQPKAQNPARL | EGSTKGEVKLQESGPGLVAPS | cagaagcccgatggcaccgtgaagctgctgat |
| | SRKELENFDVYSSLGGGSGGS | QSLSVTCTVSGVSLPDYGVSW | ctatcacacatccaggctgcactctggcgtgcc |
| | RVKFSRSADAPAYQQGQNQL | IRQPPRKGLEWLGVIWGSETT | tagccgcttcagcggctccggctctggcaccg |
| | YNELNLGRREEYDVLDKRRG | YYNSALKSRLTIIKDNSKSQVF | actactccctgacaatctctaacctggagcagg |
| | RDPEMGGKPRRKNPQEGLYN | LKMNSLQTDDTAIYYCAKHY | aggatatcgccacctatttctgccagcagggca |
| | ELQKDKMAEAYSEIGMKGER | YYGGSYAMDYWGQGTSVTV | ataccctgccatacacatttggcggcggcacaa |
| | RRGKGHDGLYQGLSTATKDT | SGILTTTTPAPRPPTPAPTIASQ | agctggagatcaccggcagcacatccggctct |
| | YDALHMQALPPRTGGGSGGS | PLSLRPEACRPAAGGAVHTRG | ggcaagccaggcagcggagagggctccacc |
| | KTYDAFVSYLKECRPENGEEH | LDFACDASSNLFVASWIAVMII | aagggagaggtgaagctgcaggagtccggac |
| | TFAVEILPRVLEKHFGYKLCIF | FRIGMAVAIFCCFFFPSGSGGS | caggcctggtggcacccagccagtccctgtct |
| | ERDVVPGGAVVDEIHSLIEKSR | GGSWRRKRKEKQSETSPKEFL | gtgacctgtacagtgtctggcgtgagcctgccc |
| | RLIIVLSKSYMSNEVRYELESG | TIYEDVKDLKTRRNHEQEQTF | gactacggcgtgtcttggatcagacagccccct |
| | LHEALVERKIKIILIEFTPVTDF | PGGGSTIYSMIQSQSSAPTSQE | agaaagggcctggagtggctgggcgtgatctg |
| | TFLPQSLKLLKSHRVLKWKAD | PAYTLYSLIQPSRKSGSRKRNH | gggcagcgagaccacatactataattctgccct |
| | KSLSYNSRFWKNLLYLM (SEQ | SPSFNSTIYEVIGKSQPKAQNP | gaagagccggctgaccatcatcaaggacaac |
| | ID NO: 86) | ARLSRKELENFDVYSSLGGGS | agcaagtcccaggtgttcctgaagatgaacag |
| | | GGSRVKFSRSADAPAYQQGQ | cctgcagaccgacgatacagccatctactattg |
| | | NQLYNELNLGRREEYDVLDK | tgccaagcactactattacggcggcagctatgc |
| | | RRGRDPEMGGKPRRKNPQEG | tatggactactggggccagggcacctccgtga |
| | | LYNELQKDKMAEAYSEIGMK | cagtgtctggaattcttaccacaaccacaccag |
| | | GERRRGKGHDGLYQGLSTAT | cacctcggccacccaccccagcaccaacaatc |
| | | KDTYDALHMQALPPRTGGGS | gccagccagcctctgtccctgaggccagagg |
| | | GGSKTYDAFVSYLKECRPENG | catgcaggccagcagcaggaggagcagtgc |
| | | EEHTFAVEILPRVLEKHFGYKL | acaccagaggcctggactttgcctgtgatgcta |
| | | CIFERDVVPGGAVVDEIHSLIE | gctccaacctgtttgtggcctcttggatcgccgt |
| | | KSRRLIIVLSKSYMSNEVRYEL | gatgatcatctttcggatcggcatggccgtggc |
| | | ESGLHEALVERKIKIILIEFTPV | catcttctgctgtttctttttccccagcggatccg |
| | | TDFTFLPQSLKLLKSHRVLKW | gcggctctggcggcagctggcggagaaagcg |
| | | KADKSLSYNSRFWKNLLYLM | gaaggagaagcagtctgagaccagccccaag |
| | | (SEQ ID NO: 104) | gagttcctgacaatctacgaggacgtgaaggat |
| | | | ctgaagaccaggcgcaaccacgagcaggag |
| | | | cagacctttcctggcggcggcagcacaatctat |
| | | | tccatgatccagtctcagagctccgcccccacc |
| | | | tcccaggagcctgcctacacactgtatagcctg |
| | | | atccagccttcccggaagtccggctctcggaa |
| | | | gagaaaccacagcccatccttcaattctacaat |
| | | | ctacgaagtgatcggcaagagccagccaaag |
| | | | gcacagaaccccgccaggctgagccgcaag |
| | | | gagctggagaattttgacgtgtattcttccctag |
| | | | gaggcggctccggcggctctagagtgaagttc |
| | | | agcaggtccgccgacgcacctgcataccagc |
| | | | agggacagaaccagctgtataacgagctgaat |
| | | | ctgggccggagagaggagtacgacgtgctgg |
| | | | ataagaggcggggccgggaccccgagatgg |
| | | | gaggcaagccacggagaaagaacccccagg |
| | | | agggcctgtacaatgagctgcagaaggacaa |
| | | | gatggccgaggcctattccgagatcggcatga |
| | | | agggagagaggcgccggggcaagggacac |
| | | | gatggcctgtaccagggcctgtctaccgccac |
| | | | aaaggacacctatgatgccctgcacatgcagg |
| | | | ccctgccccctcggaccggtggcggctctggc |
| | | | ggcagcaagacatacgacgccttcgtgagcta |
| | | | tctgaaggagtgccggccagagaacggcgag |
| | | | gagcacacctttgccgtggagatcctgccccg |
| | | | ggtgctggagaagcacttcggctacaagctgt |
| | | | gcatctttgagagagacgtggtgcccggagga |
| | | | gcagtggtggatgagatccacagcctgatcga |
| | | | gaagtcccggagactgatcatcgtgctgagca |
| | | | agtcctacatgtccaatgaggtgaggtacgagc |
| | | | tggagtctggactgcacgaggccctggtggag |
| | | | aggaagatcaagatcatcctgatcgagttcacc |
| | | | cccgtgacagacttcacctttctgcctcagtctct |
| | | | gaagctgctgaagagccacagggtgctgaagt |
| | | | ggaaggccgataagtctctgagctacaactcc |
| | | | cgcttttggaagaatctgctgtatctgatg |
| | | | (SEQ ID NO: 122) |
| CD8-aTM- | TTTTPAPRPPTPAPTIASQPLSL | MALPVTALLLPLALLLHAARP | atggcactgcctgtgaccgccctgctgctgcca |
| TLR4-CD3 | RPEACRPAAGGAVHTRGLDF | YPYDVPDYAYPYDVPDYAYP | ctggccctgctgctgcacgccgccaggccttat |
| | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | NIYDAFVIYSSQDEDWVRNEL | NWYQQKPDGTVKLLIYHTSRL | ctgattacgccggctcgagtgacatccagatga |
| | VKNLEEGVPPFQLCLHYRDFIP | HSGVPSRFSGSGSGTDYSLTIS | cccagaccacatctagcctgtccgcctctctgg |
| | GVAIAANIIHEGFHKSRKVIVV | NLEQEDIATYFCQQGNTLPYT | gcgacagggtgacaatctcctgcagagcctct |
| | VSQHFIQSRWCIFEYEIAQTWQ | FGGGTKLEITGSTSGSGKPGSG | caggatatcagcaagtatctgaactggtatcag |
| | FLSSRAGIIFIVLQKVEKTLLRQ | EGSTKGEVKLQESGPGLVAPS | cagaagcccgatggcaccgtgaagctgctgat |
| | QVELYRLLSRNTYLEWEDSVL | QSLSVTCTVSGVSLPDYGVSW | ctatcacacatccaggctgcactctggcgtgcc |
| | GRHIFWRRLRKALTGGGSGGS | IRQPPRKGLEWLGVIWGSETT | tagccgcttcagcggctccggctctggcaccg |
| | RVKFSRSADAPAYQQGQNQL | YYNSALKSRLTIIKDNSKSQVF | actactccctgacaatctctaacctggagcagg |
| | YNELNLGRREEYDVLDKRRG | LKMNSLQTDDTAIYYCAKHY | aggatatcgccacctatttctgccagcagggca |
| | RDPEMGGKPRRKNPQEGLYN | YYGGSYAMDYWGQGTSVTV | ataccctgccatacacatttggggcggcacaa |
| | ELQKDKMAEAYSEIGMKGER | SGILTTTTPAPRPPTPAPTIASQ | agctggagatcaccggcagcacatccggctct |
| | RRGKGHDGLYQGLSTATKDT | PLSLRPEACRPAAGGAVHTRG | ggcaagccaggcagcggagagggctccacc |
| | YDALHMQALPPR (SEQ ID | LDFACDASSNLFVASWIAVMII | aagggagaggtgaagctgcaggagtccggac |
| | NO: 87) | FRIGMAVAIFCCFFFPSGSGGS | caggcctggtggcacccagccagtccctgtct |
| | | GGSNIYDAFVIYSSQDEDWVR | gtgacctgtacagtgtctggcgtgagcctgccc |
| | | NELVKNLEEGVPPFQLCLHYR | gactacggcgtgtcttggatcagacagccccct |
| | | DFIPGVAIAANIIHEGFHKSRK | agaaagggcctggagtggctgggcgtgatctg |
| | | VIVVVSQHFIQSRWCIFEYEIA | gggcagcgagaccacatactataattctgccct |
| | | QTWQFLSSRAGIIFIVLQKVEK | gaagagccggctgaccatcatcaaggacaac |
| | | TLLRQQVELYRLLSRNTYLEW | agcaagtcccaggtgttcctgaagatgaacag |
| | | EDSVLGRHIFWRRLRKALTGG | cctgcagaccgacgatacagccatctactattg |
| | | GSGGSRVKFSRSADAPAYQQG | tgccaagcactactattacggcggcagctatgc |
| | | QNQLYNELNLGRREEYDVLD | tatggactactggggccagggcacctccgtga |
| | | KRRGRDPEMGGKPRRKNPQE | cagtgtctggaattcttaccacaaccacaccag |
| | | GLYNELQKDKMAEAYSEIGM | cacctcggccacccaccccagcaccaacaatc |
| | | KGERRRGKGHDGLYQGLSTA | gccagccagcctctgtccctgaggccagagg |
| | | TKDTYDALHMQALPPR (SEQ | catgcaggccagcagcaggaggagcagtgc |
| | | ID NO: 105) | acaccagaggcctggactttgcctgtgatgcta |
| | | | gctccaacctgtttgtggcctcttggatcgccgt |
| | | | gatgatcatctttcggatcggcatggccgtggc |
| | | | catcttctgctgtttctttttccccagcggatccg |
| | | | gcggctctggcggcagcaacatctacgacgcct |
| | | | tcgtgatctatagctcccaggacgaggattggg |
| | | | tgaggaacgagctggtgaagaatctggagga |
| | | | gggcgtgccccctttccagctgtgcctgcacta |
| | | | cagggacttcatcccaggagtggcaatcgccg |
| | | | ccaatatcatccacgagggctttcacaagtcca |
| | | | ggaaagtgatcgtggtggtgagccagcacttc |
| | | | atccagtcccgctggtgtatctttgagtatgagat |
| | | | cgcccagacctggcagttcctgtctagccggg |
| | | | ccggcatcatctttatcgtgctgcagaaggtgg |
| | | | agaagaccctgctgagacagcaggtggagct |
| | | | gtacaggctgctgtctcgcaacacatatctgga |
| | | | gtgggaggacagcgtgctgggccggcacatc |
| | | | ttctggcggagactgagaaaggccctgaccgg |
| | | | tggcggctccggcggctctagagtgaagttca |
| | | | gcaggtccgccgacgcacctgcataccagca |
| | | | gggacagaaccagctgtataacgagctgaatc |
| | | | tgggccggagagaggagtacgacgtgctgga |
| | | | taagaggcggggccgggaccccgagatggg |
| | | | aggcaagccacggagaaagaacccccagga |
| | | | gggcctgtacaatgagctgcagaaggacaag |
| | | | atggccgaggcctattccgagatcggcatgaa |
| | | | gggagagaggcgccggggcaagggacacg |
| | | | atggcctgtaccagggcctgtctaccgccaca |
| | | | aaggacacctatgatgccctgcacatgcaggc |
| | | | cctgccccctcgg (SEQ ID NO: 123) |
| CD8-aTM- | TTTTPAPRPPTPAPTIASQPLSL | MALPVTALLLPLALLLHAARP | atggcactgcctgtgaccgccctgctgctgcca |
| 2B4-TLR4- | RPEACRPAAGGAVHTRGLDF | YPYDVPDYAYPYDVPDYAYP | ctggccctgctgctgcacgccgccaggccttat |
| CD3 | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | WRRKRKEKQSETSPKEFLTIY | NWYQQKPDGTVKLLIYHTSRL | ctgattacgccggctcgagtgacatccagatga |
| | EDVKDLKTRRNHEQEQTFPGG | HSGVPSRFSGSGSGTDYSLTIS | cccagaccacatctagcctgtccgcctctctgg |
| | GSTIYSMIQSQSSAPTSQEPAY | NLEQEDIATYFCQQGNTLPYT | gcgacagggtgacaatctcctgcagagcctct |
| | TLYSLIQPSRKSGSRKRNHSPS | FGGGTKLEITGSTSGSGKPGSG | caggatatcagcaagtatctgaactggtatcag |
| | FNSTIYEVIGKSQPKAQNPARL | EGSTKGEVKLQESGPGLVAPS | cagaagcccgatggcaccgtgaagctgctgat |
| | SRKELENFDVYSSLGGGSGGS | QSLSVTCTVSGVSLPDYGVSW | ctatcacacatccaggctgcactctggcgtgcc |
| | NIYDAFVIYSSQDEDWVRNEL | IRQPPRKGLEWLGVIWGSETT | tagccgcttcagcggctccggctctggcaccg |
| | VKNLEEGVPPFQLCLHYRDFIP | YYNSALKSRLTIIKDNSKSQVF | actactccctgacaatctctaacctggagcagg |
| | GVAIAANIIHEGFHKSRKVIVV | LKMNSLQTDDTAIYYCAKHY | aggatatcgccacctatttctgccagcagggca |
| | VSQHFIQSRWCIFEYEIAQTWQ | YYGGSYAMDYWGQGTSVTV | ataccctgccatacacatttggcggcggcacaa |
| | FLSSRAGIIFIVLQKVEKTLLRQ | SGILTTTTPAPRPPTPAPTIASQ | agctggagatcaccggcagcacatccggctct |
| | QVELYRLLSRNTYLEWEDSVL | PLSLRPEACRPAAGGAVHTRG | ggcaagccaggcagcggagagggctccacc |
| | GRHIFWRRLRKALTGGGSGGS | LDFACDASSNLFVASWIAVMII | aagggagaggtgaagctgcaggagtccggac |
| | RVKFSRSADAPAYQQGQNQL | FRIGMAVAIFCCFFFPSGSGGS | caggcctggtggcacccagccagtccctgtct |
| | YNELNLGRREEYDVLDKRRG | GGSWRRKRKEKQSETSPKEFL | gtgacctgtacagtgtctggcgtgagcctgccc |
| | RDPEMGGKPRRKNPQEGLYN | TIYEDVKDLKTRRNHEQEQTF | gactacggcgtgtcttggatcagacagccccct |
| | ELQKDKMAEAYSEIGMKGER | PGGGSTIYSMIQSQSSAPTSQE | agaaagggcctggagtggctgggcgtgatctg |
| | RRGKGHDGLYQGLSTATKDT | PAYTLYSLIQPSRKSGSRKRNH | gggcagcgagaccacatactataattctgccct |
| | YDALHMQALPPR (SEQ ID | SPSFNSTIYEVIGKSQPKAQNP | gaagagccggctgaccatcatcaaggacaac |
| | NO: 88) | ARLSRKELENFDVYSSLGGGS | agcaagtcccaggtgttcctgaagatgaacag |
| | | GGSNIYDAFVIYSSQDEDWVR | cctgcagaccgacgatacagccatctactattg |
| | | NELVKNLEEGVPPFQLCLHYR | tgccaagcactactattacggcggcagctatgc |
| | | DFIPGVAIAANIIHEGFHKSRK | tatggactactggggccagggcacctccgtga |
| | | VIVVVSQHFIQSRWCIFEYEIA | cagtgtctggaattcttaccacaaccacaccag |
| | | QTWQFLSSRAGIIFIVLQKVEK | cacctcggccacccaccccagcaccaacaatc |
| | | TLLRQQVELYRLLSRNTYLEW | gccagccagcctctgtccctgaggccagagg |
| | | EDSVLGRHIFWRRLRKALTGG | catgcaggccagcagcaggaggagcagtgc |
| | | GSGGSRVKFSRSADAPAYQQG | acaccagaggcctggactttgcctgtgatgcta |
| | | QNQLYNELNLGRREEYDVLD | gctccaacctgtttgtggcctcttggatcgccgt |
| | | KRRGRDPEMGGKPRRKNPQE | gatgatcatctttcggatcggcatggccgtggc |
| | | GLYNELQKDKMAEAYSEIGM | catcttctgctgtttctttttccccagcggatccg |
| | | KGERRRGKGHDGLYQGLSTA | gcggctctggcggcagctggcggagaaagcg |
| | | TKDTYDALHMQALPPR (SEQ | gaaggagaagcagtctgagaccagccccaag |
| | | ID NO: 106) | gagttcctgacaatctacgaggacgtgaaggat |
| | | | ctgaagaccaggcgcaaccacgagcaggag |
| | | | cagacctttcctggcggcggcagcacaatctat |
| | | | tccatgatccagtctcagagctccgcccccacc |
| | | | tcccaggagcctgcctacacactgtatagcctg |
| | | | atccagccttcccggaagtccggctctcggaa |
| | | | gagaaaccacagcccatccttcaattctacaat |
| | | | ctacgaagtgatcggcaagagccagccaaag |
| | | | gcacagaaccccgccaggctgagccgcaag |
| | | | gagctggagaattttgacgtgtattcttccctag |
| | | | gaggcggctccggcggctctaacatctacgac |
| | | | gccttcgtgatctatagctcccaggacgaggat |
| | | | tgggtgaggaacgagctggtgaagaatctgga |
| | | | ggagggcgtgccccctttccagctgtgcctgc |
| | | | actacagggacttcatcccaggagtggcaatc |
| | | | gccgccaatatcatccacgagggctttcacaag |
| | | | tccaggaaagtgatcgtggtggtgagccagca |
| | | | cttcatccagtcccgctggtgtatctttgagtatg |
| | | | agatcgcccagacctggcagttcctgtctagcc |
| | | | gggccggcatcatctttatcgtgctgcagaagg |
| | | | tggagaagaccctgctgagacagcaggtgga |
| | | | gctgtacaggctgctgtctcgcaacacatatctg |
| | | | gagtgggaggacagcgtgctgggccggcac |
| | | | atcttctggcggagactgagaaaggccctgac |
| | | | cggtggcggctccggcggctctagagtgaagt |
| | | | tcagcaggtccgccgacgcacctgcataccag |
| | | | cagggacagaaccagctgtataacgagctgaa |
| | | | tctgggccggagagaggagtacgacgtgctg |
| | | | gataagaggcggggccgggaccccgagatg |
| | | | ggaggcaagccacggagaaagaacccccag |
| | | | gagggcctgtacaatgagctgcagaaggaca |
| | | | agatggccgaggcctattccgagatcggcatg |
| | | | aagggagagaggcgccggggcaagggaca |
| | | | cgatggcctgtaccagggcctgtctaccgcca |
| | | | caaaggacacctatgatgccctgcacatgcag |
| | | | gccctgccccctcgg (SEQ ID |
| | | | NO: 124) |
| CD8-aTM- | TTTTPAPRPPTPAPTIASQPLSL | MALPVTALLLPLALLLHAARP | atggcactgcctgtgaccgccctgctgctgcca |
| 2B4-CD3- | RPEACRPAAGGAVHTRGLDF | YPYDVPDYAYPYDVPDYAYP | ctggccctgctgctgcacgccgccaggccttat |
| TLR4 | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | WRRKRKEKQSETSPKEFLTIY | NWYQQKPDGTVKLLIYHTSRL | ctgattacgccggctcgagtgacatccagatga |
| | EDVKDLKTRRNHEQEQTFPGG | HSGVPSRFSGSGSGTDYSLTIS | cccagaccacatctagcctgtccgcctctctgg |
| | GSTIYSMIQSQSSAPTSQEPAY | NLEQEDIATYFCQQGNTLPYT | gcgacagggtgacaatctcctgcagagcctct |
| | TLYSLIQPSRKSGSRKRNHSPS | FGGGTKLEITGSTSGSGKPGSG | caggatatcagcaagtatctgaactggtatcag |
| | FNSTIYEVIGKSQPKAQNPARL | EGSTKGEVKLQESGPGLVAPS | cagaagcccgatggcaccgtgaagctgctgat |
| | SRKELENFDVYSSLGGGSGGS | QSLSVTCTVSGVSLPDYGVSW | ctatcacacatccaggctgcactctggcgtgcc |
| | RVKFSRSADAPAYQQGQNQL | IRQPPRKGLEWLGVIWGSETT | tagccgcttcagcggctccggctctggcaccg |
| | YNELNLGRREEYDVLDKRRG | YYNSALKSRLTIIKDNSKSQVF | actactccctgacaatctctaacctggagcagg |
| | RDPEMGGKPRRKNPQEGLYN | LKMNSLQTDDTAIYYCAKHY | aggatatcgccacctatttctgccagcagggca |
| | ELQKDKMAEAYSEIGMKGER | YYGGSYAMDYWGQGTSVTV | ataccctgccatacacatttggcggcggcacaa |
| | RRGKGHDGLYQGLSTATKDT | SGILTTTTPAPRPPTPAPTIASQ | agctggagatcaccggcagcacatccggctct |
| | YDALHMQALPPRTGGGSGGS | PLSLRPEACRPAAGGAVHTRG | ggcaagccaggcagcggagagggctccacc |
| | NIYDAFVIYSSQDEDWVRNEL | LDFACDASSNLFVASWIAVMII | aagggagaggtgaagctgcaggagtccggac |
| | VKNLEEGVPPFQLCLHYRDFIP | FRIGMAVAIFCCFFFPSGSGGS | caggcctggtggcacccagccagtccctgtct |
| | GVAIAANIIHEGFHKSRKVIVV | GGSWRRKRKEKQSETSPKEFL | gtgacctgtacagtgtctggcgtgagcctgccc |
| | VSQHFIQSRWCIFEYEIAQTWQ | TIYEDVKDLKTRRNHEQEQTF | gactacggcgtgtcttggatcagacagccccct |
| | FLSSRAGIIFIVLQKVEKTLLRQ | PGGGSTIYSMIQSQSSAPTSQE | agaaagggcctggagtggctgggcgtgatctg |
| | QVELYRLLSRNTYLEWEDSVL | PAYTLYSLIQPSRKSGSRKRNH | gggcagcgagaccacatactataattctgccct |
| | GRHIFWRRLRKAL (SEQ ID | SPSFNSTIYEVIGKSQPKAQNP | gaagagccggctgaccatcatcaaggacaac |
| | NO: 89) | ARLSRKELENFDVYSSLGGGS | agcaagtcccaggtgttcctgaagatgaacag |
| | | GGSRVKFSRSADAPAYQQGQ | cctgcagaccgacgatacagccatctactattg |
| | | NQLYNELNLGRREEYDVLDK | tgccaagcactactattacggcggcagctatgc |
| | | RRGRDPEMGGKPRRKNPQEG | tatggactactggggccagggcacctccgtga |
| | | LYNELQKDKMAEAYSEIGMK | cagtgtctggaattcttaccacaaccacaccag |
| | | GERRRGKGHDGLYQGLSTAT | cacctcggccacccaccccagcaccaacaatc |
| | | KDTYDALHMQALPPRTGGGS | gccagccagcctctgtccctgaggccagagg |
| | | GGSNIYDAFVIYSSQDEDWVR | catgcaggccagcagcaggaggagcagtgc |
| | | NELVKNLEEGVPPFQLCLHYR | acaccagaggcctggactttgcctgtgatgcta |
| | | DFIPGVAIAANIIHEGFHKSRK | gctccaacctgtttgtggcctcttggatcgccgt |
| | | VIVVVSQHFIQSRWCIFEYEIA | gatgatcatctttcggatcggcatggccgtggc |
| | | QTWQFLSSRAGIIFIVLQKVEK | catcttctgctgtttctttttccccagcggatccg |
| | | TLLRQQVELYRLLSRNTYLEW | gcggctctggcggcagctggcggagaaagcg |
| | | EDSVLGRHIFWRRLRKAL | gaaggagaagcagtctgagaccagccccaag |
| | | (SEQ ID NO: 107) | gagttcctgacaatctacgaggacgtgaaggat |
| | | | ctgaagaccaggcgcaaccacgagcaggag |
| | | | cagacctttcctggcggcggcagcacaatctat |
| | | | tccatgatccagtctcagagctccgcccccacc |
| | | | tcccaggagcctgcctacacactgtatagcctg |
| | | | atccagccttcccggaagtccggctctcggaa |
| | | | gagaaaccacagcccatccttcaattctacaat |
| | | | ctacgaagtgatcggcaagagccagccaaag |
| | | | gcacagaaccccgccaggctgagccgcaag |
| | | | gagctggagaattttgacgtgtattcttccctag |
| | | | gaggcggctccggcggctctagagtgaagttc |
| | | | agcaggtccgccgacgcacctgcataccagc |
| | | | agggacagaaccagctgtataacgagctgaat |
| | | | ctgggccggagagaggagtacgacgtgctgg |
| | | | ataagaggcggggccgggaccccgagatgg |
| | | | gaggcaagccacggagaaagaacccccagg |
| | | | agggcctgtacaatgagctgcagaaggacaa |
| | | | gatggccgaggcctattccgagatcggcatga |
| | | | agggagagaggcgccggggcaagggacac |
| | | | gatggcctgtaccagggcctgtctaccgccac |
| | | | aaaggacacctatgatgccctgcacatgcagg |
| | | | ccctgccccctcggaccggtggcggctctggc |
| | | | ggcagcaacatctacgacgccttcgtgatctat |
| | | | agctcccaggacgaggattgggtgaggaacg |
| | | | agctggtgaagaatctggaggagggcgtgcc |
| | | | ccctttccagctgtgcctgcactacagggacttc |
| | | | atcccaggagtggcaatcgccgccaatatcatc |
| | | | cacgagggctttcacaagtccaggaaagtgat |
| | | | cgtggtggtgagccagcacttcatccagtcccg |
| | | | ctggtgtatctttgagtatgagatcgcccagacc |
| | | | tggcagttcctgtctagccgggccggcatcatc |
| | | | tttatcgtgctgcagaaggtggagaagaccctg |
| | | | ctgagacagcaggtggagctgtacaggctgct |
| | | | gtctcgcaacacatatctggagtgggaggaca |
| | | | gcgtgctgggccggcacatcttctggcggaga |
| | | | ctgagaaaggccctg (SEQ ID |
| | | | NO: 125) |
| CD8-aTM- | TTTTPAPRPPTPAPTIASQPLSL | MALPVTALLLPLALLLHAARP | atggcactgcctgtgaccgccctgctgctgcca |
| TLR3-CD3 | RPEACRPAAGGAVHTRGLDF | YPYDVPDYAYPYDVPDYAYP | ctggccctgctgctgcacgccgccaggccttat |
| | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | FEYAAYIIHAYKDKDWVWEH | NWYQQKPDGTVKLLIYHTSRL | ctgattacgccggctcgagtgacatccagatga |
| | FSSMEKEDQSLKFCLEERDFE | HSGVPSRFSGSGSGTDYSLTIS | cccagaccacatctagcctgtccgcctctctgg |
| | AGVFELEAIVNSIKRSRKIIFVI | NLEQEDIATYFCQQGNTLPYT | gcgacagggtgacaatctcctgcagagcctct |
| | THHLLKDPLCKRFKVHHAVQ | FGGGTKLEITGSTSGSGKPGSG | caggatatcagcaagtatctgaactggtatcag |
| | QAIEQNLDSIILVFLEEIPDYKL | EGSTKGEVKLQESGPGLVAPS | cagaagcccgatggcaccgtgaagctgctgat |
| | NHALCLRRGMFKSHCILNWP | QSLSVTCTVSGVSLPDYGVSW | ctatcacacatccaggctgcactctggcgtgcc |
| | VQKERIGAFRHKLQVALTGGG | IRQPPRKGLEWLGVIWGSETT | tagccgcttcagcggctccggctctggcaccg |
| | SGGSRVKFSRSADAPAYQQGQ | YYNSALKSRLTIIKDNSKSQVF | actactccctgacaatctctaacctggagcagg |
| | NQLYNELNLGRREEYDVLDK | LKMNSLQTDDTAIYYCAKHY | aggatatcgccacctatttctgccagcagggca |
| | RRGRDPEMGGKPRRKNPQEG | YYGGSYAMDYWGQGTSVTV | ataccctgccatacacatttggcggcggcacaa |
| | LYNELQKDKMAEAYSEIGMK | SGILTTTTPAPRPPTPAPTIASQ | agctggagatcaccggcagcacatccggctct |
| | GERRRGKGHDGLYQGLSTAT | PLSLRPEACRPAAGGAVHTRG | ggcaagccaggcagcggagagggctccacc |
| | KDTYDALHMQALPPR (SEQ ID | LDFACDASSNLFVASWIAVMII | aagggagaggtgaagctgcaggagtccggac |
| | NO: 90) | FRIGMAVAIFCCFFFPSGSGGS | caggcctggtggcacccagccagtccctgtct |
| | | GGSFEYAAYIIHAYKDKDWV | gtgacctgtacagtgtctggcgtgagcctgccc |
| | | WEHFSSMEKEDQSLKFCLEER | gactacggcgtgtcttggatcagacagccccct |
| | | DFEAGVFELEAIVNSIKRSRKII | agaaagggcctggagtggctgggcgtgatctg |
| | | FVITHHLLKDPLCKRFKVHHA | gggcagcgagaccacatactataattctgccct |
| | | VQQAIEQNLDSIILVFLEEIPDY | gaagagccggctgaccatcatcaaggacaac |
| | | KLNHALCLRRGMFKSHCILN | agcaagtcccaggtgttcctgaagatgaacag |
| | | WPVQKERIGAFRHKLQVALT | cctgcagaccgacgatacagccatctactattg |
| | | GGGSGGSRVKFSRSADAPAYQ | tgccaagcactactattacggcggcagctatgc |
| | | QGQNQLYNELNLGRREEYDV | tatggactactggggccagggcacctccgtga |
| | | LDKRRGRDPEMGGKPRRKNP | cagtgtctggaattcttaccacaaccacaccag |
| | | QEGLYNELQKDKMAEAYSEI | cacctcggccacccaccccagcaccaacaatc |
| | | GMKGERRRGKGHDGLYQGLS | gccagccagcctctgtccctgaggccagagg |
| | | TATKDTYDALHMQALPPR | catgcaggccagcagcaggaggagcagtgc |
| | | (SEQ ID NO: 108) | acaccagaggcctggactttgcctgtgatgcta |
| | | | gctccaacctgtttgtggcctcttggatcgccgt |
| | | | gatgatcatctttcggatcggcatggccgtggc |
| | | | catcttctgctgtttctttttccccagcggatccg |
| | | | gcggctctggcggcagcttcgagtacgccgcct |
| | | | atatcatccacgcctacaaggacaaggattgg |
| | | | gtgtgggagcacttcagcagcatggagaagg |
| | | | aggaccagtctctgaagttttgcctggaggaga |
| | | | gggatttcgaggccggcgtgtttgagctggag |
| | | | gccatcgtgaactccatcaagaggtctcgcaa |
| | | | gatcatcttcgtgatcacccaccacctgctgaa |
| | | | ggaccccctgtgcaagcggttcaaggtgcacc |
| | | | acgccgtgcagcaggccatcgagcagaatct |
| | | | ggacagcatcatcctggtgtttctggaggagat |
| | | | ccccgattataagctgaaccacgccctgtgcct |
| | | | gcggagaggcatgttcaagtcccactgtatcct |
| | | | gaattggcctgtgcagaaggagcggatcggc |
| | | | gcctttagacacaagctgcaggtggccctgac |
| | | | cggtggcggctctggcggcagcagagtgaag |
| | | | ttcagcaggtccgccgacgcacctgcatacca |
| | | | gcagggacagaaccagctgtataacgagctg |
| | | | aatctgggccggagagaggagtacgacgtgc |
| | | | tggataagaggcggggccgggaccccgagat |
| | | | gggaggcaagccacggagaaagaaccccca |
| | | | ggagggcctgtacaatgagctgcagaaggac |
| | | | aagatggccgaggcctattccgagatcggcat |
| | | | gaagggagagaggcgccggggcaagggac |
| | | | acgatggcctgtaccagggcctgtctaccgcc |
| | | | acaaaggacacctatgatgccctgcacatgca |
| | | | ggccctgccccctcgg (SEQ ID |
| | | | NO: 126) |
| CD8-aTM- | TTTTPAPRPPTPAPTIASQPLSL | MALPVTALLLPLALLLHAARP | atggcactgcctgtgaccgccctgctgctgcca |
| 2B4-TLR3- | RPEACRPAAGGAVHTRGLDF | YPYDVPDYAYPYDVPDYAYP | ctggccctgctgctgcacgccgccaggccttat |
| CD3 | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | WRRKRKEKQSETSPKEFLTIY | NWYQQKPDGTVKLLIYHTSRL | ctgattacgccggctcgagtgacatccagatga |
| | EDVKDLKTRRNHEQEQTFPGG | HSGVPSRFSGSGSGTDYSLTIS | cccagaccacatctagcctgtccgcctctctgg |
| | GSTIYSMIQSQSSAPTSQEPAY | NLEQEDIATYFCQQGNTLPYT | gcgacagggtgacaatctcctgcagagcctct |
| | TLYSLIQPSRKSGSRKRNHSPS | FGGGTKLEITGSTSGSGKPGSG | caggatatcagcaagtatctgaactggtatcag |
| | FNSTIYEVIGKSQPKAQNPARL | EGSTKGEVKLQESGPGLVAPS | cagaagcccgatggcaccgtgaagctgctgat |
| | SRKELENFDVYSSLGGGSGGS | QSLSVTCTVSGVSLPDYGVSW | ctatcacacatccaggctgcactctggcgtgcc |
| | FEYAAYIIHAYKDKDWVWEH | IRQPPRKGLEWLGVIWGSETT | tagccgcttcagcggctccggctctggcaccg |
| | FSSMEKEDQSLKFCLEERDFE | YYNSALKSRLTIIKDNSKSQVF | actactccctgacaatctctaacctggagcagg |
| | AGVFELEAIVNSIKRSRKIIFVI | LKMNSLQTDDTAIYYCAKHY | aggatatcgccacctatttctgccagcagggca |
| | THHLLKDPLCKRFKVHHAVQ | YYGGSYAMDYWGQGTSVTV | ataccctgccatacacatttggcggcggcacaa |
| | QAIEQNLDSIILVFLEEIPDYKL | SGILTTTTPAPRPPTPAPTIASQ | agctggagatcaccggcagcacatccggctct |
| | NHALCLRRGMFKSHCILNWP | PLSLRPEACRPAAGGAVHTRG | ggcaagccaggcagcggagagggctccacc |
| | VQKERIGAFRHKLQVALTGGG | LDFACDASSNLFVASWIAVMII | aagggagaggtgaagctgcaggagtccggac |
| | SGGSRVKFSRSADAPAYQQGQ | FRIGMAVAIFCCFFFPSGSGGS | caggcctggtggcacccagccagtccctgtct |
| | NQLYNELNLGRREEYDVLDK | GGSWRRKRKEKQSETSPKEFL | gtgacctgtacagtgtctggcgtgagcctgccc |
| | RRGRDPEMGGKPRRKNPQEG | TIYEDVKDLKTRRNHEQEQTF | gactacggcgtgtcttggatcagacagccccct |
| | LYNELQKDKMAEAYSEIGMK | PGGGSTIYSMIQSQSSAPTSQE | agaaagggcctggagtggctgggcgtgatctg |
| | GERRRGKGHDGLYQGLSTAT | PAYTLYSLIQPSRKSGSRKRNH | gggcagcgagaccacatactataattctgccct |
| | KDTYDALHMQALPPR (SEQ ID | SPSFNSTIYEVIGKSQPKAQNP | gaagagccggctgaccatcatcaaggacaac |
| | NO: 91) | ARLSRKELENFDVYSSLGGGS | agcaagtcccaggtgttcctgaagatgaacag |
| | | GGSFEYAAYIIHAYKDKDWV | cctgcagaccgacgatacagccatctactattg |
| | | WEHFSSMEKEDQSLKFCLEER | tgccaagcactactattacggcggcagctatgc |
| | | DFEAGVFELEAIVNSIKRSRKII | tatggactactggggccagggcacctccgtga |
| | | FVITHHLLKDPLCKRFKVHHA | cagtgtctggaattcttaccacaaccacaccag |
| | | VQQAIEQNLDSIILVFLEEIPDY | cacctcggccacccaccccagcaccaacaatc |
| | | KLNHALCLRRGMFKSHCILN | gccagccagcctctgtccctgaggccagagg |
| | | WPVQKERIGAFRHKLQVALT | catgcaggccagcagcaggaggagcagtgc |
| | | GGGSGGSRVKFSRSADAPAYQ | acaccagaggcctggactttgcctgtgatgcta |
| | | QGQNQLYNELNLGRREEYDV | gctccaacctgtttgtggcctcttggatcgccgt |
| | | LDKRRGRDPEMGGKPRRKNP | gatgatcatctttcggatcggcatggccgtggc |
| | | QEGLYNELQKDKMAEAYSEI | catcttctgctgtttctttttccccagcggatccg |
| | | GMKGERRRGKGHDGLYQGLS | gcggctctggcggcagctggcggagaaagcg |
| | | TATKDTYDALHMQALPPR | gaaggagaagcagtctgagaccagccccaag |
| | | (SEQ ID NO: 109) | gagttcctgacaatctacgaggacgtgaaggat |
| | | | ctgaagaccaggcgcaaccacgagcaggag |
| | | | cagacctttcctggcggcggcagcacaatctat |
| | | | tccatgatccagtctcagagctccgcccccacc |
| | | | tcccaggagcctgcctacacactgtatagcctg |
| | | | atccagccttcccggaagtccggctctcggaa |
| | | | gagaaaccacagcccatccttcaattctacaat |
| | | | ctacgaagtgatcggcaagagccagccaaag |
| | | | gcacagaaccccgccaggctgagccgcaag |
| | | | gagctggagaattttgacgtgtattcttccctag |
| | | | gaggcggctccggcggctctttcgagtacgcc |
| | | | gcctatatcatccacgcctacaaggacaaggat |
| | | | tgggtgtgggagcacttcagcagcatggagaa |
| | | | ggaggaccagtctctgaagttttgcctggagga |
| | | | gagggatttcgaggccggcgtgtttgagctgg |
| | | | aggccatcgtgaactccatcaagaggtctcgc |
| | | | aagatcatcttcgtgatcacccaccacctgctga |
| | | | aggaccccctgtgcaagcggttcaaggtgcac |
| | | | cacgccgtgcagcaggccatcgagcagaatct |
| | | | ggacagcatcatcctggtgtttctggaggagat |
| | | | ccccgattataagctgaaccacgccctgtgcct |
| | | | gcggagaggcatgttcaagtcccactgtatcct |
| | | | gaattggcctgtgcagaaggagcggatcggc |
| | | | gcctttagacacaagctgcaggtggccctgac |
| | | | cggtggcggctctggcggcagcagagtgaag |
| | | | ttcagcaggtccgccgacgcacctgcatacca |
| | | | gcagggacagaaccagctgtataacgagctg |
| | | | aatctgggccggagagaggagtacgacgtgc |
| | | | tggataagaggcggggccgggaccccgagat |
| | | | gggaggcaagccacggagaaagaaccccca |
| | | | ggagggcctgtacaatgagctgcagaaggac |
| | | | aagatggccgaggcctattccgagatcggcat |
| | | | gaagggagagaggcgccggggcaagggac |
| | | | acgatggcctgtaccagggcctgtctaccgcc |
| | | | acaaaggacacctatgatgccctgcacatgca |
| | | | ggccctgccccctcgg (SEQ ID |
| | | | NO: 127) |
| CD8-aTM- | TTTTPAPRPPTPAPTIASQPLSL | MALPVTALLLPLALLLHAARP | atggccttaccagtgaccgccttgctcctgccg |
| 2B4-CD3- | RPEACRPAAGGAVHTRGLDF | YPYDVPDYAYPYDVPDYAYP | ctggccttgctgctccacgccgccaggccgta |
| TLR2 | ACDASSNLFVASWIAVMIIFRI | YDVPDYAGSSDIQMTQTTSSL | cccatacgatgttccagattacgcttatccctac |
| | GMAVAIFCCFFFPSGSGGSGGS | SASLGDRVTISCRASQDISKYL | gacgtgcctgattatgcatacccatatgatgtcc |
| | WRRKRKEKQSETSPKEFLTIY | NWYQQKPDGTVKLLIYHTSRL | ccgactatgccggctcgagtgacatccagatg |
| | EDVKDLKTRRNHEQEQTFPGG | HSGVPSRFSGSGSGTDYSLTIS | acacagactacatcctccctgtctgcctctctgg |
| | GSTIYSMIQSQSSAPTSQEPAY | NLEQEDIATYFCQQGNTLPYT | gagacagagtcaccatcagttgcagggcaagt |
| | TLYSLIQPSRKSGSRKRNHSPS | FGGGTKLEITGSTSGSGKPGSG | caggacattagtaaatatttaaattggtatcagca |
| | FNSTIYEVIGKSQPKAQNPARL | EGSTKGEVKLQESGPGLVAPS | gaaaccagatggaactgttaaactcctgatcta |
| | SRKELENFDVYSSLGGGSGGS | QSLSVTCTVSGVSLPDYGVSW | ccatacatcaagattacactcaggagtcccatc |
| | RVKFSRSADAPAYQQGQNQL | IRQPPRKGLEWLGVIWGSETT | aaggttcagtggcagtgggtctggaacagatta |
| | YNELNLGRREEYDVLDKRRG | YYNSALKSRLTIIKDNSKSQVF | ttctctcaccattagcaacctggagcaagaaga |
| | RDPEMGGKPRRKNPQEGLYN | LKMNSLQTDDTAIYYCAKHY | tattgccacttacttttgccaacagggtaatacgc |
| | ELQKDKMAEAYSEIGMKGER | YYGGSYAMDYWGQGTSVTV | ttccgtacacgttcggaggggggactaagttgg |
| | RRGKGHDGLYQGLSTATKDT | SGILTTTTPAPRPPTPAPTIASQ | aaataacaggctccacctctggatctggcaagc |
| | YDALHMQALPPRTGGGSGGSI | PLSLRPEACRPAAGGAVHTRG | ccggatctggcgagggatctaccaagggcga |
| | CYDAFVSYSERDAYWVENLM | LDFACDASSNLFVASWIAVMII | ggtgaaactgcaggagtcaggacctggcctg |
| | VQELENFNPPFKLCLHKRDFIP | FRIGMAVAIFCCFFFPSGSGGS | gtggcgccctcacagagcctgtccgtcacatg |
| | GKWIIDNIIDSIEKSHKTVFVLS | GGSWRRKRKEKQSETSPKEFL | cactgtctcaggggtctcattacccgactatggt |
| | ENFVKSEWCKYELDFSHFRLF | TIYEDVKDLKTRRNHEQEQTF | gtaagctggattcgccagcctccacgaaaggg |
| | DENNDAAILILLEPIEKKAIPQR | PGGGSTIYSMIQSQSSAPTSQE | tctggagtggctgggagtaatatggggtagtga |
| | FCKLRKIMNTKTYLEWPMDE | PAYTLYSLIQPSRKSGSRKRNH | aaccacatactataattcagctctcaaatccaga |
| | AQREGFWVNLRAAI (SEQ ID | SPSFNSTIYEVIGKSQPKAQNP | ctgaccatcatcaaggacaactccaagagcca |
| | NO: 221) | ARLSRKELENFDVYSSLGGGS | agttttcttaaaaatgaacagtctgcaaactgatg |
| | | GGSRVKFSRSADAPAYQQGQ | acacagccatttactactgtgccaaacattattac |
| | | NQLYNELNLGRREEYDVLDK | tacggtggtagctatgctatggactactggggt |
| | | RRGRDPEMGGKPRRKNPQEG | caaggaacctcagtcaccgtctccggaattctt |
| | | LYNELQKDKMAEAYSEIGMK | accaccacaacgcccgctcctcggccaccga |
| | | GERRRGKGHDGLYQGLSTAT | cgccagcgccaactattgcgagtcagcctctca |
| | | KDTYDALHMQALPPRTGGGS | gtctgcgacctgaggcttgtcgaccagcagcc |
| | | GGSICYDAFVSYSERDAYWVE | ggaggcgcagtgcacacgagggggctggac |
| | | NLMVQELENFNPPFKLCLHKR | ttcgcctgtgatgctagctccaacctttttgtggc |
| | | DFIPGKWIIDNIIDSIEKSHKTV | gtcttggatagcggtgatgataatatttaggattg |
| | | FVLSENFVKSEWCKYELDFSH | ggatggcggtagcgatattttgctgctttttcttc |
| | | FRLFDENNDAAILILLEPIEKK | cctagcggatccggaggttctggtggatcatgga |
| | | AIPQRFCKLRKIMNTKTYLEW | ggagaaagaggaaggagaagcagtcagaga |
| | | PMDEAQREGFWVNLRAAI | ccagtcccaaggaatttttgacaatttacgaaga |
| | | (SEQ ID NO: 222) | tgtcaaggatctgaaaaccaggagaaatcacg |
| | | | agcaggagcagacttttcctggaggggggag |
| | | | caccatctactctatgatccagtcccagtcttctg |
| | | | ctcccacgtcacaagaacctgcatatacattata |
| | | | ttcattaattcagccttccaggaagtctgggtcc |
| | | | aggaagaggaaccacagcccttccttcaatag |
| | | | cactatctatgaagtgattggaaagagtcaacct |
| | | | aaagcccagaaccctgctcgattgagccgcaa |
| | | | agagctggagaactttgatgtttattcctccctag |
| | | | gaggaggttctggtggatcaagagtgaagttca |
| | | | gcaggagcgcagacgcccccgcgtaccagc |
| | | | agggccagaaccagctctataacgagctcaat |
| | | | ctaggacgaagagaggagtacgatgttttgga |
| | | | caagagacgtggccgggaccctgagatgggg |
| | | | ggaaagccgagaaggaagaaccctcaggaa |
| | | | ggcctgtacaatgaactgcagaaagataagat |
| | | | ggcggaggcctacagtgagattgggatgaaa |
| | | | ggcgagcgccggaggggcaaggggcacgat |
| | | | ggcctttaccagggtctcagtacagccaccaa |
| | | | ggacacctacgacgcccttcacatgcaggccc |
| | | | tgccccctcgcaccggtggaggttctggtggat |
| | | | caatctgctatgatgcatttgtttcttacagtgag |
| | | | cgggatgcctactgggtggagaaccttatggtcc |
| | | | aggagctggagaacttcaatccccccttcaagt |
| | | | tgtgtcttcataagcgggacttcattcctggcaa |
| | | | gtggatcattgacaatatcattgactccattgaaa |
| | | | agagccacaaaactgtctttgtgctttctgaaaa |
| | | | ctttgtgaagagtgagtggtgcaagtatgaact |
| | | | ggacttctcccatttccgtctttttgatgagaaca |
| | | | atgatgctgccattctcattcttctggagcccatt |
| | | | gagaaaaaagccattccccagcgcttctgcaa |
| | | | gctgcggaagataatgaacaccaagacctacc |
| | | | tggagtggcctatggacgaggctcagcggga |
| | | | aggattttgggtaaatctgagagctgcgata |
| | | | (SEQ ID NO: 223) |
| CD8-aTM- | TTTTPAPRPPTPAPTIASQPLSL | MALPVTALLLPLALLLHAARP | atggcactgcctgtgaccgccctgctgctgcca |
| 2B4-CD3- | RPEACRPAAGGAVHTRGLDF | YPYDVPDYAYPYDVPDYAYP | ctggccctgctgctgcacgccgccaggccttat |
| TLR3 | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | WRRKRKEKQSETSPKEFLTIY | NWYQQKPDGTVKLLIYHTSRL | ctgattacgccggctcgagtgacatccagatga |
| | EDVKDLKTRRNHEQEQTFPGG | HSGVPSRFSGSGSGTDYSLTIS | cccagaccacatctagcctgtccgcctctctgg |
| | GSTIYSMIQSQSSAPTSQEPAY | NLEQEDIATYFCQQGNTLPYT | gcgacagggtgacaatctcctgcagagcctct |
| | TLYSLIQPSRKSGSRKRNHSPS | FGGGTKLEITGSTSGSGKPGSG | caggatatcagcaagtatctgaactggtatcag |
| | FNSTIYEVIGKSQPKAQNPARL | EGSTKGEVKLQESGPGLVAPS | cagaagcccgatggcaccgtgaagctgctgat |
| | SRKELENFDVYSSLGGGSGGS | QSLSVTCTVSGVSLPDYGVSW | ctatcacacatccaggctgcactctggcgtgcc |
| | RVKFSRSADAPAYQQGQNQL | IRQPPRKGLEWLGVIWGSETT | tagccgcttcagcggctccggctctggcaccg |
| | YNELNLGRREEYDVLDKRRG | YYNSALKSRLTIIKDNSKSQVF | actactccctgacaatctctaacctggagcagg |
| | RDPEMGGKPRRKNPQEGLYN | LKMNSLQTDDTAIYYCAKHY | aggatatcgccacctatttctgccagcagggca |
| | ELQKDKMAEAYSEIGMKGER | YYGGSYAMDYWGQGTSVTV | ataccctgccatacacatttggcggcggcacaa |
| | RRGKGHDGLYQGLSTATKDT | SGILTTTTPAPRPPTPAPTIASQ | agctggagatcaccggcagcacatccggctct |
| | YDALHMQALPPRTGGGSGGS | PLSLRPEACRPAAGGAVHTRG | ggcaagccaggcagcggagagggctccacc |
| | FEYAAYIIHAYKDKDWVWEH | LDFACDASSNLFVASWIAVMII | aagggagaggtgaagctgcaggagtccggac |
| | FSSMEKEDQSLKFCLEERDFE | FRIGMAVAIFCCFFFPSGSGGS | caggcctggtggcacccagccagtccctgtct |
| | AGVFELEAIVNSIKRSRKIIFVI | GGSWRRKRKEKQSETSPKEFL | gtgacctgtacagtgtctggcgtgagcctgccc |
| | THHLLKDPLCKRFKVHHAVQ | TIYEDVKDLKTRRNHEQEQTF | gactacggcgtgtcttggatcagacagccccct |
| | QAIEQNLDSIILVFLEEIPDYKL | PGGGSTIYSMIQSQSSAPTSQE | agaaagggcctggagtggctgggcgtgatctg |
| | NHALCLRRGMFKSHCILNWP | PAYTLYSLIQPSRKSGSRKRNH | gggcagcgagaccacatactataattctgccct |
| | VQKERIGAFRHKLQVAL (SEQ | SPSFNSTIYEVIGKSQPKAQNP | gaagagccggctgaccatcatcaaggacaac |
| | ID NO: 92) | ARLSRKELENFDVYSSLGGGS | agcaagtcccaggtgttcctgaagatgaacag |
| | | GGSRVKFSRSADAPAYQQGQ | cctgcagaccgacgatacagccatctactattg |
| | | NQLYNELNLGRREEYDVLDK | tgccaagcactactattacggcggcagctatgc |
| | | RRGRDPEMGGKPRRKNPQEG | tatggactactggggccagggcacctccgtga |
| | | LYNELQKDKMAEAYSEIGMK | cagtgtctggaattcttaccacaaccacaccag |
| | | GERRRGKGHDGLYQGLSTAT | cacctcggccacccaccccagcaccaacaatc |
| | | KDTYDALHMQALPPRTGGGS | gccagccagcctctgtccctgaggccagagg |
| | | GGSFEYAAYIIHAYKDKDWV | catgcaggccagcagcaggaggagcagtgc |
| | | WEHFSSMEKEDQSLKFCLEER | acaccagaggcctggactttgcctgtgatgcta |
| | | DFEAGVFELEAIVNSIKRSRKII | gctccaacctgtttgtggcctcttggatcgccgt |
| | | FVITHHLLKDPLCKRFKVHHA | gatgatcatctttcggatcggcatggccgtggc |
| | | VQQAIEQNLDSIILVFLEEIPDY | catcttctgctgtttctttttccccagcggatccg |
| | | KLNHALCLRRGMFKSHCILN | cgggctctggcggcagctggcggagaaagcg |
| | | WPVQKERIGAFRHKLQVAL | gaaggagaagcagtctgagaccagccccaag |
| | | (SEQ ID NO: 110) | gagttcctgacaatctacgaggacgtgaaggat |
| | | | ctgaagaccaggcgcaaccacgagcaggag |
| | | | cagacctttcctggcggcggcagcacaatctat |
| | | | tccatgatccagtctcagagctccgcccccacc |
| | | | tcccaggagcctgcctacacactgtatagcctg |
| | | | atccagccttcccggaagtccggctctcggaa |
| | | | gagaaaccacagcccatccttcaattctacaat |
| | | | ctacgaagtgatcggcaagagccagccaaag |
| | | | gcacagaaccccgccaggctgagccgcaag |
| | | | gagctggagaattttgacgtgtattcttccctag |
| | | | gaggcggctccggcggctctagagtgaagttc |
| | | | agcaggtccgccgacgcacctgcataccagc |
| | | | agggacagaaccagctgtataacgagctgaat |
| | | | ctgggccggagagaggagtacgacgtgctgg |
| | | | ataagaggcggggccgggaccccgagatgg |
| | | | gaggcaagccacggagaaagaacccccagg |
| | | | agggcctgtacaatgagctgcagaaggacaa |
| | | | gatggccgaggcctattccgagatcggcatga |
| | | | agggagagaggcgccggggcaagggacac |
| | | | gatggcctgtaccagggcctgtctaccgccac |
| | | | aaaggacacctatgatgccctgcacatgcagg |
| | | | ccctgccccctcggaccggtggcggctctggc |
| | | | ggcagcttcgagtacgccgcctatatcatccac |
| | | | gcctacaaggacaaggattgggtgtgggagc |
| | | | acttcagcagcatggagaaggaggaccagtct |
| | | | ctgaagttttgcctggaggagagggatttcgag |
| | | | gccggcgtgtttgagctggaggccatcgtgaa |
| | | | ctccatcaagaggtctcgcaagatcatcttcgtg |
| | | | atcacccaccacctgctgaaggaccccctgtg |
| | | | caagcggttcaaggtgcaccacgccgtgcag |
| | | | caggccatcgagcagaatctggacagcatcat |
| | | | cctggtgtttctggaggagatccccgattataag |
| | | | ctgaaccacgccctgtgcctgcggagaggcat |
| | | | gttcaagtcccactgtatcctgaattggcctgtg |
| | | | cagaaggagcggatcggcgcctttagacaca |
| | | | agctgcaggtggccctg (SEQ ID |
| | | | NO: 128) |
| CD8-aTM- | TTTTPAPRPPTPAPTIASQPLSL | MALPVTALLLPLALLLHAARP | atggcactgcctgtgaccgccctgctgctgcca |
| TLR7-CD3 | RPEACRPAAGGAVHTRGLDF | YPYDVPDYAYPYDVPDYAYP | ctggccctgctgctgcacgccgccaggccttat |
| | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | CCYDAFIVYDTKDPAVTEWV | NWYQQKPDGTVKLLIYHTSRL | ctgattacgccggctcgagtgacatccagatga |
| | LAELVAKLEDPREKHENLCLE | HSGVPSRFSGSGSGTDYSLTIS | cccagaccacatctagcctgtccgcctctctgg |
| | ERDWLPGQPVLENLSQSIQLS | NLEQEDIATYFCQQGNTLPYT | gcgacagggtgacaatctcctgcagagcctct |
| | KKTVFVMTDKYAKTENFKIAF | FGGGTKLEITGSTSGSGKPGSG | caggatatcagcaagtatctgaactggtatcag |
| | YLSHQRLMDEKVDVIILIFLEK | EGSTKGEVKLQESGPGLVAPS | cagaagcccgatggcaccgtgaagctgctgat |
| | PFQKSKFLQLRKRLCGSSVLE | QSLSVTCTVSGVSLPDYGVSW | ctatcacacatccaggctgcactctggcgtgcc |
| | WPTNPQAHPYFWQCLKNALT | IRQPPRKGLEWLGVIWGSETT | tagccgcttcagcggctccggctctggcaccg |
| | GGGSGGSRVKFSRSADAPAYQ | YYNSALKSRLTIIKDNSKSQVF | actactccctgacaatctctaacctggagcagg |
| | QGQNQLYNELNLGRREEYDV | LKMNSLQTDDTAIYYCAKHY | aggatatcgccacctatttctgccagcagggca |
| | LDKRRGRDPEMGGKPRRKNP | YYGGSYAMDYWGQGTSVTV | ataccctgccatacacatttggcggcggcacaa |
| | QEGLYNELQKDKMAEAYSEI | SGILTTTTPAPRPPTPAPTIASQ | agctggagatcaccggcagcacatccggctct |
| | GMKGERRRGKGHDGLYQGLS | PLSLRPEACRPAAGGAVHTRG | ggcaagccaggcagcggagagggctccacc |
| | TATKDTYDALHMQALPPR | LDFACDASSNLFVASWIAVMII | aagggagaggtgaagctgcaggagtccggac |
| | (SEQ ID NO: 93) | FRIGMAVAIFCCFFFPSGSGGS | caggcctggtggcacccagccagtccctgtct |
| | | GGSCCYDAFIVYDTKDPAVTE | gtgacctgtacagtgtctggcgtgagcctgccc |
| | | WVLAELVAKLEDPREKHFNL | gactacggcgtgtcttggatcagacagccccct |
| | | CLEERDWLPGQPVLENLSQSI | agaaagggcctggagtggctgggcgtgatctg |
| | | QLSKKTVFVMTDKYAKTENF | gggcagcgagaccacatactataattctgccct |
| | | KIAFYLSHQRLMDEKVDVIILI | gaagagccggctgaccatcatcaaggacaac |
| | | FLEKPFQKSKFLQLRKRLCGSS | agcaagtcccaggtgttcctgaagatgaacag |
| | | VLEWPTNPQAHPYFWQCLKN | cctgcagaccgacgatacagccatctactattg |
| | | ALTGGGSGGSRVKFSRSADAP | tgccaagcactactattacggcggcagctatgc |
| | | AYQQGQNQLYNELNLGRREE | tatggactactggggccagggcacctccgtga |
| | | YDVLDKRRGRDPEMGGKPRR | cagtgtctggaattcttaccacaaccacaccag |
| | | KNPQEGLYNELQKDKMAEAY | cacctcggccacccaccccagcaccaacaatc |
| | | SEIGMKGERRRGKGHDGLYQ | gccagccagcctctgtccctgaggccagagg |
| | | GLSTATKDTYDALHMQALPP | catgcaggccagcagcaggaggagcagtgc |
| | | R (SEQ ID NO: 111) | acaccagaggcctggactttgcctgtgatgcta |
| | | | gctccaacctgtttgtggcctcttggatcgccgt |
| | | | gatgatcatctttcggatcggcatggccgtggc |
| | | | catcttctgctgtttctttttccccagcggatccg |
| | | | gcggctctggcggcagctgctgttacgacgcctt |
| | | | tatcgtgtatgacaccaaggaccccgcagtga |
| | | | cagagtgggtgctggccgagctggtggcaaa |
| | | | gctggaggacccaagggagaagcacttcaac |
| | | | ctgtgcctcgaggagagggattggctgccagg |
| | | | acagcctgtgctggagaatctgtctcagagcat |
| | | | ccagctgagcaagaagaccgtgtttgtgatgac |
| | | | cgataagtacgccaagaccgagaacttcaaga |
| | | | tcgccttttatctgtcccaccagcggctgatgga |
| | | | cgagaaggtggatgtgatcatcctgatcttcctg |
| | | | gagaagccctttcagaagtctaagttcctgcag |
| | | | ctgcggaagagactgtgcggcagctccgtgct |
| | | | ggagtggccaaccaacccacaggcacaccct |
| | | | tacttctggcagtgtctgaagaatgccctgacc |
| | | | ggtggcggctccggcggctctagagtgaagtt |
| | | | cagcaggtccgccgacgcacctgcataccag |
| | | | cagggacagaaccagctgtataacgagctgaa |
| | | | tctgggccggagagaggagtacgacgtgctg |
| | | | gataagaggcggggccgggaccccgagatg |
| | | | ggaggcaagccacggagaaagaacccccag |
| | | | gagggcctgtacaatgagctgcagaaggaca |
| | | | agatggccgaggcctattccgagatcggcatg |
| | | | aagggagagaggcgccggggcaagggaca |
| | | | cgatggcctgtaccagggcctgtctaccgcca |
| | | | caaaggacacctatgatgccctgcacatgcag |
| | | | gccctgccccctcgg (SEQ ID |
| | | | NO: 129) |
| CD8-aTM- | TTTTPAPRPPTPAPTIASQPLSL | MALPVTALLLPLALLLHAARP | atggcactgcctgtgaccgccctgctgctgcca |
| 2B4-TLR7- | RPEACRPAAGGAVHTRGLDF | YPYDVPDYAYPYDVPDYAYP | ctggccctgctgctgcacgccgccaggccttat |
| CD3 | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | WRRKRKEKQSETSPKEFLTIY | NWYQQKPDGTVKLLIYHTSRL | ctgattacgccggctcgagtgacatccagatga |
| | EDVKDLKTRRNHEQEQTFPGG | HSGVPSRFSGSGSGTDYSLTIS | cccagaccacatctagcctgtccgcctctctgg |
| | GSTIYSMIQSQSSAPTSQEPAY | NLEQEDIATYFCQQGNTLPYT | gcgacagggtgacaatctcctgcagagcctct |
| | TLYSLIQPSRKSGSRKRNHSPS | FGGGTKLEITGSTSGSGKPGSG | caggatatcagcaagtatctgaactggtatcag |
| | FNSTIYEVIGKSQPKAQNPARL | EGSTKGEVKLQESGPGLVAPS | cagaagcccgatggcaccgtgaagctgctgat |
| | SRKELENFDVYSSLGGGSGGS | QSLSVTCTVSGVSLPDYGVSW | ctatcacacatccaggctgcactctggcgtgcc |
| | CCYDAFIVYDTKDPAVTEWV | IRQPPRKGLEWLGVIWGSETT | tagccgcttcagcggctccggctctggcaccg |
| | LAELVAKLEDPREKHFNLCLE | YYNSALKSRLTIIKDNSKSQVF | actactccctgacaatctctaacctggagcagg |
| | ERDWLPGQPVLENLSQSIQLS | LKMNSLQTDDTAIYYCAKHY | aggatatcgccacctatttctgccagcagggca |
| | KKTVFVMTDKYAKTENFKIAF | YYGGSYAMDYWGQGTSVTV | ataccctgccatacacatttggcggcggcacaa |
| | YLSHQRLMDEKVDVIILIFLEK | SGILTTTTPAPRPPTPAPTIASQ | agctggagatcaccggcagcacatccggctct |
| | PFQKSKFLQLRKRLCGSSVLE | PLSLRPEACRPAAGGAVHTRG | ggcaagccaggcagcggagagggctccacc |
| | WPTNPQAHPYFWQCLKNALT | LDFACDASSNLFVASWIAVMII | aagggagaggtgaagctgcaggagtccggac |
| | GGGSGGSRVKFSRSADAPAYQ | FRIGMAVAIFCCFFFPSGSGGS | caggcctggtggcacccagccagtccctgtct |
| | QGQNQLYNELNLGRREEYDV | GGSWRRKRKEKQSETSPKEFL | gtgacctgtacagtgtctggcgtgagcctgccc |
| | LDKRRGRDPEMGGKPRRKNP | TIYEDVKDLKTRRNHEQEQTF | gactacggcgtgtcttggatcagacagccccct |
| | QEGLYNELQKDKMAEAYSEI | PGGGSTIYSMIQSQSSAPTSQE | agaaagggcctggagtggctgggcgtgatctg |
| | GMKGERRRGKGHDGLYQGLS | PAYTLYSLIQPSRKSGSRKRNH | gggcagcgagaccacatactataattctgccct |
| | TATKDTYDALHMQALPPR | SPSFNSTIYEVIGKSQPKAQNP | gaagagccggctgaccatcatcaaggacaac |
| | (SEQ ID NO: 94) | ARLSRKELENFDVYSSLGGGS | agcaagtcccaggtgttcctgaagatgaacag |
| | | GGSCCYDAFIVYDTKDPAVTE | cctgcagaccgacgatacagccatctactattg |
| | | WVLAELVAKLEDPREKHFNL | tgccaagcactactattacggcggcagctatgc |
| | | CLEERDWLPGQPVLENLSQSI | tatggactactggggccagggcacctccgtga |
| | | QLSKKTVFVMTDKYAKTENF | cagtgtctggaattcttaccacaaccacaccag |
| | | KIAFYLSHQRLMDEKVDVIILI | cacctcggccacccaccccagcaccaacaatc |
| | | FLEKPFQKSKFLQLRKRLCGSS | gccagccagcctctgtccctgaggccagagg |
| | | VLEWPTNPQAHPYFWQCLKN | catgcaggccagcagcaggaggagcagtgc |
| | | ALTGGGSGGSRVKFSRSADAP | acaccagaggcctggactttgcctgtgatgcta |
| | | AYQQGQNQLYNELNLGRREE | gctccaacctgtttgtggcctcttggatcgccgt |
| | | YDVLDKRRGRDPEMGGKPRR | gatgatcatctttcggatcggcatggccgtggc |
| | | KNPQEGLYNELQKDKMAEAY | catcttctgctgtttctttttccccagcggatccg |
| | | SEIGMKGERRRGKGHDGLYQ | gcggctctggcggcagctggcggagaaagcg |
| | | GLSTATKDTYDALHMQALPP | gaaggagaagcagtctgagaccagccccaag |
| | | R (SEQ ID NO: 112) | gagttcctgacaatctacgaggacgtgaaggat |
| | | | ctgaagaccaggcgcaaccacgagcaggag |
| | | | cagacctttcctggcggcggcagcacaatctat |
| | | | tccatgatccagtctcagagctccgcccccacc |
| | | | tcccaggagcctgcctacacactgtatagcctg |
| | | | atccagccttcccggaagtccggctctcggaa |
| | | | gagaaaccacagcccatccttcaattctacaat |
| | | | ctacgaagtgatcggcaagagccagccaaag |
| | | | gcacagaaccccgccaggctgagccgcaag |
| | | | gagctggagaattttgacgtgtattcttccctag |
| | | | gaggcggctccggcggctcttgctgttacgac |
| | | | gcctttatcgtgtatgacaccaaggaccccgca |
| | | | gtgacagagtgggtgctggccgagctggtgg |
| | | | caaagctggaggacccaagggagaagcactt |
| | | | caacctgtgcctcgaggagagggattggctgc |
| | | | caggacagcctgtgctggagaatctgtctcaga |
| | | | gcatccagctgagcaagaagaccgtgtttgtga |
| | | | tgaccgataagtacgccaagaccgagaacttc |
| | | | aagatcgccttttatctgtcccaccagcggctga |
| | | | tggacgagaaggtggatgtgatcatcctgatctt |
| | | | cctggagaagccctttcagaagtctaagttcctg |
| | | | cagctgcggaagagactgtgcggcagctccgt |
| | | | gctggagtggccaaccaacccacaggcacac |
| | | | ccttacttctggcagtgtctgaagaatgccctga |
| | | | ccggtggcggctccggcggctctagagtgaa |
| | | | gttcagcaggtccgccgacgcacctgcatacc |
| | | | agcagggacagaaccagctgtataacgagct |
| | | | gaatctgggccggagagaggagtacgacgtg |
| | | | ctggataagaggcggggccgggaccccgag |
| | | | atgggaggcaagccacggagaaagaacccc |
| | | | caggagggcctgtacaatgagctgcagaagg |
| | | | acaagatggccgaggcctattccgagatcggc |
| | | | atgaagggagagaggcgccggggcaaggga |
| | | | cacgatggcctgtaccagggcctgtctaccgc |
| | | | cacaaaggacacctatgatgccctgcacatgc |
| | | | aggccctgccccctcgg (SEQ ID |
| | | | NO: 130) |
| CD8-aTM- | TTTTPAPRPPTPAPTIASQPLSL | MALPVTALLLPLALLLHAARP | atggcactgcctgtgaccgccctgctgctgcca |
| 2B4-CD3- | RPEACRPAAGGAVHTRGLDF | YPYDVPDYAYPYDVPDYAYP | ctggccctgctgctgcacgccgccaggccttat |
| TLR7 | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | WRRKRKEKQSETSPKEFLTIY | NWYQQKPDGTVKLLIYHTSRL | ctgattacgccggctcgagtgacatccagatga |
| | EDVKDLKTRRNHEQEQTFPGG | HSGVPSRFSGSGSGTDYSLTIS | cccagaccacatctagcctgtccgcctctctgg |
| | GSTIYSMIQSQSSAPTSQEPAY | NLEQEDIATYFCQQGNTLPYT | gcgacagggtgacaatctcctgcagagcctct |
| | TLYSLIQPSRKSGSRKRNHSPS | FGGGTKLEITGSTSGSGKPGSG | caggatatcagcaagtatctgaactggtatcag |
| | FNSTIYEVIGKSQPKAQNPARL | EGSTKGEVKLQESGPGLVAPS | cagaagcccgatggcaccgtgaagctgctgat |
| | SRKELENFDVYSSLGGGSGGS | QSLSVTCTVSGVSLPDYGVSW | ctatcacacatccaggctgcactctggcgtgcc |
| | RVKFSRSADAPAYQQGQNQL | IRQPPRKGLEWLGVIWGSETT | tagccgcttcagcggctccggctctggcaccg |
| | YNELNLGRREEYDVLDKRRG | YYNSALKSRLTIIKDNSKSQVF | actactccctgacaatctctaacctggagcagg |
| | RDPEMGGKPRRKNPQEGLYN | LKMNSLQTDDTAIYYCAKHY | aggatatcgccacctatttctgccagcagggca |
| | ELQKDKMAEAYSEIGMKGER | YYGGSYAMDYWGQGTSVTV | ataccctgccatacacatttggcggcggcacaa |
| | RRGKGHDGLYQGLSTATKDT | SGILTTTTPAPRPPTPAPTIASQ | agctggagatcaccggcagcacatccggctct |
| | YDALHMQALPPRTGGGSGGS | PLSLRPEACRPAAGGAVHTRG | ggcaagccaggcagcggagagggctccacc |
| | CCYDAFIVYDTKDPAVTEWV | LDFACDASSNLFVASWIAVMII | aagggagaggtgaagctgcaggagtccggac |
| | LAELVAKLEDPREKHFNLCLE | FRIGMAVAIFCCFFFPSGSGGS | caggcctggtggcacccagccagtccctgtct |
| | ERDWLPGQPVLENLSQSIQLS | GGSWRRKRKEKQSETSPKEFL | gtgacctgtacagtgtctggcgtgagcctgccc |
| | KKTVFVMTDKYAKTENFKIAF | TIYEDVKDLKTRRNHEQEQTF | gactacggcgtgtcttggatcagacagccccct |
| | YLSHQRLMDEKVDVIILIFLEK | PGGGSTIYSMIQSQSSAPTSQE | agaaagggcctggagtggctgggcgtgatctg |
| | PFQKSKFLQLRKRLCGSSVLE | PAYTLYSLIQPSRKSGSRKRNH | gggcagcgagaccacatactataattctgccct |
| | WPTNPQAHPYFWQCLKNAL | SPSFNSTIYEVIGKSQPKAQNP | gaagagccggctgaccatcatcaaggacaac |
| | (SEQ ID NO: 95) | ARLSRKELENFDVYSSLGGGS | agcaagtcccaggtgttcctgaagatgaacag |
| | | GGSRVKFSRSADAPAYQQGQ | cctgcagaccgacgatacagccatctactattg |
| | | NQLYNELNLGRREEYDVLDK | tgccaagcactactattacggcggcagctatgc |
| | | RRGRDPEMGGKPRRKNPQEG | tatggactactggggccagggcacctccgtga |
| | | LYNELQKDKMAEAYSEIGMK | cagtgtctggaattcttaccacaaccacaccag |
| | | GERRRGKGHDGLYQGLSTAT | cacctcggccacccaccccagcaccaacaatc |
| | | KDTYDALHMQALPPRTGGGS | gccagccagcctctgtccctgaggccagagg |
| | | GGSCCYDAFIVYDTKDPAVTE | catgcaggccagcagcaggaggagcagtgc |
| | | WVLAELVAKLEDPREKHFNL | acaccagaggcctggactttgcctgtgatgcta |
| | | CLEERDWLPGQPVLENLSQSI | gctccaacctgtttgtggcctcttggatcgccgt |
| | | QLSKKTVFVMTDKYAKTENF | gatgatcatctttcggatcggcatggccgtggc |
| | | KIAFYLSHQRLMDEKVDVIILI | catcttctgctgtttctttttccccagcggatccg |
| | | FLEKPFQKSKFLQLRKRLCGSS | gcggctctggcggcagctggcggagaaagcg |
| | | VLEWPTNPQAHPYFWQCLKN | gaaggagaagcagtctgagaccagccccaag |
| | | AL (SEQ ID NO: 113) | gagttcctgacaatctacgaggacgtgaaggat |
| | | | ctgaagaccaggcgcaaccacgagcaggag |
| | | | cagacctttcctggcggcggcagcacaatctat |
| | | | tccatgatccagtctcagagctccgcccccacc |
| | | | tcccaggagcctgcctacacactgtatagcctg |
| | | | atccagccttcccggaagtccggctctcggaa |
| | | | gagaaaccacagcccatccttcaattctacaat |
| | | | ctacgaagtgatcggcaagagccagccaaag |
| | | | gcacagaaccccgccaggctgagccgcaag |
| | | | gagctggagaattttgacgtgtattcttccctag |
| | | | gaggcggctccggcggctctagagtgaagttc |
| | | | agcaggtccgccgacgcacctgcataccagc |
| | | | agggacagaaccagctgtataacgagctgaat |
| | | | ctgggccggagagaggagtacgacgtgctgg |
| | | | ataagaggcggggccgggaccccgagatgg |
| | | | gaggcaagccacggagaaagaacccccagg |
| | | | agggcctgtacaatgagctgcagaaggacaa |
| | | | gatggccgaggcctattccgagatcggcatga |
| | | | agggagagaggcgccggggcaagggacac |
| | | | gatggcctgtaccagggcctgtctaccgccac |
| | | | aaaggacacctatgatgccctgcacatgcagg |
| | | | ccctgccccctcggaccggtggcggctctggc |
| | | | ggcagctgctgttacgacgcctttatcgtgtatg |
| | | | acaccaaggaccccgcagtgacagagtgggt |
| | | | gctggccgagctggtggcaaagctggaggac |
| | | | ccaagggagaagcacttcaacctgtgcctcga |
| | | | ggagagggattggctgccaggacagcctgtg |
| | | | ctggagaatctgtctcagagcatccagctgagc |
| | | | aagaagaccgtgtttgtgatgaccgataagtac |
| | | | gccaagaccgagaacttcaagatcgccttttat |
| | | | ctgtcccaccagcggctgatggacgagaaggt |
| | | | ggatgtgatcatcctgatcttcctggagaagcc |
| | | | ctttcagaagtctaagttcctgcagctgcggaa |
| | | | gagactgtgcggcagctccgtgctggagtggc |
| | | | caaccaacccacaggcacacccttacttctggc |
| | | | agtgtctgaagaatgccctg (SEQ ID |
| | | | NO: 131) |
| CD8-aTM- | TTTTPAPRPPTPAPTIASQPLSL | MALPVTALLLPLALLLHAARP | atggcactgcctgtgaccgccctgctgctgcca |
| TLR8-CD3 | RPEACRPAAGGAVHTRGLDF | YPYDVPDYAYPYDVPDYAYP | ctggccctgctgctgcacgccgccaggccttat |
| | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | TFYDAYISYDTKDASVTDWVI | NWYQQKPDGTVKLLIYHTSRL | ctgattacgccggctcgagtgacatccagatga |
| | NELRYHLEESRDKNVLLCLEE | HSGVPSRFSGSGSGTDYSLTIS | cccagaccacatctagcctgtccgcctctctgg |
| | RDWDPGLAIIDNLMQSINQSK | NLEQEDIATYFCQQGNTLPYT | gcgacagggtgacaatctcctgcagagcctct |
| | KTVFVLTKKYAKSWNFKTAF | FGGGTKLEITGSTSGSGKPGSG | caggatatcagcaagtatctgaactggtatcag |
| | YLALQRLMDENMDVIIFILLEP | EGSTKGEVKLQESGPGLVAPS | cagaagcccgatggcaccgtgaagctgctgat |
| | VLQHSQYLRLRQRICKSSILQ | QSLSVTCTVSGVSLPDYGVSW | ctatcacacatccaggctgcactctggcgtgcc |
| | WPDNPKAEGLFWQTLRNVVT | IRQPPRKGLEWLGVIWGSETT | tagccgcttcagcggctccggctctggcaccg |
| | GGGSGGSRVKFSRSADAPAYQ | YYNSALKSRLTIIKDNSKSQVF | actactccctgacaatctctaacctggagcagg |
| | QGQNQLYNELNLGRREEYDV | LKMNSLQTDDTAIYYCAKHY | aggatatcgccacctatttctgccagcagggca |
| | LDKRRGRDPEMGGKPRRKNP | YYGGSYAMDYWGQGTSVTV | ataccctgccatacacatttggcggcggcacaa |
| | QEGLYNELQKDKMAEAYSEI | SGILTTTTPAPRPPTPAPTIASQ | agctggagatcaccggcagcacatccggctct |
| | GMKGERRRGKGHDGLYQGLS | PLSLRPEACRPAAGGAVHTRG | ggcaagccaggcagcggagagggctccacc |
| | TATKDTYDALHMQALPPR | LDFACDASSNLFVASWIAVMII | aagggagaggtgaagctgcaggagtccggac |
| | (SEQ ID NO: 96) | FRIGMAVAIFCCFFFPSGSGGS | caggcctggtggcacccagccagtccctgtct |
| | | GGSTFYDAYISYDTKDASVTD | gtgacctgtacagtgtctggcgtgagcctgccc |
| | | WVINELRYHLEESRDKNVLLC | gactacggcgtgtcttggatcagacagccccct |
| | | LEERDWDPGLAIIDNLMQSIN | agaaagggcctggagtggctgggcgtgatctg |
| | | QSKKTVFVLTKKYAKSWNFK | gggcagcgagaccacatactataattctgccct |
| | | TAFYLALQRLMDENMDVIIFIL | gaagagccggctgaccatcatcaaggacaac |
| | | LEPVLQHSQYLRLRQRICKSSI | agcaagtcccaggtgttcctgaagatgaacag |
| | | LQWPDNPKAEGLFWQTLRNV | cctgcagaccgacgatacagccatctactattg |
| | | VTGGGSGGSRVKFSRSADAPA | tgccaagcactactattacggcggcagctatgc |
| | | YQQGQNQLYNELNLGRREEY | tatggactactggggccagggcacctccgtga |
| | | DVLDKRRGRDPEMGGKPRRK | cagtgtctggaattcttaccacaaccacaccag |
| | | NPQEGLYNELQKDKMAEAYS | cacctcggccacccaccccagcaccaacaatc |
| | | EIGMKGERRRGKGHDGLYQG | gccagccagcctctgtccctgaggccagagg |
| | | LSTATKDTYDALHMQALPPR | catgcaggccagcagcaggaggagcagtgc |
| | | (SEQ ID NO: 114) | acaccagaggcctggactttgcctgtgatgcta |
| | | | gctccaacctgtttgtggcctcttggatcgccgt |
| | | | gatgatcatctttcggatcggcatggccgtggc |
| | | | catcttctgctgtttctttttccccagcggatccg |
| | | | gcggctctggcggcagcaccttctacgacgcct |
| | | | atatcagctacgacaccaaggatgcctccgtga |
| | | | cagattgggtcatcaacgagctgcgctatcacc |
| | | | tggaggagtcccgggacaagaatgtgctgctg |
| | | | tgtctagaggagagagactgggaccccggcct |
| | | | ggccatcatcgataacctgatgcagtccatcaa |
| | | | tcagtctaagaagaccgtgttcgtgctgacaaa |
| | | | gaagtatgccaagtcttggaacttcaagacagc |
| | | | cttttacctggccctgcagaggctgatggacga |
| | | | gaatatggatgtgatcatctttatcctgctggagc |
| | | | ccgtgctgcagcacagccagtacctgcggctg |
| | | | agacagaggatctgtaagagctccatcctgca |
| | | | gtggcccgacaaccctaaggccgagggcctg |
| | | | ttttggcagaccctgagaaatgtggtgaccggt |
| | | | ggcggctctggcggcagcagagtgaagttca |
| | | | gcaggtccgccgacgcacctgcataccagca |
| | | | gggacagaaccagctgtataacgagctgaatc |
| | | | tgggccggagagaggagtacgacgtgctgga |
| | | | taagaggggggccgggaccccgagatggg |
| | | | aggcaagccacggagaaagaacccccagga |
| | | | gggcctgtacaatgagctgcagaaggacaag |
| | | | atggccgaggcctattccgagatcggcatgaa |
| | | | gggagagaggcgccggggcaagggacacg |
| | | | atggcctgtaccagggcctgtctaccgccaca |
| | | | aaggacacctatgatgccctgcacatgcaggc |
| | | | cctgccccctcgg (SEQ ID NO: 132) |
| CD8-aTM- | TTTTPAPRPPTPAPTIASQPLSL | MALPVTALLLPLALLLHAARP | atggcactgcctgtgaccgccctgctgctgcca |
| 2B4-TLR8- | RPEACRPAAGGAVHTRGLDF | YPYDVPDYAYPYDVPDYAYP | ctggccctgctgctgcacgccgccaggccttat |
| CD3 | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | WRRKRKEKQSETSPKEFLTIY | NWYQQKPDGTVKLLIYHTSRL | ctgattacgccggctcgagtgacatccagatga |
| | EDVKDLKTRRNHEQEQTFPGG | HSGVPSRFSGSGSGTDYSLTIS | cccagaccacatctagcctgtccgcctctctgg |
| | GSTIYSMIQSQSSAPTSQEPAY | NLEQEDIATYFCQQGNTLPYT | gcgacagggtgacaatctcctgcagagcctct |
| | TLYSLIQPSRKSGSRKRNHSPS | FGGGTKLEITGSTSGSGKPGSG | caggatatcagcaagtatctgaactggtatcag |
| | FNSTIYEVIGKSQPKAQNPARL | EGSTKGEVKLQESGPGLVAPS | cagaagcccgatggcaccgtgaagctgctgat |
| | SRKELENFDVYSSLGGGSGGS | QSLSVTCTVSGVSLPDYGVSW | ctatcacacatccaggctgcactctggcgtgcc |
| | TFYDAYISYDTKDASVTDWVI | IRQPPRKGLEWLGVIWGSETT | tagccgcttcagcggctccggctctggcaccg |
| | NELRYHLEESRDKNVLLCLEE | YYNSALKSRLTIIKDNSKSQVF | actactccctgacaatctctaacctggagcagg |
| | RDWDPGLAIIDNLMQSINQSK | LKMNSLQTDDTAIYYCAKHY | aggatatcgccacctatttctgccagcagggca |
| | KTVFVLTKKYAKSWNFKTAF | YYGGSYAMDYWGQGTSVTV | ataccctgccatacacatttggcggcggcacaa |
| | YLALQRLMDENMDVIIFILLEP | SGILTTTTPAPRPPTPAPTIASQ | agctggagatcaccggcagcacatccggctct |
| | VLQHSQYLRLRQRICKSSILQ | PLSLRPEACRPAAGGAVHTRG | ggcaagccaggcagcggagagggctccacc |
| | WPDNPKAEGLFWQTLRNVVT | LDFACDASSNLFVASWIAVMII | aagggagaggtgaagctgcaggagtccggac |
| | GGGSGGSRVKFSRSADAPAYQ | FRIGMAVAIFCCFFFPSGSGGS | caggcctggtggcacccagccagtccctgtct |
| | QGQNQLYNELNLGRREEYDV | GGSWRRKRKEKQSETSPKEFL | gtgacctgtacagtgtctggcgtgagcctgccc |
| | LDKRRGRDPEMGGKPRRKNP | TIYEDVKDLKTRRNHEQEQTF | gactacggcgtgtcttggatcagacagccccct |
| | QEGLYNELQKDKMAEAYSEI | PGGGSTIYSMIQSQSSAPTSQE | agaaagggcctggagtggctgggcgtgatctg |
| | GMKGERRRGKGHDGLYQGLS | PAYTLYSLIQPSRKSGSRKRNH | gggcagcgagaccacatactataattctgccct |
| | TATKDTYDALHMQALPPR | SPSFNSTIYEVIGKSQPKAQNP | gaagagccggctgaccatcatcaaggacaac |
| | (SEQ ID NO: 97) | ARLSRKELENFDVYSSLGGGS | agcaagtcccaggtgttcctgaagatgaacag |
| | | GGSTFYDAYISYDTKDASVTD | cctgcagaccgacgatacagccatctactattg |
| | | WVINELRYHLEESRDKNVLLC | tgccaagcactactattacggcggcagctatgc |
| | | LEERDWDPGLAIIDNLMQSIN | tatggactactggggccagggcacctccgtga |
| | | QSKKTVFVLTKKYAKSWNFK | cagtgtctggaattcttaccacaaccacaccag |
| | | TAFYLALQRLMDENMDVIIFIL | cacctcggccacccaccccagcaccaacaatc |
| | | LEPVLQHSQYLRLRQRICKSSI | gccagccagcctctgtccctgaggccagagg |
| | | LQWPDNPKAEGLFWQTLRNV | catgcaggccagcagcaggaggagcagtgc |
| | | VTGGGSGGSRVKFSRSADAPA | acaccagaggcctggactttgcctgtgatgcta |
| | | YQQGQNQLYNELNLGRREEY | gctccaacctgtttgtggcctcttggatcgccgt |
| | | DVLDKRRGRDPEMGGKPRRK | gatgatcatctttcggatcggcatggccgtggc |
| | | NPQEGLYNELQKDKMAEAYS | catcttctgctgtttctttttccccagcggatccg |
| | | EIGMKGERRRGKGHDGLYQG | gcggctctggcggcagctggcggagaaagcg |
| | | LSTATKDTYDALHMQALPPR | gaaggagaagcagtctgagaccagccccaag |
| | | (SEQ ID NO: 115) | gagttcctgacaatctacgaggacgtgaaggat |
| | | | ctgaagaccaggcgcaaccacgagcaggag |
| | | | cagacctttcctggcggcggcagcacaatctat |
| | | | tccatgatccagtctcagagctccgcccccacc |
| | | | tcccaggagcctgcctacacactgtatagcctg |
| | | | atccagccttcccggaagtccggctctcggaa |
| | | | gagaaaccacagcccatccttcaattctacaat |
| | | | ctacgaagtgatcggcaagagccagccaaag |
| | | | gcacagaaccccgccaggctgagccgcaag |
| | | | gagctggagaattttgacgtgtattcttccctag |
| | | | gaggcggctccggcggctctaccttctacgac |
| | | | gcctatatcagctacgacaccaaggatgcctcc |
| | | | gtgacagattgggtcatcaacgagctgcgctat |
| | | | cacctggaggagtcccgggacaagaatgtgct |
| | | | gctgtgtctagaggagagagactgggacccc |
| | | | ggcctggccatcatcgataacctgatgcagtcc |
| | | | atcaatcagtctaagaagaccgtgttcgtgctga |
| | | | caaagaagtatgccaagtcttggaacttcaaga |
| | | | cagccttttacctggccctgcagaggctgatgg |
| | | | acgagaatatggatgtgatcatctttatcctgctg |
| | | | gagcccgtgctgcagcacagccagtacctgc |
| | | | ggctgagacagaggatctgtaagagctccatc |
| | | | ctgcagtggcccgacaaccctaaggccgagg |
| | | | gcctgttttggcagaccctgagaaatgtggtga |
| | | | ccggtggcggctctggcggcagcagagtgaa |
| | | | gttcagcaggtccgccgacgcacctgcatacc |
| | | | agcagggacagaaccagctgtataacgagct |
| | | | gaatctgggccggagagaggagtacgacgtg |
| | | | ctggataagaggcggggccgggaccccgag |
| | | | atgggaggcaagccacggagaaagaacccc |
| | | | caggagggcctgtacaatgagctgcagaagg |
| | | | acaagatggccgaggcctattccgagatcggc |
| | | | atgaagggagagaggcgccggggcaaggga |
| | | | cacgatggcctgtaccagggcctgtctaccgc |
| | | | cacaaaggacacctatgatgccctgcacatgc |
| | | | aggccctgccccctcgg (SEQ ID |
| | | | NO: 133) |
| CD8-aTM- | TTTTPAPRPPTPAPTIASQPLSL | MALPVTALLLPLALLLHAARP | atggcactgcctgtgaccgccctgctgctgcca |
| 2B4-CD3- | RPEACRPAAGGAVHTRGLDF | YPYDVPDYAYPYDVPDYAYP | ctggccctgctgctgcacgccgccaggccttat |
| TLR8 | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | WRRKRKEKQSETSPKEFLTIY | NWYQQKPDGTVKLLIYHTSRL | ctgattacgccggctcgagtgacatccagatga |
| | EDVKDLKTRRNHEQEQTFPGG | HSGVPSRFSGSGSGTDYSLTIS | cccagaccacatctagcctgtccgcctctctgg |
| | GSTIYSMIQSQSSAPTSQEPAY | NLEQEDIATYFCQQGNTLPYT | gcgacagggtgacaatctcctgcagagcctct |
| | TLYSLIQPSRKSGSRKRNHSPS | FGGGTKLEITGSTSGSGKPGSG | caggatatcagcaagtatctgaactggtatcag |
| | FNSTIYEVIGKSQPKAQNPARL | EGSTKGEVKLQESGPGLVAPS | cagaagcccgatggcaccgtgaagctgctgat |
| | SRKELENFDVYSSLGGGSGGS | QSLSVTCTVSGVSLPDYGVSW | ctatcacacatccaggctgcactctggcgtgcc |
| | RVKFSRSADAPAYQQGQNQL | IRQPPRKGLEWLGVIWGSETT | tagccgcttcagcggctccggctctggcaccg |
| | YNELNLGRREEYDVLDKRRG | YYNSALKSRLTIIKDNSKSQVF | actactccctgacaatctctaacctggagcagg |
| | RDPEMGGKPRRKNPQEGLYN | LKMNSLQTDDTAIYYCAKHY | aggatatcgccacctatttctgccagcagggca |
| | ELQKDKMAEAYSEIGMKGER | YYGGSYAMDYWGQGTSVTV | ataccctgccatacacatttggcggcggcacaa |
| | RRGKGHDGLYQGLSTATKDT | SGILTTTTPAPRPPTPAPTIASQ | agctggagatcaccggcagcacatccggctct |
| | YDALHMQALPPRTGGGSGGS | PLSLRPEACRPAAGGAVHTRG | ggcaagccaggcagcggagagggctccacc |
| | TFYDAYISYDTKDASVTDWVI | LDFACDASSNLFVASWIAVMII | aagggagaggtgaagctgcaggagtccggac |
| | NELRYHLEESRDKNVLLCLEE | FRIGMAVAIFCCFFFPSGSGGS | caggcctggtggcacccagccagtccctgtct |
| | RDWDPGLAIIDNLMQSINQSK | GGSWRRKRKEKQSETSPKEFL | gtgacctgtacagtgtctggcgtgagcctgccc |
| | KTVFVLTKKYAKSWNFKTAF | TIYEDVKDLKTRRNHEQEQTF | gactacggcgtgtcttggatcagacagccccct |
| | YLALQRLMDENMDVIIFILLEP | PGGGSTIYSMIQSQSSAPTSQE | agaaagggcctggagtggctgggcgtgatctg |
| | VLQHSQYLRLRQRICKSSILQ | PAYTLYSLIQPSRKSGSRKRNH | gggcagcgagaccacatactataattctgccct |
| | WPDNPKAEGLFWQTLRNVV | SPSFNSTIYEVIGKSQPKAQNP | gaagagccggctgaccatcatcaaggacaac |
| | (SEQ ID NO: 98) | ARLSRKELENFDVYSSLGGGS | agcaagtcccaggtgttcctgaagatgaacag |
| | | GGSRVKFSRSADAPAYQQGQ | cctgcagaccgacgatacagccatctactattg |
| | | NQLYNELNLGRREEYDVLDK | tgccaagcactactattacggggcagctatgc |
| | | RRGRDPEMGGKPRRKNPQEG | tatggactactggggccagggcacctccgtga |
| | | LYNELQKDKMAEAYSEIGMK | cagtgtctggaattcttaccacaaccacaccag |
| | | GERRRGKGHDGLYQGLSTAT | cacctcggccacccaccccagcaccaacaatc |
| | | KDTYDALHMQALPPRTGGGS | gccagccagcctctgtccctgaggccagagg |
| | | GGSTFYDAYISYDTKDASVTD | catgcaggccagcagcaggaggagcagtgc |
| | | WVINELRYHLEESRDKNVLLC | acaccagaggcctggactttgcctgtgatgcta |
| | | LEERDWDPGLAIIDNLMQSIN | gctccaacctgtttgtggcctcttggatcgccgt |
| | | QSKKTVFVLTKKYAKSWNFK | gatgatcatctttcggatcggcatggccgtggc |
| | | TAFYLALQRLMDENMDVIIFIL | catcttctgctgtttctttttccccagcggatccg |
| | | LEPVLQHSQYLRLRQRICKSSI | gcggctctggcggcagctggcggagaaagcg |
| | | LQWPDNPKAEGLFWQTLRNV | gaaggagaagcagtctgagaccagccccaag |
| | | V (SEQ ID NO: 116) | gagttcctgacaatctacgaggacgtgaaggat |
| | | | ctgaagaccaggcgcaaccacgagcaggag |
| | | | cagacctttcctggcggcggcagcacaatctat |
| | | | tccatgatccagtctcagagctccgcccccacc |
| | | | tcccaggagcctgcctacacactgtatagcctg |
| | | | atccagccttcccggaagtccggctctcggaa |
| | | | gagaaaccacagcccatccttcaattctacaat |
| | | | ctacgaagtgatcggcaagagccagccaaag |
| | | | gcacagaaccccgccaggctgagccgcaag |
| | | | gagctggagaattttgacgtgtattcttccctag |
| | | | gaggcggctccggcggctctagagtgaagttc |
| | | | agcaggtccgccgacgcacctgcataccagc |
| | | | agggacagaaccagctgtataacgagctgaat |
| | | | ctgggccggagagaggagtacgacgtgctgg |
| | | | ataagaggcggggccgggaccccgagatgg |
| | | | gaggcaagccacggagaaagaacccccagg |
| | | | agggcctgtacaatgagctgcagaaggacaa |
| | | | gatggccgaggcctattccgagatcggcatga |
| | | | agggagagaggcgccggggcaagggacac |
| | | | gatggcctgtaccagggcctgtctaccgccac |
| | | | aaaggacacctatgatgccctgcacatgcagg |
| | | | ccctgccccctcggaccggtggcggctctggc |
| | | | ggcagcaccttctacgacgcctatatcagctac |
| | | | gacaccaaggatgcctccgtgacagattgggt |
| | | | catcaacgagctgcgctatcacctggaggagt |
| | | | cccgggacaagaatgtgctgctgtgtctagag |
| | | | gagagagactgggaccccggcctggccatca |
| | | | tcgataacctgatgcagtccatcaatcagtctaa |
| | | | gaagaccgtgttcgtgctgacaaagaagtatgc |
| | | | caagtcttggaacttcaagacagccttttacctg |
| | | | gccctgcagaggctgatggacgagaatatgga |
| | | | tgtgatcatctttatcctgctggagcccgtgctgc |
| | | | agcacagccagtacctgcggctgagacagag |
| | | | gatctgtaagagctccatcctgcagtggcccga |
| | | | caaccctaaggccgagggcctgttttggcaga |
| | | | ccctgagaaatgtggtg (SEQ ID |
| | | | NO: 134) |
| CD8-aTM- | TTTTPAPRPPTPAPTIASQPLSL | MALPVTALLLPLALLLHAARP | atggccttaccagtgaccgccttgctcctgccg |
| IL18R-2B4- | RPEACRPAAGGAVHTRGLDF | YPYDVPDYAYPYDVPDYAYP | ctggccttgctgctccacgccgccaggccgta |
| CD3 | ACDASSNLFVASWIAVMIIFRI | YDVPDYAGSSDIQMTQTTSSL | cccatacgatgttccagattacgcttatccctac |
| | GMAVAIFCCFFFPSGSGGSGGS | SASLGDRVTISCRASQDISKYL | gacgtgcctgattatgcatacccatatgatgtcc |
| | KTYDAFVSYLKECRPENGEEH | NWYQQKPDGTVKLLIYHTSRL | ccgactatgccggctcgagtgacatccagatg |
| | TFAVEILPRVLEKHFGYKLCIF | HSGVPSRFSGSGSGTDYSLTIS | acacagactacatcctccctgtctgcctctctgg |
| | ERDVVPGGAVVDEIHSLIEKSR | NLEQEDIATYFCQQGNTLPYT | gagacagagtcaccatcagttgcagggcaagt |
| | RLIIVLSKSYMSNEVRYELESG | FGGGTKLEITGSTSGSGKPGSG | caggacattagtaaatatttaaattggtatcagca |
| | LHEALVERKIKIILIEFTPVTDF | EGSTKGEVKLQESGPGLVAPS | gaaaccagatggaactgttaaactcctgatcta |
| | TFLPQSLKLLKSHRVLKWKAD | QSLSVTCTVSGVSLPDYGVSW | ccatacatcaagattacactcaggagtcccatc |
| | KSLSYNSRFWKNLLYLMSLG | IRQPPRKGLEWLGVIWGSETT | aaggttcagtggcagtgggtctggaacagatta |
| | GGSGGSWRRKRKEKQSETSPK | YYNSALKSRLTIIKDNSKSQVF | ttctctcaccattagcaacctggagcaagaaga |
| | EFLTIYEDVKDLKTRRNHEQE | LKMNSLQTDDTAIYYCAKHY | tattgccacttacttttgccaacagggtaatacgc |
| | QTFPGGGSTIYSMIQSQSSAPT | YYGGSYAMDYWGQGTSVTV | ttccgtacacgttcggaggggggactaagttgg |
| | SQEPAYTLYSLIQPSRKSGSRK | SGILTTTTPAPRPPTPAPTIASQ | aaataacaggctccacctctggatctggcaagc |
| | RNHSPSFNSTIYEVIGKSQPKA | PLSLRPEACRPAAGGAVHTRG | ccggatctggcgagggatctaccaagggcga |
| | QNPARLSRKELENFDVYSTGG | LDFACDASSNLFVASWIAVMII | ggtgaaactgcaggagtcaggacctggcctg |
| | GSGGSRVKFSRSADAPAYQQG | FRIGMAVAIFCCFFFPSGSGGS | gtggcgccctcacagagcctgtccgtcacatg |
| | QNQLYNELNLGRREEYDVLD | GGSKTYDAFVSYLKECRPENG | cactgtctcaggggtctcattacccgactatggt |
| | KRRGRDPEMGGKPRRKNPQE | EEHTFAVEILPRVLEKHFGYKL | gtaagctggattcgccagcctccacgaaaggg |
| | GLYNELQKDKMAEAYSEIGM | CIFERDVVPGGAVVDEIHSLIE | tctggagtggctgggagtaatatggggtagtga |
| | KGERRRGKGHDGLYQGLSTA | KSRRLIIVLSKSYMSNEVRYEL | aaccacatactataattcagctctcaaatccaga |
| | TKDTYDALHMQALPPR (SEQ | ESGLHEALVERKIKIILIEFTPV | ctgaccatcatcaaggacaactccaagagcca |
| | ID NO: 99) | TDFTFLPQSLKLLKSHRVLKW | agttttcttaaaaatgaacagtctgcaaactgatg |
| | | KADKSLSYNSRFWKNLLYLM | acacagccatttactactgtgccaaacattattac |
| | | SLGGGSGGSWRRKRKEKQSE | tacggtggtagctatgctatggactactggggt |
| | | TSPKEFLTIYEDVKDLKTRRN | caaggaacctcagtcaccgtctccggaattctt |
| | | HEQEQTFPGGGSTIYSMIQSQS | accaccacaacgcccgctcctcggccaccga |
| | | SAPTSQEPAYTLYSLIQPSRKS | cgccagcgccaactattgcgagtcagcctctca |
| | | GSRKRNHSPSFNSTIYEVIGKS | gtctgcgacctgaggcttgtcgaccagcagcc |
| | | QPKAQNPARLSRKELENFDVY | ggaggcgcagtgcacacgagggggctggac |
| | | STGGGSGGSRVKFSRSADAPA | ttcgcctgtgatgctagctccaacctttttgtggc |
| | | YQQGQNQLYNELNLGRREEY | gtcttggatagcggtgatgataatatttaggattg |
| | | DVLDKRRGRDPEMGGKPRRK | ggatggcggtagcgatattttgctgctttttcttc |
| | | NPQEGLYNELQKDKMAEAYS | cctagcggatccggaggttctggtggatcaaaa |
| | | EIGMKGERRRGKGHDGLYQG | acatatgatgcttttgtgtcttacctaaaagaatg |
| | | LSTATKDTYDALHMQALPPR | ccgacctgaaaatggagaggagcacacctttg |
| | | (SEQ ID NO: 117) | ctgtggagattttgcccagggtgttggagaaac |
| | | | attttgggtataagttatgcatatttgaaagggat |
| | | | gtagtgcctggaggagctgttgttgatgaaatc |
| | | | cactcactgatagagaaaagccgaagactaat |
| | | | cattgtcctaagtaaaagttatatgtctaatgagg |
| | | | tcaggtatgaacttgaaagtggactccatgaag |
| | | | cattggtggaaagaaaaattaaaataatcttaatt |
| | | | gaatttacacctgttactgacttcacattcttgcc |
| | | | ccaatcactaaagcttttgaaatctcacagagttc |
| | | | tgaagtggaaggccgataaatctctttcttataac |
| | | | tcaaggttctggaagaaccttctttacttaatgtc |
| | | | cctaggaggaggttctggtggatcatggagga |
| | | | gaaagaggaaggagaagcagtcagagacca |
| | | | gtcccaaggaatttttgacaatttacgaagatgt |
| | | | caaggatctgaaaaccaggagaaatcacgag |
| | | | caggagcagacttttcctggaggggggagca |
| | | | ccatctactctatgatccagtcccagtcttctgct |
| | | | cccacgtcacaagaacctgcatatacattatatt |
| | | | cattaattcagccttccaggaagtctgggtccag |
| | | | gaagaggaaccacagcccttccttcaatagca |
| | | | ctatctatgaagtgattggaaagagtcaacctaa |
| | | | agcccagaaccctgctcgattgagccgcaaag |
| | | | agctggagaactttgatgtttattccaccggtgg |
| | | | aggttctggtggatcaagagtgaagttcagcag |
| | | | gagcgcagacgcccccgcgtaccagcaggg |
| | | | ccagaaccagctctataacgagctcaatctagg |
| | | | acgaagagaggagtacgatgttttggacaaga |
| | | | gacgtggccgggaccctgagatggggggaa |
| | | | agccgagaaggaagaaccctcaggaaggcct |
| | | | gtacaatgaactgcagaaagataagatggcgg |
| | | | aggcctacagtgagattgggatgaaaggcga |
| | | | gcgccggaggggcaaggggcacgatggcct |
| | | | ttaccagggtctcagtacagccaccaaggaca |
| | | | cctacgacgcccttcacatgcaggccctgccc |
| | | | cctcgc (SEQ ID NO: 135) |
| CD8-aTM- | TTTTPAPRPPTPAPTIASQPLSL | MALPVTALLLPLALLLHAARP | atggcactgcctgtgaccgccctgctgctgcca |
| DNAM1-LFA1- | RPEACRPAAGGAVHTRGLDF | YPYDVPDYAYPYDVPDYAYP | ctggccctgctgctgcacgccgccaggccttat |
| CD3 | ACDASSNLFVASWIAVMIIFRI | YDVPDYAGSSDIQMTQTTSSL | ccatacgacgtgcctgattatgcctacccttatg |
| | GMAVAIFCCFFFPSGSGGSGGS | SASLGDRVTISCRASQDISKYL | acgtgccagattacgcctatccctacgacgtgc |
| | NRRRRRERRDLFTESWDTQK | NWYQQKPDGTVKLLIYHTSRL | ctgattacgccggctcgagtgacatccagatga |
| | APNNYRSPISTSQPTNQSMDD | HSGVPSRFSGSGSGTDYSLTIS | cccagaccacatctagcctgtccgcctctctgg |
| | TREDIYVNYPTFSRRPKTRVSL | NLEQEDIATYFCQQGNTLPYT | gcgacagggtgacaatctcctgcagagcctct |
| | GGGSGGSYKVGFFKRNLKEK | FGGGTKLEITGSTSGSGKPGSG | caggatatcagcaagtatctgaactggtatcag |
| | MEAGRGVPNGIPAEDSEQLAS | EGSTKGEVKLQESGPGLVAPS | cagaagcccgatggcaccgtgaagctgctgat |
| | GQEAGDPGCLKPLHEKDSESG | QSLSVTCTVSGVSLPDYGVSW | ctatcacacatccaggctgcactctggcgtgcc |
| | GGKDTGGGSGGSRVKFSRSA | IRQPPRKGLEWLGVIWGSETT | tagccgcttcagcggctccggctctggcaccg |
| | DAPAYQQGQNQLYNELNLGR | YYNSALKSRLTIIKDNSKSQVF | actactccctgacaatctctaacctggagcagg |
| | REEYDVLDKRRGRDPEMGGK | LKMNSLQTDDTAIYYCAKHY | aggatatcgccacctatttctgccagcagggca |
| | PRRKNPQEGLYNELQKDKMA | YYGGSYAMDYWGQGTSVTV | ataccctgccatacacatttggcggcggcacaa |
| | EAYSEIGMKGERRRGKGHDG | SGILMALPVTALLLPLALLLHA | agctggagatcaccggcagcacatccggctct |
| | LYQGLSTATKDTYDALHMQA | ARPYPYDVPDYAYPYDVPDY | ggcaagccaggcagcggagagggctccacc |
| | LPPR (SEQ ID NO: 224) | AYPYDVPDYAGSSDIQMTQTT | aagggagaggtgaagctgcaggagtccggac |
| | | SSLSASLGDRVTISCRASQDIS | caggcctggtggcacccagccagtccctgtct |
| | | KYLNWYQQKPDGTVKLLIYH | gtgacctgtacagtgtctggcgtgagcctgccc |
| | | TSRLHSGVPSRFSGSGSGTDYS | gactacggcgtgtcttggatcagacagccccct |
| | | LTISNLEQEDIATYFCQQGNTL | agaaagggcctggagtggctgggcgtgatctg |
| | | PYTFGGGTKLEITGSTSGSGKP | gggcagcgagaccacatactataattctgccct |
| | | GSGEGSTKGEVKLQESGPGLV | gaagagccggctgaccatcatcaaggacaac |
| | | APSQSLSVTCTVSGVSLPDYG | agcaagtcccaggtgttcctgaagatgaacag |
| | | VSWIRQPPRKGLEWLGVIWGS | cctgcagaccgacgatacagccatctactattg |
| | | ETTYYNSALKSRLTIIKDNSKS | tgccaagcactactattacggcggcagctatgc |
| | | QVFLKMNSLQTDDTAIYYCA | tatggactactggggccagggcacctccgtga |
| | | KHYYYGGSYAMDYWGQGTS | cagtgtctggaattcttaccaccacaacgcccg |
| | | VTVSGIL (SEQ ID | ctcctcggccaccgacgccagcgccaactatt |
| | | NO: 225) | gcgagtcagcctctcagtctgcgacctgaggct |
| | | | tgtcgaccagcagccggaggcgcagtgcaca |
| | | | cgagggggctggacttcgcctgtgatgctagct |
| | | | ccaacctttttgtggcgtcttggatagcggtgat |
| | | | gataatatttaggattgggatggcggtagcgat |
| | | | attttgctgctttttcttccctagcggatccggag |
| | | | gttctggtggatcaaacagaaggagaaggaga |
| | | | gagagaagagatctatttacagagtcctgggat |
| | | | acacagaaggcacccaataactatagaagtcc |
| | | | catctctaccagtcaacctaccaatcaatccatg |
| | | | gatgatacaagagaggatatttatgtcaactatc |
| | | | caaccttctctcgcagaccaaagactagagtttc |
| | | | cctaggaggaggttctggtggatcatataaggt |
| | | | tgggttctttaagagaaaccttaaagaaaagatg |
| | | | gaggcagggcgcggagtaccaaacggaata |
| | | | ccagccgaagactctgaacaacttgcctctggc |
| | | | caggaggctggtgatcccggatgcttgaaacc |
| | | | cctgcatgaaaaggattccgagtctggcggag |
| | | | gtaaagataccggtggaggttctggtggatcaa |
| | | | gagtgaagttcagcaggagcgcagacgcccc |
| | | | cgcgtaccagcagggccagaaccagctctata |
| | | | acgagctcaatctaggacgaagagaggagta |
| | | | cgatgttttggacaagagacgtggccgggacc |
| | | | ctgagatggggggaaagccgagaaggaaga |
| | | | accctcaggaaggcctgtacaatgaactgcag |
| | | | aaagataagatggcggaggcctacagtgagat |
| | | | tgggatgaaaggcgagcgccggaggggcaa |
| | | | ggggcacgatggcctttaccagggtctcagta |
| | | | cagccaccaaggacacctacgacgcccttcac |
| | | | atgcaggccctgccccctcgc (SEQ ID |
| | | | NO: 226) |

In each case, the CAR polypeptide may comprises, in consecutive order, a CD8 hinge domain, an artificial transmembrane domain (“aTM”), and one or more signaling domain and/or other cytoplasmic domains as indicate in the first columns of Table 4. It may have a polypeptide sequence according to the second column, or a functional variant having at least 55%, at least 70%, at least 75%, at least 80%, at least 90%, at least 95%, or 100% identity thereto. In variations, the CAR polypeptide comprises a ligand binding domain specific to CD19. Examples are provided in the third column of each table. In each case, the CAR polypeptide may comprises have a polypeptide sequence according to the third column, or a functional variant having at least 55%, at least 70%, at least 75%, at least 80%, at least 90%, at least 95%, or 100% identity thereto. The fourth column provides corresponding illustrative polynucleotide sequences for each. In some other variations, the CAR polypeptide comprises a ligand binding domain specific to CD20. In still some other variations, the CAR polypeptide comprises a ligand binding domain specific to HER2

### Co-Expressed Partners

In an aspect, the NK CAR cells of the disclosure are engineered to co-express additional proteins (either membrane bound or secreted). Exemplary compositions and methods are provided in US 2017/0073638 A1.

In some variations, the NK cells of the disclosure are engineered to express Interleukin-15 (IL-15) (either membrane bound or secreted). IL-15 is a cytokine that plays a role in regulating immune response. Without being bound by theory, expression of IL-15 may improve NK cell function. The IL-15 may the comprise polypeptide sequence: MRISKPHLRSISIQCYLCLLLNSHFLTEAGIHVFILGCFSAGLPKTEANWVNVISDLKKIED LIQSMHIDATLYTESDVHPSCKVTAMKCFLLELQVISLESGDASIHDTVENLIILANNSLSS NGNVTESGCKECEELEEKNIKEFLQSFVHIVQMFINTS (SEQ ID NO: 58). The IL-15 may have a polypeptide sequence at least 55%, at least 70%, at least 75%, at least 80%, at least 90%, at least 95%, or 100% identical to SEQ ID NO: 58. In some embodiments, the IL-15 is encoded by the nucleotide sequence set forth in SEQ ID NO: 208. In some embodiments, the IL-15 is encoded by a nucleotide sequence having at least 80%, 85%, 90%, 91%, 92%, 93%, 94%, 95%, 96%, 97%, 98% or 99% identity to SEQ ID NO: 208.

Alternatively, the IL-15 may comprise the polypeptide sequence: MALPVTALLLPLALLLHAARPNWVNVISDLKKIEDLIQSMHIDATLYTESDVHPSCKVTA MKCFLLELQVISLESGDASIHDTVENLIILANNSLSSNGNVTESGCKECEELEEKNIKEFLQ SFVHIVQMFINTSTTTPAPRPPTPAPTIASQPLSLRPEACRPAAGGAVHTRGLDFACDIYI WAPLAGTCGVLLLSLVITLYC (SEQ ID NO: 57). The IL-15 may have a polypeptide sequence at least 55%, at least 70%, at least 75%, at least 80%, at least 90%, at least 95%, or 100% identical to SEQ ID NO: 57.

In some variations, the NK cells of the disclosure are engineered to express Interleukin-18 (IL-18) (either membrane bound or secreted). IL-18 is a pro-inflammatory cytokine that plays a role in immune cell activation and response. Without being bound by theory, expression of IL-18 may enhance NK cell proliferation, maturation, and cytotoxicity. The IL-18 may comprise the polypeptide sequence: MAAEPVEDNCINFVAMKFIDNTLYFIAEDDENLESDYFGKLESKLSVIRNLNDQVLFIDQ GNRPLFEDMTDSDCRDNAPRTIFIISMYKDSQPRGMAVTISVKCEKISTLSCENKIISFKE MNPPDNIKDTKSDIIFFQRSVPGHDNKMQFESSSYEGYFLACEKERDLFKLILKKEDELG DRSIMFTVQNED (SEQ ID NO: 60). The IL-18 may have a polypeptide sequence at least 55%, at least 70%, at least 75%, at least 80%, at least 90%, at least 95%, or 100% identical to SEQ ID NO: 60. Alternatively, the IL-18 may comprise the polypeptide sequence: MWLQSLLLLGTVACSISYFGKLESKLSVIRNLNDQVLFIDQGNRPLFEDMTDSDCRDNA PRTIFIISMYKDSQPRGMAVTISVKCEKISTLSCENKIISFKEMNPPDNIKDTKSDIIFFQRS VPGHDNKMQFESSSYEGYFLACEKERDLFKLILKKEDELGDRSIMFTVQNEDSGGGSGG GGSGGGGSGGGGSGGGSLQAESCTSRPHITVVEGEPFYLKHCSCSLAHEIETTTKSWYKS SGSQEHVELNPRSSSRIALHDCVLEFWPVELNDTGSYFFQMKNYTQKWKLNVIRRNKHS CFTERQVTSKIVEVKKFFQITCENSYYQTLVNSTSLYKNCKKLLLENNKNPTIKKNAEFE DQGYYSCVHFLHHNGKLFNITKTFNITIVEDRSNIVPVLLGPKLNHVAVELGKNVRLNCS ALLNEEDVIYWMFGEENGSDPNIHEEKEMRIMTPEGKWHASKVLRIENIGESNLNVLYN CTVASTGGTDTKSFILVRKADMADIPGHVFTRGMIIAVLILVAVVCLVTVCVIYRVDLVL FYRHLTRRDETLTDGKTYDAFVSYLKECRPENGEEHTFAVEILPRVLEKHFGYKLCIFER DVVPGGAVVDEIHSLIEKSRRLIIVLSKSYMSNEVRYELESGLHEALVERKIKIILIEFTPVT DFTFLPQSLKLLKSHRVLKWKADKSLSYNSRFWKNLLYLMPAKTVKPGRDEPEVLPVL SES (SEQ ID NO: 59). The IL-18 may have a polypeptide sequence at least 55%, at least 70%, at least 75%, at least 80%, at least 90%, at least 95%, or 100% identical to SEQ ID NO: 59.

The co-expressed proteins of the NK cells of the instant disclosure may be fusion proteins. A “fusion protein”, as used herein, refers to an expression product resulting from the fusion of at least two genes. The fusion protein may be a chimeric polypeptide comprising two or more unrelated protein, or protein fragments, conjugated to one another. The co-expressed polypeptides may be linked to the polypeptide encoding the CAR of the disclosure via a self-cleaving peptide, such as a self-cleaving (2A) peptide, or functional variant thereof.

In some variations, the NK cells of the disclosure are engineered to express Interleukin-15/Interleukin-15R (IL15/IL15R) fusion protein. Interleukin-15 Receptor (IL-15R) has been shown to play a role in stimulation of proliferation of immune cells. Without being bound by theory, expression of IL-15/IL15R may improve NK cell function. The IL15/IL15R fusion protein may comprise the polypeptide sequence: MLLLVTSLLLCELPHPAFLLIPNWVNVISDLKKIEDLIQSMHIDATLYTESDVHPSCKVTA MKCFLLELQVISLESGDASIHDTVENLIILANNSLSSNGNVTESGCKECEELEEKNIKEFLQ SFVHIVQMFINTSSGGGSGGGGSGGGGSGGGGSGGGSLQITCPPPMSVEHADIWVKSYS LYSRERYICNSGFKRKAGTSSLTECVLNKATNVAHWTTPSLKCIRDPALVHQRPAPPSTV TTAGVTPQPESLSPSGKEPAASSPSSNNTAATTAAIVPGSQLMPSKSPSTGTTEISSHESSH GTPSQTTAKNWELTASASHQPPGVYPQGHSDTTVAISTSTVLLCGLSAVSLLACYLKSR QTPPLASVEMEAMEALPVTWGTSSRDEDLENCSHHLDYKDDDDKDYKDDDDKDYKD DDDK (SEQ ID NO: 62). The IL15/IL15R fusion protein may have a polypeptide sequence at least 55%, at least 70%, at least 75%, at least 80%, at least 90%, at least 95%, or 100% identical to SEQ ID NO: 62.

In some variations, the NK cells of the disclosure are engineered to express Interleukin-18/Interleukin-18R (IL18/IL18R) fusion protein. Interleukin-18 Receptor (IL-18R) has been shown to play a role in immune cell activation and promotion of cytotoxic activity. Without being bound by theory, expression of IL18/IL18R may enhance NK cell proliferation, maturation, and cytotoxicity. The IL18/IL18R fusion protein may comprise the polypeptide sequence: MLLLVTSLLLCELPHPAFLLIPYFGKLESKLSVIRNLNDQVLFIDQGNRPLFEDMTDSDCR DNAPRTIFIISMYKDSQPRGMAVTISVKCEKISTLSCENKIISFKEMNPPDNIKDTKSDIIFF QRSVPGHDNKMQFESSSYEGYFLACEKERDLFKLILKKEDELGDRSIMFTVQNEDSGGG SGGGGSGGGGSGGGGSGGGSLQAESCTSRPHITVVEGEPFYLKHCSCSLAHEIETTTKSW YKSSGSQEHVELNPRSSSRIALHDCVLEFWPVELNDTGSYFFQMKNYTQKWKLNVIRRN KHSCFTERQVTSKIVEVKKFFQITCENSYYQTLVNSTSLYKNCKKLLLENNKNPTIKKNA EFEDQGYYSCVHFLHHNGKLFNITKTFNITIVEDRSNIVPVLLGPKLNHVAVELGKNVRL NCSALLNEEDVIYWMFGEENGSDPNIHEEKEMRIMTPEGKWHASKVLRIENIGESNLNV LYNCTVASTGGTDTKSFILVRKADMADIPGHVFTRGMIIAVLILVAVVCLVTVCVIYRVD LVLFYRHLTRRDETLTDGKTYDAFVSYLKECRPENGEEHTFAVEILPRVLEKHFGYKLCI FERDVVPGGAVVDEIHSLIEKSRRLIIVLSKSYMSNEVRYELESGLHEALVERKIKIILIEFT PVTDFTFLPQSLKLLKSHRVLKWKADKSLSYNSRFWKNLLYLMPAKTVKPGRDEPEVL PVLSESDYKDDDDKDYKDDDDKDYKDDDDK (SEQ ID NO: 63). The IL18/IL18R fusion protein may have a polypeptide sequence at least 55%, at least 70%, at least 75%, at least 80%, at least 90%, at least 95%, or 100% identical to SEQ ID NO: 63.

In some variations, the NK cells of the disclosure are engineered to express OX40. OX40 (CD134) is a member of the tumor necrosis factor receptor (TNFR) family that plays a role in signal transduction and priming of immune cells. Without being bound by theory, expression of OX40 may enhance activation of MAPK/ERK, NFkB, and AKT pathways; and/or induce proliferation and cytokine release. The OX40 may comprise the polypeptide sequence: MCVGARRLGRGPCAALLLLGLGLSTVTGLHCVGDTYPSNDRCCHECRPGNGMVSRCSR SQNTVCRPCGPGFYNDVVSSKPCKPCTWCNLRSGSERKQLCTATQDTVCRCRAGTQPL DSYKPGVDCAPCPPGHFSPGDNQACKPWTNCTLAGKHTLQPASNSSDAICEDRDPPATQ PQETQGPPARPITVQPTEAWPRTSQGPSTRPVEVPGGRAVAAILGLGLVLGLLGPLAILL ALYLLRRDQRLPPDAHKPPGGGSFRTPIQEEQADAHSTLAKIDYKDDDDKDYKDDDDK DYKDDDDK (SEQ ID NO: 61). The OX40 may have a polypeptide sequence at least 55%, at least 70%, at least 75%, at least 80%, at least 90%, at least 95%, or 100% identical to SEQ ID NO: 61.

Any combination of extracellular ligand-binding domain, hinge domain, transmembrane domain, intracellular/cytoplasmic signaling domain, and co-expressed proteins described herein are envisaged within the scope of the CARs of the instant disclosure.

### Polynucleotides and Vectors

Polynucleotides of the disclosure may be delivered to cells as an isolated nucleic acid or in a vector. In an aspect, the disclosure provides a vector comprising polynucleotides comprising the CARs of the disclosure. In some embodiments, the vector comprises a polynucleotide encoding the co-expressed partners of the disclosure. Additional elements present in the vectors of the disclosure may also comprise a selectable marker and other components, such as enhancers, promoters and termination sequences, necessary for the expression of proteins. The polynucleotides of the disclosure may comprise flanking left and right arms for homology-directed repair. The polynucleotide may comprise a promoter operatively linked to the polynucleotide sequence encoding the CAR.

Polynucleotide of the disclosure may also be used for gene-editing. For example, homology directed repair may be used to insert a polynucleotide into a desired genomic loci. The polynucleotide may include a promoter and other genetic elements, or the repair template may omit these and instead rely on the host genome sequences for transcription.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). One method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or other assays.

Polynucleotides comprising the CARs, or parts thereof, are provided. Polynucleotides encoding any combination of extracellular ligand-binding domain, hinge domain, transmembrane domain, intracellular/cytoplasmic signaling domain, and co-expressed proteins described herein are envisaged within the scope of the instant disclosure.

### Modified Cell

Methods for making a modified cell (e.g., a stem cell, a progenitor cell, or an immune effector cell) are known to those of skill in the art. Methods for making cells expressing polypeptides, such as the chimeric receptors or fusion polypeptides described herein, are known in the art. Exemplary methods are described herein.

In some embodiments, the disclosure provides a modified cell (e.g., a modified stem cell, a modified progenitor cell, or a modified immune effector cells) expressing a chimeric antigen receptor described herein. In some embodiments, the disclosure provides a modified cell (e.g., a modified stem cell, a modified progenitor cell, or a modified immune effector cells) comprising a polynucleotide sequence encoding a chimeric antigen receptor described herein. In some embodiments, the modified cell (e.g., a modified stem cell, a modified progenitor cell, or a modified immune effector cells) is a CISH knock out cell.

In some embodiments, a cell comprises a chimeric antigen receptor described herein comprising a ligand binding domain, a transmembrane domain, and an intracellular signaling domain comprising a TLR2 signaling domain. In some embodiments, a cell comprises a chimeric antigen receptor described herein comprising a ligand binding domain, a transmembrane domain, and an intracellular signaling domain comprising a TLR2 signaling domain and a CD3zeta signaling domain. In some embodiments, a cell comprises a chimeric antigen receptor described herein comprising a ligand binding domain, a transmembrane domain selected from CD8 alpha chain (CD8α), CD28, CD16, NKp44, NKp46, NKG2, DNAM1, IL-2Rβ, TLR, IL-1R subfamily, or functional variant thereof, and an intracellular signaling domain comprising a TLR2 signaling domain and a CD3zeta signaling domain.

### Signaling Domains

In some embodiments, the polynucleotides of the disclosure comprise a sequence encoding a signaling domain of the disclosure. In some embodiments, the polynucleotides encode a TLR signaling domain. In some embodiments, the polynucleotides encode an IL-1R subfamily signaling domain.

In some embodiments, the polynucleotide sequence encoding the signaling domain of the disclosure comprises a nucleic acid sequence encoding a TLR2 signaling domain. The polynucleotide sequence encoding the TLR2 signaling domain may comprise the nucleic acid sequence set forth in SEQ ID NO: 12. The polynucleotide sequence encoding the TLR2 signaling domain may have a polynucleotide sequence at least 55%, at least 70%, at least 75%, at least 80%, at least 90%, at least 95%, or 100% identical to SEQ ID NO: 12.

In some embodiments, the polynucleotide sequence encoding the signaling domain of the disclosure comprises a nucleic acid sequence encoding a TLR8 signaling domain. The polynucleotide sequence encoding the TLR8 signaling domain may comprise the nucleic acid sequence set forth in SEQ ID NO: 18. The polynucleotide sequence encoding the TLR8 signaling domain may have a polynucleotide sequence at least 55%, at least 70%, at least 75%, at least 80%, at least 90%, at least 95%, or 100% identical to SEQ ID NO: 18.

In some embodiments, the polynucleotide sequence encoding the signaling domain of the disclosure comprises a nucleic acid sequence encoding a TLR3 signaling domain. The polynucleotide sequence encoding the TLR3 signaling domain may comprise the nucleic acid sequence set forth in SEQ ID NO: 13. The polynucleotide sequence encoding the TLR3 signaling domain may have a polynucleotide sequence at least 55%, at least 70%, at least 75%, at least 80%, at least 90%, at least 95%, or 100% identical to SEQ ID NO: 13.

In some embodiments, the polynucleotide sequence encoding the signaling domain of the disclosure comprises a nucleic acid sequence encoding a TLR7 signaling domain. The polynucleotide sequence encoding the TLR7 signaling domain may comprise the nucleic acid sequence set forth in SEQ ID NO: 17. The polynucleotide sequence encoding the TLR7 signaling domain may have a polynucleotide sequence at least 55%, at least 70%, at least 75%, at least 80%, at least 90%, at least 95%, or 100% identical to SEQ ID NO: 17.

In some embodiments, the polynucleotide sequence encoding the signaling domain of the disclosure comprises a nucleic acid sequence encoding a TLR4 signaling domain. The polynucleotide sequence encoding the TLR4 signaling domain may comprise the nucleic acid sequence set forth in SEQ ID NO: 14. The polynucleotide sequence encoding the TLR4 signaling domain may have a polynucleotide sequence at least 55%, at least 70%, at least 75%, at least 80%, at least 90%, at least 95%, or 100% identical to SEQ ID NO: 14.

In some embodiments, the polynucleotide sequence encoding the signaling domain of the disclosure comprises a nucleic acid sequence encoding a TLR1 signaling domain. The polynucleotide sequence encoding the TLR1 signaling domain may comprise the nucleic acid sequence set forth in SEQ ID NO: 11. The polynucleotide sequence encoding the TLR1 signaling domain may have a polynucleotide sequence at least 55%, at least 70%, at least 75%, at least 80%, at least 90%, at least 95%, or 100% identical to SEQ ID NO: 11.

In some embodiments, the polynucleotide sequence encoding the signaling domain of the disclosure comprises a nucleic acid sequence encoding a TLR5 signaling domain. The polynucleotide sequence encoding the TLR5 signaling domain may comprise the nucleic acid sequence set forth in SEQ ID NO: 15. The polynucleotide sequence encoding the TLR5 signaling domain may have a polynucleotide sequence at least 55%, at least 70%, at least 75%, at least 80%, at least 90%, at least 95%, or 100% identical to SEQ ID NO: 15.

In some embodiments, the polynucleotide sequence encoding the signaling domain of the disclosure comprises a nucleic acid sequence encoding a TLR6 signaling domain. The polynucleotide sequence encoding the TLR6 signaling domain may comprise the nucleic acid sequence set forth in SEQ ID NO: 16. The polynucleotide sequence encoding the TLR6 signaling domain may have a polynucleotide sequence at least 55%, at least 70%, at least 75%, at least 80%, at least 90%, at least 95%, or 100% identical to SEQ ID NO: 16.

In some embodiments, the polynucleotide sequence encoding the signaling domain of the disclosure comprises a nucleic acid sequence encoding a TLR9 signaling domain. The polynucleotide sequence encoding the TLR9 signaling domain may comprise the nucleic acid sequence set forth in SEQ ID NO: 19. The polynucleotide sequence encoding the TLR9 signaling domain may have a polynucleotide sequence at least 55%, at least 70%, at least 75%, at least 80%, at least 90%, at least 95%, or 100% identical to SEQ ID NO: 19.

In some embodiments, the polynucleotide sequence encoding the signaling domain of the disclosure comprises a nucleic acid sequence encoding a TLR10 signaling domain. The polynucleotide sequence encoding the TLR10 signaling domain may comprise the nucleic acid sequence set forth in SEQ ID NO: 20. The polynucleotide sequence encoding the TLR10 signaling domain may have a polynucleotide sequence at least 55%, at least 70%, at least 75%, at least 80%, at least 90%, at least 95%, or 100% identical to SEQ ID NO: 20.

In some embodiments, the polynucleotide sequence encoding the signaling domain of the disclosure comprises a nucleic acid sequence encoding an IL-1R signaling domain. The polynucleotide sequence encoding the IL-1R signaling domain may comprise a nucleic acid sequence that encodes for the polypeptide set forth in SEQ ID NO: 21. The polynucleotide sequence encoding the IL-1R signaling domain may have a polynucleotide sequence that encodes a polypeptide sequence at least 55%, at least 70%, at least 75%, at least 80%, at least 90%, at least 95%, or 100% identical to SEQ ID NO: 21.

In some embodiments, the polynucleotide sequence encoding the signaling domain of the disclosure comprises a nucleic acid sequence encoding an IL-18R signaling domain. The polynucleotide sequence encoding the IL-18R signaling domain may comprise the nucleic acid sequence set forth in SEQ ID NO: 24. The polynucleotide sequence encoding the IL-1R signaling domain may have a polynucleotide sequence at least 55%, at least 70%, at least 75%, at least 80%, at least 90%, at least 95%, or 100% identical to SEQ ID NO: 24.

TABLE 5
Signaling domains

| | | SEQ |
|---|---|---|
| | | ID |
| Domain | Sequence | NO: |
| TLR1 | tttgaatatgcagcatatataattcatgcctataaagataaggattgggtctgggaacatttctcttca | 11 |
| signaling | atggaaaaggaagaccaatctctcaaattttgtctggaagaaagggactttgaggcgggtgttttt | |
| domain | gaactagaagcaattgttaacagcatcaaaagaagcagaaaaattatttttgttataacacaccatc | |
| | tattaaaagacccattatgcaaaagattcaaggtacatcatgcagttcaacaagctattgaacaaaa | |
| | tctggattccattatattggttttccttgaggagattccagattataaactgaaccatgcactctgtttg | |
| | cgaagaggaatgtttaaatctcactgcatcttgaactggccagttcagaaagaacggataggtgc | |
| | ctttcgtcataaattgcaagtagcactt | |
| TLR2 | tgttgctatgatgcttttattgtgtatgacactaaagacccagctgtgaccgagtgggttttggctga | 12 |
| signaling | gctggtggccaaactggaagacccaagagagaaacattttaatttatgtctcgaggaaagggact | |
| domain | ggttaccagggcagccagttctggaaaacctttcccagagcatacagcttagcaaaaagacagt | |
| | gtttgtgatgacagacaagtatgcaaagactgaaaattttaagatagcattttacttgtcccatcaga | |
| | ggctcatggatgaaaaagttgatgtgattatcttgatatttcttgagaagccctttcagaagtccaag | |
| | ttcctccagctccggaaaaggctctgtgggagttctgtccttgagtggccaacaaacccgcaagc | |
| | tcacccatacttctggcagtgtctaaagaacgccctg | |
| TLR3 | actttctatgatgcttacatttcttatgacaccaaagatgcctctgttactgactgggtgataaatgag | 13 |
| signaling | ctgcgctaccaccttgaagagagccgagacaaaaacgttctcctttgtctagaggagagggattg | |
| domain | ggacccgggattggccatcatcgacaacctcatgcagagcatcaaccaaagcaagaaaacagt | |
| | atttgttttaaccaaaaaatatgcaaaaagctggaactttaaaacagctttttacttggctttgcagag | |
| | gctaatggatgagaacatggatgtgattatatttatcctgctggagccagtgttacagcattctcagt | |
| | atttgaggctacggcagcggatctgtaagagctccatcctccagtggcctgacaacccgaaggc | |
| | agaaggcttgttttggcaaactctgagaaatgtggtc | |
| TLR4 | ctgccctacgatgccttcgtggtcttcgacaaaacgcagagcgcagtggcagactgggtgtaca | 14 |
| signaling | acgagcttcgggggcagctggaggagtgccgtgggcgctgggcactccgcctgtgcctggag | |
| domain | gaacgcgactggctgcctggcaaaaccctctttgagaacctgtgggcctcggtctatggcagcc | |
| | gcaagacgctgtttgtgctggcccacacggaccgggtcagtggtctcttgcgcgccagcttcctg | |
| | ctggcccagcagcgcctgctggaggaccgcaaggacgtcgtggtgctggtgatcctgagccct | |
| | gacggccgccgctcccgctacgtgcggctgcgccagcgcctctgccgccagagtgtcctcctc | |
| | tggccccaccagcccagtggtcagcgcagcttctgggcccagctgggcatggccctg | |
| TLR5 | atctgctatgatgcatttgtttcttacagtgagcgggatgcctactgggtggagaaccttatggtcca | 15 |
| signaling | ggagctggagaacttcaatccccccttcaagttgtgtcttcataagcgggacttcattcctggcaa | |
| domain | gtggatcattgacaatatcattgactccattgaaaagagccacaaaactgtctttgtgctttctgaaa | |
| | actttgtgaagagtgagtggtgcaagtatgaactggacttctcccatttccgtctttttgatgagaac | |
| | aatgatgctgccattctcattcttctggagcccattgagaaaaaagccattccccagcgcttctgca | |
| | agctgcggaagataatgaacaccaagacctacctggagtggcctatggacgaggctcagcgg | |
| | gaaggattttgggtaaatctgagagctgcgata | |
| TLR6 | aacatctatgatgcctttgttatctactcaagccaggatgaggactgggtaaggaatgagctagta | 16 |
| signaling | aagaatttagaagaaggggtgcctccatttcagctctgccttcactacagagactttattcccggtg | |
| domain | tggccattgctgccaacatcatccatgaaggtttccataaaagccgaaaggtgattgttgtggtgtc | |
| | ccagcacttcatccagagccgctggtgtatctttgaatatgagattgctcagacctggcagtttctg | |
| | agcagtcgtgctggtatcatcttcattgtcctgcagaaggtggagaagaccctgctcaggcagca | |
| | ggtggagctgtaccgccttctcagcaggaacacttacctggagtgggaggacagtgtcctgggg | |
| | cggcacatcttctggagacgactcagaaaagccctg | |
| TLR7 | gtccgattccacgcatttatttcatacagtgaacatgattctctgtgggtgaagaatgaattgatccc | 17 |
| signaling | caatctagagaaggaagatggttctatcttgatttgcctttatgaaagctactttgaccctggcaaaa | |
| domain | gcattagtgaaaatattgtaagcttcattgagaaaagctataagtccatctttgttttgtctcccaactt | |
| | tgtccagaatgagtggtgccattatgaattctactttgcccaccacaatctcttccatgaaaattctga | |
| | tcatataattcttatcttactggaacccattccattctattgcattcccaccaggtatcataaactgaaa | |
| | gctctcctggaaaaaaaagcatacttggaatggcccaaggataggcgtaaatgtgggcttttctg | |
| | ggcaaaccttcgagctgctatt | |
| TLR8 | ctccagtttcatgcatttatttcatatagtgggcacgattctttctgggtgaagaatgaattattgccaa | 18 |
| signaling | acctagagaaagaaggtatgcagatttgccttcatgagagaaactttgttcctggcaagagcattg | |
| domain | tggaaaatatcatcacctgcattgagaagagttacaagtccatctttgttttgtctcccaactttgtcc | |
| | agagtgaatggtgccattatgaactctactttgcccatcacaatctctttcatgaaggatctaatagc | |
| | ttaatcctgatcttgctggaacccattccgcagtactccattcctagcagttatcacaagctcaaaag | |
| | tctcatggccaggaggacttatttggaatggcccaaggaaaagagcaaacgtggccttttttggg | |
| | ctaacttaagggcagccatt | |
| TLR9 | ctccagtttcatgcttttatttcatatagtgaacatgattctgcctgggtgaaaagtgaattggtacctt | 19 |
| signaling | acctagaaaaagaagatatacagatttgtcttcatgagagaaactttgtccctggcaagagcattgt | |
| domain | ggaaaatatcatcaactgcattgagaagagttacaagtccatctttgttttgtctcccaactttgtcca | |
| | gagtgagtggtgccattacgaactctattttgcccatcacaatctctttcatgaaggatctaataactt | |
| | aatcctcatcttactggaacccattccacagaacagcattcccaacaagtaccacaagctgaagg | |
| | ctctcatgacgcagcggacttatttgcagtggcccaaggagaaaagcaaacgtgggctcttttgg | |
| | gctaacattagagccgctttt | |
| TLR10 | tacaaatatgatgcctatttgtgcttcagcagcaaagacttcacatgggtgcagaatgctttgctca | 20 |
| signaling | aacacctggacactcaatacagtgaccaaaacagattcaacctgtgctttgaagaaagagacttt | |
| domain | gtcccaggagaaaaccgcattgccaatatccaggatgccatctggaacagtagaaagatcgtttg | |
| | tcttgtgagcagacacttccttagagatggctggtgccttgaagccttcagttatgcccagggcag | |
| | gtgcttatctgaccttaacagtgctctcatcatggtggtggttgggtccttgtcccagtaccagttga | |
| | tgaaacatcaatccatcagaggctttgtacagaaacagcagtatttgaggtggcctgaggatctcc | |
| | aggatgttggctggtttcttcataaactctctcaacagata | |
| IL-1R | KTYDAYILYPKTVGEGSTSDCDIFVFKVLPEVLEKQCGYKLFI | 21 |
| signaling | YGRDDYVGEDIVEVINENVKKSRRLIIILVRETSGFSWLGGSS | |
| domain | EEQIAMYNALVQDGIKVVLLELEKIQDYEKMPESIKFIKQKH | |
| | GAIRWSGDFTQGPQSAKTRFWKNVRYHM | |
| IL-18R | KTYDAFVSYLKECRPENGEEHTFAVEILPRVLEKHFGYKLCIF | 22 |
| signaling | ERDVVPGGAVVDEIHSLIEKSRRLIIVLSKSYMSNEVRYELES | |
| domain | GLHEALVERKIKIILIEFTPVTDFTFLPQSLKLLKSHRVLKWK | |
| | ADKSLSYNSRFWKNLLYLM | |
| IL-18R | aaaacatatgatgcttttgtgtcttacctaaaagaatgccgacctgaaaatggagaggagcacacc | 24 |
| signaling | tttgctgtggagattttgcccagggtgttggagaaacattttgggtataagttatgcatatttgaaag | |
| domain | ggatgtagtgcctggaggagctgttgttgatgaaatccactcactgatagagaaaagccgaaga | |
| | ctaatcattgtcctaagtaaaagttatatgtctaatgaggtcaggtatgaacttgaaagtggactcca | |
| | tgaagcattggtggaaagaaaaattaaaataatcttaattgaatttacacctgttactgacttcacatt | |
| | cttgccccaatcactaaagcttttgaaatctcacagagttctgaagtggaaggccgataaatctcttt | |
| | cttataactcaaggttctggaagaaccttctttacttaatg | |
| IL-1R4 | KLYDAYVVYPRNYKSSTDGASRVEHFVHQILPDVLENKCGY | 191 |
| signaling | TLCIYGRDMLPGEDVVTAVETNIRKSRRHIFILTPQITHNKEF | |
| domain | AYEQEVALHCALIQNDAKVILIEMEALSELDMLQAEALQDSL | |
| | QHLMKVQGTIKWREDHIANKRSLNSKFWKHVRYQM | |
| IL-1R6 | KLYDAYVLYPKPHKESQRHAVDALVLNILPEVLERQCGYKL | 192 |
| signaling | FIFGRDEFPGQAVANVIDENVKLCRRLIVIVVPESLGFGLLKN | |
| domain | LSEEQIAVYSALIQDGMKVILIELEKIEDYTVMPESIQYIKQKH | |
| | GAIRWHGDFTEQSQCMKTKFWKTVRYHM | |
| IL-1R3 | KEYDIYVSYARNAEEEEFVLLTLRGVLENEFGYKLCIFDRDS | 193 |
| signaling | LPGGIVTDETLSFIQKSRRLLVVLSPNYVLQGTQALLELKAGL | |
| domain | ENMASRGNINVILVQYKAVKETKVKELKRAKTVLTVIKWKG | |
| | EKSKYPQGRFWKQLQVAM | |
| IL-1R7 | KDFDAFVSYAKWSSFPSEATSSLSEEHLALSLFPDVLENKYG | 194 |
| signaling | YSLCLLERDVAPGGVYAEDIVSIIKRSRRGIFILSPNYVNGPSIF | |
| domain | ELQAAVNLALDDQTLKLILIKFCYFQEPESLPHLVKKALRVL | |
| | PTVTWRGLKSVPPNSRFWAKMRYHM | |
| IL-1R8 | KLYDAYVSYSDCPEDRKFVNFILKPQLERRRGYKLFLDDRDL | 195 |
| signaling | LPRAEPSADLLVNLSRCRRLIVVLSDAFLSRAWCSHSFREGL | |
| domain | CRLLELTRRPIFITFEGQRRDPAHPALRLLRQHRHLVTLLLWR | |
| | PGSVTPSSDFWKEVQLAL | |
| IL-IR9 | KDYDAYLSYTKVDPDQWNQETGEEERFALEILPDMLEKHYG | 196 |
| signaling | YKLFIPDRDLIPTGTYIEDVARCVDQSKRLIIVMTPNYVVRRG | |
| domain | WSIFELETRLRNMLVTGEIKVILIECSELRGIMNYQEVEALKH | |
| | TIKLLTVIKWHGPKCNKLNSKFWKRLQYEM | |
| IL-1R10 | KEYDAYLSYTKVDQDTLDCDNPEEEQFALEVLPDVLEKHYG | 197 |
| signaling | YKLFIPERDLIPSGTYMEDLTRYVEQSRRLIIVLTPDYILRRGW | |
| domain | SIFELESRLHNMLVSGEIKVILIECTELKGKVNCQEVESLKRSI | |
| | KLLSLIKWKGSKSSKLNSKFWKHLVYEM | |

### Other Components of CAR Cytoplasmic Portion

The cytoplasmic portion of the CAR may further comprise one or more of the domains listed in Table 6, or a functional variant having at least 55%, at least 70%, at least 75%, at least 80%, at least 90%, at least 95%, or 100% identity thereto.

TABLE 6
Further cytoplasmic domains

| | | SEQ |
|---|---|---|
| | | ID |
| Domain | SEQ ID NO: | NO: |
| CD3z (CD3ζ) | RVKFSRSADAPAYQQGQNQLYNELNLGRREEYDVLDKRRG | 64 |
| | RDPEMGGKPRRKNPQEGLYNELQKDKMAEAYSEIGMKGER | |
| | RRGKGHDGLYQGLSTATKDTYDALHMQALPPR | |
| 2B4 | WRRKRKEKQSETSPKEFLTIYEDVKDLKTRRNHEQEQTFPGG | 65 |
| | GSTIYSMIQSQSSAPTSQEPAYTLYSLIQPSRKSGSRKRNHSPS | |
| | FNSTIYEVIGKSQPKAQNPARLSRKELENFDVYS | |
| OX40 | RRDQRLPPDAHKPPGGGSFRTPIQEEQADAHSTLAKI | 66 |
| FCER1G | LRKRRDSLSLSTQRTQGPAESARNLEYVSVSPTNNTVYASVT | 67 |
| | HSNRETEIWTPRENDTITIYSTINHSKESKPTFSRAALDNV | |
| CD28 | RSKRSRLLHSDYMNMTPRRPGPTRKHYQPYAPPRDFAAYRS | 68 |
| CD28 | RSKRSRLLHSDYMNMTPRRPGPTRKHYQPYAPPRDFAAYRS | 220 |
| | L | |
| DAP10 | LCARPRRSPAQEDGKVYINMPGRG | 69 |
| DNAM1 | NRRRRRERRDLFTESWDTQKAPNNYRSPISTSQPTNQSMDDT | 70 |
| | REDIYVNYPTFSRRPKTRV | |
| CD28H | FWGRRSCQQRDSGNSPGNAFYSNVLYRPRGAPKKSEDCSGE | 71 |
| | GKDQRGQSIYSTSFPQPAPRQPHLASRPCPSPRPCPSPRPGHP | |
| | VSMVRVSPRPSPTQQPRPKGFPKVGEE | |
| 41BB | RFSVVKRGRKKLLYIFKQPFMRPVQTTQEEDGCSCRFPEEEE | 176 |
| | GGCEL | |

In some embodiments, a chimeric receptor described herein comprises a cytoplasmic domain. In some embodiments, the cytoplasmic domain is operably linked to the TIR signaling domain with a linker. In some embodiments, the cytoplasmic domain is operably linked to the TIR signaling domain without a linker. In some embodiments, the N-terminus of the cytoplasmic domain is operably linked to the C-terminus of the TIR signaling domain. In some embodiments, the C-terminus of the cytoplasmic domain is operably linked to the N-terminus of the TIR signaling domain.

In some embodiments, the cytoplasmic domain is a 2B4 signaling domain. In some embodiments, the 2B4 cytoplasmic domain comprises the amino acid sequence set forth in SEQ ID NO: 65. In some embodiments, the 2B4 cytoplasmic domain consists of amino acid sequence set forth in SEQ ID NO: 65. In some embodiments, the 2B4 cytoplasmic domain comprises an amino acid sequence 80%, 85%, 90%, 91%, 92%, 93%, 94%, 95%, 96%, 97%, 98% or 99% identical to SEQ ID NO: 65. In some embodiments, the 2B4 cytoplasmic domain is encoded by the nucleotide sequence set forth in SEQ ID NO: 179. In some embodiments, the 2B4 cytoplasmic domain is encoded by a nucleotide sequence at least 80%, 85%, 90%, 91%, 92%, 93%, 94%, 95%, 96%, 97%, 98% or 99% identical to SEQ ID NO: 179.

In some embodiments, the cytoplasmic domain is a CD3ζ cytoplasmic domain. In some embodiments, the CD3ζ cytoplasmic domain comprises the amino acid sequence set forth in SEQ ID NO: 64. In some embodiments, the CD3ζ cytoplasmic domain consists of amino acid sequence set forth in SEQ ID NO: 64. In some embodiments, the CD3ζ cytoplasmic domain comprises an amino acid sequence 80%, 85%, 90%, 91%, 92%, 93%, 94%, 95%, 96%, 97%, 98% or 99% identical to SEQ ID NO: 64. In some embodiments, the CD3ζ cytoplasmic domain is encoded by the nucleotide sequence set forth in SEQ ID NO: 181. In some embodiments, the CD3ζ cytoplasmic domain is encoded by a nucleotide sequence at least 80%, 85%, 90%, 91%, 92%, 93%, 94%, 95%, 96%, 97%, 98% or 99% identical to SEQ ID NO: 181.

In some embodiments, the cytoplasmic domain is a 41BB cytoplasmic domain. In some embodiments, the 41BB signaling domain comprises the amino acid sequence set forth in SEQ ID NO: 176. In some embodiments, the 41BB cytoplasmic domain consists of amino acid sequence set forth in SEQ ID NO: 176. In some embodiments, the 41BB cytoplasmic domain comprises an amino acid sequence 80%, 85%, 90%, 91%, 92%, 93%, 94%, 95%, 96%, 97%, 98% or 99% identical to SEQ ID NO: 176. In some embodiments, the 41BB cytoplasmic domain is encoded by the nucleotide sequence set forth in SEQ ID NO: 177. In some embodiments, the 41BB cytoplasmic domain is encoded by a nucleotide sequence at least 80%, 85%, 90%, 91%, 92%, 93%, 94%, 95%, 96%, 97%, 98% or 99% identical to SEQ ID NO: 177.

In some embodiments, the cytoplasmic domain is a CD28 cytoplasmic domain. In some embodiments, the CD28 cytoplasmic domain comprises the amino acid sequence set forth in SEQ ID NO: 68. In some embodiments, the cytoplasmic domain is a CD28 cytoplasmic domain. In some embodiments, the CD28 cytoplasmic domain comprises the amino acid sequence set forth in SEQ ID NO: 220. In some embodiments, the CD28 cytoplasmic domain consists of amino acid sequence set forth in SEQ ID NO: 68. In some embodiments, the CD28 cytoplasmic domain consists of amino acid sequence set forth in SEQ ID NO: 220. In some embodiments, the CD28 cytoplasmic domain comprises an amino acid sequence 80%, 85%, 90%, 91%, 92%, 93%, 94%, 95%, 96%, 97%, 98% or 99% identical to SEQ ID NO: 68. In some embodiments, the CD28 cytoplasmic domain comprises an amino acid sequence 80%, 85%, 90%, 91%, 92%, 93%, 94%, 95%, 96%, 97%, 98% or 99% identical to SEQ ID NO: 220. In some embodiments, the CD28 cytoplasmic domain is encoded by the nucleotide sequence set forth in SEQ ID NO: 178. In some embodiments, the CD28 cytoplasmic domain is encoded by the nucleotide sequence set forth in SEQ ID NO: 219. In some embodiments, the CD28 cytoplasmic domain is encoded by a nucleotide sequence at least 80%, 85%, 90%, 91%, 92%, 93%, 94%, 95%, 96%, 97%, 98% or 99% identical to SEQ ID NO: 178. In some embodiments, the CD28 cytoplasmic domain is encoded by a nucleotide sequence at least 80%, 85%, 90%, 91%, 92%, 93%, 94%, 95%, 96%, 97%, 98% or 99% identical to SEQ ID NO: 219.

### Co-Expressed Partners

Activity of NK cells may be increased by co-expressing various factors, including but not limited to those provided in Table 7.

TABLE 7
Co-expression partners

| Co-expression partner | SEQ ID NO: |
|---|---|
| Membrane bound interleukin | 57 |
| 15 (mbIL-15) | |
| soluble IL15 | 58 |
| Membrane bound interleukin | 59 |
| 18 (mbIL-18) | |
| soluble IL18 | 60 |
| OX40 | 61 |
| IL15/IL15R fusion | 62 |
| IL18/IL18R fusion | 63 |

### Exemplary Chimeric Antigen Receptors

In some embodiments, a chimeric antigen receptor described herein comprises a ligand binding domain, a transmembrane domain, and an intracellular signaling domain comprising a TLR2 signaling domain. In some embodiments, a chimeric antigen receptor described herein comprises a ligand binding domain, a transmembrane domain, and an intracellular signaling domain comprising a TLR2 signaling domain and a CD3zeta signaling domain. In some embodiments, a chimeric antigen receptor described herein comprises (a) a ligand binding domain, (b) a transmembrane domain selected from the group consisting of CD8 alpha chain (CD8α), CD28, CD16, NKp44, NKp46, NKG2D, DNAM1, IL-2Rβ, TLR, IL-1R subfamily, or functional variant thereof, and (c) an intracellular signaling domain comprising a TLR2 signaling domain and a CD3ζ signaling domain. Exemplary, non-limiting examples include, for example, a chimeric antigen receptor described herein comprises a ligand binding domain, an aTM domain, and an intracellular signaling domain comprising a TLR2 signaling domain and a CD3ζ signaling domain. A further exemplary chimeric antigen receptor comprises a ligand binding domain, a CD28 transmembrane domain, and an intracellular signaling domain comprising a TLR2 signaling domain and a CD3ζ signaling domain.

### Kits

In an aspect, the disclosure provides a kit comprising the CAR NK cells or the pharmaceutical composition of the disclosure and instructions for use.

### Methods of Use

In an aspect, the disclosure provides a method of treating or preventing a disease or disorder in a subject in need thereof, comprising administering the CAR NK cell of the disclosure or the pharmaceutical composition of the disclosure to the subject.

In an aspect, the disclosure provides a method of killing a target cell comprising contacting a population of target cells with a population of natural killer cells of the disclosure, wherein the chimeric antigen receptors of the natural killer cell comprise a ligand binding domain that specifically binds an antigen on the target cell, and wherein the natural killer cells induce specific killing of the target cells.

In an aspect, the disclosure provides method of killing a target cell comprising contacting a population of target cells with a population of NK cells.

Cell killing may be assessed by assays known in the art, including but not limited to those described in the examples.

The cancer may result from proliferation of tumor cells that display an antigen specifically bound by the CAR or an NK cell expressing the CAR. The CARs provided herein include CARs having a ligand-binding domain that specifically binds CD19, CD20, and/or HER2. CD19 and CD20 can be expressed on tumor cells of various indolent and aggressive subtypes of B cell lymphomas and leukemias, including Non-Hodgkin's lymphoma (NHL), B-cell Chronic lymphocytic leukemia (CLL), and non-T acute lymphoblastic leukemia (ALL). Accordingly, the CARs described herein may be used to kill CD19+ and/or CD20+ tumor cells and/or treat cancer in subjects having Non-Hodgkin's lymphoma (NHL), B-cell Chronic lymphocytic leukemia (CLL), and non-T acute lymphoblastic leukemia (ALL). HER2 expression has been found in several tumor types including, but not limited to, breast, esophageal, lung, cervical, endometrial and ovarian cancer. Accordingly, the CARs described herein may be used to kill HER2+ tumor cells and/or treat cancer in subjects having breast, esophageal, lung, cervical, endometrial and/or ovarian cancer.

In an aspect, the disclosure provides methods of treating cancer in a subject in need thereof, comprising administering a population of NK cells, or pharmaceutical compositions comprising a population of NK cells.

The terms “subject,” “patient”, and “individual” are used interchangeably herein to refer to a vertebrate, such as a mammal, e.g., a human

As used herein, the term “treatment” or “treating” embraces at least an amelioration of the symptoms associated with a disease or condition in the patient, where amelioration is used in a broad sense to refer to at least a reduction in the magnitude of a parameter, e.g., a symptom associated with the condition being treated. As such, “treatment” or “treating” also includes situations where the disease, disorder, or pathological condition, or at least symptoms associated therewith, are completely inhibited (e.g., prevented from happening) or stopped (e.g., terminated) such that the patient no longer suffers from the condition, or at least the symptoms that characterize the condition.

In some embodiments, the method of treating cancer in a subject in need thereof comprises administering a therapeutically effective amount of CAR NK cells and/or pharmaceutical composition of the disclosure to the subject.

As used herein, “therapeutically effective” refers to an amount of cells or pharmaceutical composition that is sufficient to treat or ameliorate, or in some manner reduce the symptoms associated with a disease, such as cancer/

In some embodiments, the CAR NK cells and pharmaceutical compositions comprising the same described herein are used as part of a combination therapy. In some embodiments, the CAR NK cells or pharmaceutical compositions of the instant disclosure are co-administered to the subject with tumor targeting antibodies.

In some embodiments, the CAR NK cells or pharmaceutical compositions of the instant disclosure exhibit increased cytotoxicity relative to cells or pharmaceutical compositions in which the cells have not been engineered in such a way as the instant disclosure. In some embodiments, the cells or pharmaceutical compositions of the instant disclosure exhibit increased effectiveness relative to equivalent cells or pharmaceutical compositions in cells have not been engineered in such a way as the instant disclosure.

The population of NK cells or pharmaceutical composition may be administered in an amount effective to kill target cells and/or in a therapeutically effective amount for treatment of the cancer.

### Pharmaceutical Compositions

In an aspect, the disclosure provides a pharmaceutical composition comprising the CAR NK cell or cell population of the disclosure and one or more pharmaceutically acceptable excipients or diluents.

As used herein, the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein, the term “pharmaceutically acceptable excipients or diluents” refers to an excipient, diluent, preservative, solubilizer, emulsifier, adjuvant, and/or vehicle with which a cell of the disclosure is administered. The use of such media and agents for pharmaceutically active substances is well known in the art. See, e.g., Remington, The Science and Practice of Pharmacy, 20th ed., (Lippincott, Williams & Wilkins 2003).

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions.

### Methods of Manufacture

In an aspect, the disclosure provides methods of making NK cells comprising providing a stem or progenitor cell, such as an induced pluripotent stem cell (iPSC) or embryonic stem cell (ESC) and differentiating the stem or progenitor cell into a NK cell. The stem or progenitor cell may be genetically modified to comprise a polynucleotide encoding a CAR prior to or after the differentiation stem. Known vectors may be used to deliver a polynucleotide, which may be stably maintained by the cell and/or integrated into the genome of the cell. The stem or progenitor cells may be CISH−/− iPSCs and used to generate CISH−/− iPSC-NK cells. The stem or progenitor cells may be soluble IL-15 or membrane-bound IL-15 knock-in iPSCs and used to generate IL-15 knock-in iPSC-NK cells. In some embodiments, iPSCs may be edited to not express CISH, express soluble IL-15 or membrane-bound IL-15, and express any one or more of the CAR constructs disclosed herein.

### Cancer Treatment

In some embodiments, the disclosure provides a method of treating cancer with the chimeric receptor expressing cells provided herein or pharmaceutical compositions or formulations thereof. In some embodiments, the method comprises administering the chimeric receptor expressing cells provided herein, pharmaceutical compositions or formulations described herein to a subject in need thereof. The terms “administration” and “administering,” as used herein, refer to the delivery of a bioactive composition or formulation by an administration route comprising, but not limited to intravenous, intramuscular, intraperitoneal, subcutaneous, and intramuscular, or combinations thereof. The term includes, but is not limited to, administering by a medical professional and self-administering.

In some embodiments, a cell expressing a chimeric receptor described herein exhibits enhanced anti-tumor efficacy relative to a cell lacking the chimeric receptor. Methods for measuring anti-tumor efficacy in vitro and in vivo are known to those of skill in the art and include, for example, in vitro killing assays and implanting tumors into mouse models.

Cell killing may be assessed by assays known in the art, including but not limited to those described in the examples.

In some embodiments, a cell expressing a chimeric receptor described herein exhibits enhanced cell killing by about 1%, about 5%, about 10%, about 20%, about 30%, about 40%, about 50%, about 60%, about 70%, about 80%, about 90%, about 100%, or about 1000% compared to a cell that that lacks the chimeric receptor and/or fusion polypeptide.

In some embodiments, a cell expressing a chimeric receptor described herein exhibits enhanced cell killing by between 1% to 5%, between 5% to 10%, between 10% to 20%, between 20% to 30%, between 30% to 40%, between 40% to 50%, between 50% to 60%, between 60% to 70%, between 70% to 80%, between 80% to 90%, between 90% to 100%, or between 100% to 1000% compared to a cell that that lacks the chimeric receptor and/or fusion polypeptide.

In some embodiments, a cell expressing a chimeric receptor described herein exhibits enhanced specific killing by about 1%, about 5%, about 10%, about 20%, about 30%, about 40%, about 50%, about 60%, about 70%, about 80%, about 90%, about 100%, or about 1000% compared to a cell that that lacks the chimeric receptor and/or fusion polypeptide.

In some embodiments, a cell expressing a chimeric receptor described herein exhibits enhanced specific killing by between 1% to 5%, between 5% to 10%, between 10% to 20%, between 20% to 30%, between 30% to 40%, between 40% to 50%, between 50% to 60%, between 60% to 70%, between 70% to 80%, between 80% to 90%, between 90% to 100%, or between 100% to 1000% compared to a cell that that lacks the chimeric receptor and/or fusion polypeptide.

### Definitions

All publications, patents and patent applications, including any drawings and appendices therein, are incorporated by reference in their entirety for all purposes to the same extent as if each individual publication, patent or patent application, drawing, or appendix was specifically and individually indicated to be incorporated by reference in its entirety for all purposes.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

All embodiments of any aspect of the invention can be used in combination, unless the context clearly dictates otherwise.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.”

As used herein, the singular forms “a,” “an,” and “the” include plural referents unless the context clearly dictates otherwise. “And” as used herein is interchangeably used with “or” unless expressly stated otherwise. Words using the singular or plural number also include the plural and singular number, respectively.

Section headings are for convenience only and combination of elements from different sections is contemplated. Additionally, the words “herein,” “above,” “below,” and words of similar import, when used in this application, refer to this application as a whole and not to any particular portions of the application.

The term “functional variant” refers to a homology (by sequence or structure) of a domain that retains sufficient signaling activity to activate an NK cell.

The term “sequence identity” refers to the percentage identity of a polypeptide or polynucleotide sequence of interest to a reference sequence, calculated as 100 times the number of exact matches in an optimum alignment of the sequence of interest to the reference sequence divided by the total length of the reference sequence (including gaps). An optimum alignment of the sequences may be generated using the European Molecular Biology Open Software Suite (EMBOSS) needle program available at www.ebi.ac.uk, as described in Maderia et al. Nucleic Acids Res. 47 (W1): W636-W641 (2019).

As used herein, the term “inactivating mutation” refers to a mutation in a genomic sequence that disrupts a function of a gene. The inactivating mutation can be in any sequence region (e.g., coding or non-coding) that contributes to gene expression. Examples include, but are not limited to, cis-acting elements (enhancers) or sequences that are subject to transcription (e.g., mRNA transcript sequences). An inactivating mutation includes mutations that render a gene or its encoded protein non-functional or that reduce the function of the gene or its encoded protein.

### EXAMPLES

The disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the methods of the present disclosure and practice the claimed methods. The following working examples therefore specifically point out embodiments of the present disclosure and are not to be construed as limiting in any way the remainder of the disclosure.

The materials and methods employed in these experiments are now described.

### Example 1: Screening of Chimeric Antigen Receptors Designs

This example describes design and testing of chimerical antigen receptors in induced pluripotent stem cell (iPSC)-derived natural killer (NK cells). We constructed a library of 44 novel CAR constructs containing signaling modules from diverse NK cell specific signaling receptors. Next, we developed a viral-based CAR expression protocol in mature iPSC-derived NK cells that yields high CAR expression (>75% CAR+) while maintaining high viability (>90%). We then screened for CAR activity using two co-culture target cell killing assays (eSight impedance assay and caspase 3/7 killing assay) and two NK cell-resistant CD19+ target cell lines (Raji and SupB15). The tested constructs, all with an anti-CD19 scFv as the ligand-binding domain, are provided as SEQ ID NOs: 100-117. Our screen identified 7 novel CAR signaling modules that performed better than both a second-generation T-cell CAR (CD28-zeta) and three previously described NK cell-CARs (2B4-zeta, OX40-zeta, and OX40L-zeta). These results were consistent across both functional assays and both target cell lines tested adding support to our findings.

Designed CAR sequences were C-terminal linked via a self-cleaving (2A) peptide to a membrane bound IL-15 (mbIL-15) for co-expression in natural killer cells. Lentiviral particles were generated and concentrated ˜100× to facilitate the high viral titers required to transduce iPSC-derived NK cells.

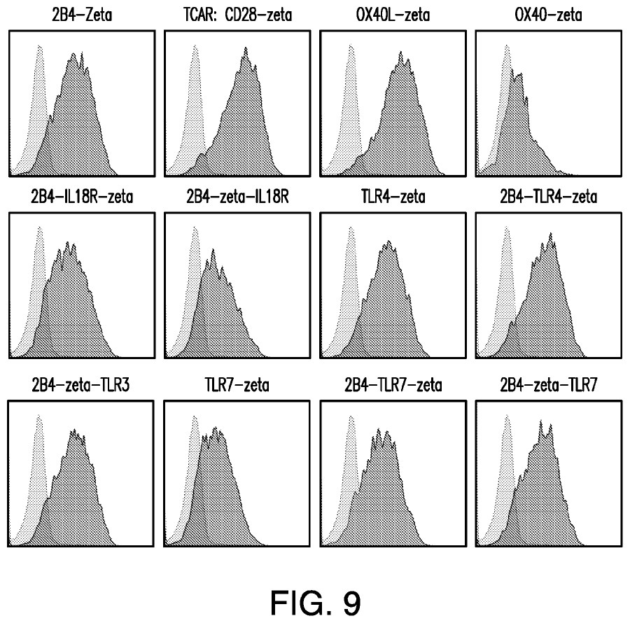

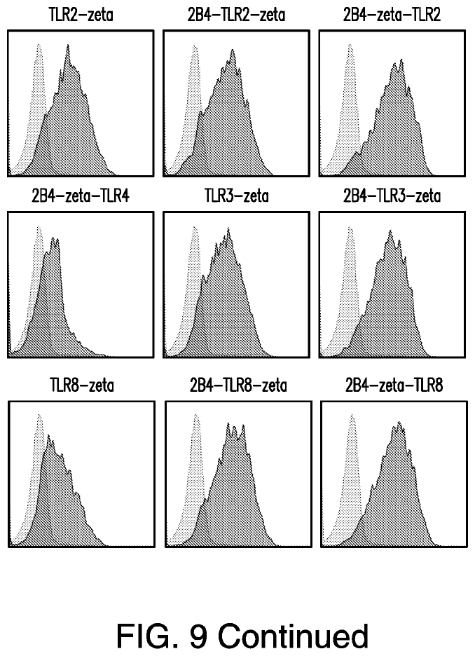

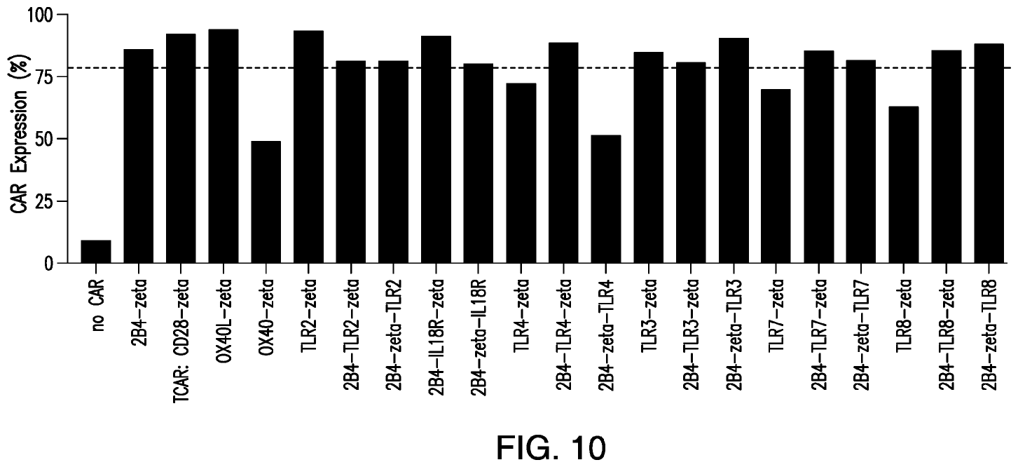

Concentrated viral stocks were loaded onto retronectin-coated 96-well plates by centrifugation, IL-2 activated iPSC-derived NK cells were added and centrifuged in the presence of viral supernatant, and incubated in the presence of IL-2 for 72 hrs. CAR expression was detected by flow cytometry using an HA-tag engineered into the extracellular portion of the CAR polypeptide (FIG. 9). CAR expression was uniform across most of the CAR constructs with average CAR expression of ˜78% (FIG. 10). We did not observe any loss of cell viability when comparing CAR transduced cells to untransduced controls.

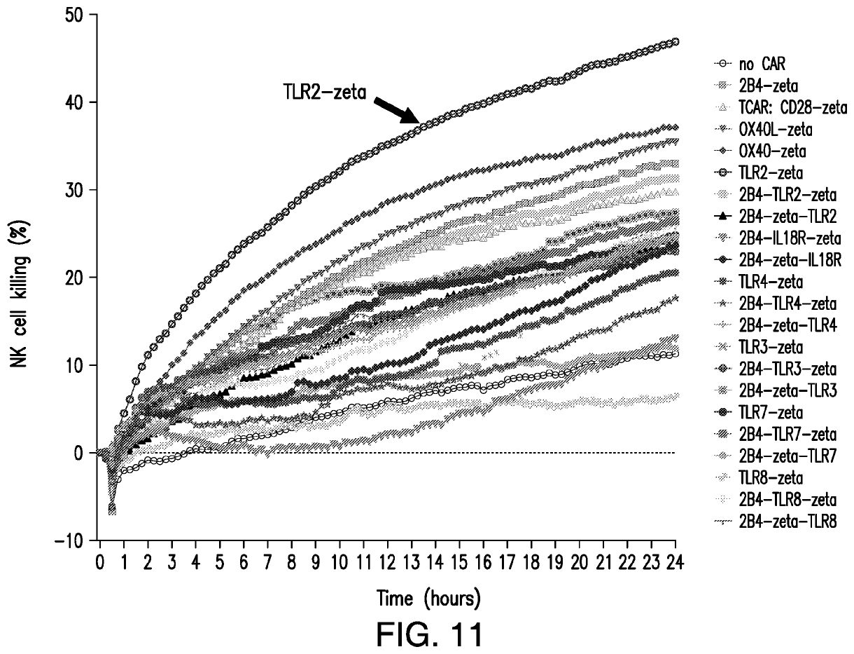

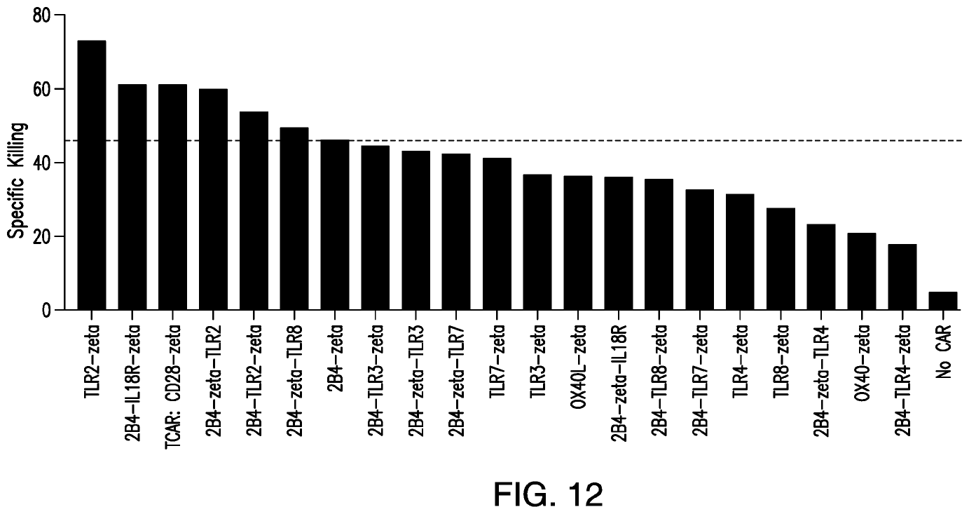

We screened for CAR activity using two co-culture target cell killing assays (eSight impedance assay and Caspase 3/7 killing assay) and two NK cell resistant CD19⁺ target cell lines (Raji and SupB15). Results of the screen are shown in FIG. 11 and FIG. 12. We identified six CAR constructs that performed better than the best comparator CAR (2B4-zeta). Twelve outperformed another comparator CAR (OX40L-zeta). All CARs tested except one outperformed a third comparator CAR (OX40-zeta).

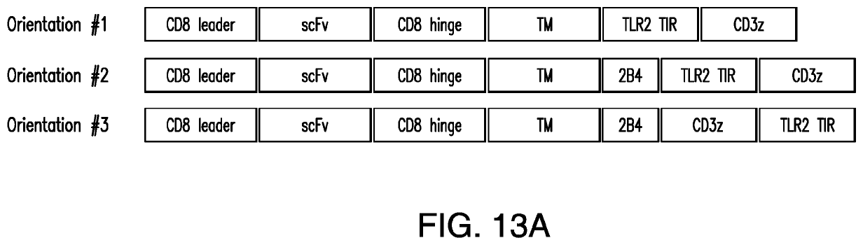

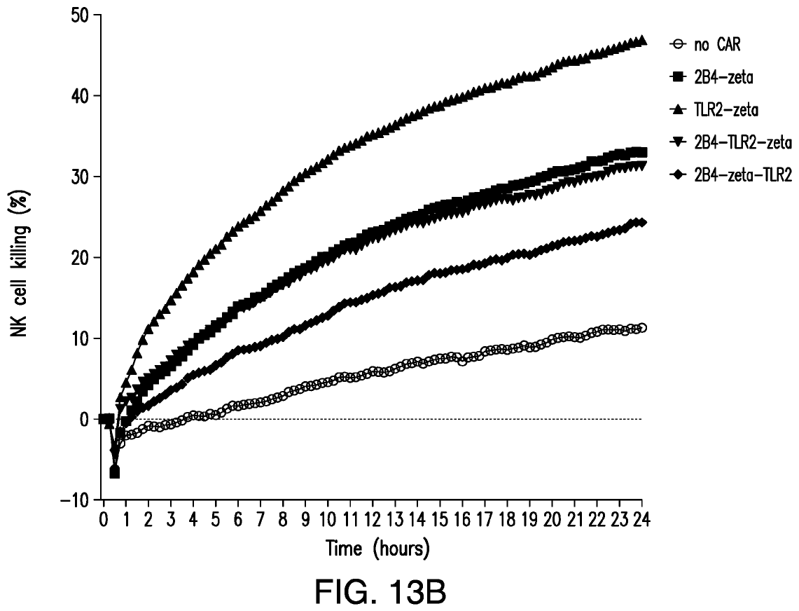

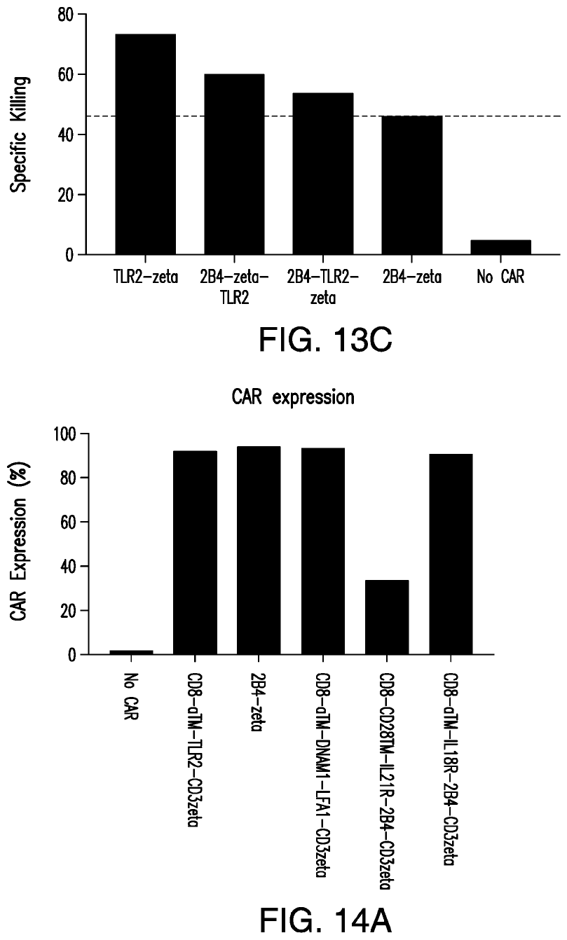

Selected data comparing CAR designs with and without a 2B4 domain is shown in FIGS. 13A-13C. 2B4-based CARs have been shown to be effective in activating NK cells. Surprisingly, our CAR TLR2-zeta are superior to both the comparator 2B4-zeta and our CAR design combining both 2B4 and TLR2 domains (2B4-zeta) in both assays (FIG. 13B and FIG. 13C).

Overall, we have successfully developed a CAR screening platform in a therapeutically relevant allogenic cell source and have identified NK optimized CARs that enable increased potency of NK cells compared to current CAR solutions. Of note, intracellular signaling modules from Toll-like receptors and IL-1R subfamily receptors showed enhanced NK CAR activity compared to CARs containing canonical NK cell activation receptor domains used in previously developed NK optimized CAR designs—i.e., 2B4 (shown in figures), FCER1G, DNAM1, DAP10. These findings validate the use of diverse immune cell signaling domains in NK optimized CARs.

### Methods

Construction of lentiviral expression vectors and viral packaging: Polypeptide sequences of all candidate CAR components were obtained from Ensemble™ database (Howe et al. Nucleic Acids Res. 2021, vol. 49 (1): 884-891) and assembled into complete CAR designs using SnapGene™ molecular biology software. Nucleic acid sequences were determined using a publicly available codon optimization tool (Integrated DNA Technologies™). All CAR constructs included, as representative ligand binding domain, a CD19 scFv binder (FMC63; SEQ ID NO: 109), mbIL-15 co-expressed partner (SEQ ID NO: 48), and an HA-tag for CAR detection. CAR constructs were synthesized and cloned into a lentiviral expression vector under the EF-1 alpha promoter (pCDH-EF1α-MCS-T2A-Puro; System Biosciences™), and maxi-preps were produced and sequence verified. Lentiviral particles were generated using the constructs described above, packaging plasmids (Rev, gag-pol, and VSVG; Cell Biolabs™), and the Lenti-X™ 293T cell line (Takara™). Viral supernatants were concentrated ˜100× using a PEG-based concentration solution (Takara™) and viral titers were determined using Lenti-X GoStix Plus™ (Takara™).

Generation of iPSC-NK cells: iPSCs are collected and transferred to culture in serum-free media and formed into embryoid bodies by spin aggregation. Upon the appearance of CD34⁺ cells inside the Embryoid Body (EB) at day 6, EB are transferred into NK cell differentiation medium containing: a 2:1 mixture of Dulbecco modified Eagle medium/Ham F12), 2 mM L-, 1% penicillin/streptomycin, 25 mM b-mercaptoethanol, 20% heat-inactivated human serum AB, 5 ng/ml sodium selenite, 50 mM ethanolamine, 20 mg/mL ascorbic acid, interleukin-3; for first week only, stem cell factor, interleukin-15, Fms-like tyrosine kinase 3 ligand, and interleukin-7. The EB is left in these conditions for 28 days receiving weekly media changes to produce NK cells (CD45⁺CD56⁺CD33⁻CD3⁻ cells, as determined by flow cytometry). Then NK cells are harvested for expansion.

Expansion: After differentiation, an immortalized leukemia cell line, K562 expressing mbIL21 and 4-1BB ligand (CD137), is used to stimulate NK cell expansion. NK cells are seeded with K562-41BBL-mbIL21 feeder cells and incubated at 1:2 ratio in Stromal Cell Growth Medium (SCGM) with 100 U/mL IL-2. Non-adherent cells are removed and analyzed by flow cytometry to determine the purity of CD56+NK cells. These cells are then stimulated with 2:1 aAPCs (irradiated at 10,000 Gy) to NK cells at 350,000 NK cells/mL of media containing RPMI 1640, 2 mM L-glutamine, 1% penicillin/streptomyocin, 1% non-essential amino acids, and 10% standard FBS or 10% human serum AB), supplemented with 50-100 U/mL IL-2.

Transduction of iPSC-NK cells: Non-treated 96-well plates were coated with Retronectin (Takara). Equal amount of concentrated lentivirus was added to plates and centrifuged at 2000×g at 32° C. for 2 hrs. iPSC-derived NK cells (6e4 cells) were added to each well in media containing IL-2 (50 U/ml) and centrifuged at 800×g at 32° C. for 1 hr. Cells were incubated at 37° C. for 48 hrs. Fresh media (with IL-2) was added, and cells were incubated for an additional 24 hrs. CAR expression was determined at 72 hrs post-transduction using flow cytometry to detect the HA-tag engineered into the extracellular domain of each CAR. CAR+ cells were used for functional assay within 24 hrs of CAR detection.

Long-term target cell killing assay using eSight™: RTCA E-plates were coated with CD40 tethering reagent (Agilent™) diluted in PBS overnight at 4° C. Plates were washed with PBS, target cells (either CD19⁺ target cells (Raji; ATCC™) or HER2⁺ target cells (BT474 clone5) were added to each well, and impedance readings were allowed to equilibrate for 2-4 hrs. CAR+ iPSC-NK cells were added to each well at the indicated E:T ratio and impedance readings were measured every 15 min for 24 hrs. NK-specific killing was determined by normalizing impedance readings to “target cells alone” control wells and plotted as frequency of target killing.

4 hr Caspase 3/7 cytotoxicity assay: SupB15 (CD19⁺ target cells) were pre-stained with CellTrace™ Violet at a final concentration of 5 μM in PBS for 15 min at 37° C., followed by washing in complete culture medium. CAR+iPSC-NK cells were added to each well at 2:1 E:T ratio in a final volume of 100 μl. Co-cultures were centrifuged at 100×g for 1 min and incubated at 37° C. for 3.5 hrs. Following incubation, CellEvent® Caspase-3/7 Green Detection Reagent and SYTOX™ AADvanced™ dead cell stain solution was added to co-cultures and incubated for an additional 30 min at 37° C. After staining, assays are read on a NovoCyte Advanteon and analyzed by FlowJo software. NK-specific killing was determined by normalizing impedance readings to “target cells alone” control wells and plotted as frequency of target killing.

### [Prophetic] Example 2: In Vivo Functional Testing of CARs Having a CD19 Binding Domain

iPSC derived NK cells expressing functional CAR constructs (determined by results from in vitro assays) are tested for in vivo efficacy using a CD19 expressing model of B cell malignancies. To initiate disease, 10-12 week-old NOD-SCID-gamma^{-/-} (NSG) mice (Jackson Laboratory) are injected intravenously with luciferase expressing Raji cells (CD19 positive). After 1 day, the mice are administered iPSC-derived NK cells engineered to express (a) no CAR (negative control); (b) a 2nd generation T cell CAR (benchmark control); or (c) an NK CAR (test group). Subject mice are administered IL-2 every other day for the remainder of the experiment. Disease progression is monitored by survival, weight loss, and bioluminescent imaging of tumor burden (measured weekly). To assess the number of infused NK cells present during the course of the experiment, CD45⁺CD56⁺CD3⁻ cells from peripheral blood are quantified weekly.

### Example 3: Functional Testing of CARs Having a HER2 Binding Domain

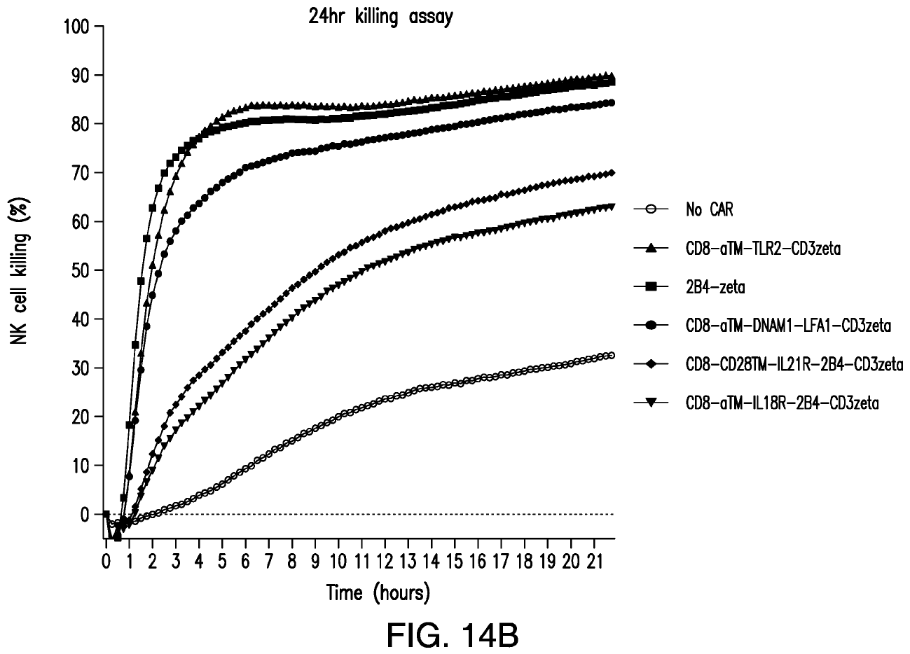

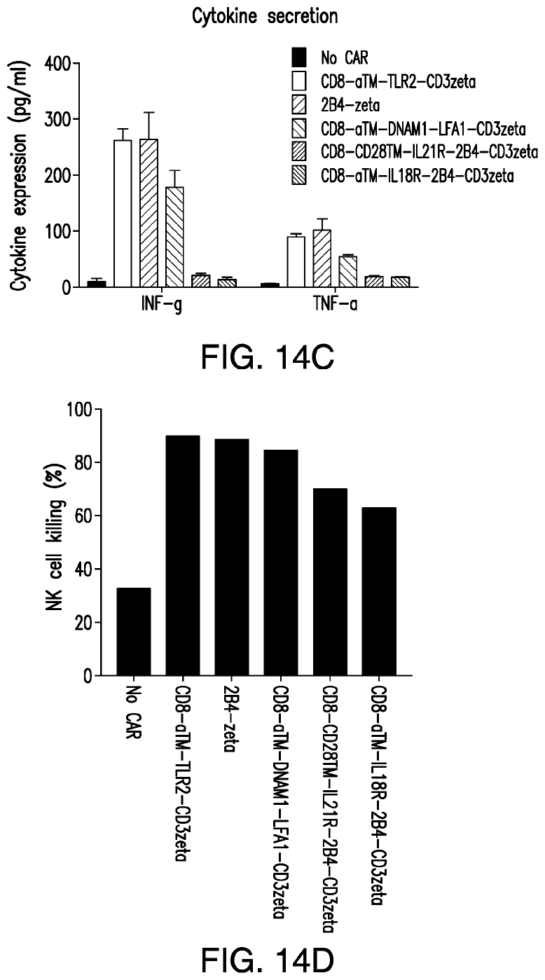

Concentrated lentiviral stocks comprising different CARs having a HER2 scFv binder were loaded onto retronectin-coated 96-well plates by centrifugation. IL-2 activated iPSC-derived NK cells were added and centrifuged in the presence of viral supernatant, and incubated in the presence of IL-2 for 72 hrs. CAR expression was detected by flow cytometry using an HA-tag engineered into the extracellular portion of the CAR polypeptide. CAR expression was uniform across most of the CAR constructs (FIG. 14A).

CAR activity was screened using a co-culture target cell killing assay (eSight impedance assay) on a NK cell resistant HER2⁺ target cell line (BT474). Results of the screen are shown in FIG. 14B and FIG. 14D. In addition, a cytokine secretion assay was performed using an ELISA on co-culture supernatants. Results of the secretion assay are shown in FIG. 14C. Results identified CD8-aTM-TLR2-CD3ζ (SEQ ID NO: 82) as the top NK optimized CAR tested and consistently performed equal/better than others across multiple CAR targets and assays. Additionally, similar levels of sIL15 (150-200 μg/ml) were observed between (1) CD8-aTM-TLR2-CD3ζ, (2) CD8-aTM-DNAM1-LFA1-CD3ζ, and (3) 2B4-zeta (lower for aTM-IL18R-2B4-CD3ζ˜100 μg/ml) (data not shown).

Similarly, the CD8-aTM-TLR2-CD3ζ construct having a CD19 scFv exhibited robust cell killing of Raji 2.0 cells at multiple effector to target cell rations (data not shown). The CD8-CD28TM-TLR2-CD3ζ construct also exhibited similar activity.

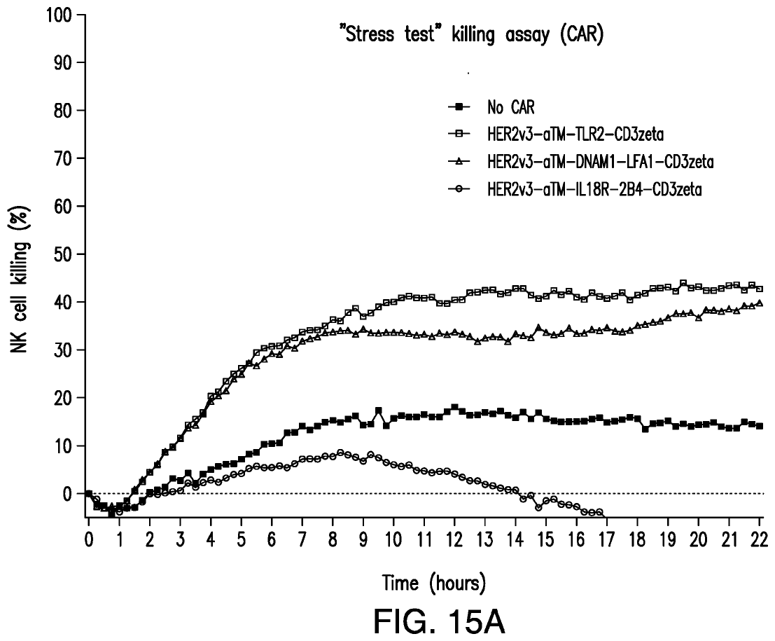

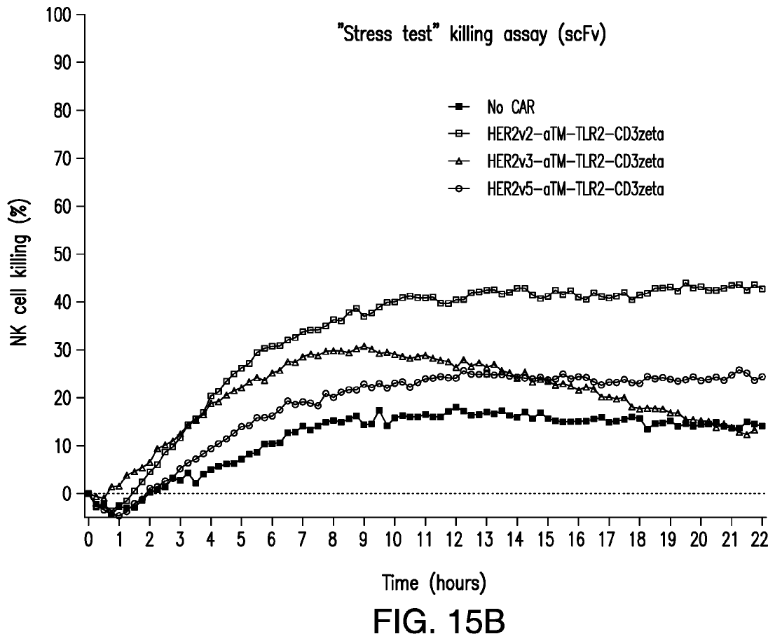

Next, CAR-HER2 activity was screened using stress test killing assays. Briefly, CISH KO iPSC-derived NK cells were transduced using retrovirus with one of three different CAR constructs having the same HER2 scFv binding domain (HER2v3), and different intracellular domains. (FIG. 15A). In a second set of experiments, CISH KO iPSC-derived NK cells were transduced using retrovirus with one of three different CAR constructs having the same intracellular domain, and different HER2 scFv binding domain (HER2v3). (FIG. 15B). The effector CISH KO iPSC-derived NK cells were co-cultured with a NK cell resistant HER2⁺ target cell line (BT474) at a 5:1 effector cells to target cell ratio without the addition of cytokines or co-expression of sIL-15, and target cell killing assays (eSight impedance assay) were employed to identify which CAR construct exhibited high killing activity.

The results using different CAR constructs having the same HER2 scFv binding domain (HER2v3) identified CD8-aTM-TLR2-CD3ζ (SEQ ID NO: 82) as the top NK optimized CAR-HER2 construct tested (FIG. 15A). The results using the same intracellular domain (CD8-aTM-TLR2-CD3ζ) having different HER2 scFv binding domains identified the HER2 scFv variant 3 (v3) as the top NK optimized CAR-HER2 construct tested. (FIG. 15B).

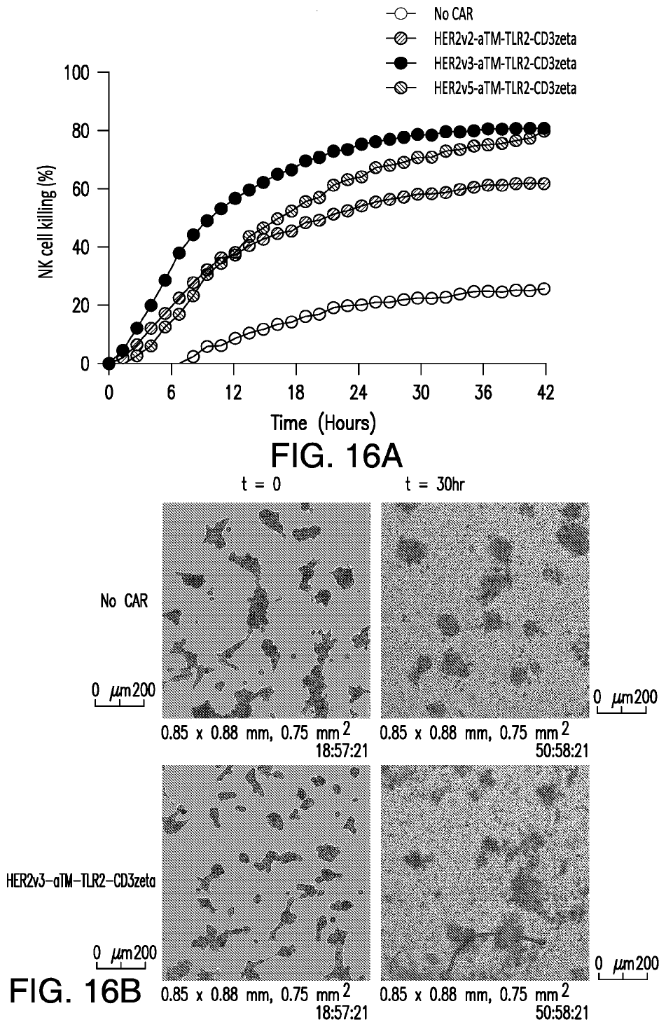

CAR activity for the CD8-aTM-TLR2-CD3ζ CAR constructs having different HER2 scFv binding domains was also assessed in a caspase 3/7 killing assay using a NK cell resistant HER2⁺ target cell line (BT474) (FIGS. 16A-16B). Briefly, Caspase 3/7 activation was measured following co-culture of CISH KO iPSC-derived NK cells expressing one of three different CAR constructs having the same intracellular domain, and different HER2 scFv binding domains (HER2v2, HER2v3, or HER2v5) with a resistant solid tumor target (2:1 E:T) (FIG. 16A). Representative images of target cell killing and Caspase 3/7 activation after 30 hr co-culture of the CISH KO iPSC-derived NK cells +/− CAR with the NK cell resistant solid tumor targets indicated robust NK cell killing upon co-culture of CISH KO iPSC-derived NK cells expressing the CAR, but not without the CAR. (FIG. 16B). Target cells were labeled with Cell Tracker Red and Caspase 3/7 activation was visualized in green.

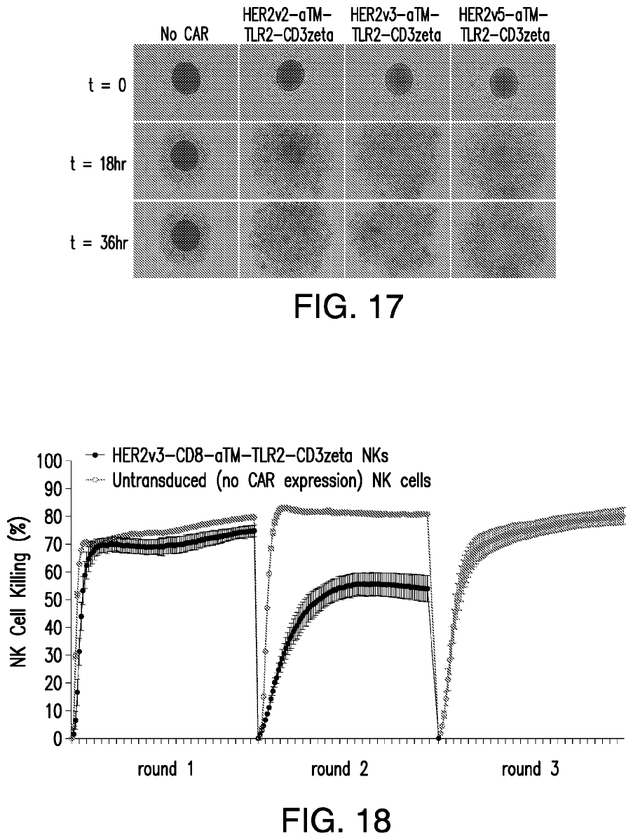

Next, the killing activity of CISH KO iPSC-derived NK cells expressing the CD8-aTM-TLR2-CD3ζ CAR and one of three different HER2 scFv binding domains (HER2v2, HER2v3, or HER2v5) were tested in 3D single tumor spheroids. In brief, BT474 cells, which are human HER+ductal carcinoma cells, were seeded and cultured using standard protocols for spheroid formation. After spheroids were formed (approximately 36 hours after seeding), cells were co-cultured with CISH KO iPSC-derived NK cells: 1) not expressing a CAR; 2) expressing a HER2v2-CD8-aTM-TLR2-CD3ζ CAR; 3) expressing a HER2v3-CD8-aTM-TLR2-CD3ζ CAR; or 4) expressing a HER2v5-CD8-aTM-TLR2-CD3ζ CAR. FIG. 17 shows representative images of NK cell resistant solid tumor target spheroids 0, 18, and 36 hours after treatment. Taken together, the data showed that CAR-HER2 NKs, particularly the HER2v3-CD8-aTM-TLR2-CD3ζ expressing NKs, potently kill HER2+ target cancer cells.

Using iPSC-derived NK cells transduced with the HER2v3-CD8-aTM-TLR2-CD3ζ construct, a cell serial killing assay (eSight impedance assay) was performed in real time. In brief, repeat killing of a HER2+ target cell line (SKOV3) and exhaustion profiles were observed by repeatedly adding HER2v3-CD8-aTM-TLR2-CD3ζ CISH KO iPSC-derived NK cells to the target cells. As shown in FIG. 18, HER2v3-CD8-aTM-TLR2-CD3ζ NK cells maintained a high percentage of NK cell killing capacity after 3 rounds whereas NK cells not having a CAR (untransduced, or “UT”), no longer killed the cells after round 2.

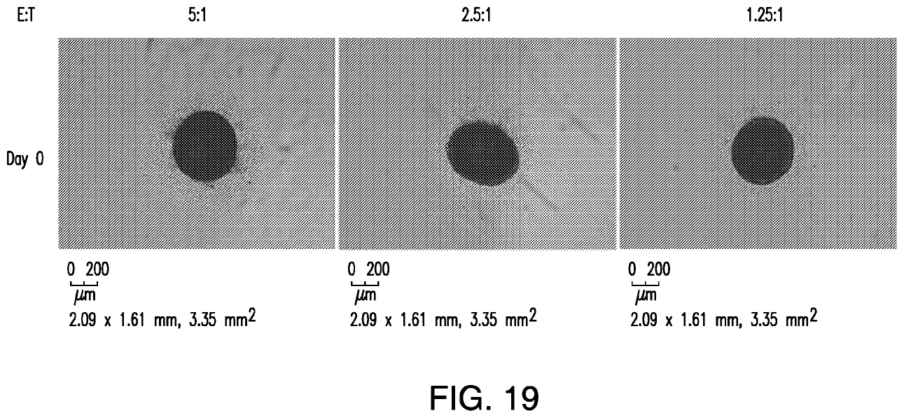

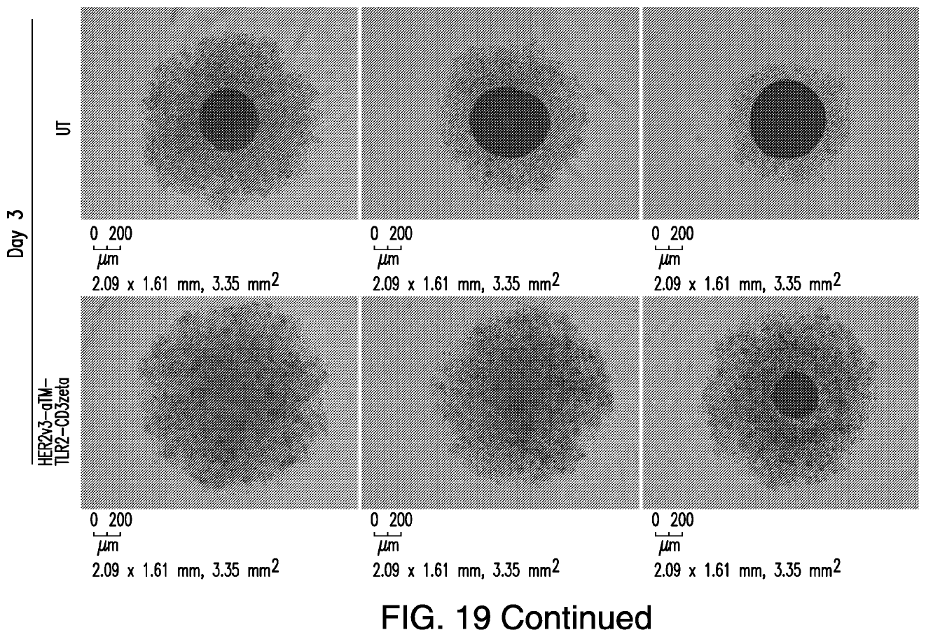

Killing activity of the HER2v3-CD8-aTM-TLR2-CD3ζ-expressing CISH KO iPSC-derived NK cells was tested in 3D single HER+ tumor spheroids. After spheroids were formed, target cells were co-cultured with different ratios of either 1) CISH KO iPSC-derived NK cells that were not transduced with a CAR construct (untransduced NKs or “UT”) or 2) CISH KO iPSC-derived NK cells that express HER2v3-CD8-aTM-TLR2-CD3ζ. The ratio of effector cells (untransduced CISH KO iPSC-derived NK cells or HER2v3-CD8-aTM-TLR2-CD3ζ CISH KO iPSC-derived NK cells) added to target cells (BT474 cells) was either 5:1, 2.5:1, or 1.25:1. Killing of spheroid cells was monitored at day 0 and day 3 after NK cells were added. FIG. 19 shows that the target BT474 cells of the HER+ spheroid treated with NKs expressing HER2v3-CD8-aTM-TLR2-CD3ζ shrank and underwent apoptosis in a concentration dependent manner at day 3.

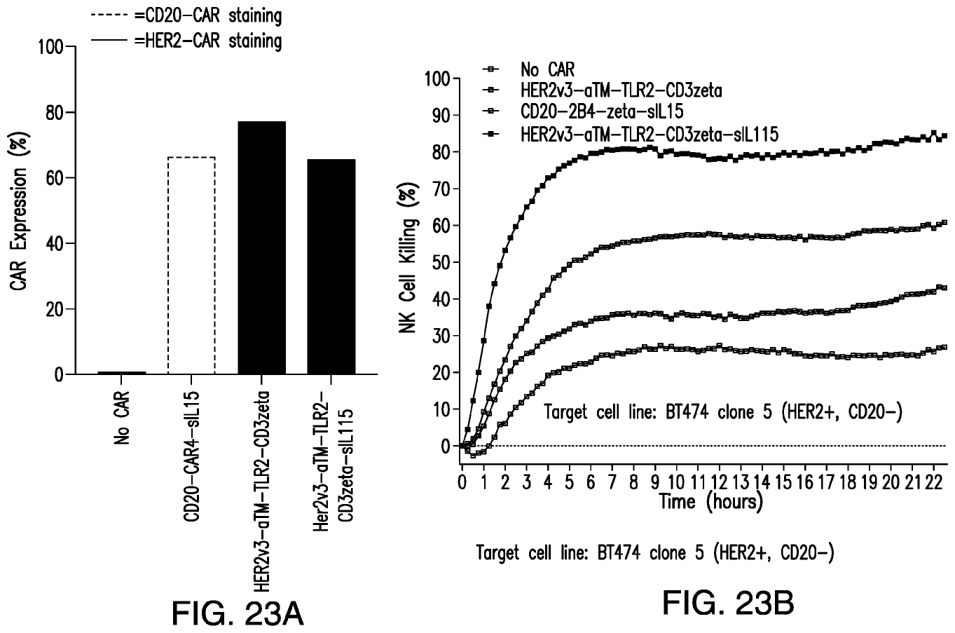

To examine the effect of sIL-15 on CAR-HER2 activity, a CAR expression assay FIG. 23A and long-term killing assay against cell line BT474 clone 5 were performed (FIG. 23B). While expression of sIL-15 did not affect CAR expression, sIL-15 expression (HER2-3-CD8-aTM-TLR2-CD3ζ-sIL15 (SEQ ID NO: 205)) dramatically increased CAR NK cell killing of BT474 cells compared to controls (CD20-2b4-zeta-sIL15 and HER2-3-CD8-aTM-TLR2-CD3ζ without sIL 15 (SEQ ID NO: 204)). After about 10 hours, about 80% NK cell killing was observed for HER2-3-CD8-aTM-TLR2-CD3ζ-sIL15 (SEQ ID NO: 205) whereas only about 55% and 30% was observed for CD20-2b4-zeta-sIL15 and HER2-3-CD8-aTM-TLR2-CD3& without sIL 15 (SEQ ID NO: 204), respectively. These results demonstrate that sIL-15 has a synergistic effect with HER2-CAR constructs in NK cell killing of HER+ breast cancer cell lines.

Taken together, these results demonstrate that CISH KO iPSC-derived NK cells expressing HER2-CD8-aTM-TLR2-CD3ζ CARs demonstrate robust activity against NK cell resistant solid tumor target cells.

### Example 4. Functional Testing of HER2-CAR CISH KO iPSC-Derived NK Cells

This example describes testing of CISH KO iPSC-derived NK cells expressing a HER2 scFv-CAR and soluble IL-15 demonstrated potent killing of solid tumor target cell lines.

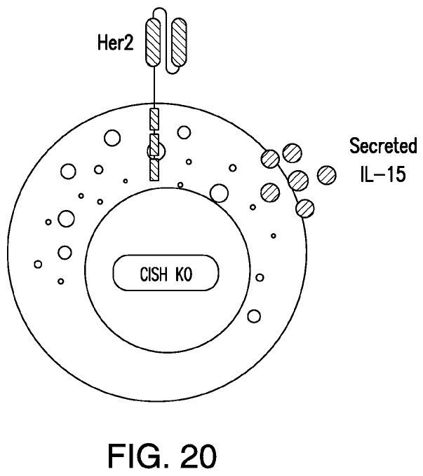

First, triple-edited (TE) iPSCs were generated using a gene editing method by introducing into CISH KO iPSCs a HER2-CAR sequence that was C-terminal linked via a self-cleaving (2A) peptide to a soluble IL-15 (sIL-15). The resulting TE iPSCs: 1) did not express the CISH gene (CISH KO); 2) expressed HER2v3-CD8-aTM-TLR2-CD3ζ (HER-CAR) and 3) expressed soluble IL-15 (SEQ ID NO: 58), allowing for IL-15 secretion (FIG. 20). TE iPSCs were next subjected to differentiation to form functional NK cells that lacked CISH, expressed HER-CAR, and exhibited IL-15 secretion (TE iPSC-derived NK cells).

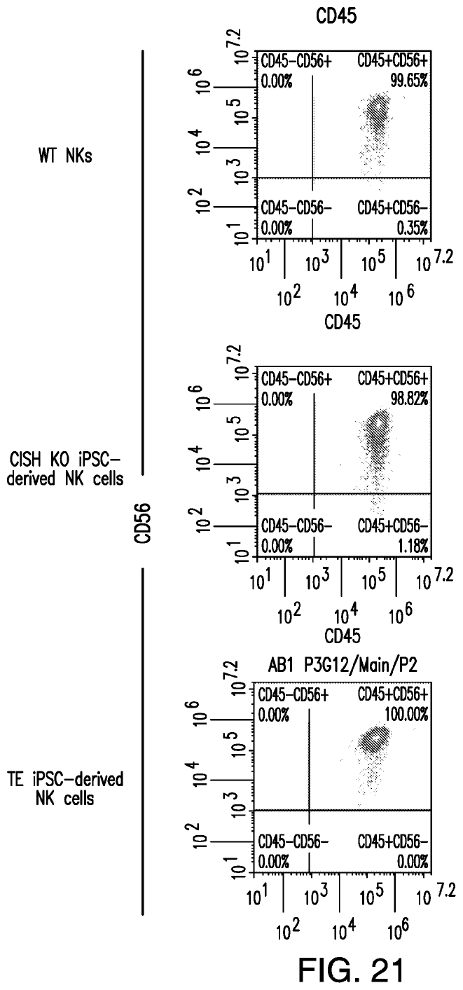

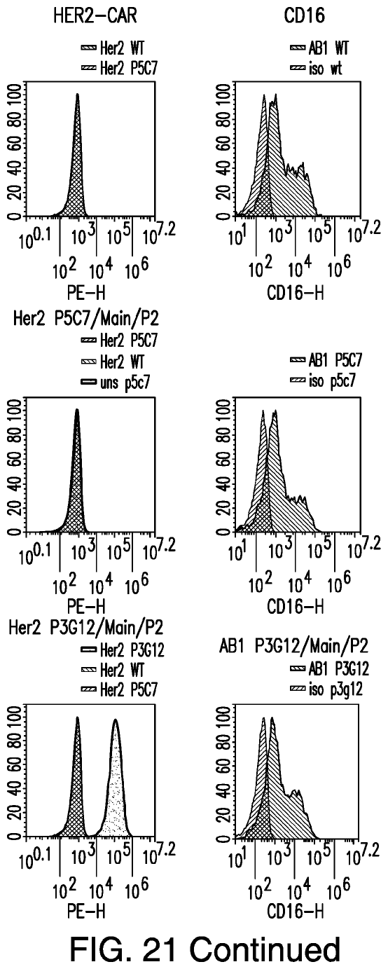

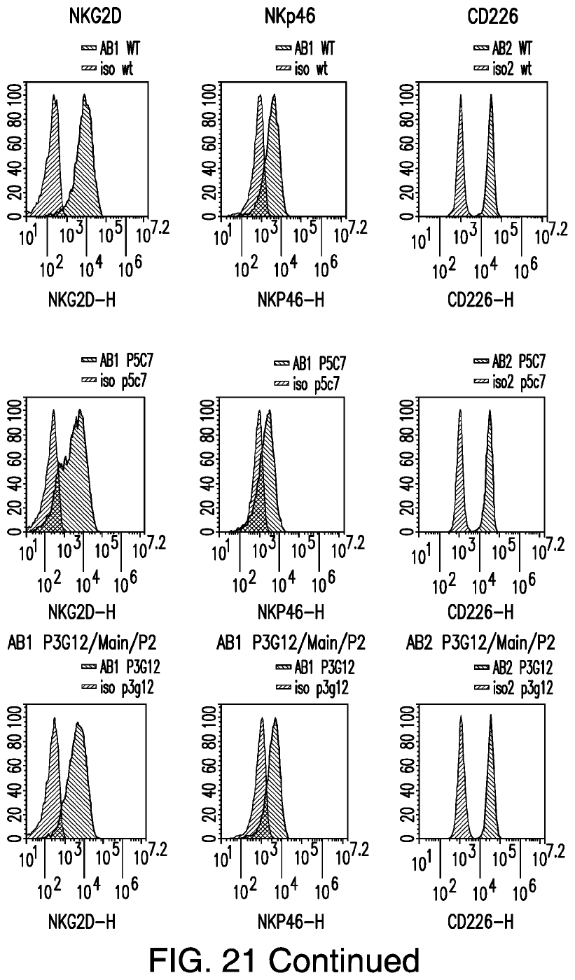

To determine if triple editing of the iPSCs resulted in an altered NK phenotype, NK cell marker expression was measured in the TE iPSC-derived NK cells by flow cytometry. As shown in FIG. 21, expression of twelve NK phenotypic markers was not significantly different between wild type (WT) NK cells, NKs differentiated from iPSCs having only the CISH KO, and NKs differentiated from the TE iPSCs assessed.

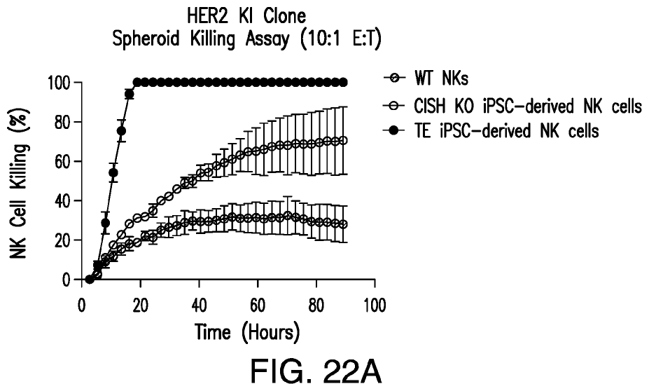

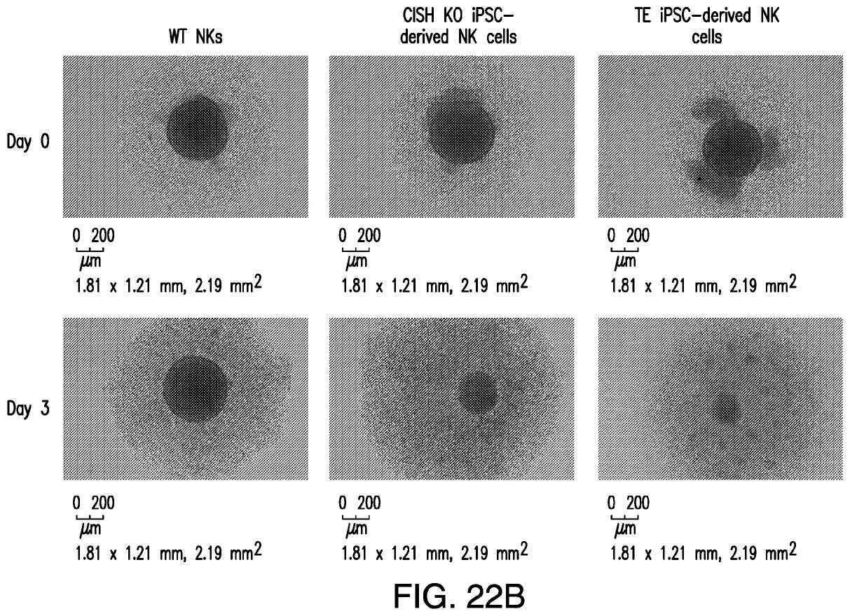

TE iPSC-derived NK cell function was assessed in a spheroid killing assay. In brief, HER2+ (BT474 clone 5) target cell spheroids were prepared as described in the examples herein. Either wild type (WT) NK cells, NKs differentiated from iPSCs having only the CISH KO, or NKs differentiated from the TE iPSCs were added to the HER2+ spheroids (10:1 E:T). NK-mediated killing of the HER2+ spheroids, monitored using an eSight impedance assay, showed that the TE iPSC-derived NK cells were effective at killing the NK resistant HER2+ solid tumor target (FIG. 22A). FIG. 22 shows that the TE iPSC-derived NK cell-treated spheroids significantly decreased in size after three days compared to spheroids treated with WT NKs or CISH KO iPSC-derived NK cells.

Taken together, the data demonstrates that TE iPSC-derived NK cells potently kill solid tumor target cell lines.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A natural killer cell, comprising a chimeric antigen receptor (CAR) comprising an extracellular portion, a transmembrane domain, and a cytoplasmic portion,
wherein the cytoplasmic portion comprises a Toll/interleukin-1 (IL-1) receptor (TIR) signaling domain.

2. The cell of claim 1, wherein the TIR signaling domain is a Toll-like receptor (TLR) signaling domain.

3. The cell of claim 2, wherein the TLR signaling domain is a TLR2 signaling domain or a functional variant thereof.

4. The cell of claim 2 or claim 3, wherein the TLR signaling domain has a polypeptide sequence at least 55%, at least 70%, at least 75%, at least 80%, at least 90%, at least 95%, or 100% identical to SEQ ID NO: 2.

5. The cell of claim 2, wherein the TLR signaling domain is a TLR8 signaling domain or a functional variant thereof.

6. The cell of claim 2 or claim 5, wherein the TLR signaling domain has a polypeptide sequence at least 55%, at least 70%, at least 75%, at least 80%, at least 90%, at least 95%, or 100% identical to SEQ ID NO: 8.

7. The cell of claim 2, wherein the TLR signaling domain is a TLR3 signaling domain or a functional variant thereof.

8. The cell of claim 2 or claim 7, wherein the TLR signaling domain has a polypeptide sequence at least 55%, at least 70%, at least 75%, at least 80%, at least 90%, at least 95%, or 100% identical to SEQ ID NO: 3.

9. The cell of claim 2, wherein the TLR signaling domain is a TLR7 signaling domain or a functional variant thereof.

10. The cell of claim 2 or claim 9, wherein the TLR signaling domain has a polypeptide sequence at least 55%, at least 70%, at least 75%, at least 80%, at least 90%, at least 95%, or 100% identical to SEQ ID NO: 7.

11. The cell of claim 2, wherein the TLR signaling domain is a TLR4 signaling domain or a functional variant thereof.

12. The cell of claim 2 or claim 11, wherein the TLR signaling domain has a polypeptide sequence at least 55%, at least 70%, at least 75%, at least 80%, at least 90%, at least 95%, or 100% identical to SEQ ID NO: 4.

13. The cell of claim 2, wherein the TLR signaling domain is selected from the group consisting of a TLR1, a TLR5, a TLR6, a TLR9, or a TLR10 signaling domain or a functional variant thereof.

14. The cell of claim 2 or claim 13, wherein the TLR signaling domain has a polypeptide sequence at least 55%, at least 70%, at least 75%, at least 80%, at least 90%, at least 95%, or 100% identical to any one of SEQ ID NOs: 1, 5, 6, 9, or 10.

15. The cell of claim 1, wherein the TIR signaling domain is a interleukin-1 receptor (IL-1R) subfamily signaling domain.

16. The cell of claim 15, wherein the IL-1R subfamily signaling domain is an interleukin-1 receptor (IL-1R) signaling domain or functional variant thereof.

17. The cell of claim 15 or claim 16, wherein the IL-1R subfamily signaling domain has a polypeptide sequence at least 55%, at least 70%, at least 75%, at least 80%, at least 90%, at least 95%, or 100% identical to SEQ ID NO: 21.

18. The cell of claim 15, wherein the IL-1R subfamily signaling domain is an interleukin-18 receptor (IL-18R) signaling domain or a functional variant thereof.

19. The cell of claim 15 or claim 18, wherein the IL-1R subfamily signaling domain has a polypeptide sequence at least 55%, at least 70%, at least 75%, at least 80%, at least 90%, at least 95%, or 100% identical to SEQ ID NO: 22.

20. The cell of claim 15, wherein the IL-1R subfamily signaling domain is selected from the group consisting of an interleukin-1 receptor 4 (IL-1R4), an interleukin-1 receptor 6 (IL-1R6), an interleukin-1 receptor 3 (IL-R3), an interleukin-1 receptor 7 (IL-1R7), an interleukin-1 receptor 8 (IL-R8), an interleukin-1 receptor 9 (IL-1R9) or an interleukin-1 receptor 10 (IL-1R10) signaling domain or functional variant thereof.

21. The cell of claim 15 or claim 20, wherein the IL-1R subfamily signaling domain has a polypeptide sequence at least 55%, at least 70%, at least 75%, at least 80%, at least 90%, at least 95%, or 100% identical to any one of SEQ ID NOs: 143, 144, 145, 146, 147, 148, or 149.

22. The cell of any one of claims 1 to 21, wherein the cytoplasmic portion comprises a CD3ζ signaling domain.

23. The cell of any one of claims 1 to 22, wherein the cytoplasmic portion comprises a 2B4 signaling domain.

24. The cell of any one of claims 1 to 22, wherein the cytoplasmic portion lacks a 2B4 signaling domain.

25. The cell of claim 22, wherein the cytoplasmic portion consists essentially of, in N- to C-terminal order, a TLR2 signaling domain and a CD3ζ signaling domain.

26. The cell of claim 22, wherein the cytoplasmic portion consists essentially of, in N- to C-terminal order, a 2B4 signaling domain, an IL-18R signaling domain, and a CD3ζ signaling domain.

27. The cell of claim 22, wherein the cytoplasmic portion consists essentially of, in N- to C-terminal order, a 2B4 signaling domain, a CD3ζ signaling domain, and a TLR2 signaling domain.

28. The cell of claim 22, wherein the cytoplasmic portion consists essentially of, in N- to C-terminal order, a 2B4 signaling domain, a TLR2 signaling domain, and a CD3ζ signaling domain.

29. The cell of any one of claims 1 to 28, wherein the extracellular protein comprises a ligand-binding domain.

30. The cell of claim 29, wherein the ligand-binding domain comprises an antibody-like domain.

31. The cell of claim 30, wherein the ligand-binding domain comprises a variable heavy (VH) domain and/or a variable light (VL) domain.

32. The cell of claim 30, wherein the ligand-binding domain comprises a single-chain variable fragment (scFv).

33. The cell of any one of claims 29 to 32, wherein the ligand-binding domain specifically binds a CD19 antigen.

34. The cell of claim 33, wherein the ligand-binding domain comprises the a CDR-H1 according to SEQ ID NO: 76; a CDR-H2 according to SEQ ID NO: 77; a CDR-H3 according to SEQ ID NO: 78; a CDR-L1 according to SEQ ID NO: 79; a CDR-L2 according to SEQ ID NO: 80; and/or a CDR-L3 according to SEQ ID NO: 81.

35. The cell of claim 33, wherein the ligand-binding domain comprises a VL according to SEQ ID NO: 74 and/or a VL according to SEQ ID NO: 75, or functional variants having polypeptide sequences at least 55%, at least 70%, at least 80%, at least 90%, at least 95%, or 100% identity thereto.

36. The cell of claim 33, wherein the ligand-binding domain comprises an scFv according to SEQ ID NO: 73, or a functional variant having a polypeptide sequence at least 55%, at least 70%, at least 80%, at least 90%, at least 95%, or 100% identity thereto.

37. The cell of any one of claims 29 to 32, wherein the ligand-binding domain specifically binds a CD20 antigen.

38. The cell of claim 37, wherein the ligand-binding domain comprises an scFv according to SEQ ID NO: 139, or a functional variant having a polypeptide sequence at least 55%, at least 70%, at least 80%, at least 90%, at least 95%, or 100% identity thereto.

39. The cell of any one of claims 29 to 32, wherein the ligand-binding domain specifically binds a HER2 antigen.

40. The cell of claim 39, wherein the ligand-binding domain comprises an scFv according to SEQ ID NOs: 140-142, or a functional variant having a polypeptide sequence at least 55%, at least 70%, at least 80%, at least 90%, at least 95%, or 100% identity thereto.

41. The cell of any one of claims 1 to 40, wherein the transmembrane domain comprises a polypeptide having the polypeptide sequence NLFVASWIAVMIIFRIGMAVAIFCCFFFP (SEQ ID NO: 41), or a variant with 1, 2, 3, 4, 5 or more substitutions.

42. The cell of any one of claims 1 to 41, wherein the transmembrane domain is a DNAM1, a CD28, a IL-2Rbeta, a TLR1, a TLR2, a TLR3, a TLR4, a TLR5, a TLR6, a TLR7, a TLR8, a TLR9, a TLR10, a IL-1R, or a IL-18R transmembrane domain, or a functional variant thereof.

43. The cell of any one of claims 1 to 42, wherein the CAR further comprises a hinge domain, optionally wherein the hinge domain is CD8a hinge, CD28 hinge, or IgG4 hinge.

44. The cell of any one of claims 1 to 43, wherein the cell comprises homozygous inactivating mutations in the cytokine-inducible SH2-containing protein (CISH) genes of the cell.

45. The cell of any one of claims 1 to 44, wherein the cell is an induced pluripotent stem cell-derived natural killer (iPSC-NK) cell.

46. A pharmaceutical composition comprising the cell of any one of claims 1 to 45 and a pharmaceutically acceptable solution.

47. A method of killing a target cell comprising contacting a population of target cells with a population of natural killer cells according to any one of claims 1 to 45,
wherein the chimeric antigen receptors of the natural killer cell comprise a ligand-binding domain that specifically binds on antigen on the target cell, and
wherein the natural killer cells induce specific killing of the target cells.

48. A method of treating cancer in a subject in need thereof, comprising administering the pharmaceutical compositions of claim 46 to the subject.

49. Use of a natural killer cell or pharmaceutical composition of any preceding claim for treatment of cancer.

50. A chimeric antigen receptor comprised in any of the preceding natural killer cells.

51. A polynucleotide comprising a polynucleotide sequence encoding a chimeric antigen receptor comprised in any of the preceding natural killer cells.

52. A vector comprising the polynucleotide of claim 51.

53. A stem or progenitor cell comprising the polynucleotide of claim 51.

54. A method of making a natural killer cell, comprising providing the stem or progenitor cell comprising the polynucleotide of claim 51 and differentiating the stem or progenitor cell into a natural killer cell.

55. A natural killer cell produced by differentiating the stem or progenitor cell comprising the polynucleotide of claim 51 into a natural killer cell.

56. The cell of any one of claims 29 to 32, wherein the ligand-binding domain specifically binds a CD19 antigen, a CD20 antigen, a Her2 antigen or a BCMA antigen.

57. A chimeric antigen receptor (CAR) comprising an extracellular portion, a transmembrane domain, and a cytoplasmic portion, wherein the cytoplasmic portion comprises a Toll/interleukin-1 (IL-1) receptor (TIR) signaling domain.

58. The CAR of claim 57, wherein the TIR signaling domain is a Toll-like receptor (TLR) signaling domain.

59. The CAR of claim 58, wherein the TLR signaling domain is a TLR2 signaling domain or a functional variant thereof.

60. The CAR of claim 58 or 59, wherein the TLR signaling domain has a polypeptide sequence at least 55%, at least 70%, at least 75%, at least 80%, at least 90%, at least 95%, or 100% identical to SEQ ID NO: 2.

61. The CAR of any one of claims 57-60, wherein the cytoplasmic portion comprises a CD3ζ signaling domain.

62. The CAR of any one of claims 57-61 wherein the cytoplasmic portion comprises a 2B4 signaling domain.

63. The CAR of any one of claims 57-61, wherein the cytoplasmic portion lacks a 2B4 signaling domain.

64. The CAR of any one of claims 57-61, wherein the cytoplasmic portion consists essentially of, in N- to C-terminal order, a 2B4 signaling domain, an IL-18R signaling domain, and a CD3ζ signaling domain.

65. The CAR of any one of claims 57-61, wherein the cytoplasmic portion consists essentially of, in N- to C-terminal order, a 2B4 signaling domain, a CD3ζ signaling domain, and a TLR2 signaling domain.

66. The CAR of any one of claims 57-61, wherein the cytoplasmic portion consists essentially of, in N- to C-terminal order, a 2B4 signaling domain, a TLR2 signaling domain, and a CD3ζ signaling domain.

67. The CAR of any one of claims 57-66, wherein the extracellular protein comprises a ligand-binding domain.

68. The CAR of claim 67, wherein the ligand-binding domain comprises an antibody-like domain.

69. The CAR of claim 67, wherein the ligand-binding domain comprises a variable heavy (VH) domain and/or a variable light (VL) domain.

70. The CAR of claim 67, wherein the ligand-binding domain comprises a single-chain variable fragment (scFv).

71. The CAR of any one of claims 57-70, wherein the ligand-binding domain specifically binds a CD19 antigen, a CD20 antigen, a Her2 antigen, or a BCMA antigen.

72. The CAR of claim 57, wherein
(i) the extracellular domain comprises a polypeptide derived from a CD19 ligand binding domain, a CD20 ligand binding domain, a BCMA binding domain, or a HER2 binding domain;
(ii) the transmembrane domain is a transmembrane domain selected from the group consisting of aTM, CD8 alpha chain (CD8α), CD28, CD16, NKp44, NKp46, NKG2, DNAM1, IL-2Rβ, TLR, and IL-1R subfamily; and
(iii) the cytoplasmic domain comprises a signaling domain of TLR2, 41BB, or 2B4.

73. The CAR of claim 72, wherein the cytoplasmic domain further comprises a signaling domain of CD3ζ.

74. The CAR of claim 57, comprising:
(i) aTM transmembrane domain, a TLR2 signaling domain, and a CD3zeta signaling domain; or
(ii) a CD28 transmembrane domain, a TLR2 signaling domain, and a CD3zeta signaling domain.

75. The CAR of any one of claims 57-74, wherein the C-terminus of the extracellular domain is operably linked to the N-terminus or C-terminus of the transmembrane domain without a linker.

76. The CAR of any one of claims 57-74, wherein the C-terminus of the extracellular domain is operably linked to the N-terminus or C-terminus of the transmembrane domain with a linker or a hinge domain.

77. The CAR of claim 76, wherein the hinge domain is a CD8a hinge.

78. The CAR of claim 77, wherein the CD8a hinge comprises the amino acid sequence set forth in SEQ ID NO: 158.

79. The CAR of any one of claims 57-78, wherein the N-terminus or C-terminus of the transmembrane domain is operably linked to the N-terminus of the cytoplasmic domain, with or without a linker.

80. The CAR of claim 79, wherein the transmembrane domain is operably linked to the cytoplasmic domain with a Gly-Ser linker.

81. A polynucleotide comprising a nucleotide sequence encoding the chimeric antigen receptor of any one of claims 57-80.

82. A population of cells comprising an immune cell comprising the chimeric receptor of any one of claims 57-80 or the polynucleotide of claim 81.

83. The population of cells of claim 82, wherein the immune cell is a natural killer (NK) cell.

84. The population of cells of claim 83, wherein the NK cell is an induced pluripotent stem cell-derived natural killer (iPSC-NK) cell.

85. The population of cells of any one of claims 82-84, wherein the immune cell comprises homozygous inactivating mutations in a cytokine-inducible SH2-containing protein (CISH) gene.

86. The population of cells of any one of claims 82-84, wherein the immune cell is CISH^{-/-}.

87. A pharmaceutical composition comprising the population of cells of any one of claims 82-86.

88. A method of killing a target cell comprising contacting a population of target cells with a population of cells according to any one of claims 82-86 or the pharmaceutical composition of claim 87, wherein the ligand-binding domain of the CAR specifically binds on antigen on the target cell, and wherein the cells induce specific killing of the target cells.

89. A method of treating cancer in a subject in need thereof, comprising administering the pharmaceutical compositions of claim 86 to the subject.
